(19) United States
(12) Patent Application Publication
(10) Pub. No.: US 2024/0082425 A1
(43) Pub. Date: Mar. 14, 2024

(54) LIPID STRUCTURES AND COMPOSITIONS COMPRISING SAME

(71) Applicant: TURN BIOTECHNOLOGIES, INC., Mountain View, CA (US)

(72) Inventors: Mouhannad Jumaa, San Jose, CA (US); Zeeshan Kamal, San Ramon, CA (US); Dipti Barman, Mountain View, CA (US)

(21) Appl. No.: 18/340,773

(22) Filed: Jun. 23, 2023

Related U.S. Application Data
(60) Provisional application No. 63/464,022, filed on May 4, 2023.
(60) Provisional application No. 63/386,482, filed on Dec. 7, 2022.
(60) Provisional application No. 63/355,024, filed on Jun. 23, 2022.

(51) Int. Cl. A61K 48/00; C07C 219/12; C07C 219/18; C07C 219/06; C07C 219/16; A61K 9/127

(52) U.S. Cl. CPC A61K 48/0033; C07C 219/12; C07C 219/18; C07C 219/06; C07C 219/16; A61K 9/127

(57) ABSTRACT

The disclosure relates to ionizable lipids and compositions comprising the ionizable lipids. Lipid-nanoparticle compositions comprised of an ionizable lipid, optionally in combination with other lipid components such as helper lipids, stabilization lipids and structural lipids, and a therapeutic agent, such as a nucleic acid, for delivery to mammalian cells or organs are described.

### CROSS-REFERENCE TO RELATED APPLICATIONS

This application claims the benefit of U.S. Provisional Application No. 63/355,024, filed Jun. 23, 2022, U.S. Provisional Application No. 63/386,482, filed Dec. 7, 2022 and U.S. Provisional Application No. 63/464,022, filed May 4, 2023, each of which is incorporated by reference herein its entirety.

### TECHNICAL FIELD

The disclosure relates to ionizable lipids and helper lipids that can be used in combination with other lipid components, such as stabilization lipids and structural lipids. The disclosure also provides lipid-nanoparticle compositions comprising such lipids towards delivery of therapeutic molecules, particularly therapeutic nucleic acids.

### BACKGROUND

Nucleic acids-based therapies have attracted attention in the recent years as there is an enormous potential to treat diseases by targeting their genetic blueprints in vivo. Nucleic acids-based therapeutics can achieve long-lasting or even curative effects via gene inhibition, addition, replacement or editing. However, the clinical translation of both nucleic acid medicines and other therapeutic molecules depends on delivery technologies that improve stability, facilitate internalization and/or increase target affinity.

A lipid-based delivery system such as, but not limited to lipid nanoparticles (LNP) may provide an approach to stabilize and deliver nucleic acids and other therapeutic molecules, and there remains a significant need towards evolution of this technology. Design features, such as optimal particle size, encapsulation efficiencies, robust manufacturing process, different lipophilicity and appropriate surface charge, can be further advanced to provide efficient lipid-based delivery systems for nucleic acids and other therapeutic molecules.

### BRIEF DESCRIPTION OF FIGURES

FIG. 1 is a schematic illustration of the experimental design for rejuvenation of human adult dermal fibroblasts.

FIG. 2 shows data from immunocytochemistry experiments demonstrating downregulation of SOD2 and MMP1, markers of skin aging and impaired skin quality, after transient reprogramming treatment of human adult dermal fibroblasts using lipid nanoparticles containing lipid formula (II) and an mRNA cocktail where each mRNA molecule encoded OCT4 (O), SOX2 (S), KLF4 (K), Lin28 (L), cMyc (M), or Nanog (N).

FIG. 3 shows data from immunocytochemistry experiments demonstrating downregulation of SOD2, MMP1, MMP2, and p16INK4A (markers of skin aging, impaired skin quality, and senescence), after transient reprogramming treatment of human adult dermal fibroblasts using lipid nanoparticles containing lipid formula (II) and an mRNA cocktail where each mRNA molecule encoded O, S, K, L, M, or N.

FIG. 4 shows data demonstrating increased proliferation of human adult dermal fibroblasts after transient reprogramming treatment using lipid nanoparticles containing lipid formula (II) and an mRNA cocktail where each mRNA molecule encoded O, S, K, L, M, or N.

FIG. 5 is a schematic illustration of an experimental design for rejuvenation of human aged skin explants, using the compositions and methods herein for epigenetic reprogramming of age (ERA).

FIG. 6 shows data from immunohistochemistry experiments demonstrating downregulation of SOD2 (a marker of skin aging and oxidative stress) and upregulation of Collagen VII (a marker of skin quality) compared to the untreated condition after transient reprogramming treatment of human adult dermal fibroblasts using lipid nanoparticles containing lipid formula (II) and an mRNA cocktail where each mRNA molecule encoded O, S, K, L, M, or N.

FIGS. 7A-7B show transfection efficiency data and transfection potency for lipid nanoparticle formulations comprising lipids of formulas (I) or (II).

FIGS. 8A-8D are bar graphs showing transfection efficiency (FIGS. 8A-8B) and cell viability (FIGS. 8C-8D) 24 hours after transfection of Pan T-cells (FIGS. 8A and 8C) or Jurkat cells (FIGS. 8B and 8D) with enhanced green fluorescent protein (eGFP) mRNA in lipid nanoparticle compositions, via electroporation (EP, at 1350V and 1600V) or via Lipofectamine™.

FIGS. 9A-9B are bar graphs showing results 24 hours after transfection of Pan T-cells with enhanced green fluorescent protein (eGFP) mRNA in lipid nanoparticle compositions, via electroporation (EP) or via Lipofectamine™, where transfection efficiency is shown in FIG. 9A as median fluorescence intensity of eGFP and percent cell viability is shown in FIG. 9B.

FIGS. 10A-10C are bar graphs showing results 18-24 hours after transfection of Pan T-cells with enhanced green fluorescent protein (eGFP) mRNA in lipid nanoparticle compositions comprising an ionizable lipid as disclosed herein, where cell viability is shown in FIG. 10A, transfection potency is shown in FIG. 10B, and transfection efficiency is shown in FIG. 10C.

### BRIEF SUMMARY

The following aspects and embodiments thereof described below are meant to be exemplary and illustrative, not limiting in scope.

In one aspect, the disclosure relates to an ionizable lipid of Formula (I)

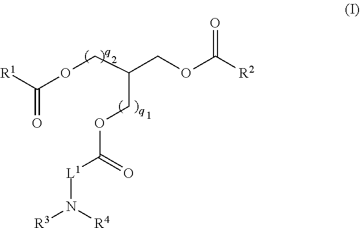

or a pharmaceutically acceptable salt or a stereoisomer thereof, wherein:

L¹ is C₁-C₆ alkylene; R¹ is C₆-C₂₀ alkenyl; R² is C₆-C₂₀ alkyl; R³ and R⁴ are each independently H or C₁-C₃ alkyl; q₁ is absent or 1; and q₂ is absent or 1.

In other aspect, the ionizable lipid of Formula (I) have one of the following structures:

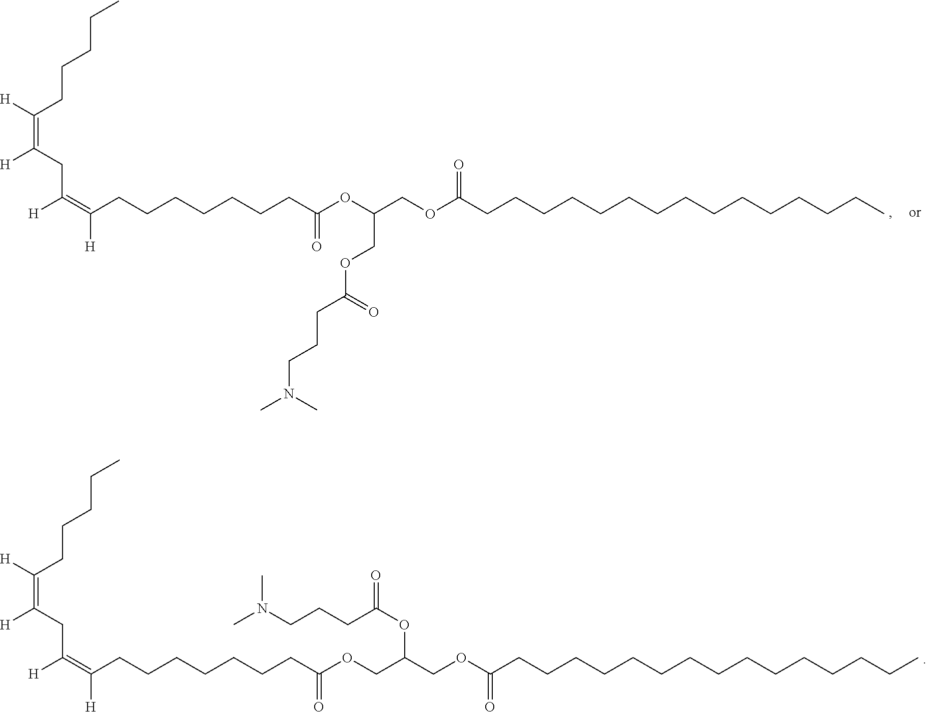 [SMILES: CCCCC/C=C\C/C=C\CCCCCCCC(=O)OC(COC(=O)CCCCCCCCCCCCCCC)COC(=O)CCCN(C)C ; CCCCC/C=C\C/C=C\CCCCCCCC(=O)OCC(COC(=O)CCCCCCCCCCCCCCC)OC(=O)CCCN(C)C]

In one aspect, the disclosure relates to an ionizable lipid of Formula (I-A)

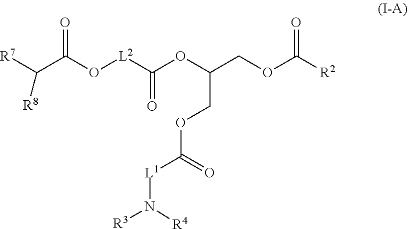

or a pharmaceutically acceptable salt or a stereoisomer thereof, wherein:

L¹ is C₁-C₆ alkylene; L² is C₁-C₈ alkylene; R² is C₆-C₂₀ alkyl; R³ and R⁴ are each independently H or C₁-C₃ alkyl; R⁷ is C₄-C₂₀ alkyl; and R⁸ is C₄-C₂₀ alkyl.

In other aspect, the ionizable lipid of Formula (I-A) have the following structure:

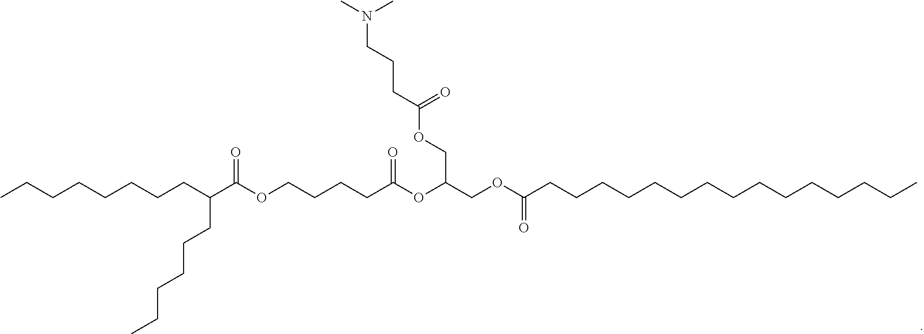 [SMILES: CCCCCCCCCCCCCCCC(=O)OCC(COC(=O)CCCN(C)C)OC(=O)CCCCOC(=O)C(CCCCCC)CCCCCCCC]

In one aspect, the disclosure relates to an ionizable lipid of Formula (I-B)

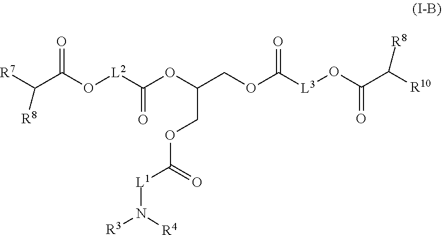

or a pharmaceutically acceptable salt or a stereoisomer thereof, wherein:

L¹ is C₁-C₆ alkylene; L² is C₁-C₈ alkylene; L³ is C₁-C₈ alkylene; R³ and R⁴ are each independently H or C₁-C₃ alkyl; R⁶ is C₄-C₂₀ alkyl; R⁷ is C₄-C₂₀ alkyl; R⁸ is C₄-C₂₀ alkyl; and R¹⁰ is C₄-C₂₀ alkyl.

In other aspect, the ionizable lipid of Formula (I-B) have the following structure:

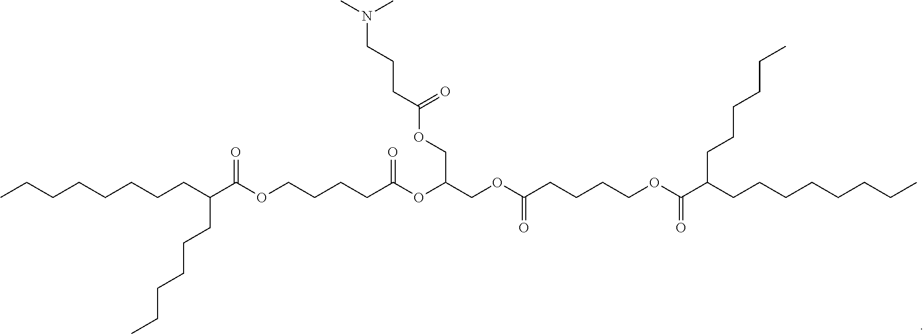 [SMILES: CCCCCCCCC(CCCCCC)C(=O)OCCCCC(=O)OCC(COC(=O)CCCN(C)C)OC(=O)CCCCOC(=O)C(CCCCCC)CCCCCCCC]

In one aspect, the disclosure relates to an ionizable lipid of Formula (I-B)

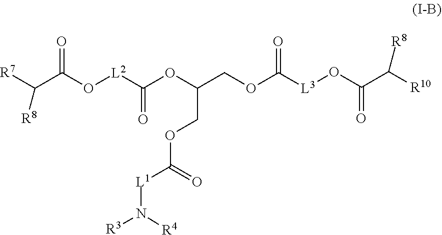

or a pharmaceutically acceptable salt or a stereoisomer thereof, wherein: L¹ is C₁-C₆ alkylene; L² is C₁-C₈ alkylene; L³ is C₁-C₈ alkylene; R³ and R⁴ are each independently H, C₁-C₄ alkyl, —CH₂-cyclopropyl, —(CH₂)ₙOH, or R³ and R⁴ together form a N-heterocycle; R⁶ is C₄-C₂₀ alkyl; R⁷ is C₄-C₂₀ alkyl; R⁸ is C₄-C₂₀ alkyl; R¹⁰ is C₄-C₂₀ alkyl; and n is 2, 3, or 4.

In other aspect, the ionizable lipid of Formula (I-B) has one of the following structures:

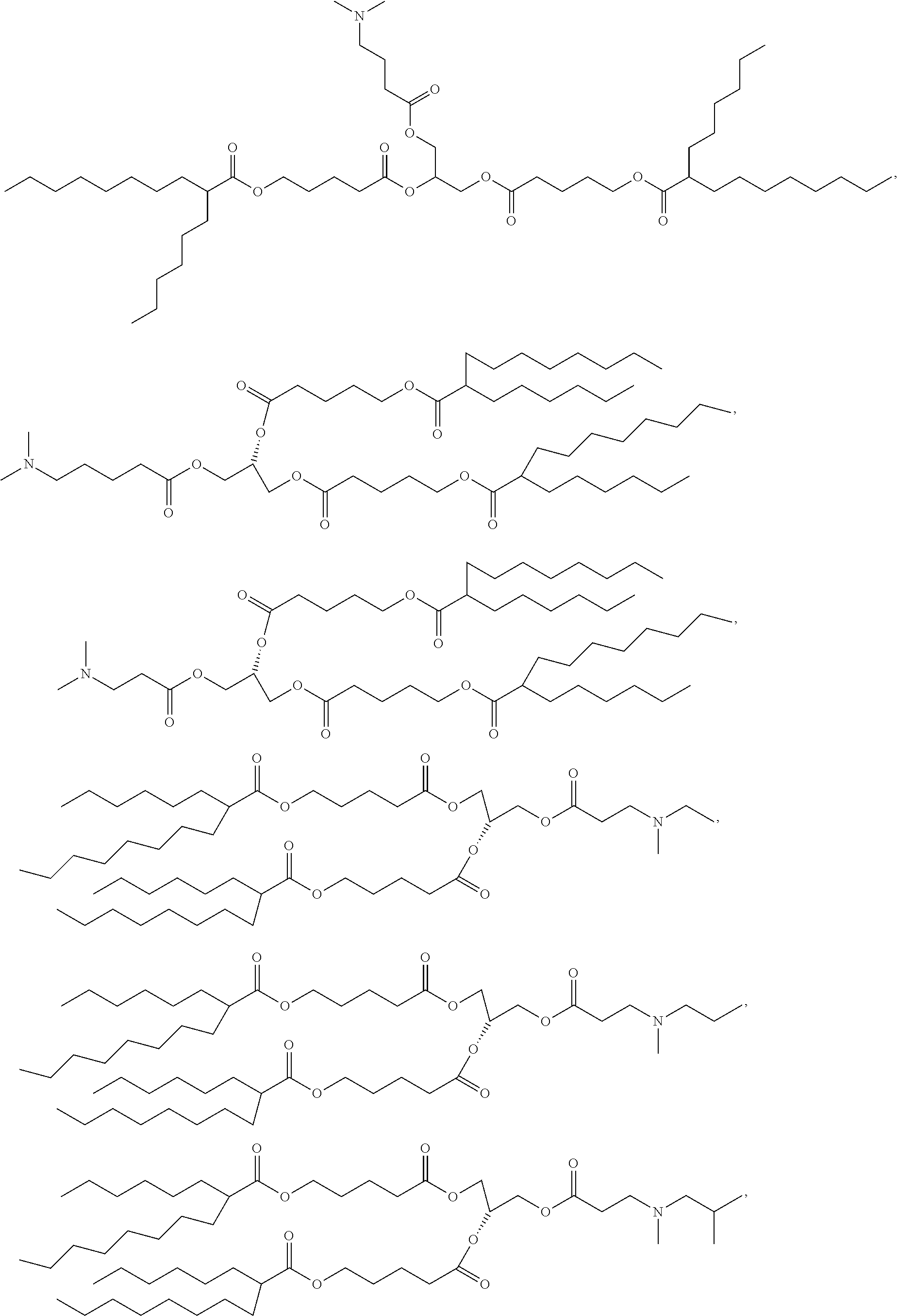

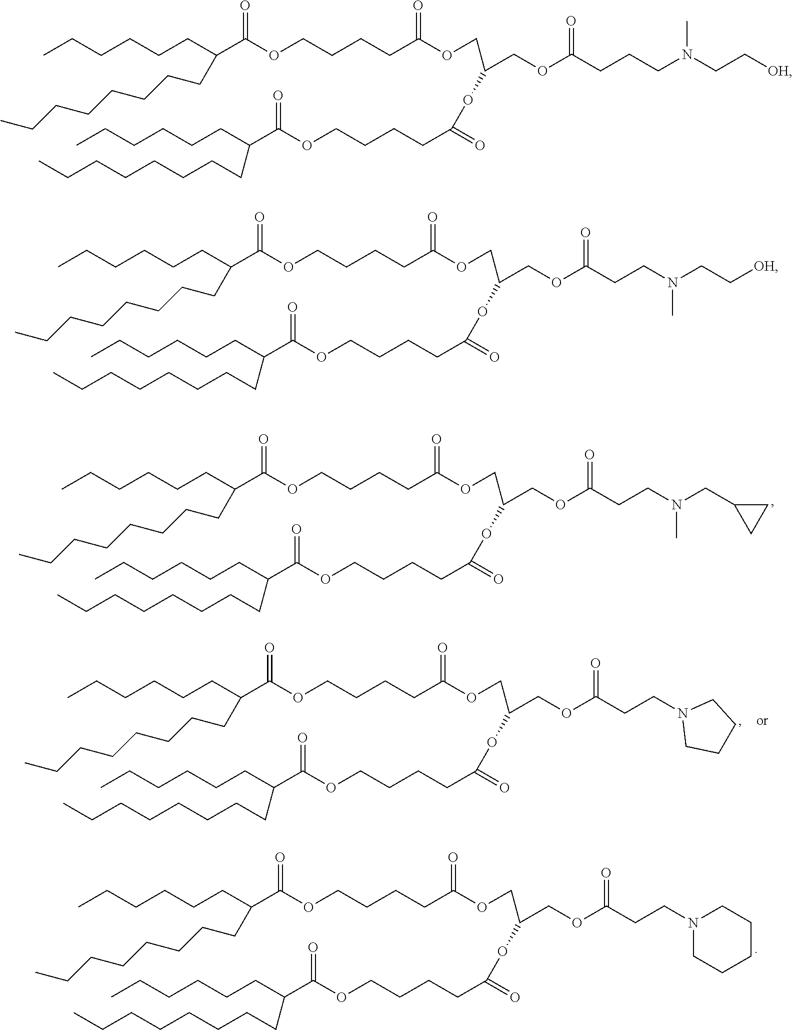

In one aspect, the disclosure relates to an ionizable lipid of Formula (II)

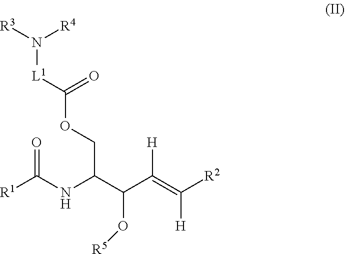

or a pharmaceutically acceptable salt or a stereoisomer thereof, wherein:

L¹ is C₁-C₆ alkylene; R¹ is C₆-C₂₀ alkenyl; R² is C₆-C₂₀ alkyl; R³ and R⁴ are each independently H or C₁-C₃ alkyl; and R⁵ is H or C₁-C₃ alkyl.

In other aspect, the ionizable lipid of Formula (II) have one of the following structures:

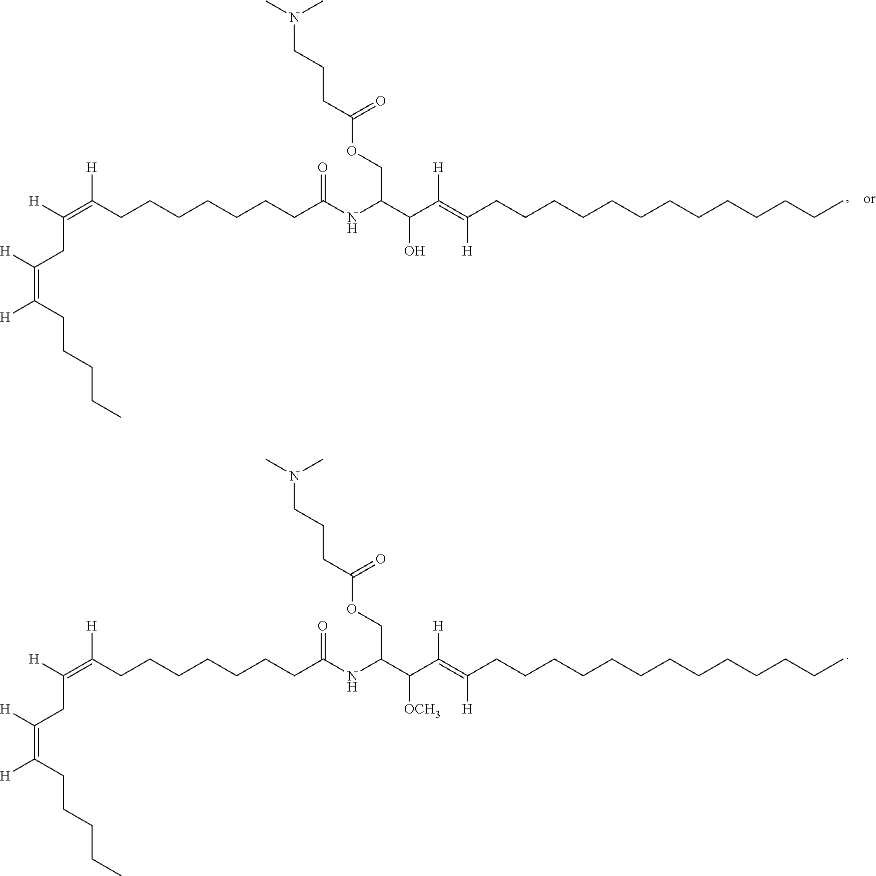 [SMILES: CCCCC/C=C\C/C=C\CCCCCCCC(=O)NC(COC(=O)CCCN(C)C)C(O)/C=C/CCCCCCCCCCCCC ; CCCCC/C=C\C/C=C\CCCCCCCC(=O)NC(COC(=O)CCCN(C)C)C(/C=C/CCCCCCCCCCCCC)OC]

In one aspect, the disclosure relates to an ionizable lipid of Formula (III)

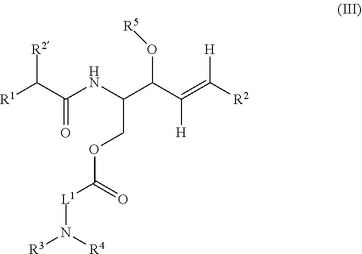

or a pharmaceutically acceptable salt or a stereoisomer thereof, wherein:

L¹ is C₁-C₆ alkylene; R¹ is C₆-C₂₀ alkenyl; R² is C₆-C₂₀ alkyl; R^{2′} is C₆-C₂₀ alkyl; R³ and R⁴ are each independently H or C₁-C₃ alkyl; and R⁵ is H or C₁-C₃ alkyl.

In other aspect, the ionizable lipid of Formula (III) have one of the following structures:

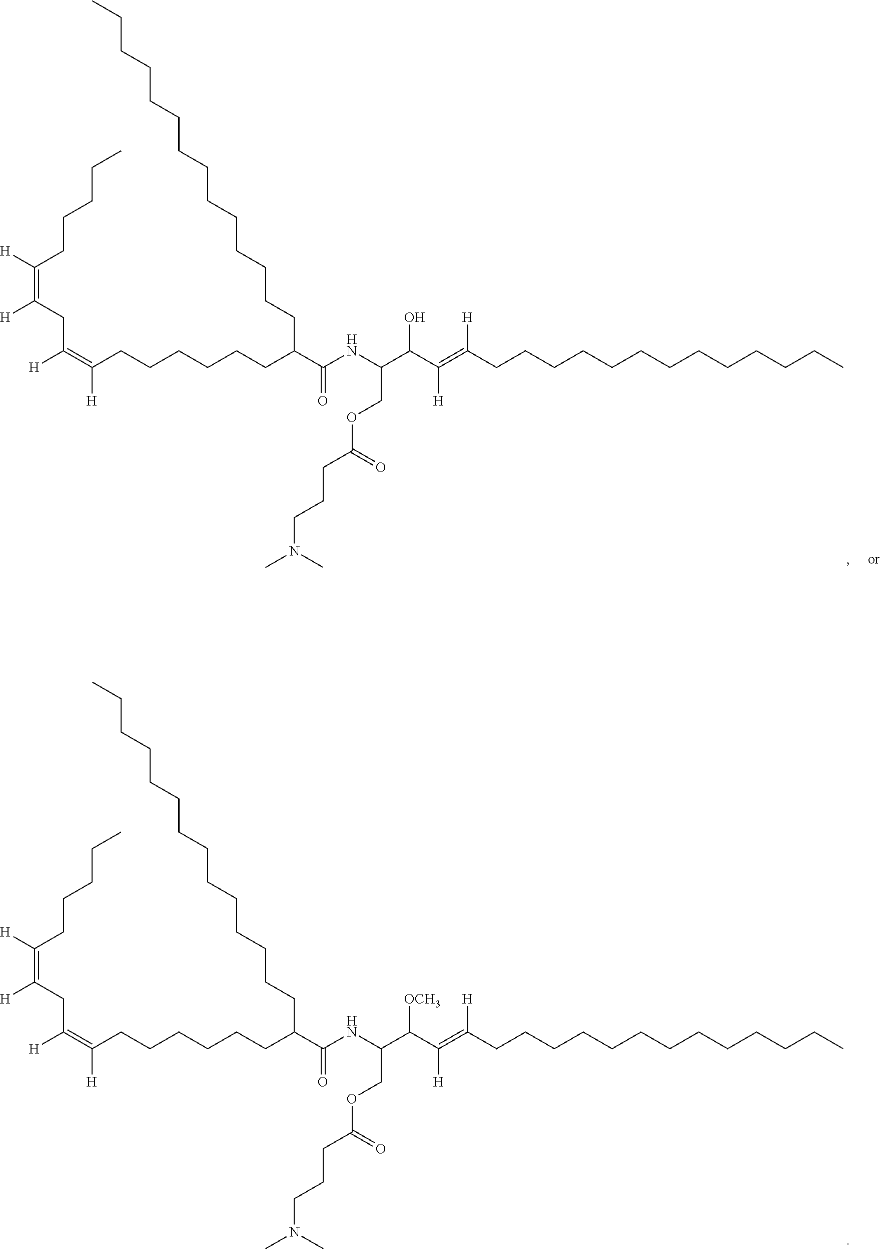 [SMILES: CCCCC/C=C\C/C=C\CCCCCCC(CCCCCCCCCCCCCC)C(=O)NC(COC(=O)CCCN(C)C)C(O)/C=C/CCCCCCCCCCCCC ; CCCCC/C=C\C/C=C\CCCCCCC(CCCCCCCCCCCCCC)C(=O)NC(COC(=O)CCCN(C)C)C(/C=C/CCCCCCCCCCCCC)OC]

In one aspect, the disclosure relates to an ionizable lipid of Formula (IV)

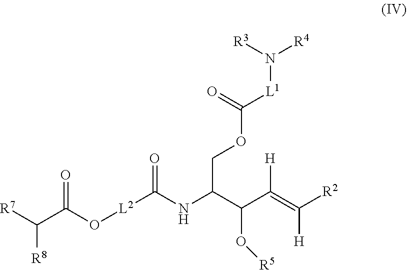

or a pharmaceutically acceptable salt or a stereoisomer thereof, wherein:

L¹ is C₁-C₆ alkylene; L² is C₁-C₈ alkylene; R² is C₆-C₂₀ alkyl; R³ and R⁴ are each independently H or C₁-C₃ alkyl; R⁵ is H or C₁-C₃ alkyl; R⁷ is C₄-C₂₀ alkyl; and R⁸ is C₄-C₂₀ alkyl.

In other aspect, the ionizable lipid of Formula (IV) have one of the following structures:

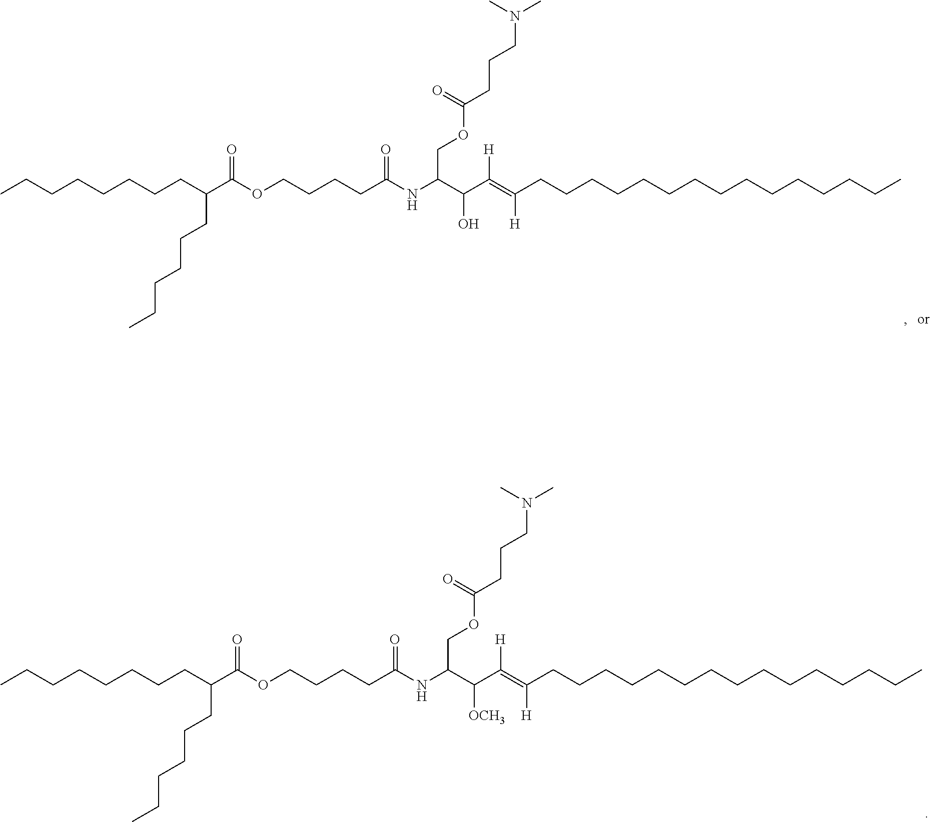

In one aspect, the disclosure relates to an ionizable lipid of Formula (V)

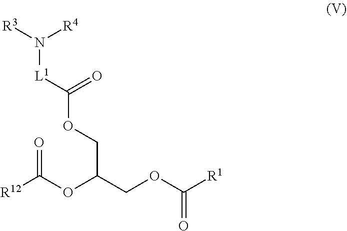

or a pharmaceutically acceptable salt or a stereoisomer thereof, wherein:

L¹ is C₁-C₆ alkylene; R¹ is C₆-C₂₀ alkenyl; R³ and R⁴ are each independently H or C₁-C₃ alkyl; and R¹² is C₆-C₂₀ alkenyl.

In other aspect, the ionizable lipid of Formula (V) have the following structure:

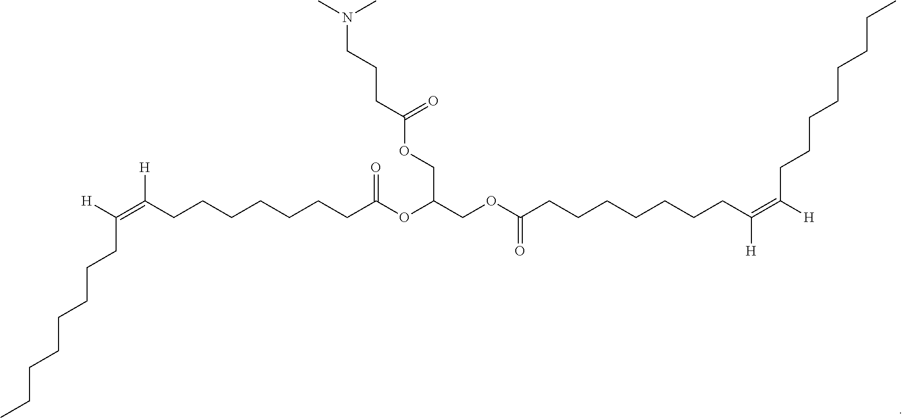 [SMILES: CCCCCCCC/C=C\CCCCCCCC(=O)OCC(COC(=O)CCCN(C)C)OC(=O)CCCCCCC/C=C\CCCCCCCC]

In one aspect, the disclosure relates to an ionizable lipid of Formula (VI)

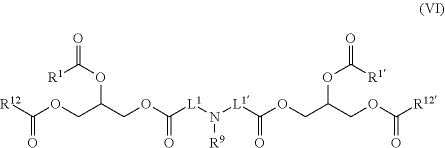

or a pharmaceutically acceptable salt or a stereoisomer thereof, wherein:

L¹ is C₁-C₆ alkylene; L^{1′} is C₁-C₆ alkylene; R¹ is C₆-C₂₀ alkenyl; R^{1′} is C₆-C₂₀ alkenyl; R⁹ is H, C₁-C₆ alkyl or —(CH₂)ₙOH; R¹² is C₆-C₂₀ alkenyl; R^{12′} is C₆-C₂₀ alkenyl; and n is 2, 3 or 4.

In other aspect, the ionizable lipid of Formula (VI) have one of the following structures:

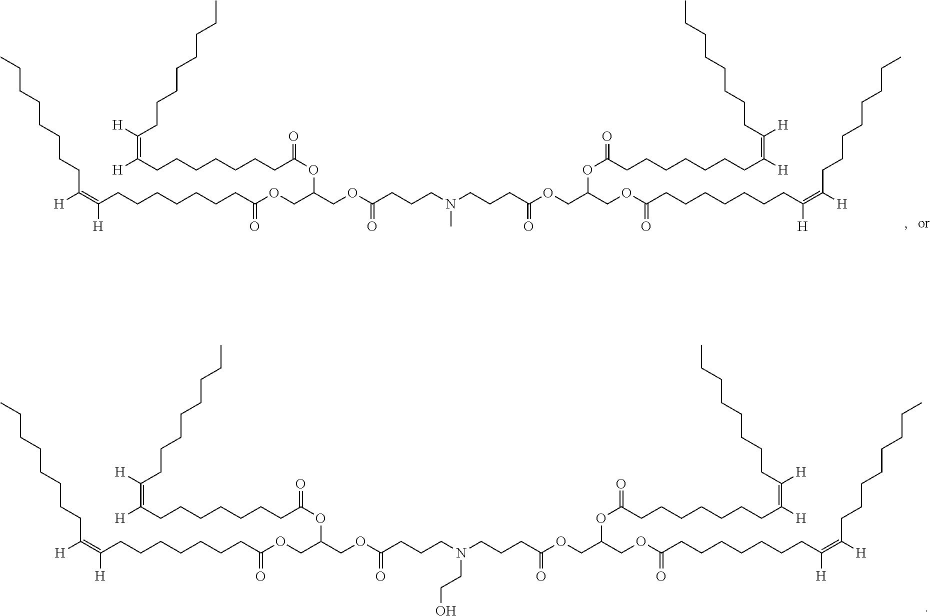

In one aspect, the disclosure relates to an ionizable lipid of Formula (VII)

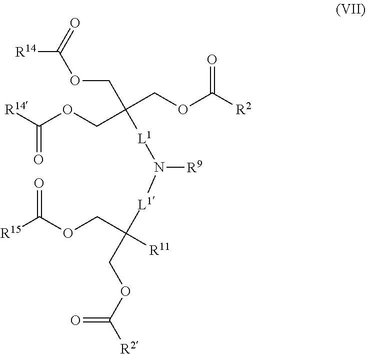

or a pharmaceutically acceptable salt or a stereoisomer thereof, wherein:

L¹ is C₁-C₆ alkylene; L^{1′} is C₁-C₆ alkylene; R² is C₆-C₂₀ alkyl; R^{2′} is C₆-C₂₀ alkyl; R⁹ is H, C₁-C₆ alkyl or —(CH₂)ₙOH; R¹¹ is H or —CH₂)OC(═O)R¹⁶; R¹⁴ is C₆-C₂₀ alkyl; R^{14′} is C₆-C₂₀ alkyl; R¹⁵ is C₆-C₂₀ alkyl; R¹⁶ is C₆-C₂₀ alkyl; and n is 2, 3 or 4.

In other aspect, the ionizable lipid of Formula (VII) have one of the following structures:

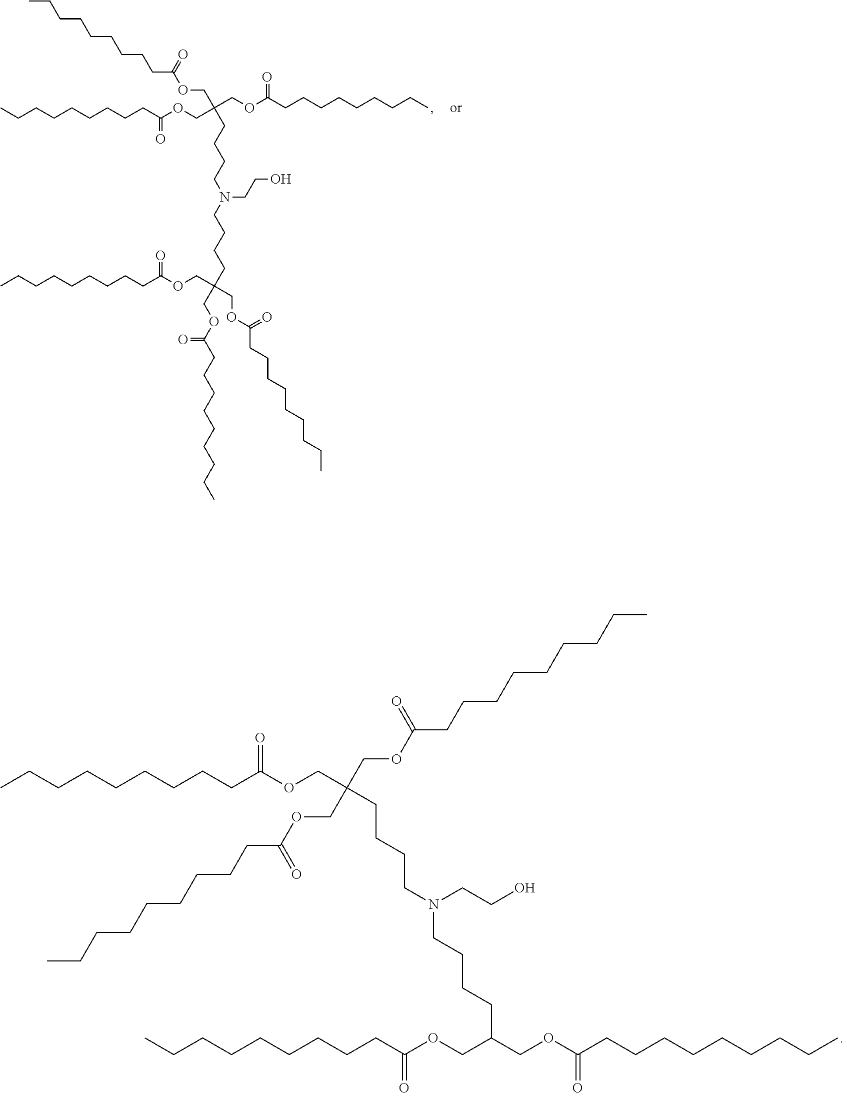 [SMILES: CCCCCCCCCC(=O)OCC(CCCCN(CCO)CCCCC(COC(=O)CCCCCCCCC)(COC(=O)CCCCCCCCC)COC(=O)CCCCCCCCC)(COC(=O)CCCCCCCCC)COC(=O)CCCCCCCCC ; CCCCCCCCCC(=O)OCC(CCCCN(CCO)CCCCC(COC(=O)CCCCCCCCC)(COC(=O)CCCCCCCCC)COC(=O)CCCCCCCCC)COC(=O)CCCCCCCCC]

In one aspect, the disclosure relates to an ionizable lipid of Formula (VIII)

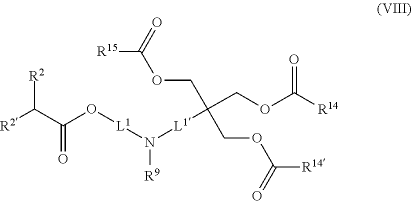

or a pharmaceutically acceptable salt or a stereoisomer thereof, wherein:

L¹ is C₁-C₆ alkylene; L^{1′} is C₁-C₆ alkylene; R² is C₆-C₂₀ alkyl; R^{2′} is C₆-C₂₀ alkyl; R⁹ is H, C₁-C₆ alkyl or —(CH₂)ₙOH; R¹⁴ is C₆-C₂₀ alkyl; R^{14′} is C₆-C₂₀ alkyl; R¹⁵ is C₆-C₂₀ alkyl; and n is 2, 3 or 4.

In other aspect, the ionizable lipid of Formula (VIII) have the following structure:

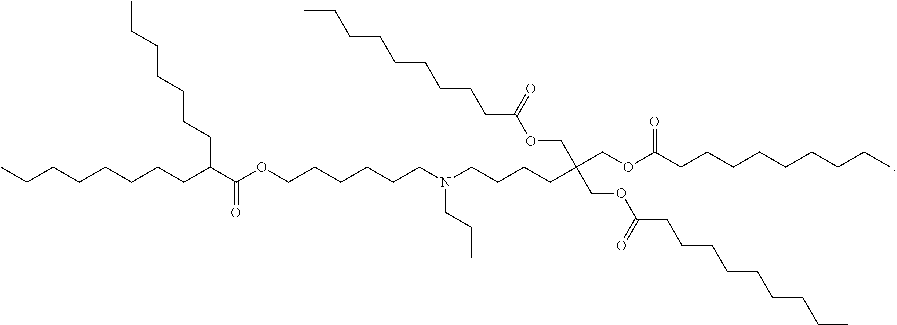 [SMILES: CCCCCCCCCC(=O)OCC(CCCCN(CCC)CCCCCCOC(=O)C(CCCCCCC)CCCCCCCC)(COC(=O)CCCCCCCCC)COC(=O)CCCCCCCCC]

In one aspect, the disclosure relates to an ionizable lipid of Formula (IX)

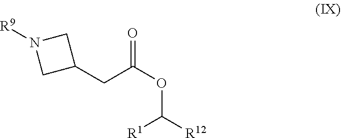

or a pharmaceutically acceptable salt or a stereoisomer thereof, wherein:

R¹ is C₆-C₂₀ alkenyl; R⁹ is H, C₁-C₆ alkyl or —(CH₂)ₙOH; R¹² is C₆-C₂₀ alkenyl; and n is 2, 3 or 4.

In other aspect, the ionizable lipid of Formula (IX) have one of the following structures:

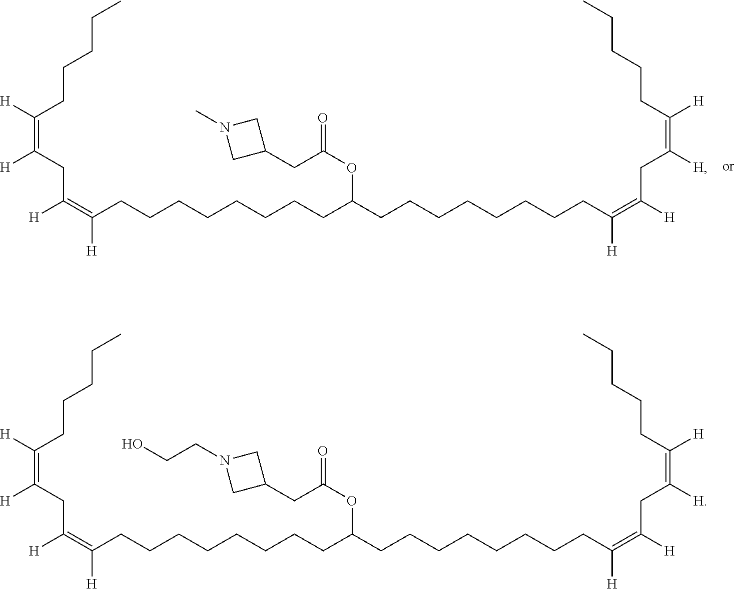

In one aspect, the disclosure relates to an ionizable lipid of Formula (X)

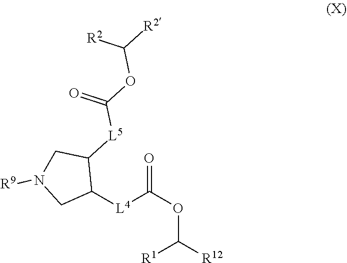

or a pharmaceutically acceptable salt or a stereoisomer thereof, wherein:

L⁴ is absent or C₁-C₆ alkylene; L⁵ is absent or C₁-C₆ alkylene; R¹ is C₆-C₂₀ alkenyl; R² is C₆-C₂₀ alkyl; R^{2′} is C₆-C₂₀ alkyl; R⁹ is H, C₁-C₆ alkyl or —(CH₂)ₙOH; R¹² is C₆-C₂₀ alkenyl; and n is 2, 3 or 4.

In other aspect, the ionizable lipid of Formula (X) have one of the following structures:

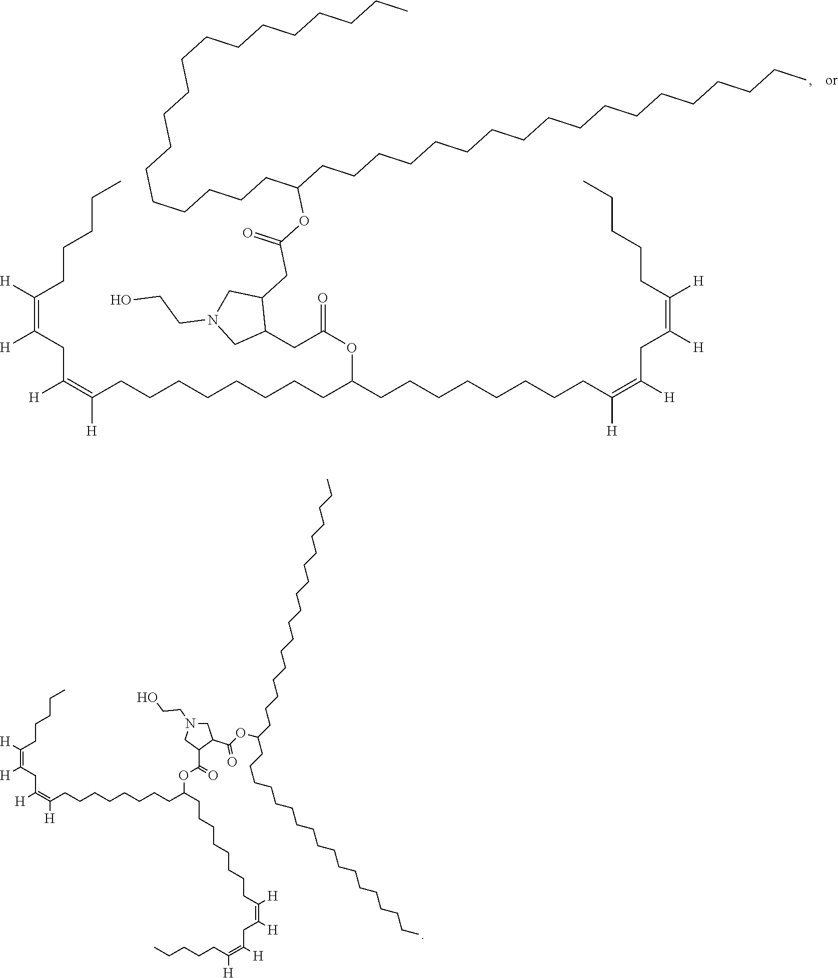 [SMILES: CCCCC/C=C\C/C=C\CCCCCCCCC(CCCCCCCC/C=C\C/C=C\CCCCC)OC(=O)CC1CN(CCO)CC1CC(=O)OC(CCCCCCCCCCCCCCCCCC)CCCCCCCCCCCCCCCCCC ; CCCCC/C=C\C/C=C\CCCCCCCCC(CCCCCCCC/C=C\C/C=C\CCCCC)OC(=O)C1CN(CCO)CC1C(=O)OC(CCCCCCCCCCCCCCCCCC)CCCCCCCCCCCCCCCCCC]

In one aspect, the disclosure relates to an ionizable lipid of Formula (XI)

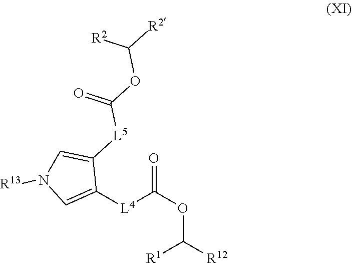

or a pharmaceutically acceptable salt or a stereoisomer thereof, wherein:

L⁴ is absent or C₁-C₆ alkylene; L⁵ is absent or C₁-C₆ alkylene; R¹ is C₆-C₂₀ alkenyl; R² is C₆-C₂₀ alkyl; R^{2′} is C₆-C₂₀ alkyl; R¹² is C₆-C₂₀ alkenyl; R¹³ is H, C₁-C₆ alkyl, —(CH₂)ₙOH or —(CH₂)_{q}N(CH₃)₂; n is 2, 3 or 4; and q is 2, 3 or 4.

In other aspect, the ionizable lipid of Formula (XI) have the following structure:

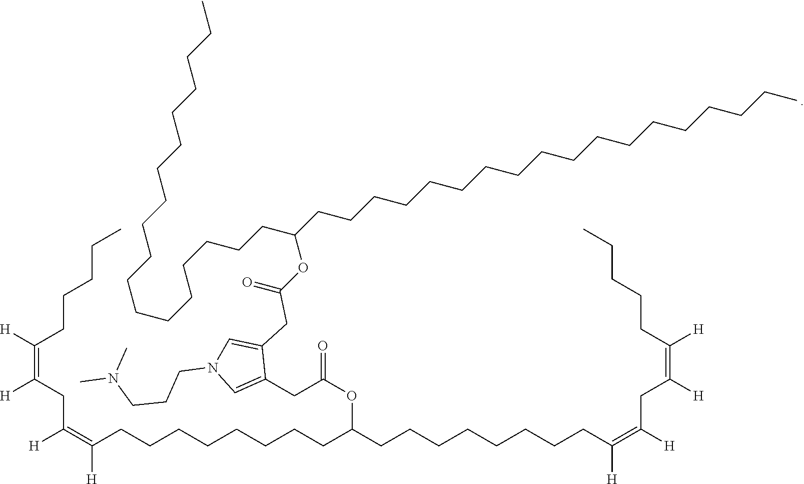 [SMILES: CCCCC/C=C\C/C=C\CCCCCCCCC(CCCCCCCC/C=C\C/C=C\CCCCC)OC(=O)Cc1cn(CCCN(C)C)cc1CC(=O)OC(CCCCCCCCCCCCCCCCCC)CCCCCCCCCCCCCCCCCC]

In one aspect, the disclosure relates to an ionizable lipid of Formula (XII)

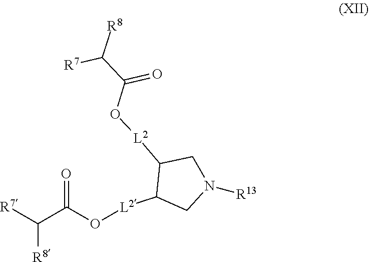

or a pharmaceutically acceptable salt or a stereoisomer thereof, wherein:

L² is C₁-C₈ alkylene; L^{2′} is C₁-C₈ alkylene; R⁷ is C₄-C₂₀ alkyl; R^{7′} is C₄-C₂₀ alkyl; R⁸ is C₄-C₂₀ alkyl; R^{8′} is C₄-C₂₀ alkyl; R¹³ is H, C₁-C₆ alkyl, —(CH₂)ₙOH, or —(CH₂)_{q}N(CH₃)₂; n is 2, 3 or 4; and q is 2, 3 or 4.

In another aspect, the ionizable lipid of Formula (XII) have the following structure:

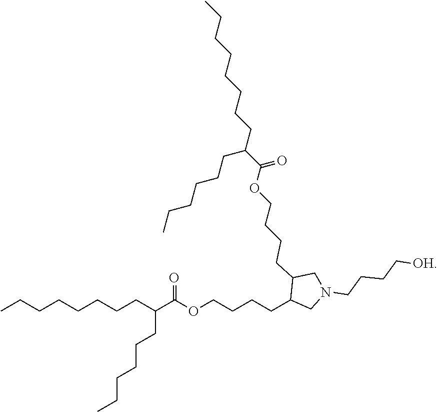 [SMILES: CCCCCCCCC(CCCCCC)C(=O)OCCCCC1CN(CCCCO)CC1CCCCOC(=O)C(CCCCCC)CCCCCCCC]

In one aspect, the disclosure relates to an ionizable lipid of Formula (XIII)

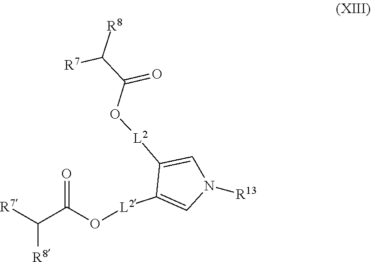

or a pharmaceutically acceptable salt or a stereoisomer thereof, wherein:

L² is C₁-C₈ alkylene; L^{2′} is C₁-C₈ alkylene; R⁷ is C₄-C₂₀ alkyl; R^{7′} is C₄-C₂₀ alkyl; R⁸ is C₄-C₂₀ alkyl; R_{8′} is C₄-C₂₀ alkyl; R¹³ is H, C₁-C₆ alkyl, —(CH₂)ₙOH, or —(CH₂)_{q}N(CH₃)₂; n is 2, 3 or 4; and q is 2, 3 or 4.

In some embodiments, L² is C₁-C₈ alkylene; L^{2′} is C₁-C₈ alkylene; R⁷ is C₄-C₂₀ alkyl; R^{7′} is C4-C20 alkyl; R8 is C4-C20 alkyl; R8′ is C4-C20 alkyl; and R13 is H in Formula (XIII).

In some embodiments, L² is C1-C8 alkylene; L^{2′} is C1-C8 alkylene; R7 is C4-C20 alkyl; R7′ is C4-C20 alkyl; R8 is C4-C20 alkyl; R8′ is C4-C20 alkyl; and R13 is methyl in Formula (XIII).

In some embodiments, L² is C1-C8 alkylene; L^{2′} is C1-C8 alkylene; R7 is C4-C20 alkyl; R7′ is C4-C20 alkyl; R8 is C4-C20 alkyl; R8′ is C4-C20 alkyl; R13 is —(CH2)nOH; and n is 2 in Formula (XIII).

In some embodiments, L² is C1-C8 alkylene; L^{2′} is C1-C8 alkylene; R7 is C4-C20 alkyl; R7′ is C4-C20 alkyl; R8 is C4-C20 alkyl; R8′ is C4-C20 alkyl; R13 is —(CH2)qN(CH3)2; and q is 3 in Formula (XIII).

Exemplary examples of ionizable lipid of Formula (XIII) can include, but not limited to the following:

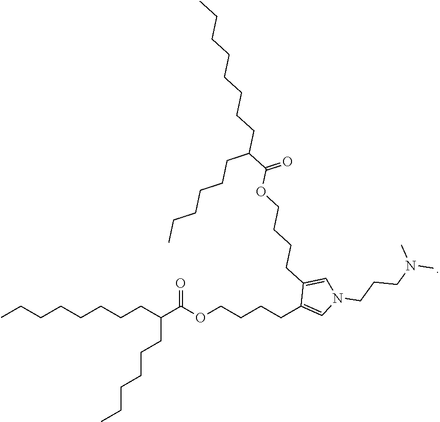 [SMILES: CCCCCCCCC(CCCCCC)C(=O)OCCCCc1cn(CCCN(C)C)cc1CCCCOC(=O)C(CCCCCC)CCCCCCCC]

In one embodiment, the ionizable lipid is of Formula (XIV)

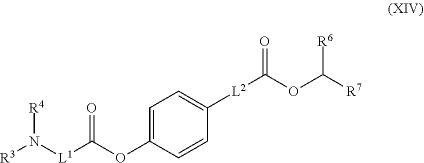

or a pharmaceutically acceptable salt or a stereoisomer thereof, wherein:

L1 is C1-C6 alkylene; L2 is C1-C8 alkylene; R3 and R4 are each independently H or C1-C3 alkyl; R6 is C4-C20 alkyl; and R7 is C4-C20 alkyl.

In some embodiments, L1 is C1-C6 alkylene; L2 is C1-C8 alkylene; R3 is methyl; R4 is methyl; R6 is C4-C20 alkyl; and R7 is C4-C20 alkyl in Formula (XIV).

Exemplary examples of ionizable lipid of Formula (XIV) can include, but not limited to the following

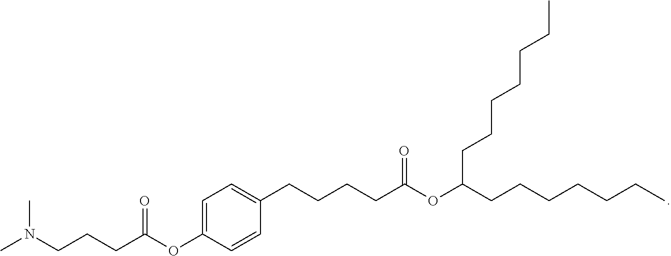 [SMILES: CCCCCCCC(CCCCCCC)OC(=O)CCCCc1ccc(OC(=O)CCCN(C)C)cc1]

In one embodiment, the ionizable lipid is of Formula (XV)

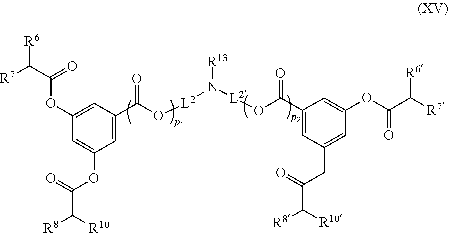

or a pharmaceutically acceptable salt or a stereoisomer thereof, wherein:

L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R8 is C4-C20 alkyl; R8′ is C4-C20 alkyl; R10 is C4-C20 alkyl; R10′ is C4-C20 alkyl; R13 is H, C1-C6 alkyl, —(CH2)nOH, or —(CH2)qN(CH3)2; n is 2, 3 or 4; p1 is absent or 1; p2 is absent or 1; and q is 2, 3, or 4.

In some embodiments, L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R8 is C4-C20 alkyl; R8′ is C4-C20 alkyl; R10 is C4-C20 alkyl; R10′ is C4-C20 alkyl; R13 is H; p1 is absent; and p2 is absent in Formula (XV).

In some embodiments, L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R8 is C4-C20 alkyl; R8′ is C4-C20 alkyl; R10 is C4-C20 alkyl; R10′ is C4-C20 alkyl; R13 is H; p1 is 1; and p2 is 1 in Formula (XV).

In some embodiments, L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R8 is C4-C20 alkyl; R8′ is C4-C20 alkyl; R10 is C4-C20 alkyl; R10′ is C4-C20 alkyl; R13 is methyl; p1 is absent; and p2 is absent in Formula (XV).

In some embodiments, L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R8 is C4-C20 alkyl; R8′ is C4-C20 alkyl; R10 is C4-C20 alkyl; R10′ is C4-C20 alkyl; R13 is methyl; p1 is 1; and p2 is 1 in Formula (XV).

In some embodiments, L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R8 is C4-C20 alkyl; R8′ is C4-C20 alkyl; R10 is C4-C20 alkyl; R10′ is C4-C20 alkyl; R13 is —(CH2)nOH; n is 4; p1 is absent; and p2 is absent in Formula (XV).

In some embodiments, L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R8 is C4-C20 alkyl; R8′ is C4-C20 alkyl; R10 is C4-C20 alkyl; R10′ is C4-C20 alkyl; R13 is —(CH2)nOH; n is 4; p1 is 1; and p2 is 1 in Formula (XV).

Exemplary examples of ionizable lipid of Formula (XV) can include, but not limited to the following:

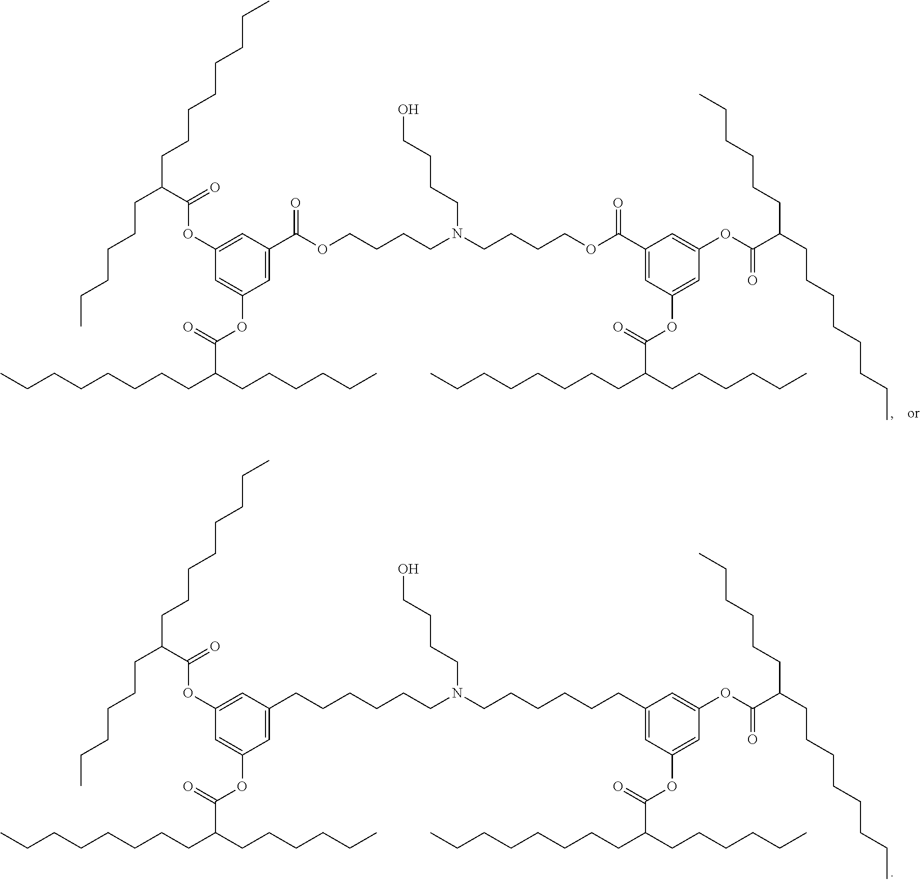

In one embodiment, the ionizable lipid is of Formula (XVI)

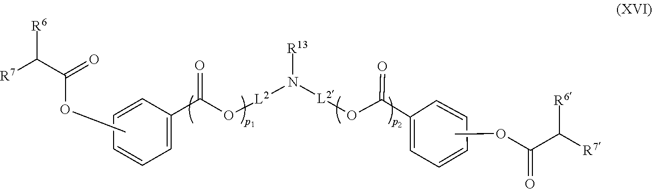

or a pharmaceutically acceptable salt or a stereoisomer thereof, wherein:

L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R13 is H, C1-C6 alkyl, —(CH2)nOH, or —(CH2)qN(CH3)2; n is 2, 3 or 4; p1 is absent or 1; p2 is absent or 1; and q is 2, 3 or 4.

In one embodiment, the ionizable lipid is of Formula (XVI-A):

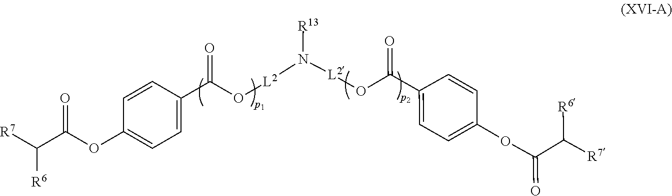

or a pharmaceutically acceptable salt or a stereoisomer thereof, wherein:

L² is C₁-C₈ alkylene; L^{2′} is C₁-C₈ alkylene; R6 is C₄-C₂₀ alkyl; R6′ is C₄-C₂₀ alkyl; R7 is C₄-C₂₀ alkyl; R^{7′} is C₄-C₂₀ alkyl; R¹³ is H, C₁-C₆ alkyl, —(CH₂)ₙOH, or —(CH₂)_{q}N(CH₃)₂; n is 2, 3 or 4; p₁ is absent or 1; p₂ is absent or 1; and q is 2, 3 or 4.

In some embodiments, L² is C₁-C₈ alkylene; L^{2′} is C₁-C₈ alkylene; R⁶ is C₄-C₂₀ alkyl; R^{6′} is C₄-C₂₀ alkyl; R⁷ is C₄-C₂₀ alkyl; R^{7′} is C₄-C₂₀ alkyl; R¹³ is H; p₁ is absent or 1; and p₂ is absent or 1 in Formula (XVI) and Formula (XVI-A).

In some embodiments, L² is C₁-C₈ alkylene; L^{2′} is C₁-C₈ alkylene; R⁶ is C₄-C₂₀ alkyl; R^{6′} is C₄-C₂₀ alkyl; R⁷ is C₄-C₂₀ alkyl; R^{7′} is C₄-C₂₀ alkyl; R¹³ is methyl; p₁ is absent or 1; and p₂ is absent or 1 in Formula (XVI) and Formula (XVI-A). In other embodiments, L² is C₁-C₈ alkylene; L^{2′} is C₁-C₈ alkylene; R⁶ is C₄-C₂₀ alkyl; R^{6′} is C₄-C₂₀ alkyl; R⁷ is C₄-C₂₀ alkyl; R^{7′} is C₄-C₂₀ alkyl; R¹³ is —(CH₂)ₙOH; n is 4; p₁ is absent; and p₂ is absent in Formula (XVI) and Formula (XVI-A).

In some embodiments, L² is C₁-C₈ alkylene; L^{2′} is C₁-C₈ alkylene; R⁶ is C₄-C₂₀ alkyl; R^{6′} is C₄-C₂₀ alkyl; R⁷ is C₄-C₂₀ alkyl; R^{7′} is C₄-C₂₀ alkyl; R¹³ is —(CH₂)ₙOH; n is 4; p₁ is 1; and p₂ is 1 in Formula (XVI) and Formula (XVI-A).

Exemplary examples of ionizable lipid of Formula (XVI) and Formula (XVI-A) can include, but not limited to the following:

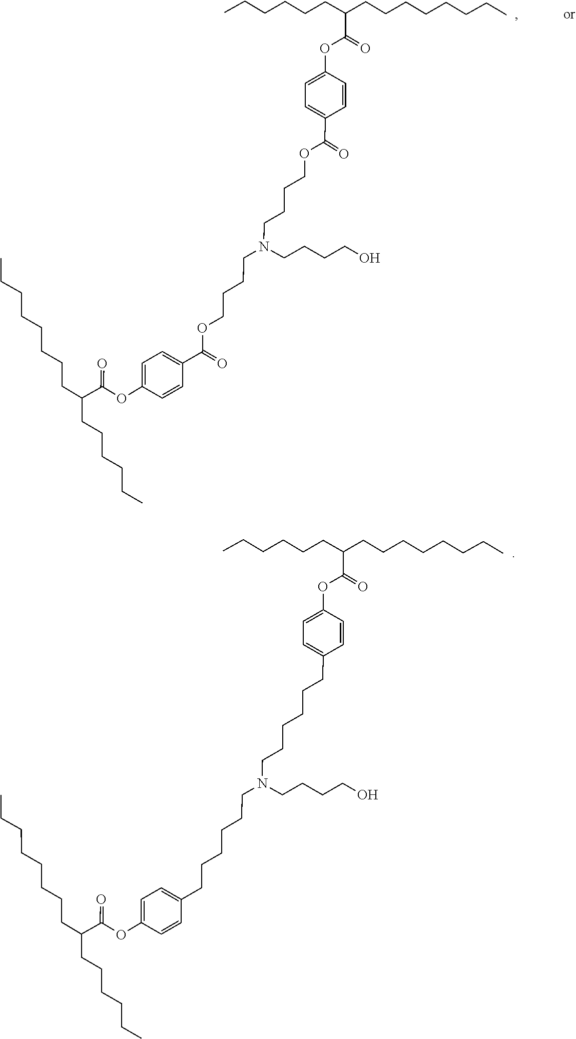

In one embodiment, an ionizable lipid of Formula (I-C):

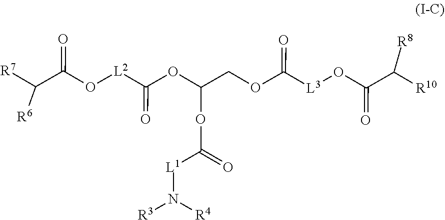

or a pharmaceutically acceptable salt or a stereoisomer thereof, wherein: L¹ is C₁-C₆ alkylene; L² is C₁-C₈ alkylene; L³ is C₁-C₈ alkylene; R³ and R⁴ are each independently H, C₁-C₄ alkyl, or —(CH₂)ₙOH; R⁶ is C₄-C₂₀ alkyl; R⁷ is C₄-C₂₀ alkyl; R⁸ is C₄-C₂₀ alkyl; R¹⁰ is C₄-C₂₀ alkyl; and n is 2, 3, or 4.

In some embodiments, the ionizable lipid has the following structure:

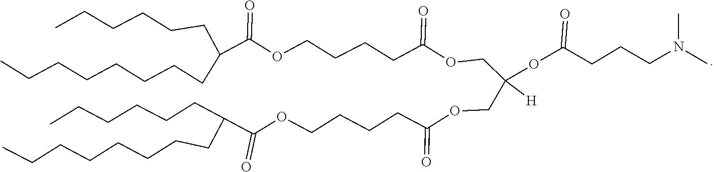 [SMILES: CCCCCCCCC(CCCCCC)C(=O)OCCCCC(=O)OCC(COC(=O)CCCCOC(=O)C(CCCCCC)CCCCCCCC)OC(=O)CCCN(C)C]

In one embodiment, an ionizable lipid of Formula (XXII):

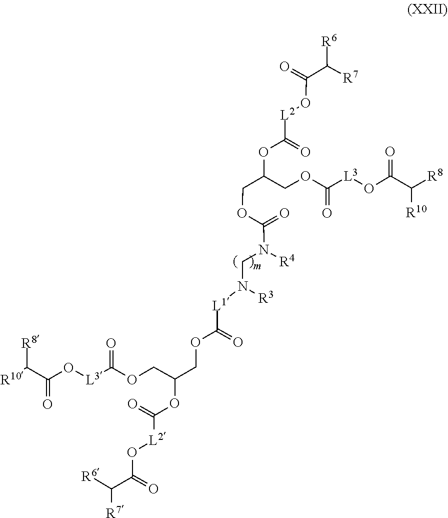

or a pharmaceutically acceptable salt or a stereoisomer thereof, wherein: L¹ is C₁-C₆ alkylene; L^{1′} is C₁-C₆ alkylene; L² is C₁-C₈ alkylene; L^{2′} is C₁-C₈ alkylene; L³ is C₁-C₈ alkylene; L^{3′} is C₁- C₈ alkylene; R³ and R⁴ are each independently H, C₁-C₄ alkyl, —CH₂-cyclopropyl, or —(CH₂)ₙOH; R⁶ is C₄-C₂₀ alkyl; R^{6′} is C₄-C₂₀ alkyl; R⁷ is C₄-C₂₀ alkyl; R^{7′} is C₄-C₂₀ alkyl; R⁸ is C₄-C₂₀ alkyl; R^{7′} is C₄-C₂₀ alkyl; R¹⁰ is C₄-C₂₀ alkyl; R^{10′} is C₄-C₂₀ alkyl; n is 2, 3, or 4; and m is 1, 2, 3, 4, or 5.

In some embodiments, the ionizable lipid has the following structure:

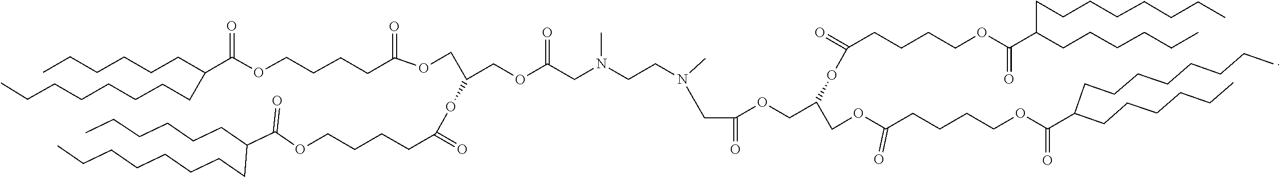

In one aspect, the disclosure relates to a lipid-nanoparticle composition comprising an ionizable lipid of any one of Formula (I) to Formula (XXII). Throughout the disclosure herein, “lipid-nanoparticle composition” can refer to a composition comprising lipid nanoparticles or to the lipid nanoparticles themselves. The lipid-nanoparticle composition can further comprise a helper lipid, a stabilization lipid, a structural lipid, and an active agent, where the active agent is a nucleic acid, small molecule, protein or peptide, or combination thereof. In embodiments, the lipid-nanoparticle composition excludes a stabilization lipid. In embodiments, the lipid-nanoparticle composition excludes a lipid conjugated to polyethylene glycol (a PEG-lipid).

In some aspects, the helper lipid in the lipid-nanoparticle composition is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and mixtures thereof.

In some aspects, the stabilization lipid in the lipid-nanoparticle composition is 1-(monomethoxy-polyethyleneglycol)-2,3-dimyristoylglycerol (PEG-DMG), with an average PEG molecular weight of about 2000 daltons. In some aspects, the stabilization lipid is a polysarcosine-lipid conjugate. In some embodiments, the polysarcosine-lipid conjugate does not associate with RNA in the lipid-nanoparticle composition. In some embodiments, the polysarcosine-lipid conjugate does not form an RNA particle in the lipid-nanoparticle composition. In some embodiments, the polysarcosine-lipid conjugate does not associate with RNA to form an RNA particle in the lipid-nanoparticle composition.

In some aspects, the structural lipid in the lipid-nanoparticle composition is selected from the group consisting of cholesterol, cholesterol derivatives, fecosterol, sitosterol, ergosterol, campesterol, stigmasterol, brassicasterol, tomatidine, ursolic acid, alpha tocopherol, and mixtures thereof.

In some aspects, the nucleic acid is selected from a group consisting of small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), a guide RNA (gRNA), a plasmid DNA (pDNA), an antisense oligodeoxynucleotide (ODN), RNA or DNA vaccine, and mixtures thereof.

In some aspects, the nucleic acid or mRNA is a self-amplifying RNA. In some aspects, the nucleic acid or mRNA is a polycistronic RNA. In some aspects, the nucleic acid or mRNA is a self-amplifying polycistronic RNA. In some aspects, the nucleic acid or mRNA is a circular RNA. In some aspects, the nucleic acid or mRNA expresses a protein or peptide. In some aspects, the protein or peptide expressed from the nucleic acid or mRNA is an antibody, human antibody, camelid antibody, nanobody, humanized antibody, bispecific antibody, enzyme, genome editing enzyme or nuclease, growth factor, cytokine, chemokine, small molecule-mimicking peptide, transcription factor, structural molecule, signaling molecule, reprogramming factor, vaccine antigen, or combination thereof. In some aspects, the mRNA encodes a protein or peptide that acts intracellularly. In some aspects, the mRNA encodes at least one reprogramming factor.

In some aspects, the protein or peptide expressed from the nucleic acid or mRNA is at least one extracellular matrix protein. In some embodiments, the extracellular matrix protein is collagen, laminin, elastin, fibronectin, integrin, tenascin, proteoglycan, fibrin, or combinations thereof. In some embodiments, the collagen is collagen I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, or combinations thereof. In some embodiments, the collagen is collagen VII. In some embodiments, the collagen VII is used in methods of rejuvenating, treating, remodeling, or improving skin or extracellular matrix. In some embodiments, the collagen VII is used in methods of wound healing.

In some aspects, the protein or peptide expressed from the nucleic acid or mRNA is a growth factor, cytokine, or combination thereof. In some embodiments, the growth factor is EGF, FGF, NGF, CNTF, PDGF, VEGF, IGF, GMCSF, GCSF, TGF, Erythropoietin, Ephrin, GDNF, GDF9, KGF, Angiopoeitin, TPO, BMP, HGF, BDNF, GDF, HGH (somatotropin), Neurotrophins, MSF, SGF, GDF (including GDF11), TGF (including TGF-b), or combinations thereof. In some embodiments, the cytokine is IL-1, IL-2, IL-3, IL-4, IL-5, IL-6, IL-7, IL-8, IL-9, IL-10, IL-11, TNF-α, IL-12, IL-13, IL-14, IL-15, IL-16, IL-17, CXCL8 (formerly IL-18), IL-19, IL-20, IL-21, IL-22, IL-23, IL-24, IL-25, IL-26, IL-27, IL-28, IL-29, IL-30, IL-31, IL-32, IL-33, or combinations thereof.

In some aspects, the protein or peptide expressed from the nucleic acid or mRNA is a human antibody, humanized antibody, camelid antibody, companion animal antibody, or nanobody. In some embodiments, the protein or peptide expressed from the nucleic acid or mRNA is an enzyme such as a nuclease, for example a nuclease used in genome editing. In some aspects, the protein or peptide expressed from the nucleic acid or mRNA acts intracellularly. In some aspects, the protein or peptide expressed from the nucleic acid or mRNA is secreted. In some embodiments, the antibody is at least one of or substantially similar to Trastuzumab, Glofitamab, Mirikizumab, Mirvetuximab, Nirsevimab, Tremelimumab, Teclistamab, Donanemab, Spesolimab, Lecanemab, Tislelizumab, Penpulimab, Sintilimab, Teplizumab, Toripalimab, Omburtamab, Retifanlimab, Ublituximab, Inolimomab, Oportuzumab, Narsoplimab, Mosunetuzumab, Tixagevimab, Cilgavimab, Relatlimab, Tebentafusp, Faricimab, Sutimlimab, Sotrovimab, Regdanvimab, Casirivimab, Imdevimab, Tezepelumab, Tisotumab, Amivantamab, Anifrolumab, Loncastuximab, Bimekizumab, Tralokinumab, Evinacumab, Sacituzumab, Teprotumumab, Isatuximab, Eptinezumab, Dostarlimab, Ansuvimab, Margetuximab, Naxitamab, Atoltivimab, Maftivimab, Odesivimab, Belantamab, Tafasitamab, Satralizumab, Inebilizumab, Enfortumab, Crizanlizumab, Brolucizumab, Polatuzumab, Risankizumab, Romosozumab, Caplacizumab, Ravulizumab, Emapalumab, Cemiplimab, Fremanezumab, Moxetumomab, Galcanezumab, Lanadelumab, Mogamulizumab, Erenumab, Tildrakizumab, Ibalizumab, Burosumab, Durvalumab, Emicizumab, Benralizumab, Ocrelizumab, Guselkumab, Inotuzumab, Sarilumab, Dupilumab, Avelumab, Brodalumab, Atezolizumab, Bezlotoxumab, Olaratumab, Reslizumab, Obiltoxaximab, Ixekizumab, Daratumumab, Elotuzumab, Necitumumab, Idarucizumab, Alirocumab, Mepolizumab, Evolocumab, Dinutuximab, Secukinumab, Nivolumab, Blinatumomab, Pembrolizumab, Ramucirumab, Vedolizumab, Siltuximab, Obinutuzumab, Raxibacumab, Pertuzumab, Brentuximab, Belimumab, Ipilimumab, Denosumab, Tocilizumab, Ofatumumab, Canakinumab, Golimumab, Ustekinumab, Certolizumab, Catumaxomab, Eculizumab, Ranibizumab, Panitumumab, Natalizumab, Bevacizumab, Cetuximab, Efalizumab, Omalizumab, Tositumomab, Ibritumomab, Adalimumab, Alemtuzumab, Gemtuzumab, Infliximab, Palivizumab, Basiliximab, Daclizumab, Rituximab, Abciximab, Edrecolomab, Nebacumab, or Muromonab. In some embodiments, the protein or peptide is used in methods of treating human or veterinary diseases.

In some aspects, the small molecule is a chemotherapeutic agent, a GPCR agonist or antagonist, a transcription regulator, or an RNA splicing regulator. In some aspects, the small molecule acts intracellularly.

In some aspects, the protein or peptide is an antibody, humanized antibody, bispecific antibody, enzyme, genome editing enzyme or nuclease, growth factor, cytokine, chemokine, small molecule-mimicking peptide, transcription factor, structural molecule, signaling molecule, reprogramming factor, vaccine antigen, or combination thereof. In some aspects, the protein or peptide acts intracellularly.

In some aspects, the small molecule, protein, or peptide incorporated in the lipid nanoparticle and/or delivered by the lipid nanoparticle composition is a component of “artificial niche” used to maintain quiescence of progenitor cells. In some embodiments, the artificial niche component is selected from the group consisting of elcatonin; MGCD-265, JNJ-7706621, forskolin, fomatostatin, SB203580, SU5402, TGF-β, insulin-transferrin-selenium, and combinations thereof.

These and other artificial niche components are disclosed in U.S. Pat. No. 10,688,136, incorporated herein by reference.

In some aspects, the disclosure relates to a pharmaceutical composition comprising the lipid-nanoparticle composition and a pharmaceutically acceptable carrier thereof.

In aspects, the methods and compositions provided herein are applied to cells, tissue, or organs of the nervous system, muscular system, respiratory system, cardiovascular system, skeletal system, reproductive system, integumentary system, lymphatic system, excretory system, immune system, endocrine system (e.g., endocrine and exocrine), or digestive system. Any type of cell can potentially be rejuvenated, as described herein, including, but not limited to, epithelial cells (e.g., squamous, cuboidal, columnar, and pseudostratified epithelial cells), endothelial cells (e.g., vein, artery, and lymphatic vessel endothelial cells), and cells of connective tissue, muscles, and the nervous system. Such cells may include, but are not limited to, epidermal cells, fibroblasts, chondrocytes, skeletal muscle cells, satellite cells, heart muscle cells, smooth muscle cells, keratinocytes, basal cells, ameloblasts, exocrine secretory cells, myoepithelial cells, osteoblasts, osteoclasts, neurons (e.g., sensory neurons, motor neurons, and interneurons), glial cells (e.g., oligodendrocytes, astrocytes, ependymal cells, microglia, Schwann cells, and satellite cells), pillar cells, adipocytes, pericytes, stellate cells, pneumocytes, blood and immune system cells (e.g., erythrocytes, monocytes, dendritic cells, macrophages, neutrophils, eosinophils, mast cells, T cells, B cells, natural killer cells), hormone-secreting cells, germ cells, interstitial cells, lens cells, photoreceptor cells, taste receptor cells, and olfactory cells; as well as cells and/or tissue from the kidney, liver, pancreas, stomach, spleen, gall bladder, intestines, bladder, lungs, prostate, breasts, urogenital tract, pituitary cells, oral cavity, esophagus, skin, hair, nail, thyroid, parathyroid, adrenal gland, eyes, nose, or brain.

In some aspects, the cells are selected from fibroblasts, endothelial cells, chondrocytes, skeletal muscle stem cells, keratinocytes, mesenchymal stem cells and corneal epithelial cells. In embodiments, the cells are fibroblasts. In embodiments, the cells are endothelial cells. In embodiments, the cells are chondrocytes. In embodiments, the cells are skeletal muscle stem cells. In embodiments, the cells are keratinocytes. In embodiments, the cells are mesenchymal stem cells. In embodiments, the cells are corneal epithelial cells.

In some aspects, methods and compositions of the present technology are applied to immune cells including, but not limited to, lymphocytes, granulocytes, monocytes, macrophages, microglia, or dendritic cells. In some embodiments, the lymphocyte is a T-cell, a B-cell or a natural killer (NK) cell. In some embodiments, the lymphocyte is a tumor-infiltrating lymphocyte.

In some embodiments, the lymphocyte is a T-cell. In some embodiments, the T-cell is a cytotoxic T cell (CD8+), a helper T cell (CD4+), a suppressor or regulatory T cell (Treg), a memory T cell, a natural killer T cell (NKT cell), or a gamma delta T cell. In other embodiments, the helper T cell is a Th1, Th2, Th17, Th9, or Tfh T-cell. In some embodiments, the memory T cell is a central memory T cell, an effector memory T cell, a tissue resident memory T cell, or a virtual memory T cell. In some embodiments, suppressor or regulatory T cells of the present technology are FOXP3+T cells or FOXP3− T cells. In some embodiments, the NKT cell is a subset of CD1d-restricted T cells.

In some embodiments, a granulocyte of the present technology is a neutrophil, an eosinophil, a basophil, or a mast cell.

In other embodiments, a lymphocyte of the present technology is a B-cell. In some embodiments, a B-cell is a memory B-cell or a plasma cell.

In other embodiments, the immune cell is a monocyte, macrophage, microglial cell, or dendritic cell.

In some embodiments, the methods and compositions described herein may be used wherein the cell is an immune cell, such as a natural immune cell or an engineered immune cell. In some embodiments, the methods and compositions described herein are used in parallel or in series with methods of engineering cells, including engineered immune cells, such that the methods are performed before, during, and/or after the engineering of the cells. In some embodiments, the methods and compositions described herein are used for engineering cells, including engineered immune cells. In some embodiments, such engineering includes engineering so that the cells express chimeric antigen receptors, such as chimeric antigen expressing immune cells. In some embodiments, such chimeric antigen receptors target CD19, CD30, CD33, CD123, FLT3, BCMA, GD2, or any other antigen suitable for immunotherapy. In some embodiments, such engineering includes engineering cells, including immune cells, to express other proteins or peptides, such as growth factors and cytokines. In some embodiments, said cytokines include IL-15. In some embodiments, such engineering of cells, such as immune cells, is performed ex vivo, e.g., in the manufacturing of a cellular therapy product, such as an autologous or allogenic chimeric antigen receptor (CAR)-T, CAR-NK, CAR-M, or CAR-NKT cells. In some embodiments, the CAR-NKT cells provided herein are targeted to GD2, by the chimeric antigen receptor, and engineered to express IL-15. In such embodiments, the immune cell rejuvenation methods described herein are performed ex vivo during or after the manufacturing of the cell therapy product. In other embodiments, such engineering of cells, and/or immune cells is performed ex vivo, e.g., in so-called “in situ” generation of CAR-engineered cells. In such embodiments, RNA and/or mRNA encoding CARs or growth factors or cytokines contained in lipid-containing compositions or lipid-nanoparticle compositions of the disclosure is injected in vivo into a subject or patient, for example for CAR engineering of the patient's immune cells, such as T cells, NK cells, macrophages, tumor infiltrating lymphocytes, dendritic cells and/or NKT cells “in situ,” i.e., inside the patient's body without having to remove cells for ex vivo transfection. In such embodiments, the immune cell rejuvenation methods described herein are also performed in vivo, where mRNA encoding the reprogramming factor or factors is injected into the patient before, concurrently with, or after the mRNA encoding CARs or other cell engineering molecules. In some embodiments, the lipids and lipid-nanoparticle compositions of the disclosure are selected for targeted delivery to any cell, including immune cells, in vivo, such as T cells, NK cells, macrophages, tumor infiltrating cells, dendritic cells, and/or NKT cells in vivo. In still other embodiments, in vivo treatment is performed in the absence of any other in vivo cell engineering, to enhance or restore the potency of the immune system and treat diseases associated with immune dysfunction or dysregulation, such as improving the effect of the immune system against cancer or infection, or reducing inflammation.

In some embodiments, the immune cell to be rejuvenated is a non-adherent cell, such as a non-adherent immune cell. In some embodiments, non-adherent cells, including non-adherent immune cells, are treated, transiently reprogrammed, rejuvenated, or manufactured in a manner wherein the cells remain non-adherent, without adhering to a tissue culture substrate or forming or giving rise to cells or colonies of cells that adhere to a tissue culture substrate. In some embodiments, the reprogramming interval and factors are selected such that cells are rejuvenated with retention of cellular identity, wherein the cells remain non-adherent, without adhering to a tissue culture substrate or forming or giving rise to cells or colonies of cells that adhere to a tissue culture substrate. Accordingly, in some embodiments, the present technology provides lipid-containing compositions and lipid-nanoparticle compositions to be used for delivering mRNA encoding at least one reprogramming factor for cell rejuvenation wherein cells, including any non-adherent cells and/or non-adherent immune cells (e.g., non-adherent T cells, NK cells, macrophages, tumor infiltrating cells, dendritic cells, and/or NKT cells), are reprogrammed in a manner wherein the cells are rejuvenated with retention of cellular identity, and wherein the cells stay in suspension and are not adherent, nor do they become or give rise to cells that are adherent, become adherent, or form adherent colonies.

In some embodiments, the methods described herein, including methods of rejuvenating immune cells; methods of reversing, preventing, or inhibiting exhaustion in immune cells; or inducing proliferation in immune cells comprise administering the lipid-containing compositions or lipid-nanoparticle compositions of the disclosure comprising mRNA encoding at least one reprogramming factor to an immune cell 1, 2, 3, 4, 5, 6, 7, 8, 9, or 10 times over a period of 1, 2, 3, 4, 5, 6, 7, 8, 9, or 10 days. For example, the mRNA could be administered once on the first or second day of a five- or six-day period, or it could be administered once on the first day and once on the third day of a five- or six-day period, or it could be administered once in a one-day period.

In some embodiments, the mRNA is administered to an immune cell 1, 2, 3, 4, 5, or 6 times over a period of 1, 2, 3, 4, 5, or 6 days. In some embodiments, the mRNA is administered after an immune cell activation step. In some embodiments, the immune cell activation step comprises activating the immune cells for 1, 2, or 3 days. In some embodiments, the immune cell activation step comprises activating the immune cells using at least one of CD3, CD28, and IL-2. In some embodiments, the immune cells are activated with CD3 and CD28. In some embodiments, the mRNA administration period occurs immediately after the immune cell activation step. In some embodiments, the mRNA administration period occurs 1, 2, 3, 4, 5, 6, 7, 8, 9, 10, 11, 12, 13, or 14 days after the immune cell activation step. In some embodiments, the administration of the mRNA encoding reprogramming factors reverses immune cell exhaustion caused by the immune cell activation step. In some embodiments, the administration of the mRNA encoding reprogramming factors reverses immune cell exhaustion in immune cells from an aged patient or donor. In some embodiments, the administration of the mRNA is performed during a manufacturing process to make immune cells for transplantation, for example CAR-T, CAR-M, or CAR-NK cells.

In some embodiments, the use of the lipids or lipid-nanoparticles of the disclosure for delivery of mRNA results in enhanced rejuvenation, proliferation, recovery from or prevention of exhaustion, therapeutic effects, anti-pathogenic effects, anti-cancer effects, anti-immunogenic effects or anti-inflammatory effects in a cell treated or rejuvenated using the methods or compositions herein compared to using a different delivery mechanism for the mRNA. In some embodiments, such enhanced rejuvenation, proliferation, recovery from or prevention of exhaustion, therapeutic effects, anti-pathogenic effects, anti-cancer effects, anti-immunogenic effects or anti-inflammatory effects result from lower toxicity, immunogenicity, and/or lower physiological impact on the cell when compared to the different delivery mechanism. In some embodiments, the different delivery mechanism is electroporation such that the use of the lipids or lipid-nanoparticles compositions of the disclosure for delivery of the mRNA results in enhanced rejuvenation, proliferation, recovery from or prevention of exhaustion, therapeutic effects, anti-pathogenic effects, anti-cancer effects, anti-immunogenic effects or anti-inflammatory effects in cells treated or rejuvenated using the methods or compositions herein compared to when using electroporation. This improvement compared to electroporation can result from reduced toxicity or reduced physiological impact on the cell compared to electroporation.

In some aspects, the lipids or lipid-nanoparticle compositions of the disclosure are used in methods of delivery of therapeutic or diagnostic agents to the skin, comprising administering lipid-containing compositions or lipid-nanoparticle compositions comprising at least one therapeutic or diagnostic agent. In some embodiments, the lipids or lipid-nanoparticle compositions of the present technology provide delivery of therapeutic or diagnostic agents, such as reprogramming factors, in a manner that achieves transient reprogramming of a cell, such as a skin cell or immune cell. In some embodiments, transient reprogramming of a cell provides transient expression of a therapeutic or diagnostic agent, such as a reprogramming factor, wherein the agent is expressed in a cell for a duration sufficient to reprogram and/or rejuvenate without changing the identity of the cell, i.e., rejuvenating a skin or immune cell to exhibit features or a younger skin or immune cell while retaining the identity of a skin or immune cell.

In some aspects, the therapeutic agent is mRNA as disclosed herein. In some aspects the lipids or lipid-nanoparticle compositions of the disclosure are used in methods of treating or preventing dermatological diseases or conditions, treating or preventing diseases or conditions of the skin, or for cosmetic applications in skin, comprising administering lipid-containing compositions or lipid-nanoparticle compositions comprising at least one therapeutic or diagnostic agent. In some aspects the lipids or lipid-nanoparticle compositions of the disclosure are used in methods of treating or preventing dermatological diseases or conditions or diseases or conditions of the skin, comprising administering lipid-containing compositions or lipid-nanoparticle compositions comprising at least one therapeutic or diagnostic agent. In some aspects, the therapeutic agent is mRNA as disclosed herein. In some aspects, the mRNA encodes at least one reprogramming factor. In some aspects the lipids or lipid-nanoparticle compositions of the disclosure are used for cosmetic applications in skin, comprising administering lipid-containing compositions or lipid-nanoparticle compositions comprising at least one therapeutic or diagnostic agent. In some aspects, the therapeutic agent is mRNA as disclosed herein. In some aspects, the mRNA encodes at least one reprogramming factor. In some aspects the lipids or lipid-nanoparticle compositions of the disclosure are used in methods of rejuvenating skin, comprising administering lipid-containing compositions or lipid-nanoparticle compositions comprising a therapeutic or diagnostic agent. In some aspects the lipids or lipid-nanoparticle compositions of the disclosure are used in methods of rejuvenating skin, comprising administering lipid-containing compositions or lipid-nanoparticle compositions comprising mRNA encoding at least one reprogramming factor. In such methods, the mRNA can be bound to the lipids or contained in the lipid-nanoparticles. In some aspects, such methods further comprise transfecting skin cells with the lipid-containing compositions or lipid-nanoparticle compositions to deliver the mRNA. In some aspects, the lipids or lipid-nanoparticle compositions of the disclosure are used in methods of rejuvenating skin, comprising administering lipid-containing compositions or lipid-nanoparticle compositions comprising mRNA encoding at least one reprogramming factor to achieve rejuvenation of the skin while retaining cell identity. In such methods, the mRNA can be bound to the lipids or contained in the lipid-nanoparticles. In some aspects, the lipids or lipid-nanoparticle compositions of the disclosure are used in methods of rejuvenating skin, comprising administering lipid-containing compositions or lipid-nanoparticle compositions comprising mRNA encoding at least one reprogramming factor to skin cells, wherein expression of the at least one reprogramming factor in skin cells results in increased fibroblast proliferation with retention of skin cell identity. In some aspects, the lipids or lipid-nanoparticle compositions of the disclosure are used in methods of increasing skin thickness, comprising administering lipid-containing compositions or lipid-nanoparticle compositions comprising a therapeutic or diagnostic agent. In some aspects, the therapeutic agent is mRNA. In some aspects, the lipids or lipid-nanoparticle compositions of the disclosure are used in methods of increasing skin thickness, comprising administering lipid-containing compositions or lipid-nanoparticle compositions comprising mRNA encoding at least one reprogramming factor to skin cells, wherein expression of the at least one reprogramming factor in skin cells results in increased skin thickness with retention of skin cell identity. In some aspects, the lipids or lipid-nanoparticle compositions of the disclosure are used in methods of increasing skin elasticity, comprising administering lipid-containing compositions or lipid-nanoparticle compositions comprising a therapeutic or diagnostic agent. In some aspects, the therapeutic agent is mRNA. In some aspects, the lipids or lipid-nanoparticle compositions of the disclosure are used in methods of increasing skin elasticity, comprising administering lipid-containing compositions or lipid-nanoparticle compositions comprising mRNA encoding at least one reprogramming factor to skin cells, wherein expression of the at least one reprogramming factor in skin cells results in increased skin elasticity with retention of skin cell identity. In such methods, the mRNA can be bound to the lipids or contained in the lipid-nanoparticles.

In some aspects the lipids or lipid-nanoparticle compositions of the disclosure are used in methods of wound healing, comprising administering lipid-containing compositions or lipid-nanoparticle compositions comprising a therapeutic or diagnostic agent. In some aspects, the lipids or lipid-nanoparticle compositions of the disclosure are used in methods of wound healing, comprising administering lipid-containing compositions or lipid-nanoparticle compositions comprising mRNA encoding at least one reprogramming factor. In such methods, the mRNA can be bound to the lipids or contained in the lipid-nanoparticles. In some aspects the lipids or lipid-nanoparticle compositions of the disclosure are used in methods of wound healing, wherein lipid-containing compositions or lipid-nanoparticle compositions deliver mRNA encoding at least one reprogramming factor to achieve wound healing while retaining cell identity. In such methods, the mRNA can be bound to the lipids or contained in the lipid-nanoparticles. In some aspects, the lipids or lipid-nanoparticle compositions of the disclosure are used in methods of wound healing, comprising administering lipid-containing compositions or lipid-nanoparticle compositions comprising mRNA encoding at least one reprogramming factor to skin cells, wherein expression of the at least one reprogramming factor in skin cells results in increased fibroblast proliferation. In such methods, the mRNA can be bound to the lipids or contained in the lipid-nanoparticles.

In some aspects the lipids or lipid-nanoparticle compositions of the disclosure are used in methods of treating or preventing dermatological diseases or conditions, treating or preventing diseases or conditions of the skin, or for cosmetic applications in skin, comprising administering lipid-containing compositions or lipid-nanoparticle compositions comprising a therapeutic agent to achieve reversal of at least one skin aging marker. In some aspects, the lipids or lipid-nanoparticle compositions of the disclosure are used in methods of rejuvenating skin, comprising administering lipid-containing compositions or lipid-nanoparticle compositions to deliver mRNA encoding at least one reprogramming factor to skin cells to achieve reversal of at least one skin aging marker while retaining cell identity. In some aspects, the lipids or lipid-nanoparticle compositions of the disclosure are used in methods of wound healing, comprising administering lipid-containing compositions or lipid-nanoparticle compositions to deliver mRNA encoding at least one reprogramming factor to skin cells to achieve reversal of at least one skin aging marker while retaining cell identity. In some embodiments, reversal of at least one skin aging marker refers to producing a rejuvenated cell that expresses at least one skin aging marker in a manner similar to the expression of said marker seen in a youthful skin cell as compared to an aged skin cell.

In some aspects, the marker is mRNA or protein expression of IL6, CXCL8, CSF3, CXCL1, SERPINB2, LIF, IL11, CXCL2, IL24, PTGS2, MMP3, CCL2, TFPI2, IER3, ACKR3, PTGES, SLC16A6, TNFAIP6, PTPRN, IL1RN, IL1B, CXCL5, CXCL6, HAS1, HSD11B1, CH₂₅H, ADGRD1, C₃, RASD1, NR4A3, STC1, TCIM, SRGN, AC003092.1, LRRN3, CHI3L¹, NR4A2, NAMPT, PRSS23, MMP1, SOD2, LOXL4, MMP11, ELN, CREG1, C₁₅orf48, NFKBIZ, PID1, or any combination thereof. In some aspects, reversal of at least one skin aging marker is downregulation of mRNA or protein expression of IL6, CXCL8, CSF3, CXCL1, SERPINB2, LIF, IL11, CXCL2, IL24, PTGS2, MMP3, CCL2, TFPI2, IER3, ACKR3, PTGES, SLC16A6, TNFAIP6, PTPRN, IL1RN, IL1B, CXCL5, CXCL6, HAS1, HSD11B1, CH₂₅H, ADGRD1, C₃, RASD1, NR4A3, STC1, TCIM, SRGN, AC003092.1, LRRN3, CHI3L¹, NR4A2, NAMPT, MMP1, SOD2, CREG1, C₁₅orf48, NFKBIZ, PID1, or any combination thereof. In some aspects, reversal of at least one skin aging marker is upregulation of mRNA or protein expression of PRSS23, LOXL4, MMP11, ELN, or any combination thereof. In some aspects, reversal of at least one skin aging marker is upregulation of mRNA or protein expression of PRSS23. In some aspects, reversal of at least one skin aging marker is upregulation of mRNA or protein expression of LOXL4.

In some aspects, reversal of at least one skin aging marker is upregulation of mRNA or protein expression of MMP11. In some aspects, reversal of at least one skin aging marker is upregulation of mRNA or protein expression of ELN. In some aspects, reversal of at least one skin aging marker is downregulation of mRNA or protein expression of MMP3, MMP1, SOD2, or any combination thereof. In some aspects, reversal of at least one skin aging marker is downregulation of mRNA or protein expression of MMP3. In some aspects, reversal of at least one skin aging marker is downregulation of mRNA or protein expression of MMPL. In some aspects, reversal of at least one skin aging marker is downregulation of mRNA or protein expression of SOD2. In some aspects, reversal of at least one skin aging marker is upregulation of mRNA or protein expression of at least one of PRSS23, LOXL4, MMP11, or ELN; downregulation of mRNA or protein expression of at least one of MMP3, MMP1, SOD2; or any combination thereof.

In some aspects the lipids or lipid-nanoparticle compositions of the disclosure are used in methods of treating or preventing dermatological diseases or conditions, treating or preventing diseases or conditions of the skin, or for cosmetic applications in skin, comprising administering lipid-containing compositions or lipid-nanoparticle compositions comprising a therapeutic agent to achieve improvement of at least one skin quality marker. In some aspects, the lipids or lipid-nanoparticle compositions of the disclosure are used in methods of increasing skin thickness, comprising administering lipid-containing compositions or lipid-nanoparticle compositions comprising mRNA encoding at least one reprogramming factor to skin cells, wherein expression of the at least one reprogramming factor in skin cells results in increased skin thickness with retention of skin cell identity. In some aspects, the lipids or lipid-nanoparticle compositions of the disclosure are used in methods of increasing skin elasticity, comprising administering lipid-containing compositions or lipid-nanoparticle compositions comprising mRNA encoding at least one reprogramming factor to skin cells, wherein expression of the at least one reprogramming factor in skin cells results in increased skin elasticity with retention of skin cell identity. In some aspects, the lipids or lipid-nanoparticle compositions of the disclosure are used in methods of rejuvenating skin, comprising administering lipid-containing compositions or lipid-nanoparticle compositions comprising mRNA encoding at least one reprogramming factor to skin cells to achieve improvement of at least one skin quality marker. In some embodiments, the lipids or lipid-nanoparticle compositions of the disclosure are used in methods of rejuvenating skin, comprising administering lipid-containing compositions or lipid-nanoparticle compositions comprising mRNA encoding at least one reprogramming factor to skin cells to achieve improvement of at least one skin quality marker, while retaining cell identity. In some aspects, the at least one skin quality marker comprises skin thickness. In some aspects, skin thickness is increased. In some aspects, the at least one skin quality marker is skin elasticity. In some aspects, skin elasticity is increased. In some aspects, the at least one skin quality marker is transepidermal water loss. Skin thickness, skin elasticity, and transepidermal water loss can be measured according to any method known to those skilled in the art.

In some aspects, the lipids or lipid-nanoparticle compositions of the disclosure are used in methods of wound healing, comprising administering lipid-containing compositions or lipid-nanoparticle compositions comprising mRNA encoding at least one reprogramming factor to skin cells to achieve improvement of at least one skin quality marker. In some aspects, the marker is mRNA or protein expression of Collagen Type I, Collagen Type III, Collagen Type V, Collagen Type VI, Collagen Type XI, Elastin, Microfibril-Associated Protein 5, Periostin, Versican, Connective Tissue Growth Factor, Lysyl Oxidase, SPARC, Secreted Phosphoprotein 1, Cartilage Oligomeric Matrix Protein, MMP1, MMP3, MMP12, SOD2 or any combination thereof. In some aspects, improvement of at least one skin quality marker is downregulation of mRNA or protein expression of MMP1, MMP3, MMP12, SOD2 or any combination thereof. In some aspects, improvement of at least one skin quality marker is upregulation of mRNA or protein expression of Collagen Type I, Collagen Type III, Collagen Type IV, Collagen Type V, Collagen Type VI, Collagen Type XI, Elastin, Microfibril-Associated Protein 5, Periostin, Versican, Connective Tissue Growth Factor, Lysyl Oxidase, SPARC, Secreted Phosphoprotein 1, Cartilage Oligomeric Matrix Protein, or any combination thereof. In some aspects, improvement of at least one skin quality marker is upregulation of mRNA or protein expression of Collagen. In some aspects, improvement of at least one skin quality marker is upregulation of mRNA or protein expression of Collagen Type I. In some aspects, improvement of at least one skin quality marker is upregulation of mRNA or protein expression of Collagen Type III. In some aspects, improvement of at least one skin quality marker is upregulation of mRNA or protein expression of Collagen Type IV. In some aspects, improvement of at least one skin quality marker is upregulation of mRNA or protein expression of Collagen Type V. In some aspects, improvement of at least one skin quality marker is upregulation of mRNA or protein expression of Collagen Type VI. In some aspects, improvement of at least one skin quality marker is upregulation of mRNA or protein expression of Collagen Type XI. In some aspects, improvement of at least one skin quality marker is upregulation of mRNA or protein expression of Collagen Type XI. In some aspects, improvement of at least one skin quality marker is upregulation of mRNA or protein expression of Elastin. In some aspects, improvement of at least one skin quality marker is upregulation of mRNA or protein expression of Microfibril-associated Protein 5. In some aspects, improvement of at least one skin quality marker is upregulation of mRNA or protein expression of Periostin. In some aspects, improvement of at least one skin quality marker is upregulation of mRNA or protein expression of Versican. In some aspects, improvement of at least one skin quality marker is upregulation of mRNA or protein expression of Connective Tissue Growth Factor. In some aspects, improvement of at least one skin quality marker is upregulation of mRNA or protein expression of Lysyl Oxidase. In some aspects, improvement of at least one skin quality marker is upregulation of mRNA or protein expression of SPARC. In some aspects, improvement of at least one skin quality marker is upregulation of mRNA or protein expression of Secreted Phosphoprotein 1. In some aspects, improvement of at least one skin quality marker is upregulation of mRNA or protein expression of Cartilage Oligomeric Matrix Protein. In some aspects, improvement of at least one skin quality marker is downregulation of mRNA or protein expression of MMP3, MMP1, SOD2, or any combination thereof. In some aspects, improvement of at least one skin quality marker is downregulation of mRNA or protein expression of MMP3. In some aspects, improvement of at least one skin quality marker is downregulation of mRNA or protein expression of MMP1. In some aspects, improvement of at least one skin quality marker is downregulation of mRNA or protein expression of SOD2. In some aspects, improvement of at least one skin quality marker is upregulation of mRNA or protein expression of Collagen VII and Elastin. In some aspects, improvement of at least one skin quality marker is upregulation of mRNA or protein expression of at least one of Collagen VII and Elastin; downregulation of mRNA or protein expression of at least one of MMP3, MMP1, SOD2; or any combination thereof.

In some aspects, the lipid-nanoparticle compositions of the disclosure or compositions containing the lipids of the disclosure are administered to the skin topically. In some aspects, the lipid-nanoparticle compositions of the disclosure or compositions containing the lipids of the disclosure are administered to the skin in an ointment, cream, or salve. In some aspects, the lipid-nanoparticle compositions of the disclosure or compositions containing the lipids of the disclosure are administered to the skin via dermal, intradermal, or subcutaneous injection. In some aspects, the lipid-nanoparticle compositions of the disclosure or compositions containing the lipids of the disclosure are administered to the skin via a gel. In some aspects, the lipid-nanoparticle compositions of the disclosure or compositions containing the lipids of the disclosure are administered in vivo, in vitro, or ex vivo. In some aspects, the lipid-nanoparticle compositions of the disclosure or compositions containing the lipids of the disclosure are administered in vivo. In some aspects, the lipid-nanoparticle compositions of the disclosure or compositions containing the lipids of the disclosure are to a human or animal subject.

In some aspects, the lipid-nanoparticle compositions of the disclosure or compositions containing the lipids of the disclosure transfect skin cells to deliver at least one therapeutic or diagnostic agent into skin cells. In some aspects, the therapeutic agent is a nucleic acid. In some aspects, the therapeutic agent is mRNA. In some aspects, the therapeutic agent is a combination of mRNA and siRNA. In some aspects, the therapeutic agent is a combination of mRNA and miRNA. In some aspects, the skin cell is a keratinocyte, melanocyte, Langerhans cell, follicle cell, fibroblast, endothelial cell, smooth muscle cell, Merkel cell, basal cell, squamous cell, apocrine gland cell, eccrine gland cell, sebaceous gland cell, lymphatic endothelial cell, or combination thereof. In some aspects, the lipid or lipid-nanoparticle composition is selected to provide selective transfection to a particular cell type or cell types. In some aspects, the lipid or lipid-nanoparticle composition is selected to provide diffusion within the skin or within at least one layer of the skin.

In some aspects, the dermatological disease or condition or disease or condition of the skin treated that is treated or prevented using the lipid compositions or lipid-nanoparticle compositions of the present disclosure is dermatoporosis or chronic wounds. In some aspects the dermatoporosis or chronic wound is a diabetic, ischemic, and pressure ulcer. In some aspects, the dermatological disease or condition or disease or condition of the skin treated or prevented using the lipid compositions or lipid-nanoparticle compositions of the disclosure is an inflammatory skin disease. In some aspects, the inflammatory skin disease is psoriasis, atopic dermatitis, vitiligo, alopecia areata, or hidradenitis suppurativa. In some aspects, the dermatological disease or condition or disease or condition of the skin treated or prevented using the lipid compositions or lipid-nanoparticle compositions of the disclosure is a hair disorder. In some aspects, the hair disorder is non-scarring or scarring alopecia, hair greying, hirsutism. In some aspects, the hair disorder is non-scarring alopecia is androgenic alopecia. In some aspects, scarring alopecia is lichen planopilaris. In some aspects, the dermatological disease or condition or disease or condition of the skin treated or prevented using the lipid compositions or lipid-nanoparticle compositions of the disclosure is a skin cancer. In some aspects, the skin cancer is basal cell carcinoma, squamous cell carcinoma, or actinic keratosis. In some aspects, the dermatological disease or condition or disease or condition of the skin treated or prevented using the lipid compositions or lipid-nanoparticle compositions of the disclosure is prurigo nodularis, acne, rosacea, or solar lentigines. In some aspects, a method of treating any of the above dermatological diseases or conditions or diseases comprises administering compositions containing lipids of the disclosure or lipid-nanoparticle compositions of the disclosure comprising a therapeutic agent. In some aspects, the therapeutic agent is mRNA. In some aspects, the therapeutic agent is mRNA encoding an antibody, human antibody, humanized antibody, nanobody, camelid antibody, bispecific antibody, enzyme, genome editing enzyme or nuclease, growth factor, cytokine, chemokine, transcription factor, structural molecule, signaling molecule, reprogramming factor. In some aspects, the therapeutic agent is mRNA encoding a protein or peptide that acts intracellularly. In some aspects, the therapeutic agent is mRNA encoding at least one reprogramming factor.

In some aspects, the lipid compositions or lipid-nanoparticle compositions of the disclosure are used in methods of wound healing wherein the wound is dermatoporosis or a chronic wound. In some aspects the dermatoporosis or chronic wound is a diabetic, ischemic, and pressure ulcer. In some aspects, the lipid compositions or lipid-nanoparticle compositions of the disclosure are used in methods of wound healing wherein the wound is a lesion from a skin cancer. In some aspects, the skin cancer is basal cell carcinoma, squamous cell carcinoma, or actinic keratosis. In some aspects, any of the above wound healing methods comprises administering compositions containing lipids of the disclosure or lipid-nanoparticle compositions of the disclosure comprising a therapeutic agent. In some aspects, the therapeutic agent is mRNA. In some aspects, the therapeutic agent is mRNA encoding an antibody, human antibody, humanized antibody, nanobody, camelid antibody, bispecific antibody, enzyme, genome editing enzyme or nuclease, growth factor, cytokine, chemokine, transcription factor, structural molecule, signaling molecule, reprogramming factor. In some aspects, the therapeutic agent is mRNA encoding a protein or peptide that acts intracellularly. In some aspects, the therapeutic agent is mRNA encoding at least one reprogramming factor.

In some aspects, some lipid-nanoparticle compositions of the disclosure containing lipid formula (II) have higher transfection efficiency in skin cells compared to other lipid-nanoparticle compositions containing lipid formula (II) or to other lipid-nanoparticle compositions containing other lipids of the disclosure and when used to transfect at least one reprogramming factor produce greater improvement of at least one skin quality marker. In some aspects, the at least one skin quality marker is skin thickness, skin elasticity, transepidermal water loss, or any combination thereof.

In some aspects, the lipid-nanoparticle compositions of the disclosure or compositions containing the lipids of the disclosure transfect skin cells to deliver at least one therapeutic or diagnostic agent into eye cells. In some aspects, the therapeutic agent is a nucleic acid. In some aspects, the therapeutic agent is mRNA. In some aspects, the therapeutic agent is a combination of mRNA and siRNA. In some aspects, the therapeutic agent is a combination of mRNA and miRNA. In some aspects, the eye cell is a retinal pigment epithelial cell, ganglion cell, photoreceptor cell, rod cell, cone cell, choroid cell, corneal cell, conjunctival cells, corneal epithelial cell, eye muscle cell, or combination thereof. In some aspects, the lipid or lipid-nanoparticle composition is selected to provide selective transfection to a particular cell type or cell types. In some aspects, the lipid or lipid-nanoparticle composition is selected to provide diffusion within the eye, such as within the vitreous humor or within the retina.

In some aspects the lipids or lipid-nanoparticle compositions of the disclosure are used in methods of treating or preventing ocular diseases or conditions or treating or preventing diseases or conditions of the eye, comprising administering lipid-containing compositions or lipid-nanoparticle compositions comprising a therapeutic agent to the eye.

In some aspects, the ocular disease or condition or disease or condition of the eye treated that is treated or prevented using the lipid compositions or lipid-nanoparticle compositions of the present disclosure is age-related macular degeneration, glaucoma, a cataract, dry eye, diabetic retinopathy, vision loss, myopia, presbyopia, dry macular degeneration, or wet macular degeneration. In some aspects, a method of treating any of the above ocular diseases or conditions or diseases comprises administering compositions containing lipids of the disclosure or lipid-nanoparticle compositions of the disclosure comprising a therapeutic agent. In some aspects, the therapeutic agent is mRNA. In some aspects, the therapeutic agent is mRNA encoding an antibody, human antibody, humanized antibody, nanobody, camelid antibody, bispecific antibody, enzyme, genome editing enzyme or nuclease, growth factor, cytokine, chemokine, transcription factor, structural molecule, signaling molecule, reprogramming factor. In some aspects, the therapeutic agent is mRNA encoding a protein or peptide that acts intracellularly. In some aspects, the therapeutic agent is mRNA encoding at least one reprogramming factor.

The methods provided herein include using the lipids or lipid-nanoparticle compositions to transfect cells with one or more non-integrative messenger RNAs encoding one or more cellular reprogramming factors, thereby producing rejuvenated cells. The cells to be rejuvenated may be of any cell type. In embodiments, the cells are contacted with, exposed to, or transfected with the mRNA for not more than about 20, 19, 18, 17, 16, 15, 14, 13, 12, 11, 10, 9, 8, 7, 6, 5, 4, 3, 2, or 1 days, or for less than 1 day. In embodiments, the cells are contacted with, exposed to, or transfected with the mRNA for not more than about 14, 13, 12, 11, 10, 9, 8, 7, 6, 5, 4, 3, 2, or 1 days, or for less than one day. In embodiments, the cells are contacted with, exposed to, or transfected with the mRNA for not more than about 10, 9, 8, 7, 6, 5, 4, 3, 2, or 1 days, or for less than one day. In embodiments, the cells are contacted with, exposed to, or transfected with the mRNA for not more than about 7, 6, 5, 4, 3, 2, or 1 days, or for less than one day. In embodiments, the cells are contacted with, exposed to, or transfected with the mRNA for not more than about 5, 4, 3, 2, or 1 days, or for less than one day. In embodiments, the at least one reprogramming factor is expressed from the transfected mRNA within cells, or the cells are exposed to the at least one reprogramming factor expressed from the transfected mRNA, for not more than about 20, 19, 18, 17, 16, 15, 14, 13, 12, 11, 10, 9, 8, 7, 6, 5, 4, 3, 2, or 1 days, or for less than 1 day. In embodiments, the at least one reprogramming factor is expressed from the transfected mRNA within cells, or the cells are exposed to the at least one reprogramming factor expressed from the transfected mRNA, for not more than about 14, 13, 12, 11, 10, 9, 8, 7, 6, 5, 4, 3, 2, or 1 days, or for less than one day. In embodiments, the at least one reprogramming factor is expressed from the transfected mRNA within cells, or the cells are exposed to the at least one reprogramming factor expressed from the transfected mRNA, for at least about 2 days and not more than about 14, 13, 12, 11, 10, 9, 8, 7, 6, 5, 4, 3, or 2 days. In embodiments, the at least one reprogramming factor is expressed from the transfected mRNA within cells, or the cells are exposed to the at least one reprogramming factor expressed from the transfected mRNA, for not more than about 10, 9, 8, 7, 6, 5, 4, 3, 2, or 1 days, or for less than one day. In embodiments, the at least one reprogramming factor is expressed from the transfected mRNA within cells, or the cells are exposed to the at least one reprogramming factor expressed from the transfected mRNA, for at least about 2 days and not more than about 10, 9, 8, 7, 6, 5, 4, 3, or 2 days. In embodiments, the at least one reprogramming factor is expressed from the transfected mRNA within cells, or the cells are exposed to the at least one reprogramming factor expressed from the transfected mRNA, for not more than about 7, 6, 5, 4, 3, 2, or 1 days, or for less than one day. In embodiments, the at least one reprogramming factor is expressed from the transfected mRNA within cells, or the cells are exposed to the at least one reprogramming factor expressed from the transfected mRNA, for at least about 2 days and not more than about 7, 6, 5, 4, 3, or 2 days. In embodiments, the at least one reprogramming factor is expressed from the transfected mRNA within cells, or the cells are exposed to the at least one reprogramming factor expressed from the transfected mRNA, for not more than about 5, 4, 3, 2, or 1 days, or for less than one day. In embodiments, the at least one reprogramming factor is expressed from the transfected mRNA within cells, or the cells are exposed to the at least one reprogramming factor expressed from the transfected mRNA, for at least about 2 days and not more than about 5, 4, 3, or 2 days. In embodiments, the rejuvenated cells have a phenotype or activity profile similar to a young cell. The phenotype or activity profile includes one or more of the transcriptomic profile, gene expression of one or more nuclear and/or epigenetic markers, proteolytic activity, mitochondrial health and function, SASP cytokine expression, and methylation landscape.

In some embodiments, the rejuvenated cells have a transcriptomic profile that is more similar to the transcriptomic profile of young cells. In embodiments, the transcriptomic profile of the rejuvenated cells includes an increase in gene expression of one or more genes selected from RPL37, RHOA, SRSF3, EPHB4, ARHGAP18, RPL31, FKBP2, MAP1LC3B2, Elfl, Phf8, Pol2s2, Tafl and Sin3a.

In some embodiments, the rejuvenated cells exhibit increased gene expression of one or more nuclear and/or epigenetic markers compared to a reference value. In embodiments, the one or more nuclear and/or epigenetic markers is selected from Hplgamma, H3K9me3, lamina support protein LAP2alpha, and SIRTl protein. In embodiments, the rejuvenated cells have a proteolytic activity that is more similar to the proteolytic activity of young cells. In embodiments, the proteolytic activity is measured as increased cell autophagosome formation, increased chymotrypsin-like proteasome activity, or a combination thereof. In embodiments, the rejuvenated cells exhibit improved mitochondria health and function compared to a reference value. In embodiments, improved mitochondria health and function is measured as increased mitochondria membrane potential, decreased reactive oxygen species (ROS), or a combination thereof.

In some embodiments, the rejuvenated cells exhibit decreased expression of one or more SASP cytokines compared to a reference value. In embodiments, the one or more SASP cytokines include IL18, ILIA, GROA, IL22, and IL9. In embodiments, the rejuvenated cells exhibit reversal of the methylation landscape. In embodiments, the reversal of the methylation landscape is measured by Horvath clock estimation. In some embodiments, a reference value is obtained from an aged cell.

In embodiments, cells are rejuvenated by transient reprogramming with mRNAs encoding one or more cellular reprogramming factors transfected into the cells using the lipids or lipid-nanoparticle compositions of the disclosure. Transient reprogramming is accomplished, in some embodiments, by transfecting cells with non-integrative mRNAs for not more than about 20, 19, 18, 17, 16, 15, 14, 13, 12, 11, 10, 9, 8, 7, 6, 5, 4, 3, 2, or 1 days, or less than 1 day. Transient reprogramming is accomplished, in some embodiments, by transfecting cells with non-integrative mRNAs for not more than about 14, 13, 12, 11, 10, 9, 8, 7, 6, 5, 4, 3, 2, or 1 days, or less than 1 day. Transient reprogramming is accomplished, in some embodiments, by transfecting cells with non-integrative mRNAs for not more than about 9, 8, 7, 6, 5, 4, 3, 2, or 1 days, or less than 1 day.

Transient reprogramming is accomplished, in some embodiments, by transfecting cells with non-integrative mRNAs for not more than about 6, 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for not more than about 20, 19, 18, 17, 16, 15, 14, 13, 12, 11, 10, 9, 8, 7, 6, 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for at least 2 days and not more than about 20, 19, 18, 17, 16, 15, 14, 13, 12, 11, 10, 9, 8, 7, 6, 5, 4, 3, or 2 days. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for not more than about 14, 13, 12, 11, 10, 9, 8, 7, 6, 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for at least 2 days and not more than about 14, 13, 12, 11, 10, 9, 8, 7, 6, 5, 4, 3, or 2 days. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for not more than about 10, 9, 8, 7, 6, 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for at least 2 days and not more than about 10, 9, 8, 7, 6, 5, 4, 3, or 2 days. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for not more than about 7, 6, 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for at least 2 days and not more than about 7, 6, 5, 4, 3, or 2 days. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for not more than about 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for at least 2 days and not more than about 5, 4, 3, or 2 days. In embodiments, transient reprogramming of cells eliminates various hallmarks of aging while avoiding complete dedifferentiation of the cells into stem cells.

In embodiments of the methods and compositions provided herein, cellular age-reversal, or rejuvenation, is achieved by transient overexpression of one or more mRNAs encoding cellular reprogramming factors, delivered by the lipids or lipid-nanoparticle compositions of the disclosure. Such cellular reprogramming factors may include transcription factors, epigenetic remodelers, or small molecules affecting mitochondrial function, proteolytic activity, heterochromatin levels, histone methylation, nuclear lamina polypeptides, cytokine secretion, or senescence. In embodiments, the cellular reprogramming factors include one or more of OCT4, SOX2, KLF4, c-MYC, LIN28 and NANOG. In embodiments, the cellular reprogramming factors are applied in different molar ratios, for example OCT4, SOX2, KLF4, c-MYC, LIN28, and NANOG at molar ratios of a:b:c:d:e:f, wherein a, b, c, d, e, and f can all be the same number (for example, 1:1:1:1:1:1), some the same number and some different numbers (for example, 3:1:1:1:1:1, 2:1:1:1:1:1, 2:2:1:1:1:1, 2:2:2:1:1:1, 2:2:2:2:1:1, 2:2:2:2:2:1, 3:3:3:3:2:2), or all different numbers (for example 6:4:5:3:2:1), and wherein a, b, c, d, e, and f are each 1-7, i.e., 1-7:1-7:1-7:1-7:1-7:1-7 (or 1-7:1-7:1-7:1-7:1-7, 1-7:1-7:1-7:1-7, 1-7:1-7:1-7, 1-7:1-7, or 1-7:1 in the case of combinations with fewer than 6 factors).

In embodiments, the methods and compositions provided herein may be applied to any type of cell, tissue or organs in need of rejuvenation. The methods and compositions of the disclosure can be used to rejuvenate cells in culture (e.g., ex vivo or in vitro) to improve function and potency for use in cell therapy. The cells used in treatment of a patient may be autologous or allogeneic. The cells can be derived from the patient or a matched donor, or they can be obtained from a cell bank or derived from iPS cells. For example, in autologous ex vivo therapy, cells can be obtained directly from the patient to be treated, transfected with mRNAs encoding cellular reprogramming factors, as described herein, and reimplanted in the patient. Such cells can be obtained, for example, from a biopsy or surgical procedure performed on the patient. Alternatively, in allogeneic ex vivo therapy, cells can be obtained from a cell bank or a cell line derived from iPS cells, transfected with mRNAs encoding cellular reprogramming factors, as described herein, and reimplanted in the patient. Alternatively, cells in need of rejuvenation can be transfected directly in vivo with mRNAs encoding cellular reprogramming factors.

In another aspect, provided herein are pharmaceutical compositions including rejuvenated cells obtained by transfecting cells with lipid-containing compositions or lipid-nanoparticle compositions of the disclosure comprising one or more non-integrative messenger RNAs encoding one or more cellular reprogramming factors for not more than 4, 5, 6, 7, 8, 9, 10, 11, 12, 13, or 14 continuous days, to transiently reprogram the cells for rejuvenation. In another aspect, provided herein are pharmaceutical compositions including rejuvenated cells obtained by transfecting cells with lipid-containing compositions or lipid-nanoparticle compositions of the disclosure comprising one or more non-integrative messenger RNAs encoding one or more cellular reprogramming factors for not more than 4, 5, 6, or 7 continuous days, to transiently reprogram the cells for rejuvenation.

In some embodiments, lipid-containing compositions or lipid-nanoparticle compositions of the disclosure are used for delivery of mRNA expressing reprogramming factors that provide more robust cellular rejuvenation because the reprogramming factors have been optimized to decrease any triggered immune response to the protein/polypeptide, increase stability of the protein/polypeptide, and altered protein/polypeptide activity, such as increased activity when compared to wild-type reprogramming factors.

In some embodiments, the methods of the disclosure comprise administering lipid-containing compositions or lipid-nanoparticle compositions of the disclosure comprising RNA to a cell or subject, or treating or transfecting a cell with lipid-containing compositions or lipid-nanoparticle compositions of the disclosure comprising RNA for a dosing interval of not more than 25, 24, 23, 22, 21, 20, 19, 18, 17, 16, 15, 14, 13, 12, 11, 10, 9, 8, 7, 6, 5, 4, or 3 consecutive days.

In some embodiments, the dosing of the lipid-containing compositions or lipid-nanoparticle compositions of the disclosure comprising RNA is performed at least once daily during the dosing interval. In some embodiments, the dosing is performed less frequently than once per day during the dosing interval, for example once every two days, once every three days, once every four days, once every x days, where x is a number from 4 to 25. Thus, in such embodiments, for example, dosing lipid-containing compositions or lipid-nanoparticle compositions of the disclosure comprising RNA once every 5 days in a 5 day dosing interval means that the RNA is dosed once in the interval, i.e., once in the total treatment period of 5 days, whereas dosing RNA twice daily in a 5 day dosing interval means that the RNA is dosed 10 times in the interval, i.e., 10 times in the 5 days. In some embodiments, the methods of the disclosure comprise administering lipid-containing compositions or lipid-nanoparticle compositions of the disclosure comprising RNA to a cell or subject, or treating or transfecting a cell with lipid-containing compositions or lipid-nanoparticle compositions of the disclosure comprising RNA for not more than 21, 18, 14, 10, 7, or 5 consecutive days. In some embodiments, the methods of the disclosure comprise administering lipid-containing compositions or lipid-nanoparticle compositions of the disclosure comprising RNA to a cell or subject, or treating or transfecting a cell with lipid-containing compositions or lipid-nanoparticle compositions of the disclosure comprising RNA, for not more than 18 consecutive days In some embodiments, the methods of the disclosure comprise administering lipid-containing compositions or lipid-nanoparticle compositions of the disclosure comprising RNA to a cell or subject, or treating or transfecting a cell with lipid-containing compositions or lipid-nanoparticle compositions of the disclosure comprising RNA for not more than 14 consecutive days. In some embodiments, the methods of the disclosure comprise administering lipid-containing compositions or lipid-nanoparticle compositions of the disclosure comprising RNA to a cell or subject, or treating or transfecting a cell with lipid-containing compositions or lipid-nanoparticle compositions of the disclosure comprising RNA for not more than 10 consecutive days. In some embodiments, the methods of the disclosure comprise administering lipid-containing compositions or lipid-nanoparticle compositions of the disclosure comprising RNA to a cell or subject, or treating or transfecting a cell with lipid-containing compositions or lipid-nanoparticle compositions of the disclosure comprising RNA for not more than 7 consecutive days. In some embodiments, the methods of the disclosure comprise administering lipid-containing compositions or lipid-nanoparticle compositions of the disclosure comprising RNA to a cell or subject, or treating or transfecting a cell with lipid-containing compositions or lipid-nanoparticle compositions of the disclosure comprising RNA for not more than 5 consecutive days. In other embodiments, said exposing comprises interrupting said exposing and repeating said exposing after said interrupting.

In some embodiments, said exposing, treating, transfecting, expressing, or administering comprises exposing, treating, transfecting, expressing, or administering for between about 2-5 consecutive days, between about 5-7 consecutive days, between about 7-10 consecutive days, between about 10-12 consecutive days, between about 12-14 consecutive days, between about 14-17 consecutive days, between about 17-19 consecutive, or between about 19-21 consecutive days and in some embodiments, further comprises interrupting said exposing and repeating said exposing after said interrupting.

In some embodiments, the duration of exposure is controlled by mechanisms such as self-amplifying RNA, circular RNA, B18R and other decoys, and/or on/off switches such as L⁷Ae or its family members. In some embodiments, said repeating is performed any number of times, for example 1, 2, 3, 4, 5, 6, 7, 8, 9, or 10 times, or up to 20 times, or up to 30 times, or more. For in vivo applications, said repeating may continue for any duration of time, for example until a disease is successfully treated or cured, or throughout the life of a subject or patient. In some embodiments, said repeating is performed any time after said interrupting, for example 0, 1, 2, 3, 4, 5, 6, 7, 8, 9, or 10 days, up to 20 days, up to 30 days, up to 3 months, up to 6 months, or up to 1 year after said interrupting. One exposure period is considered to be a dosing interval, such that, for example, a sequence of exposure-interruption-repeat exposure contains two dosing intervals.

In one embodiment, a compound having a structure of Formula (XVII) is provided:

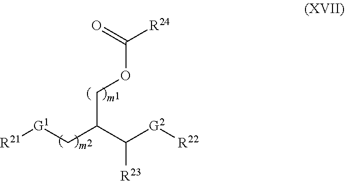

or a stereoisomer, salt, or tautomer thereof, G¹, G², R²¹, R²², R²³, R²⁴, m¹, and m² are as defined herein.

In one embodiment, a compound having a structure of Formula (XVIII) is provided:

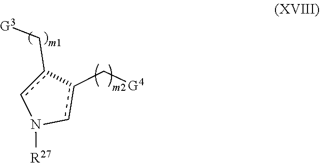

or a stereoisomer, salt, or tautomer thereof, G³, G⁴, R²⁷, m¹, and m² are as defined herein.

In one embodiment, a compound having a structure of Formula (XIX) is provided:

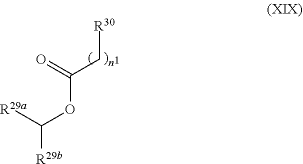

or a stereoisomer, salt, or tautomer thereof, R^{29a}, R^{29b}, R³⁰, and n are as defined herein.

Pharmaceutical compositions comprising one or more of the foregoing compounds of Formula (XVII)-(XIX) and a therapeutic agent are also provided.

In other embodiments, methods of treatment by administering the foregoing compounds of Formula (I) or the pharmaceutical compositions comprising a compound of Formula (XVII)-(XIX), to a subject in need thereof to treat a disease is provide.

In one embodiment, a lipid nanoparticle comprising one or more of the foregoing compounds of Formula (XVII)-(XIX) and a therapeutic agent comprising a nucleic acid are also provided.

Various aspects and embodiments now will be described more fully hereinafter. Such aspects and embodiments make take many different forms and the exemplary ones disclosed herein should not be construed as limiting; rather, these embodiments are provided so that this disclosure will be thorough and complete, and will fully convey its scope to those skilled in the art.

### DETAILED DESCRIPTION

The present disclosure, at least in part, relates to ionizable lipids that can provide certain advantages when used in nanoparticle compositions for delivery of an active or therapeutic agent such as a nucleic acid into a cell.

The ionizable lipids of the disclosure comprise an ionizable group, one or more ester groups and one or more hydrophobic tail groups. The ionizable group is an amine containing head group with an acid dissociation constant (pKa) above 7. Without wishing to be bound by any theory, it is thought that high pKa allows ionizable lipids to be positively charged at acidic pH (<6.0) and neutral at physiological pH (7.4). This in turn results in high encapsulation efficiencies for nucleic acids at acidic pH. Ionizable lipids and other helper lipids also interact with negatively charged membranes of the endosome that results in their membrane disruption and thereby facilitate the release of nucleic acids.

The one or more ester groups in the ionizable lipids of the disclosure bestows biodegradability, as it is a key feature for the clinical translation of the ionizable lipids. The inclusion of ester bonds into the ionizable groups reduce accumulation and potential side effects, thereby leading to the degradation of the ionizable lipids into non-toxic metabolites after successful delivery of the intracellular cargo, such as a nucleic acid, small molecule, or peptide or protein.

The one or more hydrophobic tail groups comprise of aliphatic carbon chains. The aliphatic carbon chains can further include unsaturated bonds, such as double bonds or triple bonds. The hydrophobic tail can further include branched tails. It is believed that tail length and tail saturation can greatly influence the fluidity and delivery efficiency of ionizable lipids. The molecular hypothesis is that the ionizable lipids of the disclosure are expected to produce a cone-shape structure with enhanced endosome-disrupting ability due to increased cross-section of the tail region. This in turn facilitates the endosomal release due to protonation of the ionizable lipids at the endosomal pH. The nanoparticle compositions of the disclosure comprising the ionizable lipids are extremely useful for systemic delivery applications, as they can exhibit extended circulation lifetimes and can mediate expression of the transfected gene or silencing of target gene expression in vivo. The nanoparticle compositions of the disclosure comprising the ionizable lipids are extremely useful for localized delivery applications, as their properties such as but not limited to ionization constant, size, and surface charge can be tuned for delivery and transfection to specific tissues.

The design of ionizable lipids is inspired by lipid based natural products such as Cephalin and Sphingomyelin and other common compounds like glycerides to target better in vivo delivery and clearance thereafter. It is also believed that such lipids could also help in higher transfection efficiency and cell selectivity.

Introduction of aromatic rings in the hydrophobic chains can introduce more organized architecture by virtue of pi-stacking which can lead to better packing and stability of LNPs. Aromatic rings also provide an easy handle for further structural modifications to alter the cone shape by introducing more aliphatic chains and/or by altering the point of attachment at different positions.

Use of heterocyclics in lipid head groups is intended to provide more defined cone shaped lipids and better nucleic acid encapsulation.

### I. DEFINITIONS

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Where a range of values is provided, it is intended that each intervening value between the upper and lower limit of that range and any other stated or intervening value in that stated range is encompassed within the disclosure. For example, if a range of 1 mg to 8 mg is stated, it is intended that 2 mg, 3 mg, 4 mg, 5 mg, 6 mg, and 7 mg are also explicitly disclosed, as well as the range of values greater than or equal to 1 mg and the range of values less than or equal to 8 mg.

As used herein, the term “Cn-m alkyl”, refers to a saturated hydrocarbon group that may be straight-chain or branched. An alkyl group formally corresponds to an alkane with one C—H bond replaced by the point of attachment of the alkyl group to the remainder of the compound. The term “Cn-m alkyl” refers to an alkyl group having n to m carbon atoms. Examples of alkyl groups include, but are not limited to, chemical groups such as methyl, ethyl, n-propyl, iso-propyl, n-butyl, tert-butyl, iso-butyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms. In some embodiments, the alkyl group contains from 6 to 20 carbon atoms, from 6 to 18 carbon atoms, from 6 to 17 carbon atoms, from 6 to 15 carbon atoms, from 6 to 14 carbon atoms, from 6 to 13 carbon atoms, from 6 to 9 carbon atoms, from 6 to 8 carbon atoms, or 6 to 7 carbon atoms. In some embodiments, the alkyl group is optionally substituted with 1, 2, 3 or more halo groups. In some embodiments, the alkyl group is optionally substituted with 1, 2, 3 or more hetero groups.

As used herein, the term “Cn-m alkenyl”, refers to an unsaturated hydrocarbon group that may be straight-chain or branched corresponding to an alkyl group having one or more carbon-carbon double bonds. An alkenyl group formally corresponds to an alkene with one C—H bond replaced by the point of attachment of the alkenyl group to the remainder of the compound. The term “Cn-m alkenyl” refers to an alkenyl group having n to m carbons. Examples of alkenyl groups include, but are not limited to, chemical groups such as ethenyl, propenyl, iso-propenyl, n-butenyl, sec-butenyl and the like. In some embodiments, the alkenyl group contains from 6 to 20 carbon atoms, from 6 to 18 carbon atoms, from 6 to 17 carbon atoms, from 6 to 15 carbon atoms, from 6 to 14 carbon atoms, from 6 to 13 carbon atoms, from 6 to 9 carbon atoms, from 6 to 8 carbon atoms, or 6 to 7 carbon atoms. In some embodiments, the alkenyl group is optionally substituted with 1, 2, 3 or more halo groups. In some embodiments, the alkenyl group is optionally substituted with 1, 2, 3 or more hetero groups.

As used herein, the term “Cn-m alkylene”, refers to a divalent alkyl linking group. An alkylene group formally corresponds to an alkane with two C—H bonds replaced by points of attachment of the alkylene group to the remainder of the compound. The term “Cn-m alkylene” refers to an alkylene group having n to m carbon atoms. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl and the like. In some embodiments, the alkylene group is optionally substituted with 1, 2, 3 or more halo groups. In some embodiments, the alkylene group is optionally substituted with 1, 2, 3 or more hetero groups.

As used herein, the terms “approximately” and “about” refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a lipid component of a nanoparticle composition, “about” may mean+/−10% of the recited value. For instance, a nanoparticle composition including a lipid component having about 40% of a given lipid may include 30-50% of the lipid.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

As used herein, the term “encapsulated” can refer to a lipid particle that provides a payload such as a nucleic acid (e.g., an interfering RNA, plasmid or oligonucleotide DNA, or mRNA), with full encapsulation, partial encapsulation, or both. In one embodiment, the nucleic acid is fully encapsulated in the lipid particle to form a lipid nanoparticle (LNP). The term “fully encapsulated” indicates that the nucleic acid in the lipid particle is not significantly degraded after exposure to serum or a nuclease or protease assay that would significantly degrade free RNA, or protein. Full encapsulation may be determined by an Oligreen® assay. Oligreen® is an ultra-sensitive fluorescent nucleic acid stain for quantitating RNA in solution (available from Invitrogen Corporation; Carlsbad, California). “Fully encapsulated” also indicates that the lipid particles are serum-stable, that is, that they do not rapidly decompose into their component parts upon in vivo administration. In one embodiment, the nucleic acid is at least 50% encapsulated in the lipid. In one embodiment, the nucleic acid is at least 75% encapsulated in the lipid. In one embodiment, the nucleic acid is at least 90% encapsulated in the lipid. In one embodiment, the nucleic acid is completely encapsulated in the lipid.

As used herein, the terms “halo” or “halogen” refers to F, Cl, Br, or I.

As used herein, the term “hetero” refers to heteroatoms selected from oxygen, nitrogen and sulfur.

As used herein, the term “interfering RNA” refers to single-stranded RNA (e.g., mature miRNA, circular RNA, guide RNA) or double-stranded RNA (i.e., duplex RNA such as siRNA, aiRNA, or pre-miRNA), or an RNA vector that is capable of reducing or inhibiting the expression of a target gene or sequence (e.g., by mediating the degradation or inhibiting the translation of mRNAs which are complementary to the interfering RNA sequence) when the interfering RNA is in the same cell as the target gene or sequence. Interfering RNA thus refers to the single-stranded RNA that is complementary to a target mRNA sequence or to the double-stranded RNA formed by two complementary strands or by a single, self-complementary strand. Interfering RNA may have substantial or complete identity to the target gene or sequence, or may comprise a region of mismatch (i.e., a mismatch motif). The sequence of the interfering RNA can correspond to the full-length target gene, or a subsequence thereof.

Interfering RNA includes “small-interfering RNA” or “siRNA,” e.g., interfering RNA of about 15-60, 15-50, or 15-40 (duplex) nucleotides in length, more typically about 15-30, 15-25, or 19-25 (duplex) nucleotides in length, and is preferably about 20-24, 21-22, or 21-23 (duplex) nucleotides in length (e.g., each complementary sequence of the double-stranded siRNA is 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 18-22, 19-20, or 19-21 base pairs in length). siRNA duplexes may comprise 3′ overhangs of about 1 to about 4 nucleotides or about 2 to about 3 nucleotides and 5′ phosphate termini. Examples of siRNA include, without limitation, a double-stranded polynucleotide molecule assembled from two separate stranded molecules, wherein one strand is the sense strand and the other is the complementary antisense strand; a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule.

siRNA can also be chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Natl. Acad. Sci. USA, 99:9942-9947 (2002); Calegari et al., Proc. Natl. Acad. Sci. USA, 99: 14236 (2002); Byrom et al., Ambion TechNotes, 10(1):4-6 (2003); Kawasaki et al., Nucleic Acids Res., 31:981-987 (2003); Knight et al., Science, 293:2269-2271 (2001); and Robertson et al., J. Biol. Chem., 243:82 (1968)). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

As used herein, the term “in vitro” refers to events that occur in an artificial environment, e.g., in a test tube or reaction vessel, in cell culture, in a petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “rejuvenated cell(s)” refers to aged cells that have been treated or transiently reprogrammed with one or more cellular reprogramming factors such that the cells have a transcriptomic profile of a younger cell while still retaining one or more cell identity markers. In some embodiments, treated cells are rejuvenated and reprogrammed to express markers and a transcriptomic profile of a younger cell while still retaining cell identity markers, such as rejuvenated cells being reprogrammed to express at least 1%, 2%, 3%, 4%, 5%, 10%, 15%, 20%, 25%, 30%, 40%, 50%, 60%, 70%, 75% or more than 75% increase/improvement in expression of at least one rejuvenation marker compared to untreated cells.

As used herein, the terms “subject,” “individual,” and “patient,” are used interchangeably herein and refer to any vertebrate subject, including, without limitation, humans and other primates, including non-human primates such as chimpanzees and other apes and monkey species; farm animals such as cattle, sheep, pigs, goats and horses; domestic mammals such as dogs and cats; rodents such as mice, rats, rabbits, hamsters, and guinea pigs; birds, including domestic, wild and game birds such as chickens, turkeys and other gallinaceous birds, ducks, geese, and the like. In some cases, the methods of the disclosure find use in experimental animals, in veterinary application, and in the development of animal models for disease. The term does not denote a particular age. Thus, adult, juvenile, and newborn individuals are intended to be covered.

As used herein, the term “transfection” refers to the uptake of exogenous DNA or RNA by a cell. A cell has been “transfected” when exogenous DNA or RNA has been introduced inside the cell membrane.

As used herein, the term “transfection efficiency” refers to the extent to which exogenous DNA or RNA is taken up by cells being transfected. Transfection efficiency can be measured, for example, as the percentage of cells in a sample that express the gene product of transfected exogenous DNA or RNA. The percentage of cells in a sample that express the gene product of transfected exogenous DNA or RNA can be measured by determining the percentage of cells in the sample expressing a reporter gene such a green fluorescent protein at 18-24 hours after transfection using flow cytometry according to methods well known to those skilled in the art.

As used herein, the term “transfection potency” refers to the mean expression level of the gene product of transfected exogenous DNA or RNA per cell in a sample. The mean expression level of the gene product of transfected exogenous DNA or RNA per cell in a sample can be measured at 18-24 hours after transfection by using flow cytometry to determine the total fluorescence intensity of cells in the sample expressing a reporter gene, such a green fluorescent protein, divided by the number of cells in the sample, according to methods well known to those skilled in the art.

As used herein, the term “viability” refers to the extent to which cells remain alive after transfection and is measured as the percentage of cells remaining alive in a sample subjected to transfection. Viability can be measured at 18-24 hours after transfection as the percentage of cells in a sample that stain positive for propium iodide using flow cytometry according to methods well known to those skilled in the art.

As used herein, the term “transient reprogramming” refers to exposure of cells to cellular reprogramming factors for a period of time sufficient to rejuvenate cells (i.e., eliminate all or some hallmarks of aging), but not long enough to cause dedifferentiation into stem cells. Such transient reprogramming results in rejuvenated cells that retain their identity (i.e., differentiated cell-type).

The term “treating” is used herein, for instance, in reference to methods of treating a cell, a tissue or a subject, and generally includes the administration of a compound or composition which reduces the frequency of, or delays the onset of, symptoms of aging or of a medical condition in a subject relative to a subject not receiving the compound or composition. This can include reversing, reducing, or arresting the symptoms, clinical signs, and underlying pathology of a condition in a manner to improve or stabilize a subject's condition.

As used herein, the term “isomer” means any geometric isomer, tautomer, zwitterion, stereoisomer, enantiomer, or diastereomer of a compound. Compounds may include one or more chiral centers and/or double bonds and may thus exist as stereoisomers, such as double-bond isomers (i.e., geometric E/Z isomers) or diastereomers (e.g., enantiomers (i.e., (+) or (−) or cis/trans isomers). The present disclosure encompasses any and all isomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known.

In the present disclosure, the structural formula of the lipids may represent a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. In addition, a crystal polymorphism may be present for the lipids represented by the formulae. It is noted that any crystal form, crystal form mixture, or anhydride or hydrate thereof is included in the scope of the present disclosure.

As used herein, the term “lipid-based delivery system” includes, but is not limited to, liposomes, polyplexes, lipoplexes, and lipid nanoparticles towards delivery of any payload described herein, including but not limited to nucleic acids.

As used herein, the term “lipid nanoparticle” or “LNP” refers to a nanoparticle formed from at least one lipid or a nanoparticle that comprises at least one lipid. “Lipid-nanoparticle compositions” refers to compositions comprising lipid nanoparticles or to the lipid nanoparticles themselves. In some embodiments, the lipid nanoparticle is a lipid-nucleic acid particle or a nucleic acid-lipid particle (e.g., a stable nucleic acid-lipid particle). A LNP can be a particle formed from or comprising the ionizable lipids of the disclosure, and a cargo that is encapsulated within the lipid. In some embodiments, the cargo is a nucleic acid.

The LNP typically can have a mean diameter of from about 10 nm to about 200 nm, from about 15 nm to about 150 nm, from about 20 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

As used herein, the term “mammal” includes humans and both domestic animals such as laboratory animals and household pets (e.g., cats, dogs, swine, cattle, sheep, goats, horses, rabbits), and non-domestic animals such as wildlife and the like.

As used herein, the term “nanoparticle composition” refers to a composition comprising one or more lipids. Nanoparticle compositions are typically sized on the order of micrometers or smaller and may include a lipid bilayer. Nanoparticle compositions encompass lipid nanoparticles (LNPs), liposomes (e.g., lipid vesicles), polyplexes, and lipoplexes. For example, a nanoparticle composition may be a lipid nanoparticle with a diameter of 500 nm or less.

As used herein, the term “nucleic acid” refers to a polymer containing at least two deoxyribonucleotides or ribonucleotides in either single- or double-stranded form and includes DNA and RNA. DNA may be in the form of, e.g., antisense molecules, plasmid DNA, precondensed DNA, a PCR product, vectors (PI, PAC, BAC, YAC, artificial chromosomes), expression cassettes, chimeric sequences, chromosomal DNA, or derivatives and combinations of these groups. RNA may be in the form of siRNA, asymmetrical interfering RNA (aiRNA), microRNA (miRNA), mRNA, tRNA, rRNA, tRNA, viral RNA (vRNA), self-amplifying RNA, polycistronic RNA, self-amplifying polycistronic RNA, circular RNA, and combinations thereof.

Nucleic acids include nucleic acids containing known nucleotide analogs or modified backbone residues or linkages, which are synthetic, naturally occurring, and non-naturally occurring, and which have similar binding properties as the reference nucleic acid. Examples of such modifications or analogs include, without limitation, 5-methylcytidine, 5-methyluridine, 2-thiouridine, N6-methyladenosine, pseudouridine, and N1-methylpseudouridine. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et ah, J. Biol. Chem., 260:2605-2608 (1985); Rossolini et ah, Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkyl halides.

As used herein, the term “optionally substituted” means unsubstituted or substituted. As used herein, the term “substituted” means that a hydrogen atom is removed and replaced by a substituent. It is to be understood that substitution at a given atom is limited by valency.

As used herein, the term “pharmaceutically acceptable” refer to those compounds, salts, compositions, dosage forms, etc., which are—within the scope of sound medical judgment—suitable for use in contact with the tissues of human beings and/or other mammals without excessive toxicity, irritation, allergic response, or other problem or complication, commensurate with a reasonable benefit/risk ratio. In some aspects, “pharmaceutically acceptable” means approved by a regulatory agency of the federal or a state government, or listed in the U.S. Pharmacopeia or other generally recognized pharmacopeia for use in mammals (e.g., animals), and more particularly, in humans.

As used herein, the term “pharmaceutically acceptable carrier” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals. In some aspects, the “pharmaceutically acceptable carrier” or carrier, adjuvant, excipient therein is a component of “artificial niche” used to maintain quiescence of progenitor cells. In some embodiments, the artificial niche component is selected from the group consisting of elcatonin; MGCD-265, JNJ-7706621, forskolin, fomatostatin, SB203580, SU5402, TGF-□, insulin-transferrin-selenium, and combinations thereof. These and other “artificial niche” components are disclosed in U.S. Pat. No. 10,688,136, incorporated herein by reference. When used as an excipient, such artificial niche components may be encapsulated by a lipid nanoparticle, present in the shell of a lipid nanoparticle, or included in the formulation separately from a lipid nanoparticle.

As used herein, the term “pharmaceutically acceptable salt” includes both acid and base addition salts.

As used herein, the term “pharmaceutical composition” refers to the nanoparticle composition and a medium generally accepted in the art for the delivery of the biologically active compound to mammals, such as pharmaceutically acceptable carriers.

As used herein, the term “subject” or “patient” refers to any organism to which a composition in accordance with the disclosure may be administered, e.g., for experimental, diagnostic, prophylactic, and/or therapeutic purposes. Typical subjects include animals, such as, but not limited to mammals, such as, but not limited to mice, rats, rabbits, non-human primates, and humans.

As used herein, the term “systemic delivery” refers to the delivery of a therapeutic product that can result in a broad exposure of an active agent within an organism. Some techniques of administration can lead to the systemic delivery of certain agents, but not others. Systemic delivery means that a useful, preferably therapeutic, amount of an agent is exposed to most parts of the body.

Systemic delivery of the compositions of the present disclosure, e.g., lipid nanoparticles, can be by any means known in the art including, for example, intravenous, intraarterial, subcutaneous, and intra peritoneal delivery. In some embodiments, systemic delivery of lipid nanoparticles is by intravenous delivery.

As used herein, the term “local delivery” refers to the delivery of a therapeutic product that can result in a localized exposure of an active agent within a particular location, tissue, organ, or cell type within an organism. Some ionized lipid compositions, including some lipid nanoparticles, can lead to the localized delivery of certain agents, but not others. Localized delivery means that a useful, preferably therapeutic, amount of an agent is exposed to a particular location, tissue, organ, or cell type within an organism. Localized delivery of the compositions of the present disclosure, e.g., lipid nanoparticles, can be by any means known in the art including, for example, intravenous, intraarterial, subcutaneous, intradermal, dermal, topical, intra-tissue, intra-organ, and intra peritoneal delivery. In some embodiments, localized delivery of the compositions of the present disclosure, e.g., lipid nanoparticles, occurs by intravenous delivery, where properties of nanoparticles such as size, shape, ionization constant, and surface charge determine the particular location, tissue, organ, or cell type targeted by the LNCs. In some embodiments, localized delivery of the compositions of the present disclosure, e.g., lipid nanoparticles, occurs by local injection into the targeted location, tissue, or organ, where the compositions of the present disclosure, e.g., lipid nanoparticles, then enable transfection in that local environment. In some embodiments, localized delivery of the compositions of the present disclosure, e.g., lipid nanoparticles, occurs by dermal, intradermal, or subcutaneous injection, where the compositions of the present disclosure, e.g., lipid nanoparticles, then enable transfection in that local environment. In some embodiments, localized delivery of the compositions of the present disclosure, e.g., lipid nanoparticles, occurs by topical administration, where the compositions of the present disclosure, e.g., lipid nanoparticles, then enable transfection in that local environment.

As used herein, the term “therapeutic agent” or “prophylactic agent” refers to any agent that, when administered to a subject, has a therapeutic, diagnostic, and/or prophylactic effect and/or elicits a desired biological and/or pharmacological effect. Therapeutic agents are also referred to as “actives” or “active agents.”

As used herein, the term “effective amount” or “therapeutically effective amount” refers to that amount of the nanoparticle composition which, when administered to a mammal, preferably a human, is sufficient to effect treatment in the mammal, preferably a human. The amount of a lipid nanoparticle of the disclosure which constitutes a “therapeutically effective amount” will vary depending on the nature of the compositions of the present disclosure, e.g., the particular lipid nanoparticles used, the condition and its severity, the manner of administration, and the age of the subject to be treated, but can be determined routinely by one of ordinary skill in the art having regard to his own knowledge and to this disclosure.

As used herein, the term “treating” or “treatment” as used herein covers the treatment of the disease or condition of interest in a subject, preferably a mammal, more preferably a human, having the disease or condition of interest, and includes preventing the disease or condition from occurring, in particular, when such subject is predisposed to the condition but has not yet been diagnosed as having it; inhibiting the disease or condition, i.e., arresting its development; relieving the disease or condition, i.e., causing regression of the disease or condition; or relieving symptoms resulting from the disease or condition, e.g., relieving pain without addressing the underlying disease or condition.

As used herein, the term “zeta potential” refers to the electrokinetic potential of a lipid in the nanoparticle composition or the electrokinetic potential of a nanoparticle, for example that of a lipid nanoparticle.

The compositions of the present disclosure can comprise, consist essentially of, or consist of, the components disclosed.

All percentages, parts and ratios are based upon the total weight of the compositions and all measurements made are at about 25° C., unless otherwise specified.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

### II. IONIZABLE LIPIDS

The ionizable lipids of the disclosure are positively charged at acidic pH to condense nucleic acids such as RNAs into lipid nanoparticles (LNPs). The ionizable lipids are neutral at physiological pH to minimize toxicity. They can be protonated in the acidic endosome after cellular uptake, and interact with anionic endosomal phospholipids to form cone-shaped ion pairs that are not compatible with a bilayer. These cationic-anionic lipid pairs drive the transition from the bilayer structure to the inverted hexagonal HII phase, which facilitates membrane fusion/disruption, endosomal escape and cargo release into the cytosol (Semple, S. C. et al., Nat. Biotechnol. 2010, 28, 172-176).

The pKa of the ionizable lipids disclosed herein is about 8.5 to about 9.5. Lipid nanoparticles comprising one or more of the ionizable lipids described herein can have a pKa of about 6.5 to about 7.5.

The disclosure provides ionizable lipids of Formula (I) to Formula (XVI). These lipids may have a positive or partial positive charge at physiological pH.

In one embodiment, the ionizable lipid is of Formula (I):

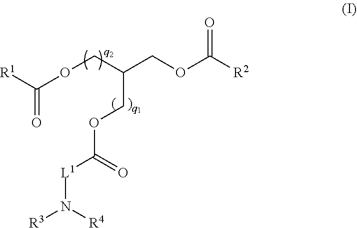

or a pharmaceutically acceptable salt or a stereoisomer thereof, wherein:

L¹ is C₁-C₆ alkylene; R¹ is C₆-C₂₀ alkenyl; R² is C₆-C₂₀ alkyl; R³ and R⁴ are each independently H or C₁-C₃ alkyl; q₁ is absent or 1; and q₂ is absent or 1.

In some embodiments, L1 is C1-C6 alkylene; R1 is C6-C20 alkenyl; R2 is C6-C20 alkyl; R3 is methyl; R4 is methyl; q1 is absent; and q2 is absent in Formula (I).

In some embodiments, L1 is C1-C6 alkylene; R1 is C6-C20 alkenyl; R2 is C6-C20 alkyl; R3 is methyl; R4 is methyl; q1 is 1; and q2 is 1 in Formula (I).

In some embodiments, L1 is C1-C6 alkylene; R1 is C6-C20 alkenyl; R2 is C6-C20 alkyl; R3 is methyl; R4 is methyl; q1 is absent; and q2 is 1 in Formula (I).

In some embodiments, L1 is C1-C6 alkylene; R1 is C6-C20 alkenyl; R2 is C6-C20 alkyl; R3 is methyl; R4 is methyl; q1 is 1; and q2 is absent in Formula (I).

Exemplary examples of ionizable lipid of Formula (I) can include, but not limited to the following:

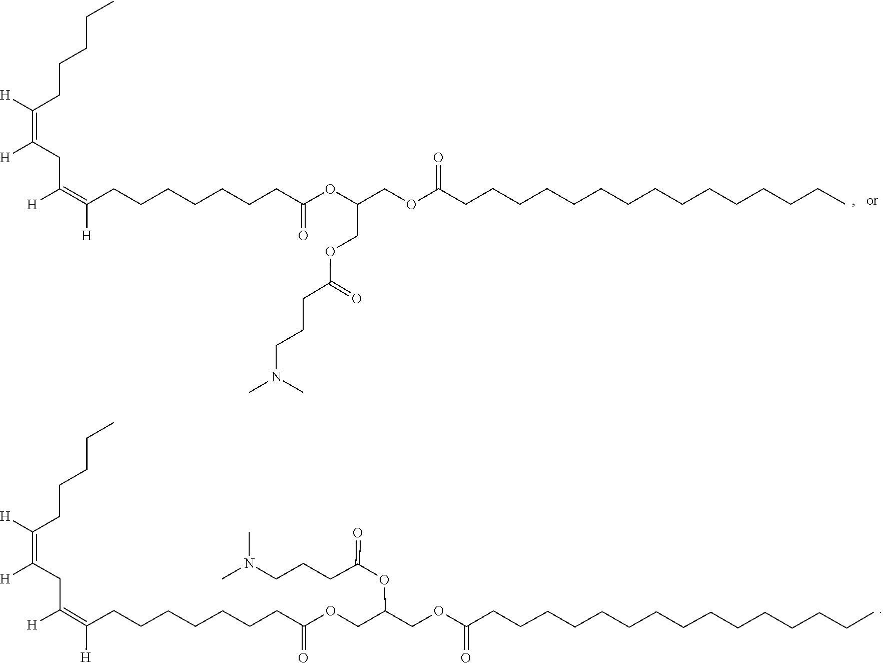 [SMILES: CCCCC/C=C\C/C=C\CCCCCCCC(=O)OC(COC(=O)CCCCCCCCCCCCCCC)COC(=O)CCCN(C)C ; CCCCC/C=C\C/C=C\CCCCCCCC(=O)OCC(COC(=O)CCCCCCCCCCCCCCC)OC(=O)CCCN(C)C]

In one embodiment, the ionizable lipid is of Formula (I-A):

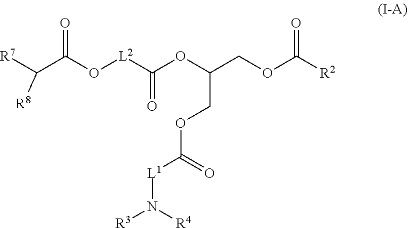

or a pharmaceutically acceptable salt or a stereoisomer thereof, wherein:

L¹ is C₁-C₆ alkylene; L² is C₁-C₈ alkylene; R² is C₆-C₂₀ alkyl; R³ and R⁴ are each independently H or C₁-C₃ alkyl; R⁷ is C₄-C₂₀ alkyl; and R⁸ is C₄-C₂₀ alkyl.

In some embodiments, L¹ is C₁-C₆ alkylene; L² is C₁-C₈ alkylene; R² is C₆-C₂₀ alkyl; R³ is methyl; R⁴ is methyl; R⁷ is C₄-C₂₀ alkyl; and R⁸ is C₄-C₂₀ alkyl in Formula (I-A).

Exemplary examples of ionizable lipid of Formula (I-A) can include, but not limited to the following:

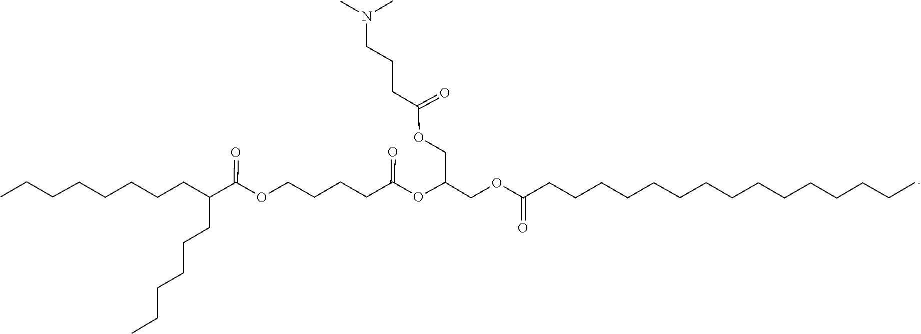 [SMILES: CCCCCCCCCCCCCCCC(=O)OCC(COC(=O)CCCN(C)C)OC(=O)CCCCOC(=O)C(CCCCCC)CCCCCCCC]

In one embodiment, the ionizable lipid is of Formula (I-B):

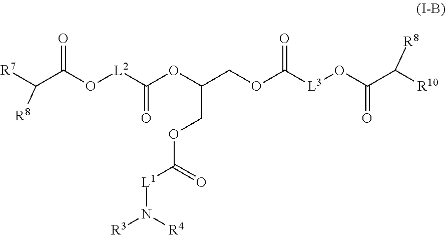

or a pharmaceutically acceptable salt or a stereoisomer thereof, wherein:

L¹ is C₁-C₆ alkylene; L² is C₁-C₈ alkylene; L³ is C₁-C₈ alkylene; R³ and R⁴ are each independently H or C₁-C₃ alkyl; R⁶ is C₄-C₂₀ alkyl; R⁷ is C₄-C₂₀ alkyl; R⁸ is C₄-C₂₀ alkyl; and R¹⁰ is C₄-C₂₀ alkyl.

In some embodiments, L¹ is C₁-C₆ alkylene; L² is C₁-C₈ alkylene; L³ is C₁-C₈ alkylene; R³ is methyl; R⁴ is methyl; R⁶ is C₄-C₂₀ alkyl; R⁷ is C₄-C₂₀ alkyl; R⁸ is C₄-C₂₀ alkyl; and R¹⁰ is C₄-C₂₀ alkyl in Formula (I-B).

Exemplary examples of ionizable lipid of Formula (I-B) can include, but not limited to the following:

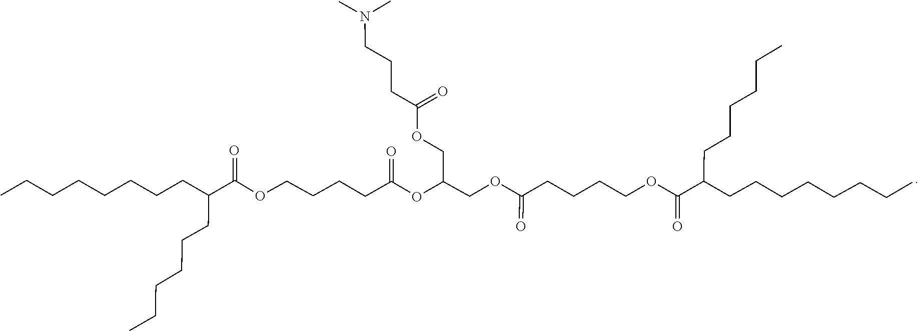 [SMILES: CCCCCCCCC(CCCCCC)C(=O)OCCCCC(=O)OCC(COC(=O)CCCN(C)C)OC(=O)CCCCOC(=O)C(CCCCCC)CCCCCCCC]

In one embodiment, the ionizable lipid is of Formula (II):

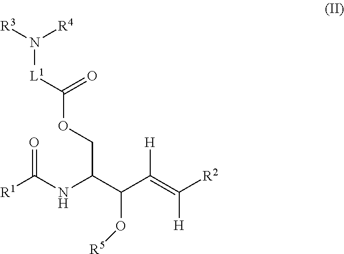

or a pharmaceutically acceptable salt or a stereoisomer thereof, wherein:

L¹ is C₁-C₆ alkylene; R¹ is C₆-C₂₀ alkenyl; R² is C₆-C₂₀ alkyl; R³ and R⁴ are each independently H or C₁-C₃ alkyl; and R⁵ is H or C₁-C₃ alkyl.

In some embodiments, L¹ is C₁-C₆ alkylene; R¹ is C₆-C₂₀ alkenyl; R² is C₆-C₂₀ alkyl; R³ is methyl; R⁴ is methyl; and R⁵ is H in Formula (II).

In some embodiments, L¹ is C₁-C₆ alkylene; R¹ is C₆-C₂₀ alkenyl; R² is C₆-C₂₀ alkyl; R³ is methyl; R⁴ is methyl; and R⁵ is methyl in Formula (II).

Exemplary examples of ionizable lipid of Formula (II) can include, but not limited to the following:

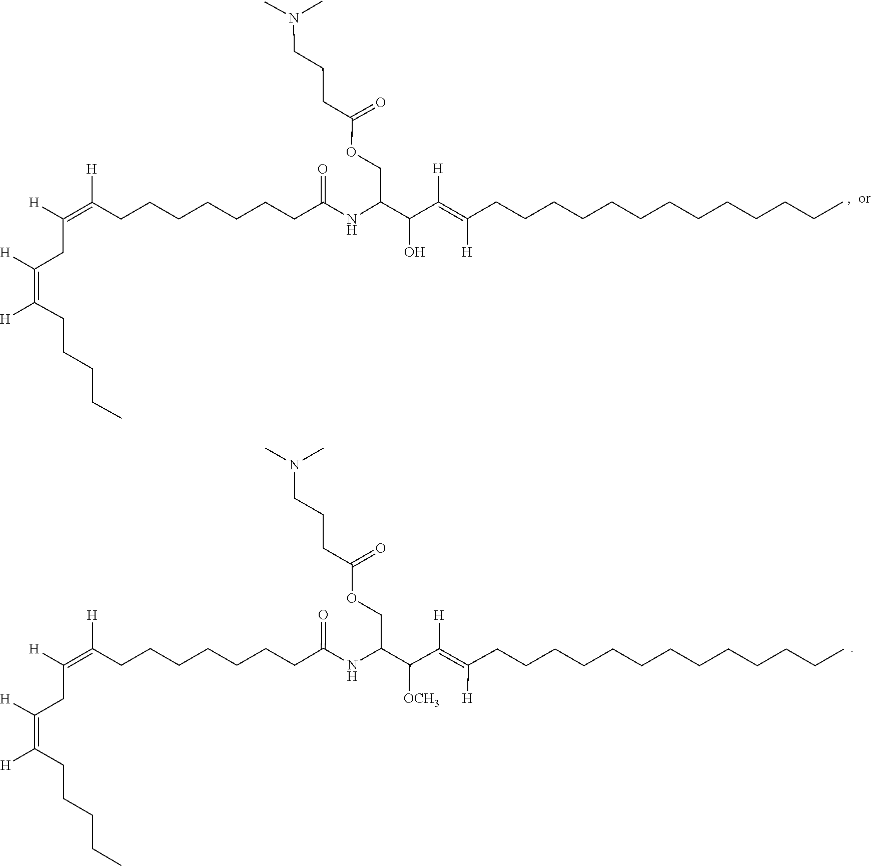 [SMILES: CCCCC/C=C\C/C=C\CCCCCCCC(=O)NC(COC(=O)CCCN(C)C)C(O)/C=C/CCCCCCCCCCCCC ; CCCCC/C=C\C/C=C\CCCCCCCC(=O)NC(COC(=O)CCCN(C)C)C(/C=C/CCCCCCCCCCCCC)OC]

In one embodiment, the ionizable lipid is of Formula (III):

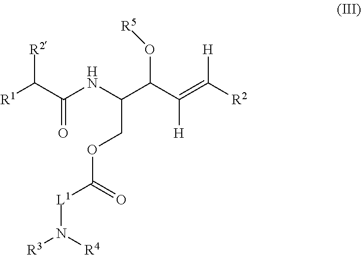

or a pharmaceutically acceptable salt or a stereoisomer thereof, wherein:

L¹ is C₁-C₆ alkylene; R¹ is C₆-C₂₀ alkenyl; R² is C₆-C₂₀ alkyl; R^{2′} is C₆-C₂₀ alkyl; R³ and R⁴ are each independently H or C₁-C₃ alkyl; and R⁵ is H or C₁-C₃ alkyl.

In some embodiments, L¹ is C₁-C₆ alkylene; R¹ is C₆-C₂₀ alkenyl; R² is C₆-C₂₀ alkyl; R^{2′} is C₆-C₂₀ alkyl; R³ is methyl; R⁴ is methyl; and R⁵ is H in Formula (III).

In some embodiments, L¹ is C₁-C₆ alkylene; R¹ is C₆-C₂₀ alkenyl; R² is C₆-C₂₀ alkyl; R^{2′} is C₆-C₂₀ alkyl; R³ is methyl; R⁴ is methyl; and R⁵ is methyl in Formula (III).

Exemplary examples of ionizable lipid of Formula (III) can include, but not limited to the following:

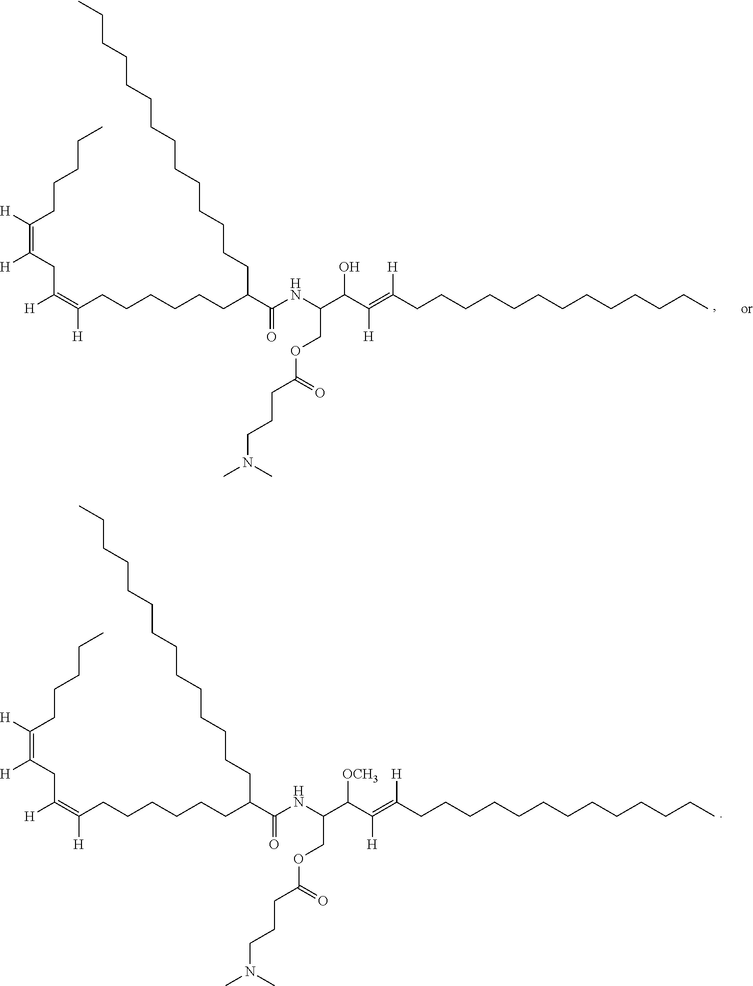 [SMILES: CCCCC/C=C\C/C=C\CCCCCCC(CCCCCCCCCCCCCC)C(=O)NC(COC(=O)CCCN(C)C)C(O)/C=C/CCCCCCCCCCCCC ; CCCCC/C=C\C/C=C\CCCCCCC(CCCCCCCCCCCCCC)C(=O)NC(COC(=O)CCCN(C)C)C(/C=C/CCCCCCCCCCCCC)OC]

In one embodiment, the ionizable lipid is of Formula (IV):

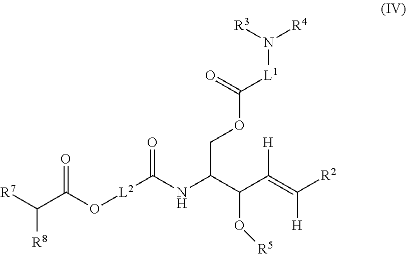

or a pharmaceutically acceptable salt or a stereoisomer thereof, wherein:

L¹ is C₁-C₆ alkylene; L² is C₁-C₈ alkylene; R² is C₆-C₂₀ alkyl; R³ and R⁴ are each independently H or C₁-C₃ alkyl; R⁵ is H or C₁-C₃ alkyl; R⁷ is C₄-C₂₀ alkyl; and R⁸ is C₄-C₂₀ alkyl.

In some embodiments, L¹ is C₁-C₆ alkylene; L² is C₁-C₈ alkylene; R² is C₆-C₂₀ alkyl; R³ is methyl; R⁴ is methyl; R⁵ is H; R⁷ is C₄-C₂₀ alkyl; and R⁸ is C₄-C₂₀ alkyl in Formula (IV).

In some embodiments, L¹ is C₁-C₆ alkylene; L² is C₁-C₈ alkylene; R² is C₆-C₂₀ alkyl; R³ is methyl; R⁴ is methyl; R⁵ is methyl; R⁷ is C₄-C₂₀ alkyl; and R⁸ is C₄-C₂₀ alkyl in Formula (IV).

Exemplary examples of ionizable lipid of Formula (IV) can include, but not limited to the following:

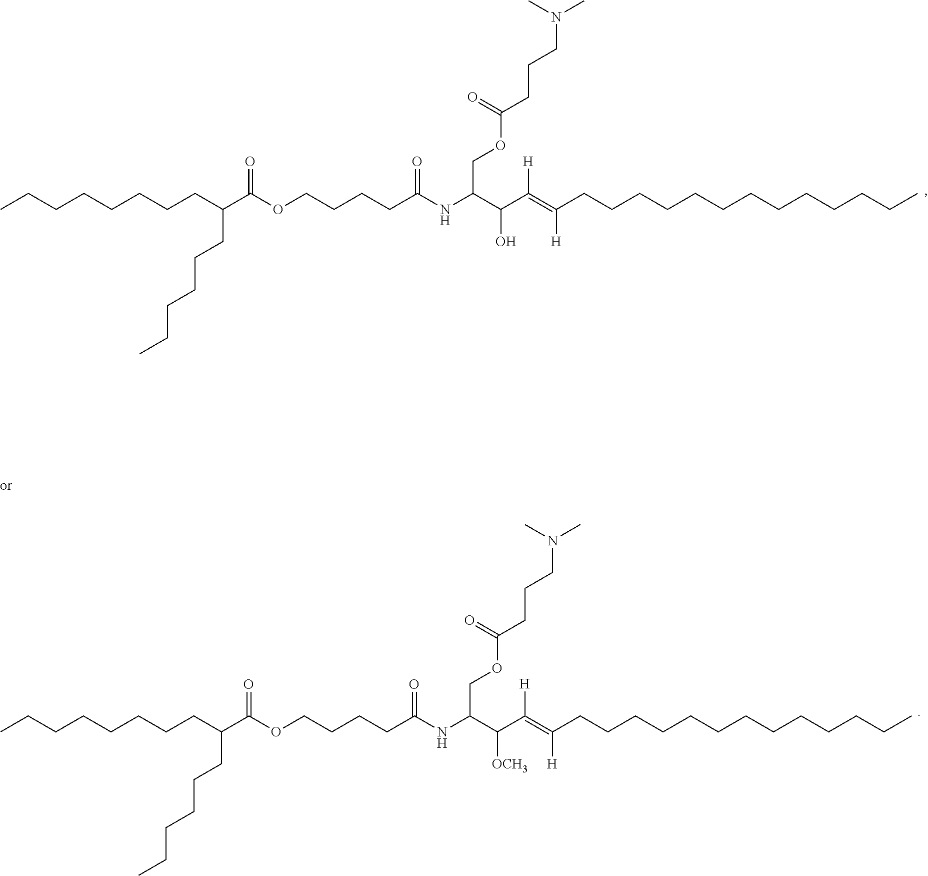

In one embodiment, the ionizable lipid is of Formula (V):

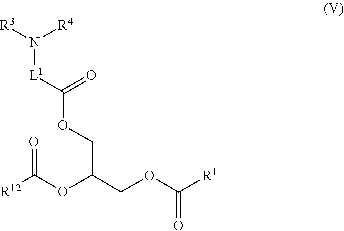

or a pharmaceutically acceptable salt or a stereoisomer thereof, wherein:

L¹ is C₁-C₆ alkylene; R¹ is C₆-C₂₀ alkenyl; R³ and R⁴ are each independently H or C₁-C₃ alkyl; and R¹² is C₆-C₂₀ alkenyl.

In some embodiments, L¹ is C₁-C₆ alkylene; R¹ is C₆-C₂₀ alkenyl; R³ is methyl; R⁴ is methyl; and R¹² is C₆-C₂₀ alkenyl in Formula (V).

Exemplary examples of ionizable lipid of Formula (V) can include, but not limited to the following:

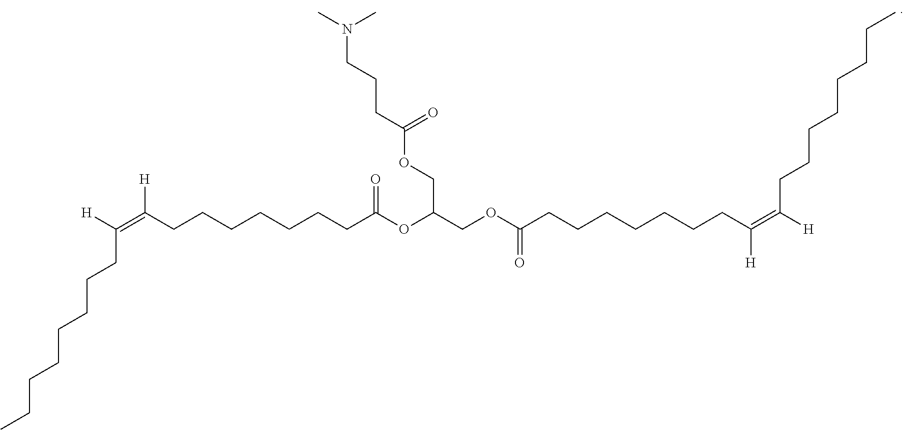 [SMILES: CCCCCCCC/C=C\CCCCCCCC(=O)OCC(COC(=O)CCCN(C)C)OC(=O)CCCCCCC/C=C\CCCCCCCC]

In one embodiment, the ionizable lipid is of Formula (VI):

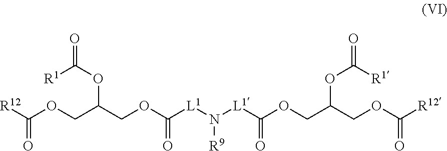

or a pharmaceutically acceptable salt or a stereoisomer thereof, wherein:

L¹ is C₁-C₆ alkylene; L^{1′} is C₁-C₆ alkylene; R¹ is C₆-C₂₀ alkenyl; R^{1′} is C₆-C₂₀ alkenyl; R⁹ is H, C₁-C₆ alkyl or —(CH₂)ₙOH; R¹² is C₆-C₂₀ alkenyl; R^{12′} is C₆-C₂₀ alkenyl; and n is 2, 3 or 4.

In some embodiments, L¹ is C₁-C₆ alkylene; L^{1′} is C₁-C₆ alkylene; R¹ is C₆-C₂₀ alkenyl; R^{1′} is C₆-C₂₀ alkenyl; R⁹ is H; R¹² is C₆-C₂₀ alkenyl; and R^{12′} is C₆-C₂₀ alkenyl in Formula (VI).

In some embodiments, L¹ is C₁-C₆ alkylene; L^{1′} is C₁-C₆ alkylene; R¹ is C₆-C₂₀ alkenyl; R^{1′} is C₆-C₂₀ alkenyl; R⁹ is methyl; R¹² is C₆-C₂₀ alkenyl; and R^{12′} is C₆-C₂₀ alkenyl in Formula (VI).

In some embodiments, L¹ is C₁-C₆ alkylene; L^{1′} is C₁-C₆ alkylene; R¹ is C₆-C₂₀ alkenyl; R^{1′} is C₆-C₂₀ alkenyl; R⁹ is —(CH₂)ₙOH; R¹² is C₆-C₂₀ alkenyl; R^{12′} is C₆-C₂₀ alkenyl; and n is 2, 3 or 4 in Formula (VI).

In some embodiments, L¹ is C₁-C₆ alkylene; L^{1′} is C₁-C₆ alkylene; R¹ is C₆-C₂₀ alkenyl; R^{1′} is C₆-C₂₀ alkenyl; R⁹ is —(CH₂)ₙOH; R¹² is C₆-C₂₀ alkenyl; R^{12′} is C₆-C₂₀ alkenyl; and n is 2 in Formula (VI).

Exemplary examples of ionizable lipid of Formula (VI) can include, but not limited to the following:

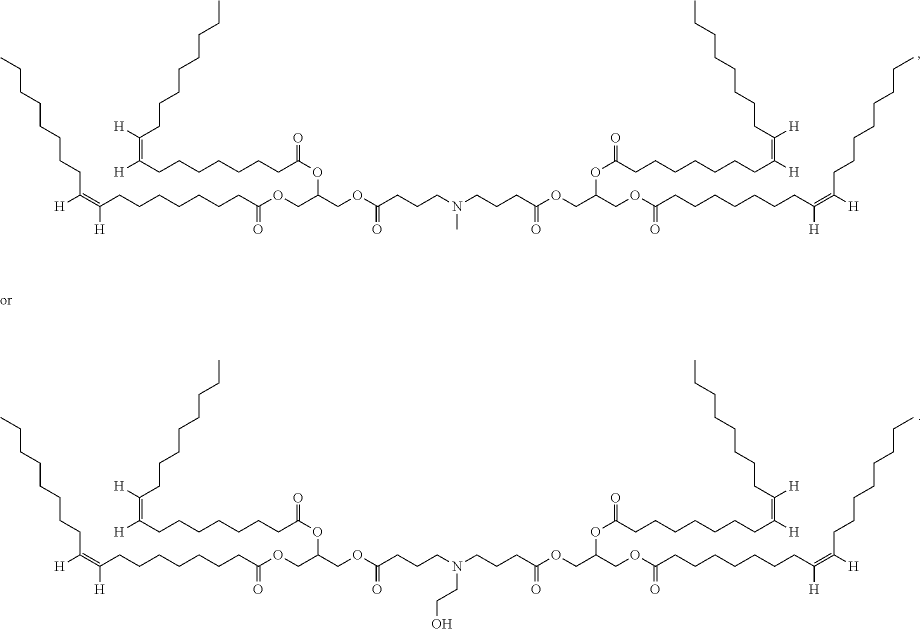

In one embodiment, the ionizable lipid is of Formula (VII):

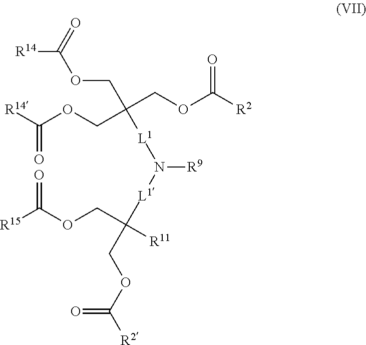

or a pharmaceutically acceptable salt or a stereoisomer thereof, wherein:

L¹ is C₁-C₆ alkylene; L^{1′} is C₁-C₆ alkylene; R² is C₆-C₂₀ alkyl; R^{2′} is C₆-C₂₀ alkyl; R⁹ is H, C₁-C₆ alkyl or —(CH₂)ₙOH; R¹¹ is H or —CH₂)OC(═O)R¹⁶; R¹⁴ is C₆-C₂₀ alkyl; R^{14′} is C₆-C₂₀ alkyl; R¹⁵ is C₆-C₂₀ alkyl; R¹⁶ is C₆-C₂₀ alkyl; and n is 2, 3 or 4.

In some embodiments, L¹ is C₁-C₆ alkylene; L^{1′} is C₁-C₆ alkylene; R² is C₆-C₂₀ alkyl; R^{2′} is C₆-C₂₀ alkyl; R⁹ is H; R¹¹ is H; R¹⁴ is C₆-C₂₀ alkyl; R^{14′} is C₆-C₂₀ alkyl; and R¹⁵ is C₆-C₂₀ alkyl in Formula (VII).

In some embodiments, L¹ is C₁-C₆ alkylene; L^{1′} is C₁-C₆ alkylene; R² is C₆-C₂₀ alkyl; R^{2′} is C₆-C₂₀ alkyl; R⁹ is methyl; R¹¹ is H; R¹⁴ is C₆-C₂₀ alkyl; R^{14′} is C₆-C₂₀ alkyl; and R¹⁵ is C₆-C₂₀ alkyl in Formula (VII).

In some embodiments, L₁ is C1-C6 alkylene; L1′ is C1-C6 alkylene; R2 is C6-C20 alkyl; R2′ is C6-C20 alkyl; R9 is —(CH2)nOH; R11 is H; R14 is C6-C20 alkyl; R14′ is C6-C20 alkyl; R15 is C6-C20 alkyl; and n is 2 in Formula (VII).

Exemplary examples of ionizable lipid of Formula (VII) can include, but not limited to the following:

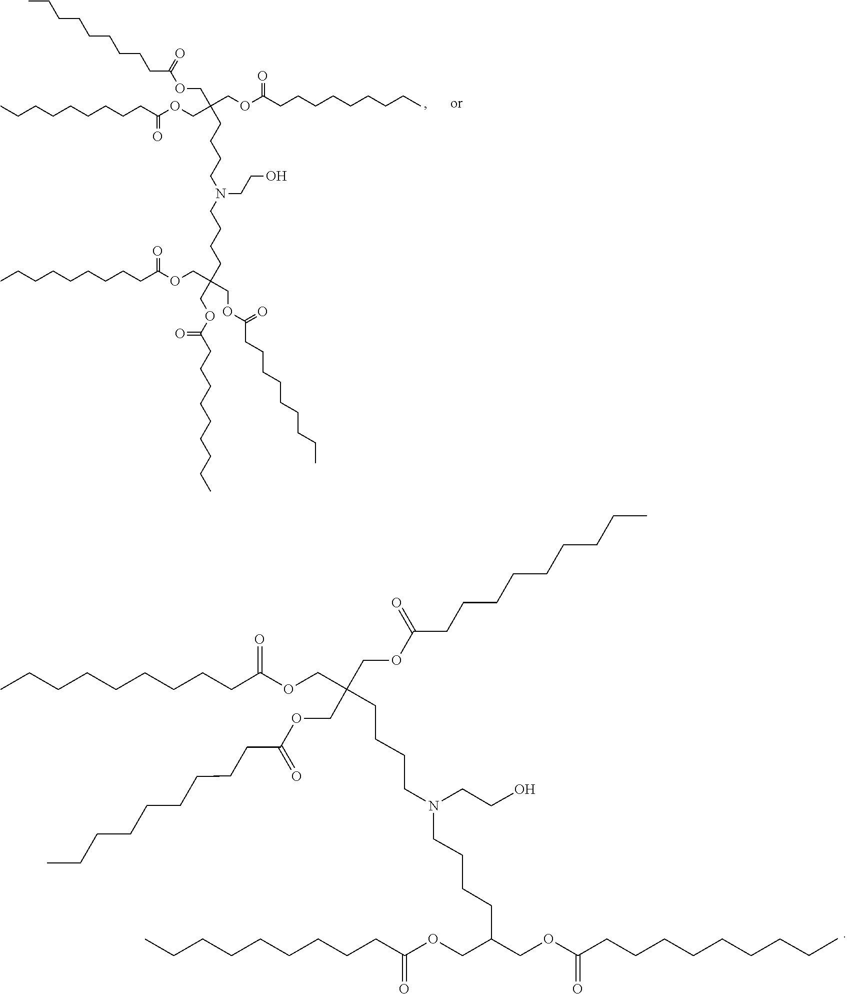 [SMILES: CCCCCCCCCC(=O)OCC(CCCCN(CCO)CCCCC(COC(=O)CCCCCCCCC)(COC(=O)CCCCCCCCC)COC(=O)CCCCCCCCC)(COC(=O)CCCCCCCCC)COC(=O)CCCCCCCCC ; CCCCCCCCCC(=O)OCC(CCCCN(CCO)CCCCC(COC(=O)CCCCCCCCC)(COC(=O)CCCCCCCCC)COC(=O)CCCCCCCCC)COC(=O)CCCCCCCCC]

In one embodiment, the ionizable lipid is of Formula (VIII):

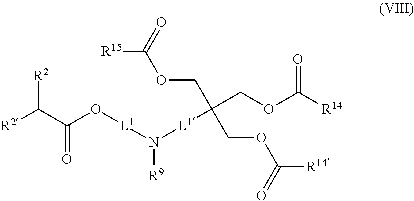

or a pharmaceutically acceptable salt or a stereoisomer thereof, wherein:

L¹ is C₁-C₆ alkylene; L^{1′} is C₁-C₆ alkylene; R² is C₆-C₂₀ alkyl; R^{2′} is C₆-C₂₀ alkyl; R⁹ is H, C₁-C₆ alkyl or —(CH₂)ₙOH; R¹⁴ is C₆-C₂₀ alkyl; R^{14′} is C₆-C₂₀ alkyl; R¹⁵ is C₆-C₂₀ alkyl; and n is 2, 3 or 4.

In some embodiments, L1 is C1-C6 alkylene; L1′ is C1-C6 alkylene; R2 is C6-C20 alkyl; R2′ is C6-C20 alkyl; R9 is H; R14 is C6-C20 alkyl; R14′ is C6-C20 alkyl; and R15 is C6-C20 alkyl in Formula (VIII).

In some embodiments, L1 is C1-C6 alkylene; L1′ is C1-C6 alkylene; R2 is C6-C20 alkyl; R2′ is C6-C20 alkyl; R9 is methyl; R14 is C6-C20 alkyl; R14′ is C6-C20 alkyl; and R15 is C6-C20 alkyl in Formula (VIII).

In some embodiments, L1 is C1-C6 alkylene; L1′ is C1-C6 alkylene; R2 is C6-C20 alkyl; R2′ is C6-C20 alkyl; R9 is —(CH2)nOH; R14 is C6-C20 alkyl; R14′ is C6-C20 alkyl; R15 is C6-C20 alkyl; and n is 2 in Formula (VIII).

Exemplary examples of ionizable lipid of Formula (VIII) can include, but not limited to the following:

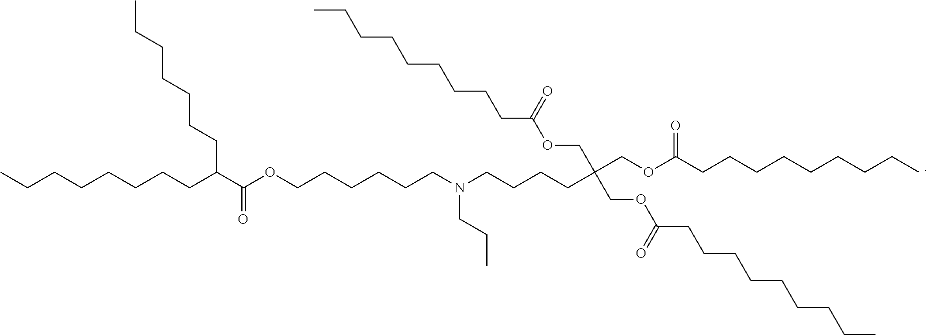 [SMILES: CCCCCCCCCC(=O)OCC(CCCCN(CCC)CCCCCCOC(=O)C(CCCCCCC)CCCCCCCC)(COC(=O)CCCCCCCCC)COC(=O)CCCCCCCCC]

In one embodiment, the ionizable lipid is of Formula (IX):

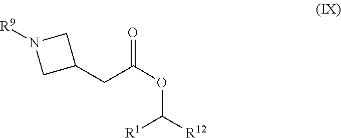

or a pharmaceutically acceptable salt or a stereoisomer thereof, wherein:

R¹ is C₆-C₂₀ alkenyl; R⁹ is H, C₁-C₆ alkyl or —(CH₂)ₙOH; R¹² is C₆-C₂₀ alkenyl; and n is 2, 3 or 4.

In some embodiments, R¹ is C₆-C₂₀ alkenyl; R⁹ is H; and R¹² is C₆-C₂₀ alkenyl in Formula (IX).

In some embodiments, R¹ is C₆-C₂₀ alkenyl; R⁹ is methyl; and R¹² is C₆-C₂₀ alkenyl in Formula (IX).

In some embodiments, R¹ is C₆-C₂₀ alkenyl; R⁹ is —(CH₂)nOH; R¹² is C₆-C₂₀ alkenyl; and n is 2 in Formula (IX).

Exemplary examples of ionizable lipid of Formula (IX) can include, but not limited to the following:

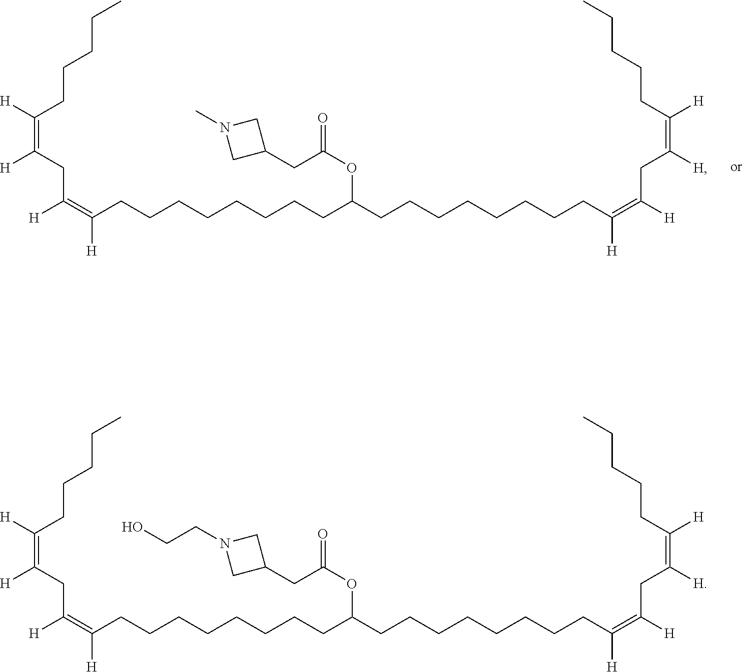

In one embodiment, the ionizable lipid is of Formula (X):

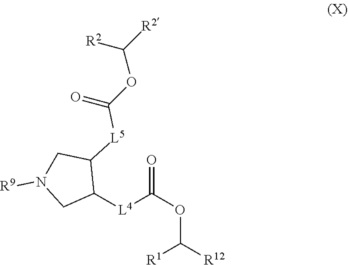

or a pharmaceutically acceptable salt or a stereoisomer thereof, wherein:

L⁴ is absent or C₁-C₆ alkylene; L⁵ is absent or C₁-C₆ alkylene; R¹ is C₆-C₂₀ alkenyl; R² is C₆-C₂₀ alkyl; R^{2′} is C₆-C₂₀ alkyl; R⁹ is H, C₁-C₆ alkyl or —(CH₂)ₙOH; R¹² is C₆-C₂₀ alkenyl; and n is 2, 3 or 4.

In some embodiments, L4 is absent; L5 is absent; R1 is C6-C20 alkenyl; R2 is C6-C20 alkyl; R2′ is C6-C20 alkyl; R9 is H, C1-C6 alkyl or —(CH2)nOH; R12 is C6-C20 alkenyl; and n is 2, 3 or 4 in Formula (X).

In some embodiments, L4 is C1-C6 alkylene; L5 is C1-C6 alkylene; R1 is C6-C20 alkenyl; R2 is C6-C20 alkyl; R2′ is C6-C20 alkyl; R9 is H, C1-C6 alkyl or —(CH2)nOH; R12 is C6-C20 alkenyl; and n is 2, 3 or 4 in Formula (X).

In some embodiments, L4 is absent; L5 is C1-C6 alkylene; R1 is C6-C20 alkenyl; R2 is C6-C20 alkyl; R2′ is C6-C20 alkyl; R9 is H, C1-C6 alkyl or —(CH2)nOH; R12 is C6-C20 alkenyl; and

n is 2, 3 or 4 in Formula (X).

In some embodiments, L4 is C1-C6 alkylene; L5 is absent; R1 is C6-C20 alkenyl; R2 is C6-C20 alkyl; R2′ is C6-C20 alkyl; R9 is H, C1-C6 alkyl or —(CH2)nOH; R12 is C6-C20 alkenyl; and

n is 2, 3 or 4 in Formula (X).

In some embodiments, L4 is absent or C1-C6 alkylene; L5 is absent or C1-C6 alkylene; R1 is C6-C20 alkenyl; R2 is C6-C20 alkyl; R2′ is C6-C20 alkyl; R9 is H; and R12 is C6-C20 alkenyl in Formula (X).

In some embodiments, L⁴ is absent or C₁-C₆ alkylene; L⁵ is absent or C₁-C₆ alkylene; R¹ is C₆-C₂₀ alkenyl; R² is C₆-C₂₀ alkyl; R^{2′} is C₆-C₂₀ alkyl; R⁹ is methyl; and R¹² is C₆-C₂₀ alkenyl in Formula (X).

In some embodiments, L⁴ is absent or C₁-C₆ alkylene; L⁵ is absent or C₁-C₆ alkylene; R¹ is C₆-C₂₀ alkenyl; R² is C₆-C₂₀ alkyl; R^{2′} is C₆-C₂₀ alkyl; R⁹ is —(CH₂)ₙOH; R¹² is C₆-C₂₀ alkenyl; and n is 2 in Formula (X).

Exemplary examples of ionizable lipid of Formula (X) can include, but not limited to the following:

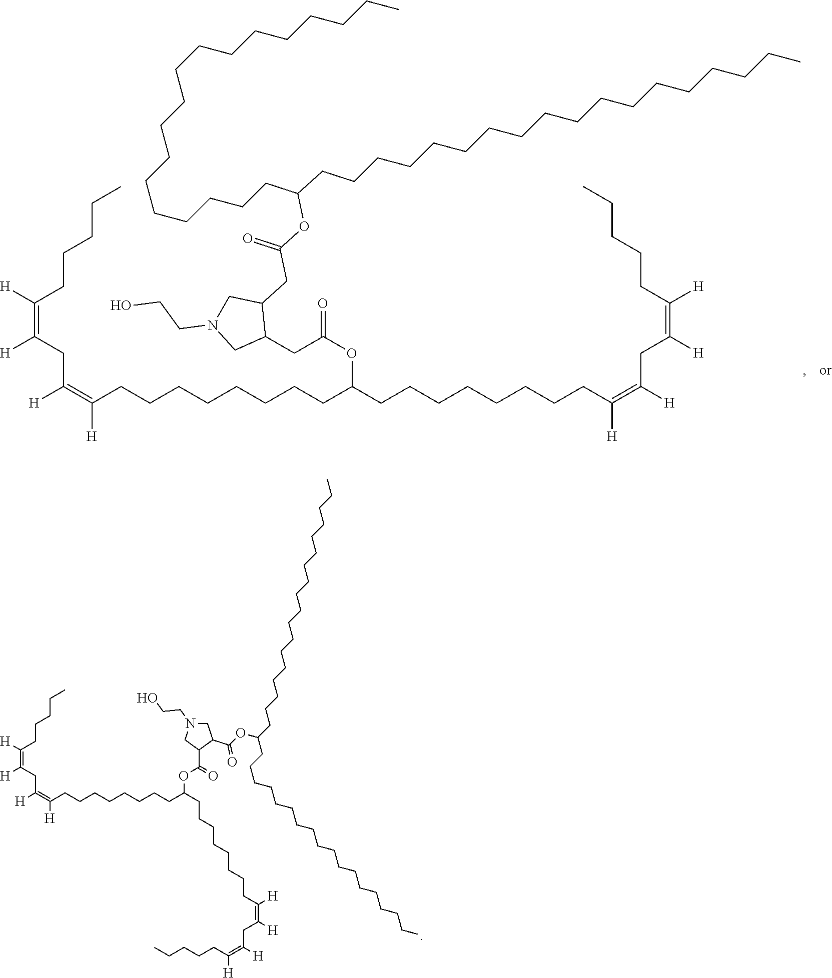 [SMILES: CCCCC/C=C\C/C=C\CCCCCCCCC(CCCCCCCC/C=C\C/C=C\CCCCC)OC(=O)CC1CN(CCO)CC1CC(=O)OC(CCCCCCCCCCCCCCCCCC)CCCCCCCCCCCCCCCCCC ; CCCCC/C=C\C/C=C\CCCCCCCCC(CCCCCCCC/C=C\C/C=C\CCCCC)OC(=O)C1CN(CCO)CC1C(=O)OC(CCCCCCCCCCCCCCCCCC)CCCCCCCCCCCCCCCCCC]

In one embodiment, the ionizable lipid is of Formula (XI):

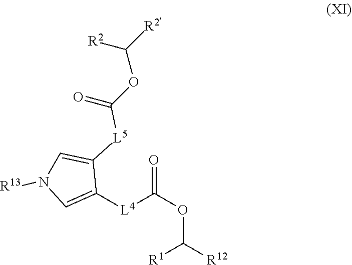

or a pharmaceutically acceptable salt or a stereoisomer thereof, wherein:

L⁴ is absent or C₁-C₆ alkylene; L⁵ is absent or C₁-C₆ alkylene; R¹ is C₆-C₂₀ alkenyl; R² is C₆-C₂₀ alkyl; R^{2′} is C₆-C₂₀ alkyl; R¹² is C₆-C₂₀ alkenyl; R¹³ is H, C₁-C₆ alkyl, —(CH₂)ₙOH or —(CH₂)_{q}N(CH₃)2; n is 2, 3 or 4; and q is 2, 3 or 4.

In some embodiments, L3 is absent; L4 is absent; R1 is C6-C20 alkenyl; R2 is C6-C20 alkyl; R2′ is C6-C20 alkyl; R12 is C6-C20 alkenyl; R13 is H, C1-C6 alkyl, —(CH2)nOH or —(CH2)qN(CH3)2; n is 2, 3 or 4; and q is 2, 3 or 4 in Formula (XI).

In some embodiments, L3 is C1-C6 alkylene; L4 is C1-C6 alkylene; R1 is C6-C20 alkenyl; R2 is C6-C20 alkyl; R2′ is C6-C20 alkyl; R12 is C6-C20 alkenyl; R13 is H, C1-C6 alkyl, —(CH2)nOH or —(CH2)qN(CH3)2; n is 2, 3 or 4; and q is 2, 3 or 4 in Formula (XI).

In some embodiments, L3 is C1-C6 alkylene; L4 is absent; R1 is C6-C20 alkenyl; R2 is C6-C20 alkyl; R2′ is C6-C20 alkyl; R12 is C6-C20 alkenyl; R13 is H, C1-C6 alkyl, —(CH2)nOH or —(CH2)qN(CH3)2; n is 2, 3 or 4; and q is 2, 3 or 4 in Formula (XI).

In some embodiments, L3 is absent; L4 is C1-C6 alkylene; R1 is C6-C20 alkenyl; R2 is C6-C20 alkyl; R2′ is C6-C20 alkyl; R12 is C6-C20 alkenyl; R13 is H, C1-C6 alkyl, —(CH2)nOH or —(CH2)qN(CH3)2; n is 2, 3 or 4; and q is 2, 3 or 4 in Formula (XI).

In some embodiments, L3 is absent or C1-C6 alkylene; L4 is absent or C1-C6 alkylene; R1 is C6-C20 alkenyl; R2 is C6-C20 alkyl; R2′ is C6-C20 alkyl; R12 is C6-C20 alkenyl; and R13 is H in Formula (XI).

In some embodiments, L3 is absent or C1-C6 alkylene; L4 is absent or C1-C6 alkylene; R1 is C6-C20 alkenyl; R2 is C6-C20 alkyl; R2′ is C6-C20 alkyl; R12 is C6-C20 alkenyl; and R13 is methyl in Formula (XI).

In some embodiments, L3 is absent or C1-C6 alkylene; L4 is absent or C1-C6 alkylene; R1 is C6-C20 alkenyl; R2 is C6-C20 alkyl; R2′ is C6-C20 alkyl; R12 is C6-C20 alkenyl; R13 is —(CH2)nOH; and n is 2 in Formula (XI).

In some embodiments, L3 is absent or C1-C6 alkylene; L4 is absent or C1-C6 alkylene; R1 is C6-C20 alkenyl; R2 is C6-C20 alkyl; R2′ is C6-C20 alkyl; R12 is C6-C20 alkenyl; R13 is —(CH2)qN(CH3)2; and q is 3 in Formula (XI).

Exemplary examples of ionizable lipid of Formula (XI) can include, but not limited to the following:

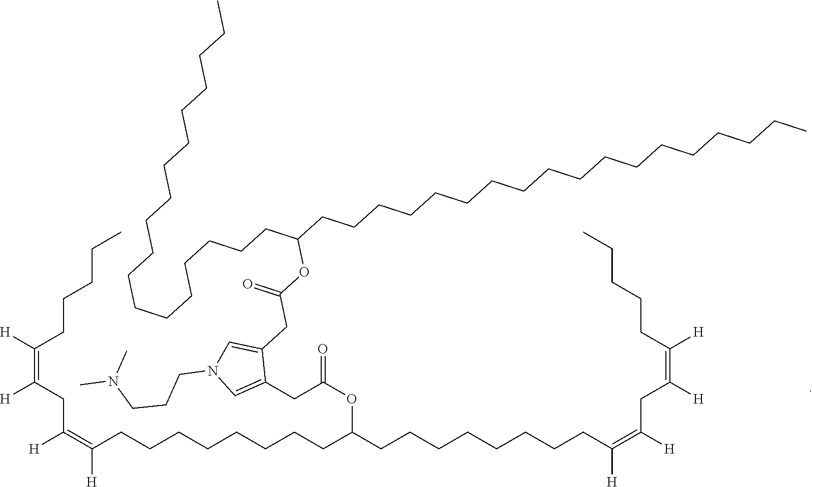 [SMILES: CCCCC/C=C\C/C=C\CCCCCCCCC(CCCCCCCC/C=C\C/C=C\CCCCC)OC(=O)Cc1cn(CCCN(C)C)cc1CC(=O)OC(CCCCCCCCCCCCCCCCCC)CCCCCCCCCCCCCCCCCC]

In one embodiment, the ionizable lipid is of Formula (XII):

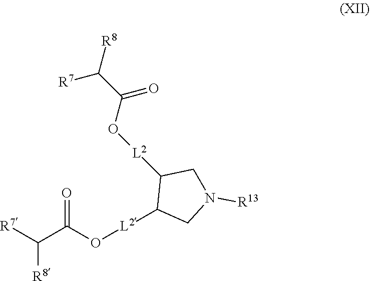

or a pharmaceutically acceptable salt or a stereoisomer thereof, wherein:

L² is C₁-C₈ alkylene; L^{2′} is C₁-C₈ alkylene; R⁷ is C₄-C₂₀ alkyl; R^{7′} is C₄-C₂₀ alkyl; R⁸ is C₄-C₂₀ alkyl; R^{8′} is C₄-C₂₀ alkyl; R¹³ is H, C₁-C₆ alkyl, —(CH₂)ₙOH, or —(CH₂)_{q}N(CH₃)₂; n is 2, 3 or 4; and q is 2, 3 or 4.

In some embodiments, L2 is C1-C8 alkylene; L2′ is C1-C8 alkylene; R7 is C4-C20 alkyl; R7′ is C4-C20 alkyl; R8 is C4-C20 alkyl; R8′ is C4-C20 alkyl; and R13 is H in Formula (XII).

In some embodiments, L2 is C1-C8 alkylene; L2′ is C1-C8 alkylene; R7 is C4-C20 alkyl; R7′ is C4-C20 alkyl; R8 is C4-C20 alkyl; R8′ is C4-C20 alkyl; and R13 is methyl in Formula (XII).

In some embodiments, L2 is C1-C8 alkylene; L2′ is C1-C8 alkylene; R7 is C4-C20 alkyl; R7′ is C4-C20 alkyl; R8 is C4-C20 alkyl; R8′ is C4-C20 alkyl; R13 is —(CH2)nOH; and n is 4 in Formula (XII).

Exemplary examples of ionizable lipid of Formula (XII) can include, but not limited to the following:

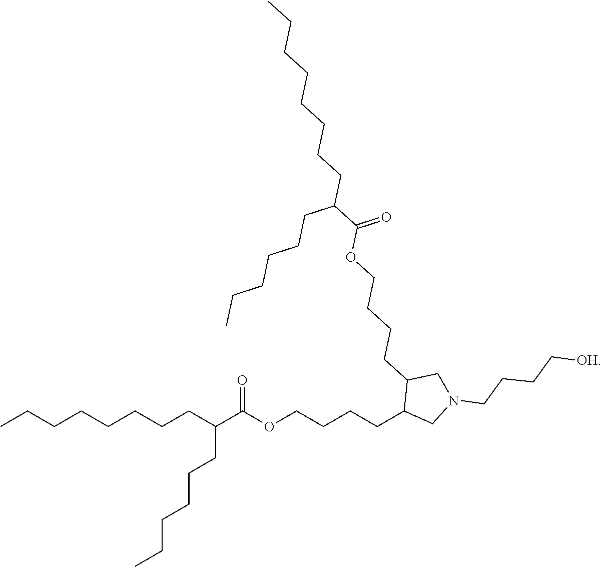 [SMILES: CCCCCCCCC(CCCCCC)C(=O)OCCCCC1CN(CCCCO)CC1CCCCOC(=O)C(CCCCCC)CCCCCCCC]

In one embodiment, the ionizable lipid is of Formula (XIII):

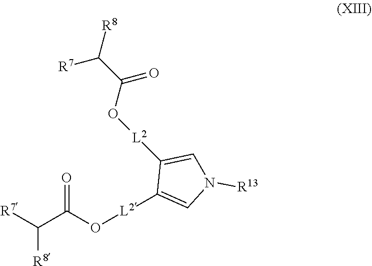

or a pharmaceutically acceptable salt or a stereoisomer thereof, wherein:

L² is C₁-C₈ alkylene; L^{2′} is C₁-C₈ alkylene; R⁷ is C₄-C₂₀ alkyl; R^{7′} is C₄-C₂₀ alkyl; R⁸ is C₄-C₂₀ alkyl; R_{8′} is C₄-C₂₀ alkyl; R¹³ is H, C₁-C₆ alkyl, —(CH₂)ₙOH, or —(CH₂)_{q}N(CH₃)2; n is 2, 3 or 4; and q is 2, 3 or 4.

In some embodiments, L2 is C1-C8 alkylene; L2′ is C1-C8 alkylene; R7 is C4-C20 alkyl; R7′ is C4-C20 alkyl; R8 is C4-C20 alkyl; R8′ is C4-C20 alkyl; and R13 is H in Formula (XIII).

In some embodiments, L2 is C1-C8 alkylene; L2′ is C1-C8 alkylene; R7 is C4-C20 alkyl; R7′ is C4-C20 alkyl; R8 is C4-C20 alkyl; R8′ is C4-C20 alkyl; and R13 is methyl in Formula (XIII).

In some embodiments, L2 is C1-C8 alkylene; L2′ is C1-C8 alkylene; R7 is C4-C20 alkyl; R7′ is C4-C20 alkyl; R8 is C4-C20 alkyl; R8′ is C4-C20 alkyl; R13 is —(CH2)nOH; and n is 2 in Formula (XIII).

In some embodiments, L2 is C1-C8 alkylene; L2′ is C1-C8 alkylene; R7 is C4-C20 alkyl; R7′ is C4-C20 alkyl; R8 is C4-C20 alkyl; R8′ is C4-C20 alkyl; R13 is —(CH2)qN(CH3)2; and q is 3 in Formula (XIII).

Exemplary examples of ionizable lipid of Formula (XIII) can include, but not limited to the following:

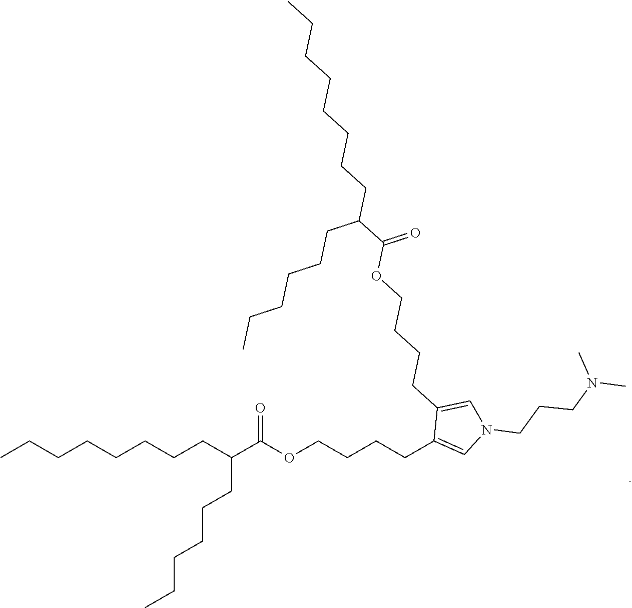 [SMILES: CCCCCCCCC(CCCCCC)C(=O)OCCCCc1cn(CCCN(C)C)cc1CCCCOC(=O)C(CCCCCC)CCCCCCCC]

In one embodiment, the ionizable lipid is of Formula (XIV):

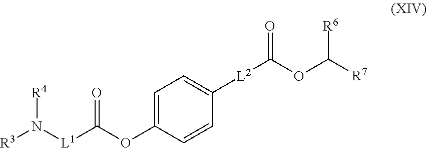

or a pharmaceutically acceptable salt or a stereoisomer thereof, wherein:

L¹ is C₁-C₆ alkylene; L² is C₁-C₈ alkylene; R³ and R⁴ are each independently H or C₁-C₃ alkyl; R⁶ is C₄-C₂₀ alkyl; and R⁷ is C₄-C₂₀ alkyl.

In some embodiments, L¹ is C₁-C₆ alkylene; L² is C₁-C₈ alkylene; R³ is methyl; R⁴ is methyl; R⁶ is C₄-C₂₀ alkyl; and R⁷ is C₄-C₂₀ alkyl in Formula (XIV).

Exemplary examples of ionizable lipid of Formula (XIV) can include, but not limited to the following:

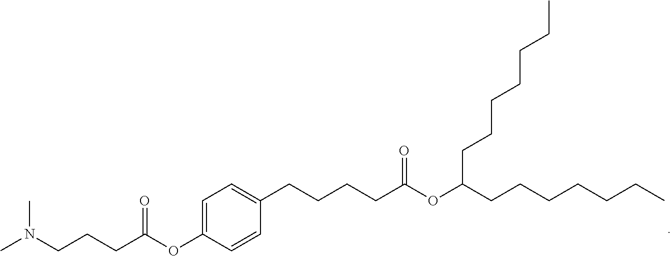 [SMILES: CCCCCCCC(CCCCCCC)OC(=O)CCCCc1ccc(OC(=O)CCCN(C)C)cc1]

In one embodiment, the ionizable lipid is of Formula (XV):

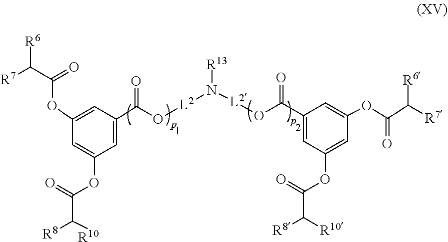

or a pharmaceutically acceptable salt or a stereoisomer thereof, wherein:

L² is C₁-C₈ alkylene; L^{2′} is C₁-C₈ alkylene; R⁶ is C₄-C₂₀ alkyl; R^{6′} is C₄-C₂₀ alkyl; R⁷ is C₄-C₂₀ alkyl; R^{7′} is C₄-C₂₀ alkyl; R⁸ is C₄-C₂₀ alkyl; R^{8′} is C₄-C₂₀ alkyl; R¹⁰ is C₄-C₂₀ alkyl; R^{10′} is C₄-C₂₀ alkyl; R¹³ is H, C₁-C₆ alkyl, —(CH₂)ₙOH, or —(CH₂)_{q}N(CH₃)₂; n is 2, 3 or 4; p₁ is absent or 1; p₂ is absent or 1; and q is 2, 3, or 4.

In some embodiments, L² is C₁-C₈ alkylene; L^{2′} is C₁-C₈ alkylene; R⁶ is C₄-C₂₀ alkyl; R^{6′} is C₄-C₂₀ alkyl; R⁷ is C₄-C₂₀ alkyl; R^{7′} is C₄-C₂₀ alkyl; R⁸ is C₄-C₂₀ alkyl; R^{8′} is C₄-C₂₀ alkyl; R¹⁰ is C₄-C₂₀ alkyl; R^{10′} is C₄-C₂₀ alkyl; R¹³ is H; p₁ is absent; and p₂ is absent in Formula (XV).

In some embodiments, L² is C₁-C₈ alkylene; L^{2′} is C₁-C₈ alkylene; R⁶ is C₄-C₂₀ alkyl; R^{6′} is C₄-C₂₀ alkyl; R⁷ is C₄-C₂₀ alkyl; R^{7′} is C₄-C₂₀ alkyl; R⁸ is C₄-C₂₀ alkyl; R^{8′} is C₄-C₂₀ alkyl; R¹⁰ is C₄-C₂₀ alkyl; R^{10′} is C₄-C₂₀ alkyl; R¹³ is H; p₁ is 1; and p₂ is 1 in Formula (XV).

In some embodiments, L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R8 is C4-C20 alkyl; R8′ is C4-C20 alkyl; R10 is C4-C20 alkyl; R10′ is C4-C20 alkyl; R13 is methyl; p1 is absent; and p2 is absent in Formula (XV).

In some embodiments, L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R8 is C4-C20 alkyl; R8′ is C4-C20 alkyl; R10 is C4-C20 alkyl; R10′ is C4-C20 alkyl; R13 is methyl; p1 is 1; and p2 is 1 in Formula (XV).

In some embodiments, L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R8 is C4-C20 alkyl; R8′ is C4-C20 alkyl; R10 is C4-C20 alkyl; R10′ is C4-C20 alkyl; R13 is —(CH2)nOH; n is 4; p1 is absent; and p2 is absent in Formula (XV).

In some embodiments, L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R8 is C4-C20 alkyl; R8′ is C4-C20 alkyl; R10 is C4-C20 alkyl; R10′ is C4-C20 alkyl; R13 is —(CH2)nOH; n is 4; p1 is 1; and p2 is 1 in Formula (XV).

Exemplary examples of ionizable lipid of Formula (XV) can include, but not limited to the following:

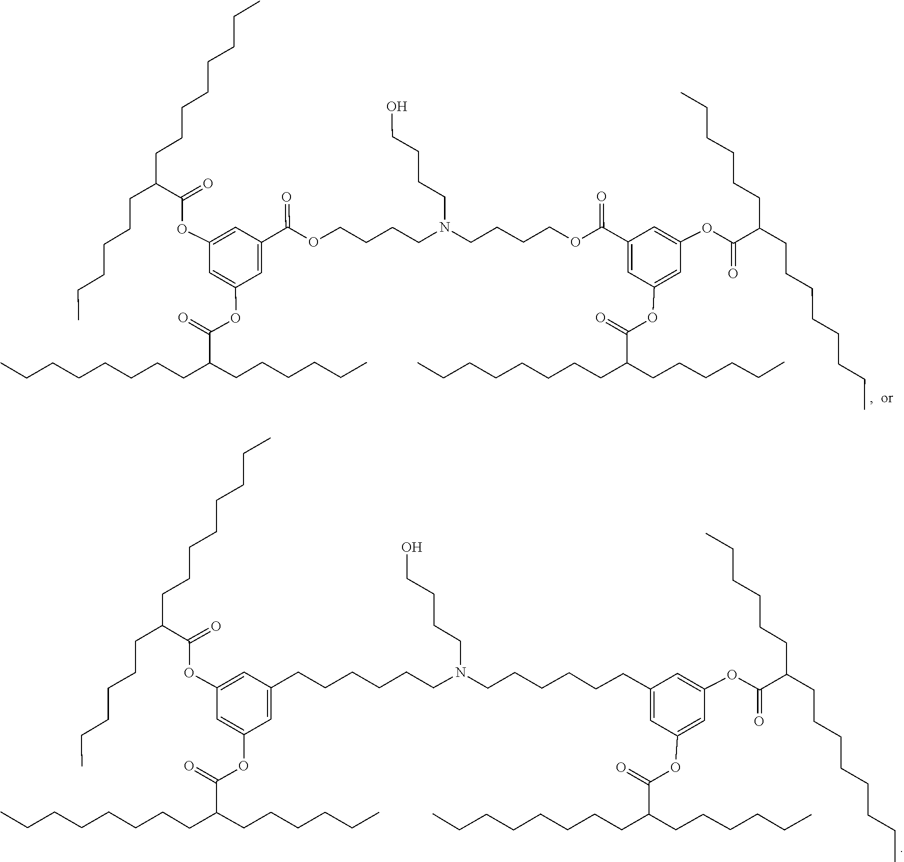 [SMILES: CCCCCCCCC(CCCCCC)C(=O)Oc1cc(OC(=O)C(CCCCCC)CCCCCCCC)cc(C(=O)OCCCCN(CCCCO)CCCCOC(=O)c2cc(OC(=O)C(CCCCCC)CCCCCCCC)cc(OC(=O)C(CCCCCC)CCCCCCCC)c2)c1 ; CCCCCCCCC(CCCCCC)C(=O)Oc1cc(CCCCCCN(CCCCO)CCCCCCc2cc(OC(=O)C(CCCCCC)CCCCCCCC)cc(OC(=O)C(CCCCCC)CCCCCCCC)c2)cc(OC(=O)C(CCCCCC)CCCCCCCC)c1]

In one embodiment, the ionizable lipid is of Formula (XVI):

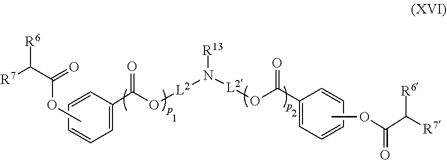

or a pharmaceutically acceptable salt or a stereoisomer thereof, wherein:

L² is C₁-C₈ alkylene; L^{2′} is C₁-C₈ alkylene; R⁶ is C₄-C₂₀ alkyl; R^{6′} is C₄-C₂₀ alkyl; R⁷ is C₄-C₂₀ alkyl; R^{7′} is C₄-C₂₀ alkyl; R¹³ is H, C₁-C₆ alkyl, —(CH₂)ₙOH, or —(CH₂)_{q}N(CH₃)2; n is 2, 3 or 4; p₁ is absent or 1; p₂ is absent or 1; and q is 2, 3 or 4.

In one embodiment, the ionizable lipid is of Formula (XVI-A):

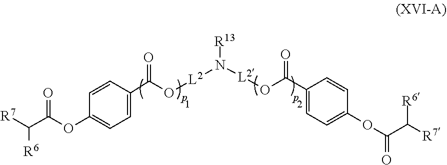

or a pharmaceutically acceptable salt or a stereoisomer thereof, wherein:

L² is C₁-C₈ alkylene; L^{2′} is C₁-C₈ alkylene; R⁶ is C₄-C₂₀ alkyl; R^{6′} is C₄-C₂₀ alkyl; R⁷ is C₄-C₂₀ alkyl; R^{7′} is C₄-C₂₀ alkyl; R¹³ is H, C₁-C₆ alkyl, —(CH₂)ₙOH, or —(CH₂)_{q}N(CH₃)2; n is 2, 3 or 4; p₁ is absent or 1; p₂ is absent or 1; and q is 2, 3 or 4.

In some embodiments, L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R13 is H; p1 is absent or 1; and p2 is absent or 1 in Formula (XVI) and Formula (XVI-A).

In some embodiments, L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R13 is methyl; p1 is absent or 1; and p2 is absent or 1 in Formula (XVI) and Formula (XVI-A).

L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R13 is —(CH2)nOH; n is 4; p1 is absent; and p2 is absent in Formula (XVI) and Formula (XVI-A).

In some embodiments, L2 is C1-C8 alkylene; L2′ is C1-C8 alkylene; R6 is C4-C20 alkyl; R6′ is C4-C20 alkyl; R7 is C4-C20 alkyl; R7′ is C4-C20 alkyl; R13 is —(CH2)nOH; n is 4; p1 is 1; and p2 is 1 in Formula (XVI) and Formula (XVI-A).

Exemplary examples of ionizable lipid of Formula (XVI) and Formula (XVI-A) can include, but not limited to the following:

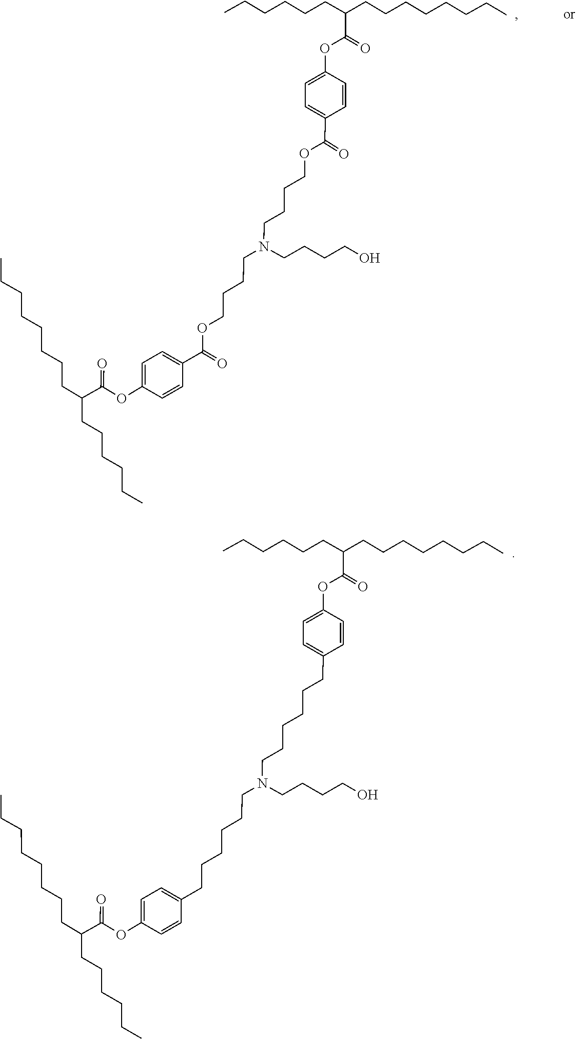

In one embodiment, a compound has the following structure of Formula (XVII):

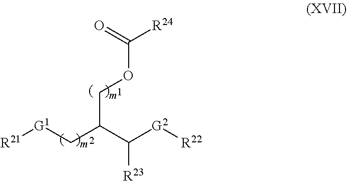

or a stereoisomer, salt or tautomer thereof, wherein: G¹ and G² are each independently —OC(═O)—, —NR²⁵C(═O)—, or —CH═CH—; R²¹ and R²² are each independently C₁-C₆ alkyl, linear C₁₀-C₂₀ alkyl, linear C₁₀-C₂₀ alkenyl, or branched C₁₀-C₃₅ alkenyl, wherein the C₁-C₆ alkyl is substituted with —OC(═O)R²⁶; R²³ is H, OH, or OCH₃; R²⁴ is C₁-C₅ heteroalkyl; R²⁵ is H or C₁-C₄ alkyl; R²⁶ is branched C₁-C₃₀ alkyl; and m¹ and m² are each independently an integer of 0 or 1.

In one embodiment, G¹ and G² are each independently —OC(═O)—, —NR²⁵C(═O)—, or —CH═CH—. In some embodiments, G¹ and G² are each —OC(═O)—. In some embodiments, one of G¹ or G² is —NR²⁵C(═O)— and the other one of G¹ or G² is —CH═CH—.

In one embodiment, m² is 0. In some embodiments, m² is 1.

In one embodiment, the compound has one of the following structures of Formula (XVIIA)-(XVIIB):

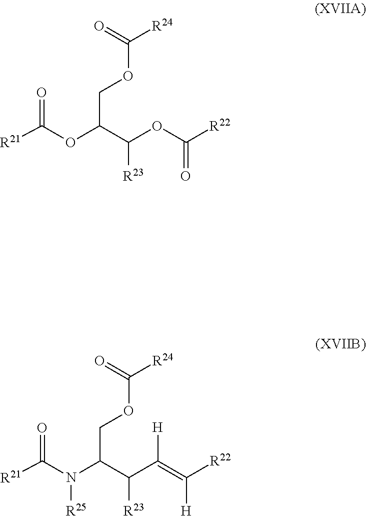

or a stereoisomer, salt or tautomer thereof. In some embodiments, the compound is

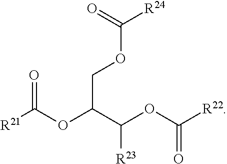

In some embodiments, the compound is

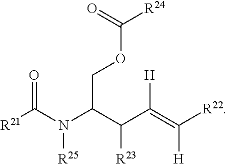

In one embodiment, R24 is C1-C8 heteroalkyl. In some embodiments, R24 is C3-C7 heteroalkyl. In some embodiments, R24 is C3 heteroalkyl. In some embodiments, R24 is C4 heteroalkyl. In some embodiments, R24 is C5 heteroalkyl. In some embodiments, R24 is C6 heteroalkyl. In some embodiments, R24 is C7 heteroalkyl. In some embodiments, R24 is C4 alkylamine. In some embodiments, R24 is C5 alkylamine. In some embodiments, R24 is C6 alkylamine. In some embodiments, R24 is C7 alkylamine. In some certain embodiments, R24 is

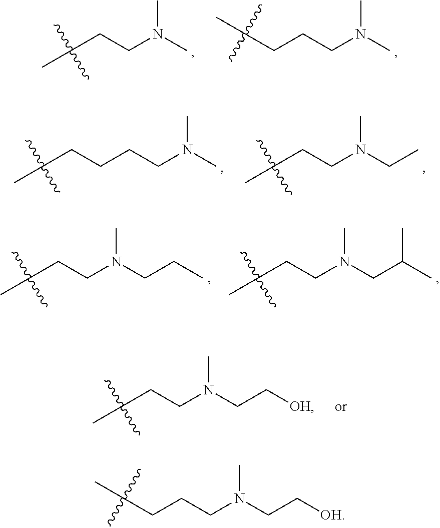

In some embodiments, R24 is

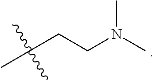 [SMILES: *CCN(C)C]

In some embodiments, R24 is

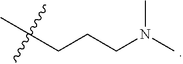 [SMILES: *CCCN(C)C]

In some embodiments, R24 is

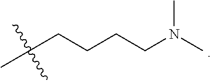 [SMILES: *CCCCN(C)C]

In some embodiments, R24 is

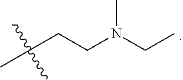 [SMILES: *CCN(C)CC]

In some embodiments, R24 is

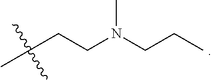 [SMILES: *CCN(C)CCC]

In some embodiments, R24 is

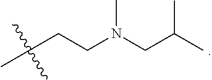 [SMILES: *CCN(C)CC(C)C]

In some embodiments, R24 is

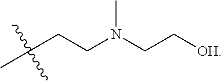 [SMILES: *CCN(C)CCO]

In some embodiments, R24 is

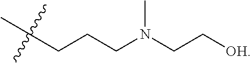 [SMILES: *CCCN(C)CCO]

In some embodiments, C1-C8 heteroalkyl of R24 is further substituted with a cycloalkyl. In some certain embodiments, R24 is

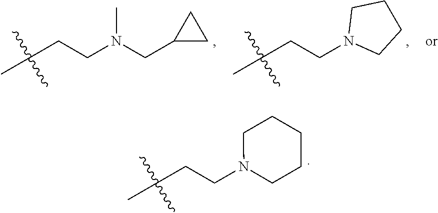

In some embodiments, R24 is

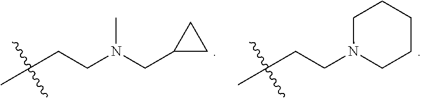

In some embodiments, R24 is

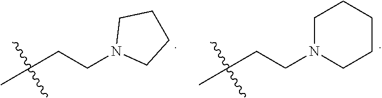 [SMILES: *CCN1CCCC1 ; *CCN1CCCCC1]

In some embodiments, R²⁴ is

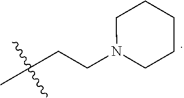 [SMILES: *CCN1CCCCC1]

In one embodiment, R²⁵ is H or C₁-C₄ alkyl. In some embodiments, R is H. In some embodiments, R²⁵ is C₁-C₄ alkyl. In some embodiments, R²⁵ is C₁ alkyl. In some embodiments, R²⁵ is C₂ alkyl. In some embodiments, R²⁵ is C₃ alkyl. In some embodiments, R²⁵ is C₄ alkyl. In some certain embodiments, R²⁵ is —CH₃. In some other certain embodiments, R²⁵ is —CH₂CH₃.

In one embodiment, R²¹ and R²² are each independently C₁-C₆ alkyl, linear C₁₀-C₂₀ alkyl, linear C₁₀-C₂₀ alkenyl, or branched C₁₀-C₃₅ alkenyl, wherein the C₁-C₆ alkyl is substituted with —OC(═O)R²⁶. In some embodiments, R²¹ and R²² are each independently C₂-C₅ alkyl substituted with —OC(═O)R²⁶, linear C₁₂-C₁₈ alkyl, linear C₁₂-C₁₈ alkenyl, or branched C₁₄-C₃₂ alkenyl. In some embodiments, one of R²¹ or R²² is linear C₁₂-C₁₈ alkyl and the other one of R²¹ or R²² is linear C₁₂-C₁₈ alkenyl. In some embodiments, one of R²¹ or R²² is C₂-C₅ alkyl substituted with —OC(═O)R²⁶ and the other one of R²¹ or R²² is linear C₁₂-C₁₈ alkyl. In some embodiments, one of R²¹ or R²² is linear C₁₂-C₁₈ alkyl and the other one of R²¹ or R²² is branched C₁₄-C₃₂ alkenyl. In some embodiments, R²¹ and R²² are each C₂-C₅ alkyl substituted with —OC(═O)R²⁶. In some embodiments, R²¹ and R²² are each C₂ alkyl substituted with —OC(═O)R²⁶. In some embodiments, R²¹ and R²² are each C₃ alkyl substituted with —OC(═O)R²⁶. In some embodiments, R²¹ and R²² are each C₄ alkyl substituted with —OC(═O)R²⁶. In some embodiments, R²¹ and R²² are each C₅ alkyl substituted with —OC(═O)R²⁶. In some embodiments, R²¹ and R²² are each linear C₁₂-C₁₈ alkenyl. In some embodiments, R²¹ and R²² are each linear C₁₂ alkenyl. In some embodiments, R²¹ and R²² are each linear C₁₃ alkenyl. In some embodiments, R²¹ and R²² are each linear C₁₄ alkenyl. In some embodiments, R²¹ and R²² are each linear C₁₅ alkenyl. In some embodiments, R²¹ and R²² are each linear C₁₆ alkenyl. In some embodiments, R²¹ and R²² are each linear C₁₇ alkenyl. In some embodiments, R²¹ and R²² are each linear C₁₈ alkenyl.

In one embodiment, R²⁶ is branched C₁₀-C₃₀ alkyl. In some embodiments, R²⁶ is branched C₁₀-C₂₀ alkyl. In some certain embodiments, R²⁶ is branched C₁₂-C₁₈ alkyl. In some certain embodiments, R²⁶ is branched C₁₂ alkyl. In some certain embodiments, R²⁶ is branched C₁₃ alkyl. In some certain embodiments, R²⁶ is branched C₁₄ alkyl. In some certain embodiments, R²⁶ is branched C₁₅ alkyl. In some certain embodiments, R²⁶ is branched C₁₆ alkyl. In some certain embodiments, R²⁶ is branched C₁₇ alkyl. In some certain embodiments, R²⁶ is branched C₁₈ alkyl.

In some embodiments, R²¹ and R²² each independently have one of the following structures:

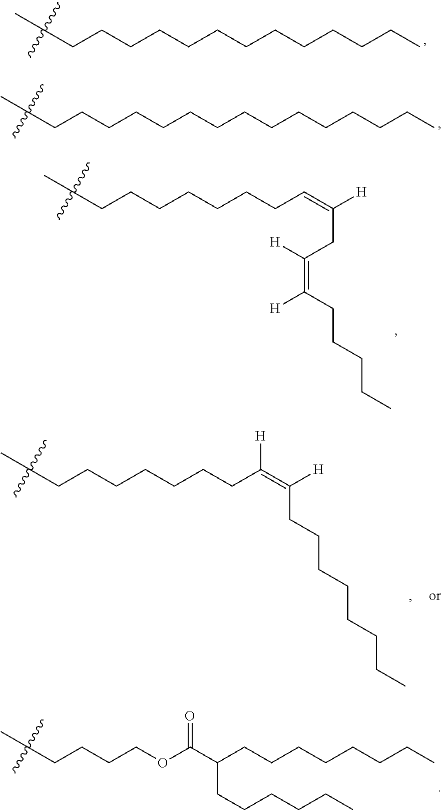 [SMILES: *CCCCCCCCCCCCC ; *CCCCCCCCCCCCCCC ; *CCCCCCC/C=C\C/C=C\CCCCC ; *CCCCCCC/C=C\CCCCCCCC ; *CCCCOC(=O)C(CCCCCC)CCCCCCCC]

In some embodiments, R²¹ or R²² is

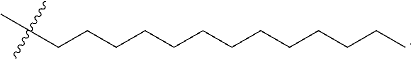 [SMILES: *CCCCCCCCCCCCC]

In some embodiments, R²¹ or R²² is

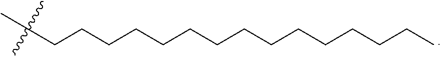 [SMILES: *CCCCCCCCCCCCCCC]

In some embodiments, R²¹ or R²² is

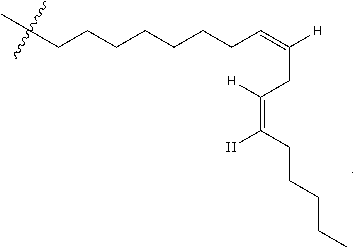 [SMILES: *CCCCCCC/C=C\C/C=C\CCCCC]

In some embodiments, R²¹ or R²² is

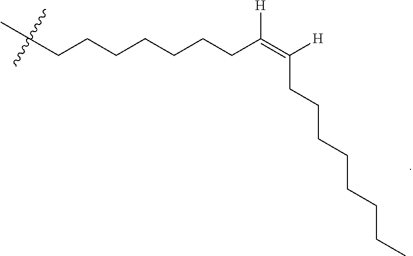 [SMILES: *CCCCCCC/C=C\CCCCCCCC]

In some embodiments, R²¹ or²² is

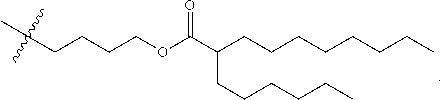 [SMILES: *CCCCOC(=O)C(CCCCCC)CCCCCCCC]

In one embodiment, the compound has one of the following structures shown in Table A below.

TABLE A
List of Compounds of (XVII)-(XVIIB)

| Compound | |
|---|---|
| ID | Structure |
| 24 | 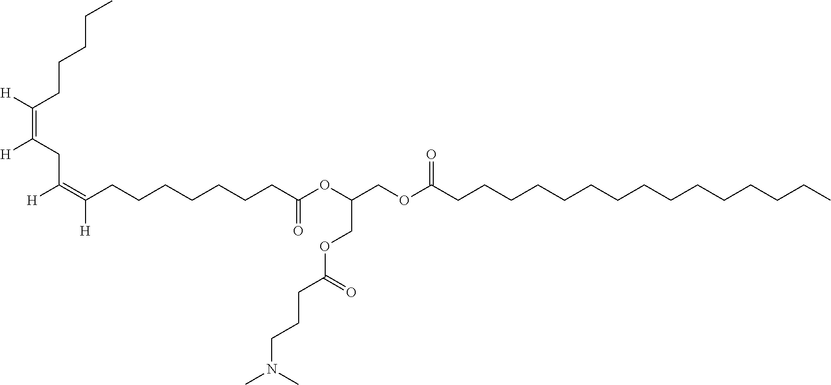 [SMILES: CCCCC/C=C\C/C=C\CCCCCCCC(=O)OC(COC(=O)CCCCCCCCCCCCCCC)COC(=O)CCCN(C)C] |
| 25 | 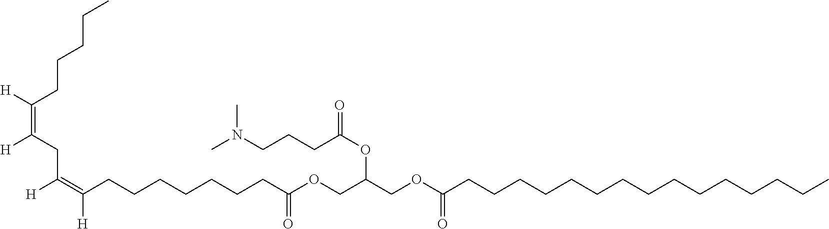 [SMILES: CCCCC/C=C\C/C=C\CCCCCCCC(=O)OCC(COC(=O)CCCCCCCCCCCCCCC)OC(=O)CCCN(C)C] |
| 26 | 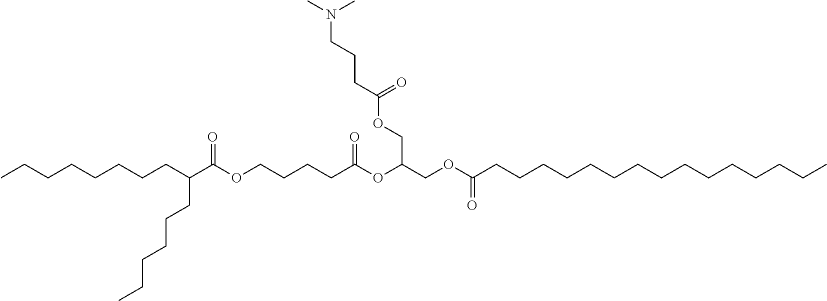 [SMILES: CCCCCCCCCCCCCCCC(=O)OCC(COC(=O)CCCN(C)C)OC(=O)CCCCOC(=O)C(CCCCCC)CCCCCCCC] |
| 27 | 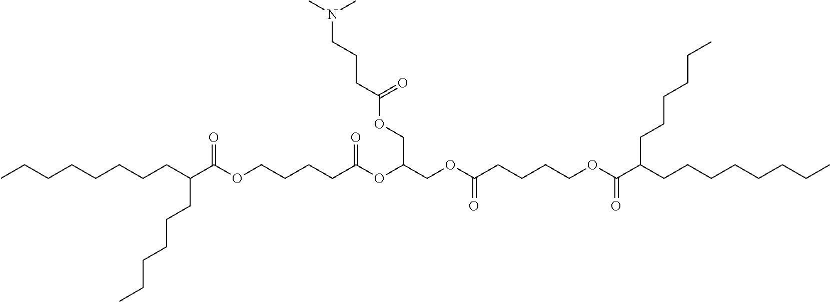 [SMILES: CCCCCCCCC(CCCCCC)C(=O)OCCCCC(=O)OCC(COC(=O)CCCN(C)C)OC(=O)CCCCOC(=O)C(CCCCCC)CCCCCCCC] |
| 2 | 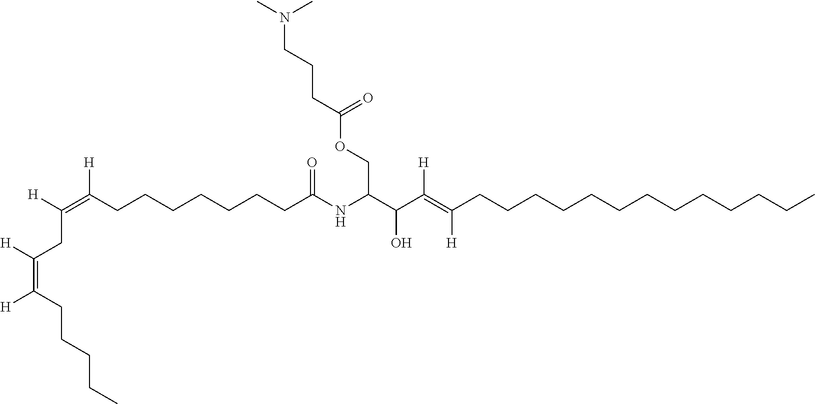 [SMILES: CCCCC/C=C\C/C=C\CCCCCCCC(=O)NC(COC(=O)CCCN(C)C)C(O)/C=C/CCCCCCCCCCCCC] |
| 3 | 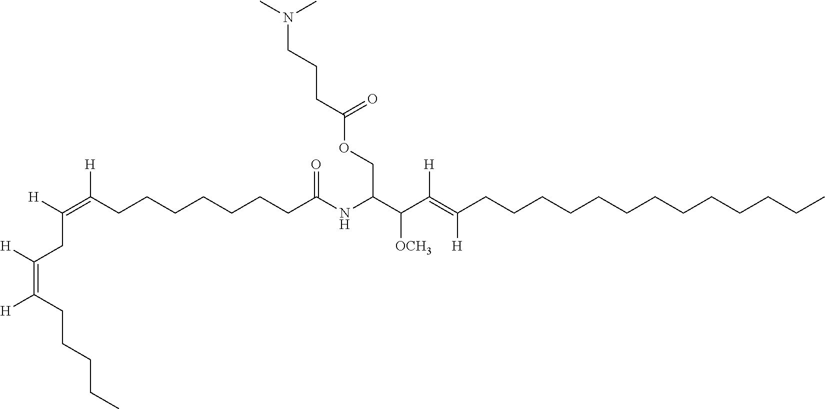 [SMILES: CCCCC/C=C\C/C=C\CCCCCCCC(=O)NC(COC(=O)CCCN(C)C)C(/C=C/CCCCCCCCCCCCC)OC] |
| 4OH | 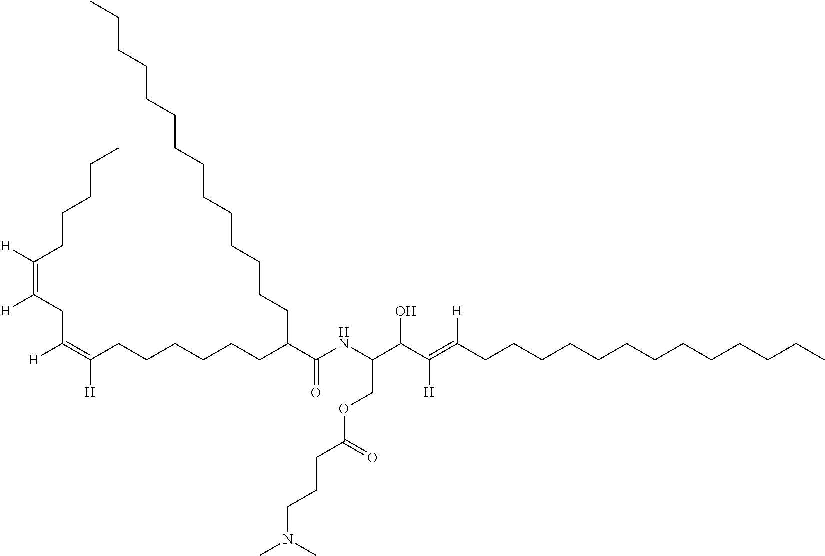 [SMILES: CCCCC/C=C\C/C=C\CCCCCCC(CCCCCCCCCCCCCC)C(=O)NC(COC(=O)CCCN(C)C)C(O)/C=C/CCCCCCCCCCCCC] |
| 4OMe | 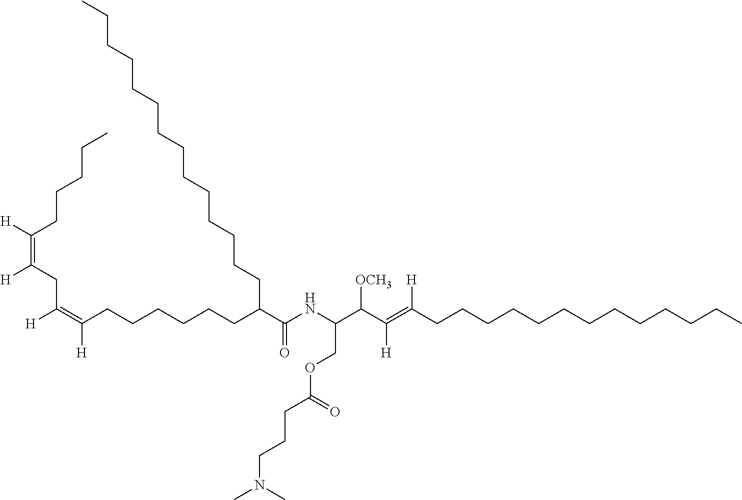 [SMILES: CCCCC/C=C\C/C=C\CCCCCCC(CCCCCCCCCCCCCC)C(=O)NC(COC(=O)CCCN(C)C)C(/C=C/CCCCCCCCCCCCC)OC] |
| 5OH | 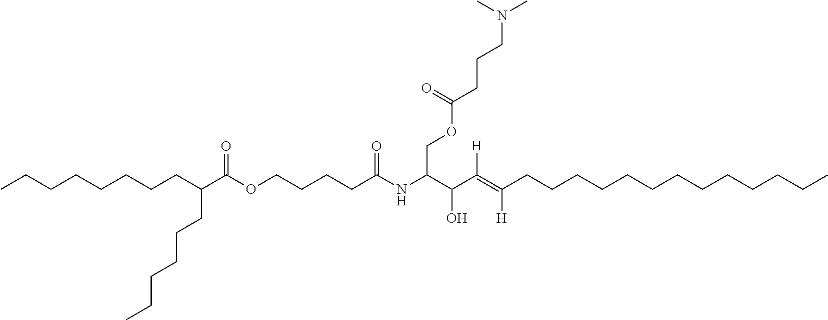 [SMILES: CCCCCCCCCCCCC/C=C/C(O)C(COC(=O)CCCN(C)C)NC(=O)CCCCOC(=O)C(CCCCCC)CCCCCCCC] |
| 5OMe | 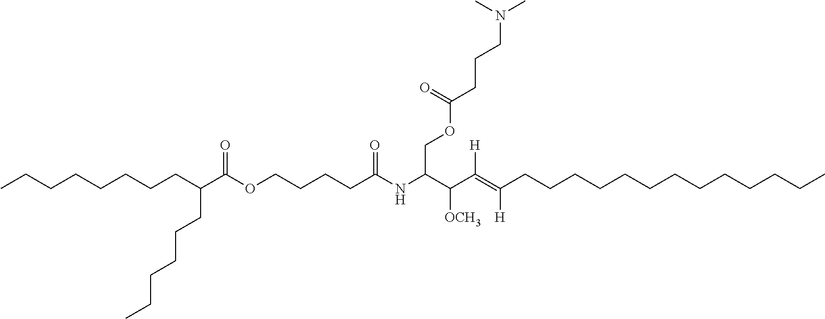 [SMILES: CCCCCCCCCCCCC/C=C/C(OC)C(COC(=O)CCCN(C)C)NC(=O)CCCCOC(=O)C(CCCCCC)CCCCCCCC] |
| 6 | 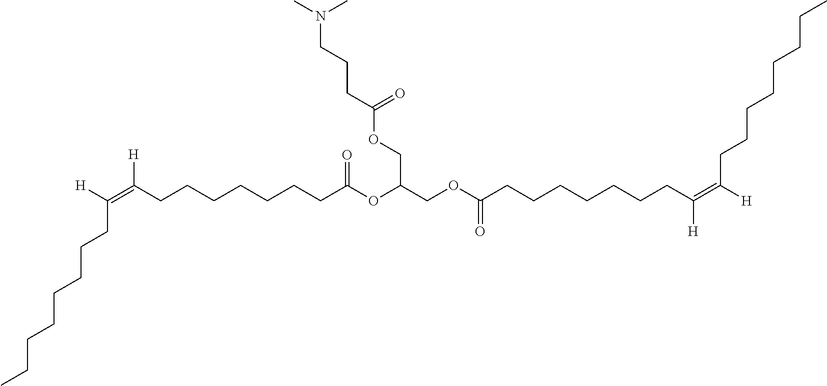 [SMILES: CCCCCCCC/C=C\CCCCCCCC(=O)OCC(COC(=O)CCCN(C)C)OC(=O)CCCCCCC/C=C\CCCCCCCC] |
| 45 | 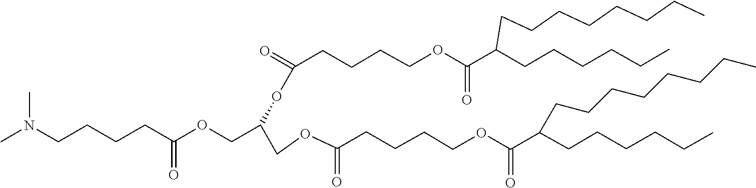 |
| 46 | 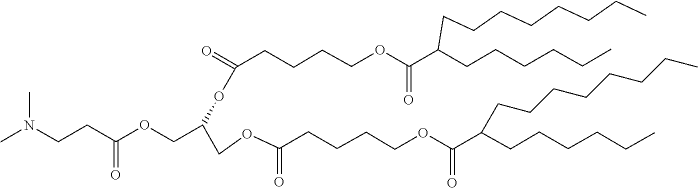 |
| 47 | 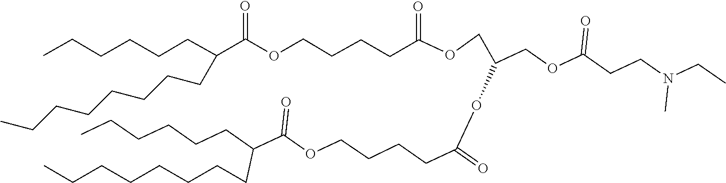 |
| 48 | 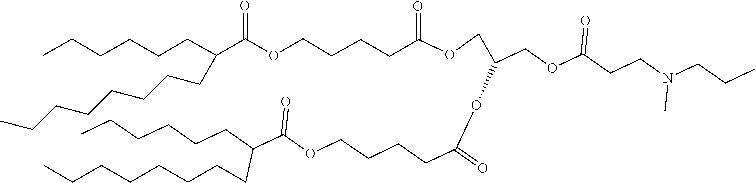 |
| 49 | 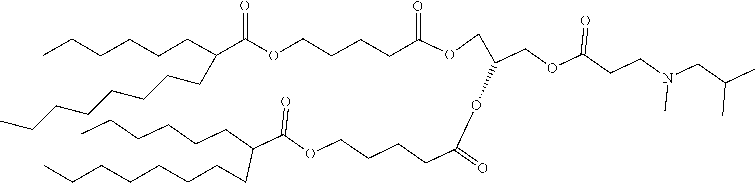 |
| 50 | 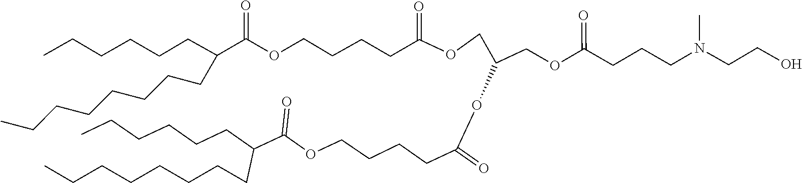 |
| 51 | 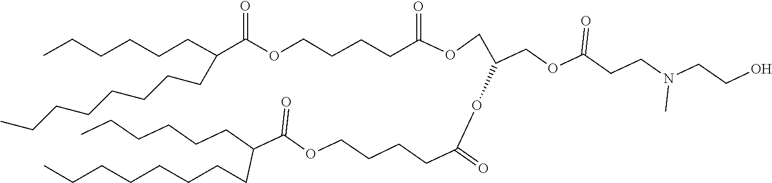 |
| 52 | 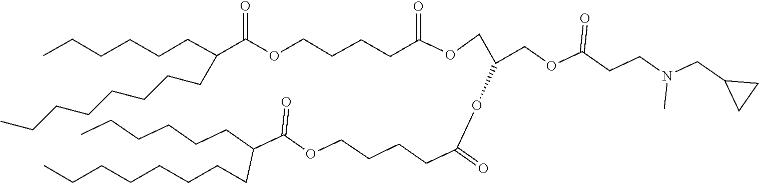 |
| 53 | 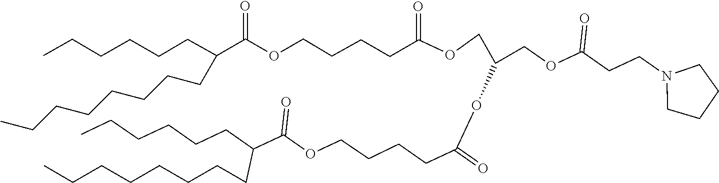 |
| 54 | 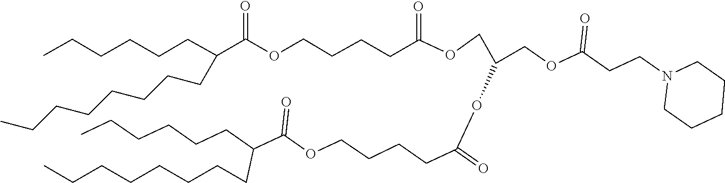 |
| 55 | 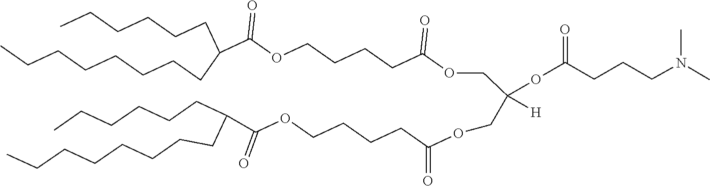 [SMILES: CCCCCCCCC(CCCCCC)C(=O)OCCCCC(=O)OCC(COC(=O)CCCCOC(=O)C(CCCCCC)CCCCCCCC)OC(=O)CCCN(C)C] |

In one embodiment, a compound has the following structure of Formula (XVIII):

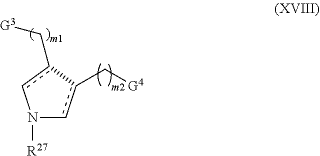

or a stereoisomer, salt or tautomer thereof, wherein: G³ and G⁴ are each independently —OC(═O)R²⁸, —C(═O)OR²⁸, —CH(CH₂OC(═O)R²⁸)₂, —C(CH₂OC(═O)R²⁸)₃, -PhOC(═O)R²⁸, -Ph(OC(═O)R²⁸)₂, —OC(═O)Ph(OC(═O)R²⁸)₂, or —C(═O)OCH₂C(OC(═O)R²⁸)(CH₂OC(═O)R²⁸); R²⁷ is H, C₁-C₆ alkyl, or C₁-C₆ heteroalkyl; R²⁸ is each independently linear C₆-C₁₂ alkyl, branched C₁₀-C₄₀ alkyl, linear C₁₅-C₂₀ alkenyl, or branched C₂₀-C₄₀ alkenyl; m¹ and m² are each independently an integer from 1 to 6; [symbol] is a direct bond or absent; and [symbol] is a single bond or a double bond.

In one embodiment, the compound has one of the following structures of Formula (XVIIIA)-(XVIIIC):

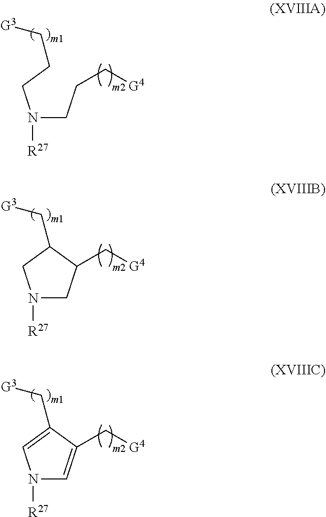

or a stereoisomer, salt or tautomer thereof. In some embodiments, the compound is

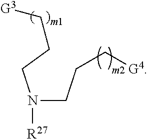

In some embodiments, the compound is

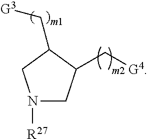

In some embodiments, the compound is

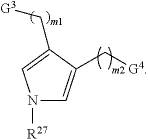

In one embodiment, R²⁷ is H, C₁-C₆ alkyl, or C₁-C₆ heteroalkyl. In some embodiments, R²⁷ is H, C₁-C₃ alkyl, or C₁-C₅ heteroalkyl. In some embodiments, R²⁷ is C₁-C₃ alkyl, C₂-C₄ alkylalcohol, or C₅ alkylamine. In some certain embodiments, R²⁷ has one of the following structures:

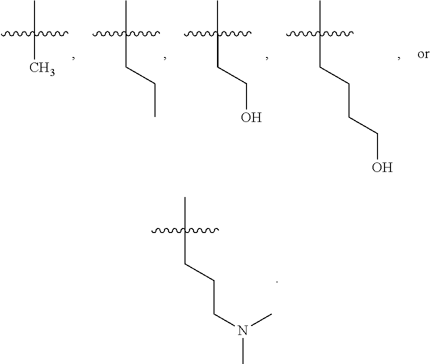

In some embodiment, R²⁷ is

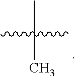

In some embodiment, R²⁷ is

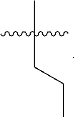 [SMILES: *CCC]

In some embodiment, R²⁷ is

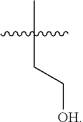 [SMILES: *CCO]

In some embodiment, R²⁷ is

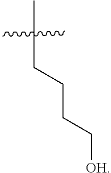 [SMILES: *CCCCO]

In some embodiment, R²⁷ is

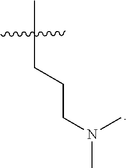 [SMILES: *CCCN(C)C]

In one embodiment, G³ and G⁴ are each independently —OC(═O)R²⁸, —C(═O)OR²⁸, —CH(CH₂OC(═O)R²⁸)₂, —C(CH₂OC(═O)R²⁸)₃, -PhOC(═O)R²⁸, -Ph(OC(═O)R²⁸)₂, —OC(═O)Ph(OC(═O)R²⁸)₂, or —C(═O)OCH₂C(OC(═O)R²⁸)(CH₂OC(═O)R²⁸). In some embodiments, G³ and G⁴ each have one of the following structures:

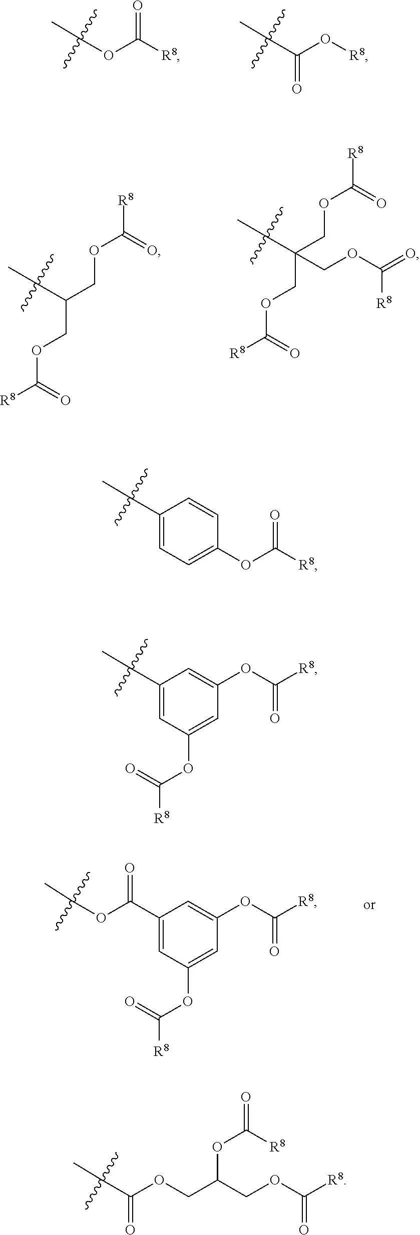

In some embodiments, G³ or G⁴ has

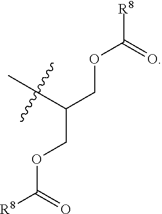

In some embodiments, G³ or G⁴ has

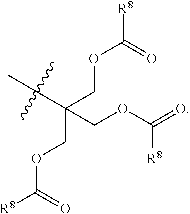

In some embodiments, G³ or G⁴ has

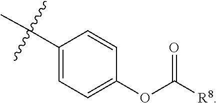

In some embodiments, G³ or G⁴ has

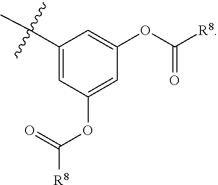

In some embodiments, G³ or G⁴ has

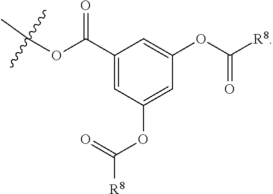

In some embodiments, G³ or G⁴ has

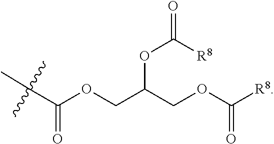

In one embodiment, R²⁸ is each independently linear C₆-C₁₂ alkyl, branched C₁₀-C₄₀ alkyl, linear C₁₅-C₂₀ alkenyl, or branched C₂₀-C₄₀ alkenyl. In some embodiments, R²⁸ is each independently linear C₇-C₁₀ alkyl, branched C₁₄-C₄₀ alkyl, linear C₁₅-C₂₀ alkenyl, or branched C₃₀-C₄₀ alkenyl. In some embodiments, R²⁸ is each independently linear C₇ alkyl. In some embodiments, R²⁸ is each independently linear C₈ alkyl. In some embodiments, R²⁸ is each independently linear C₉ alkyl. In some embodiments, R²⁸ is each independently linear C₁₀ alkyl. In some embodiments, R²⁸ is each independently branched C₁₄-alkyl. In some embodiments, R²⁸ is each independently branched C₁₅ alkyl. In some embodiments, R²⁸ is each independently branched C₁₆ alkyl. In some embodiments, R²⁸ is each independently branched C₁₇ alkyl. In some embodiments, R²⁸ is each independently branched C₁₈ alkyl. In some embodiments, R²⁸ is each independently branched C₁₉ alkyl. In some embodiments, R²⁸ is each independently branched C₂₀ alkyl. In some embodiments, R²⁸ is each independently branched C₂₁ alkyl. In some embodiments, R²⁸ is each independently branched C₂₂ alkyl. In some embodiments, R²⁸ is each independently branched C₂₃ alkyl. In some embodiments, R²⁸ is each independently branched C₂₄ alkyl. In some embodiments, R²⁸ is each independently branched C₂₅ alkyl. In some embodiments, R²⁸ is each independently branched C₂₆ alkyl. In some embodiments, R²⁸ is each independently branched C₂₇ alkyl. In some embodiments, R²⁸ is each independently branched C₂₈ alkyl. In some embodiments, R²⁸ is each independently branched C₂₉ alkyl. In some embodiments, R²⁸ is each independently branched C₃₀ alkyl. In some embodiments, R²⁸ is each independently branched C₃₁ alkyl. In some embodiments, R²⁸ is each independently branched C₃₂ alkyl. In some embodiments, R²⁸ is each independently branched C₃₃ alkyl. In some embodiments, R²⁸ is each independently branched C₃₄ alkyl. In some embodiments, R²⁸ is each independently branched C₃₅ alkyl. In some embodiments, R²⁸ is each independently branched C₃₆ alkyl. In some embodiments, R²⁸ is each independently branched C₃₇ alkyl. In some embodiments, R²⁸ is each independently branched C₃₈ alkyl. In some embodiments, R²⁸ is each independently branched C₃₉ alkyl. In some embodiments, R²⁸ is each independently branched C₄₀ alkyl. In some embodiments, R²⁸ is each independently linear C₁₅ alkenyl. In some embodiments, R²⁸ is each independently linear C₁₆ alkenyl. In some embodiments, R²⁸ is each independently linear C₁₇ alkenyl. In some embodiments, R²⁸ is each independently linear C₁₈ alkenyl. In some embodiments, R²⁸ is each independently linear C₁₉ alkenyl. In some embodiments, R²⁸ is each independently linear C₂₀ alkenyl. In some embodiments, R²⁸ is each independently branched C₃₀ alkenyl. In some embodiments, R²⁸ is each independently branched C₃₁ alkenyl. In some embodiments, R²⁸ is each independently branched C₃₂ alkenyl. In some embodiments, R²⁸ is each independently branched C₃₃ alkenyl. In some embodiments, R²⁸ is each independently branched C₃₄ alkenyl. In some embodiments, R²⁸ is each independently branched C₃₅ alkenyl. In some embodiments, R²⁸ is each independently branched C₃₆ alkenyl. In some embodiments, R²⁸ is each independently branched C₃₇ alkenyl. In some embodiments, R²⁸ is each independently branched C₃₈ alkenyl. In some embodiments, R²⁸ is each independently branched C₃₉ alkenyl. In some embodiments, R²⁸ is each independently branched C₄₀ alkenyl.

In some certain embodiments, R²⁸ each independently has one of the following structures:

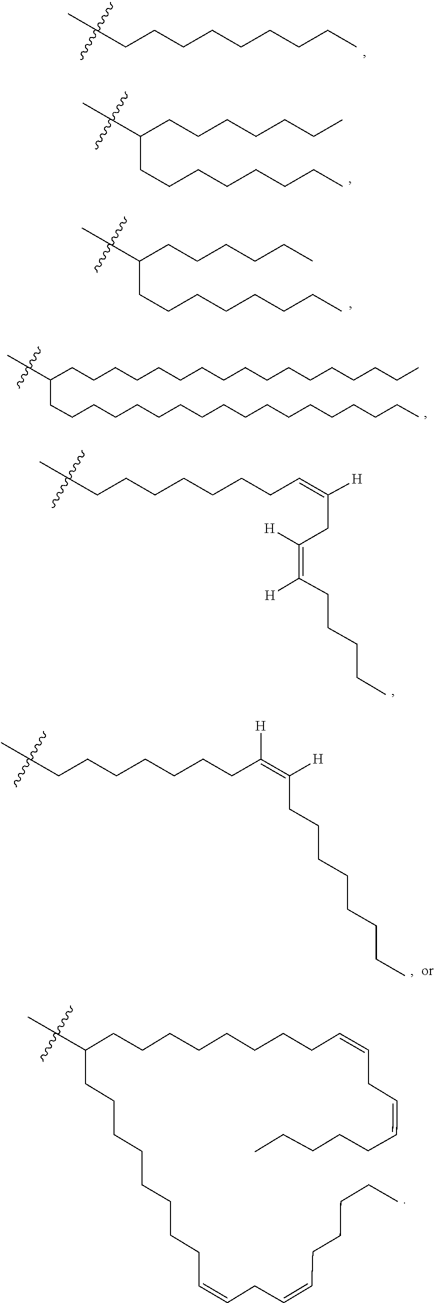

In some embodiments, R²⁸ is

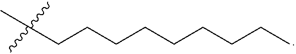 [SMILES: *CCCCCCCCC]

In some embodiments, R²⁸ is

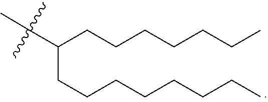 [SMILES: *C(CCCCCCC)CCCCCCCC]

In some embodiments, R²⁸ is

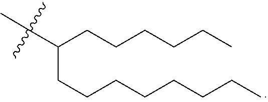 [SMILES: *C(CCCCCC)CCCCCCCC]

In some embodiments, R²⁸ is

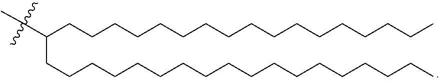 [SMILES: *C(CCCCCCCCCCCCCCCCC)CCCCCCCCCCCCCCCCCC]

In some embodiments, R²⁸ is

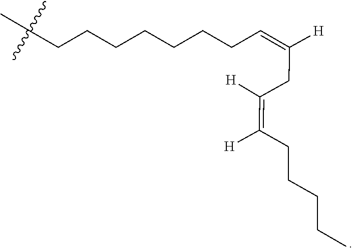 [SMILES: *CCCCCCC/C=C\C/C=C\CCCCC]

In some embodiments, R²⁸ is

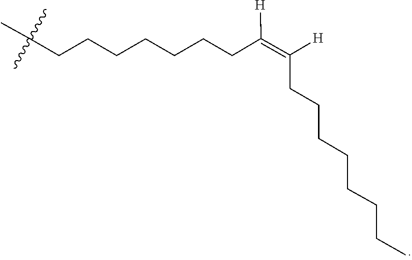 [SMILES: *CCCCCCC/C=C\CCCCCCCC]

In some embodiments, R²⁸ is

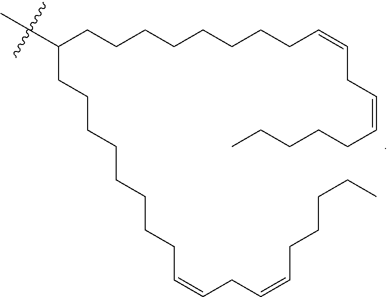 [SMILES: *C(CCCCCCCC/C=C\C/C=C\CCCCC)CCCCCCCC/C=C\C/C=C\CCCCC ; "."]

In one embodiment, m¹ and m² are each independently an integer from 1 to 6. In some embodiments, m¹ and m² are each independently an integer from 1 to 4. In some certain embodiments, m¹ or m² is 1. In some certain embodiments, m¹ or m² is 2. In some certain embodiments, m¹ or m² is 3. In some certain embodiments, m¹ or m² is 4. In some certain embodiments, m¹ or m² is 5. In some certain embodiments, m¹ or m² is 6.

In one embodiment, the compound has one of the following structures shown in Table B below.

TABLE B
List of Compounds of (XVIII)-(XVIIIC)

| Compound | |
|---|---|
| ID | Structure |
| 9 | 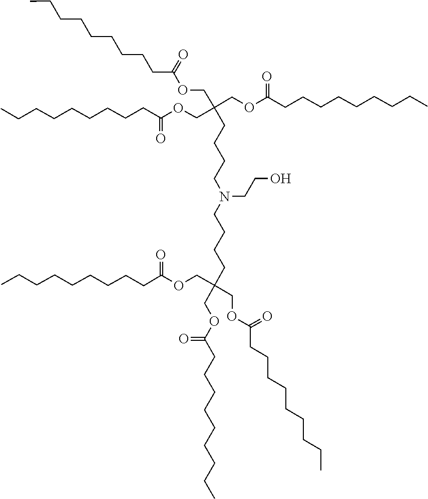 [SMILES: CCCCCCCCCC(=O)OCC(CCCCN(CCO)CCCCC(COC(=O)CCCCCCCCC)(COC(=O)CCCCCCCCC)COC(=O)CCCCCCCCC)(COC(=O)CCCCCCCCC)COC(=O)CCCCCCCCC] |
| 10 | 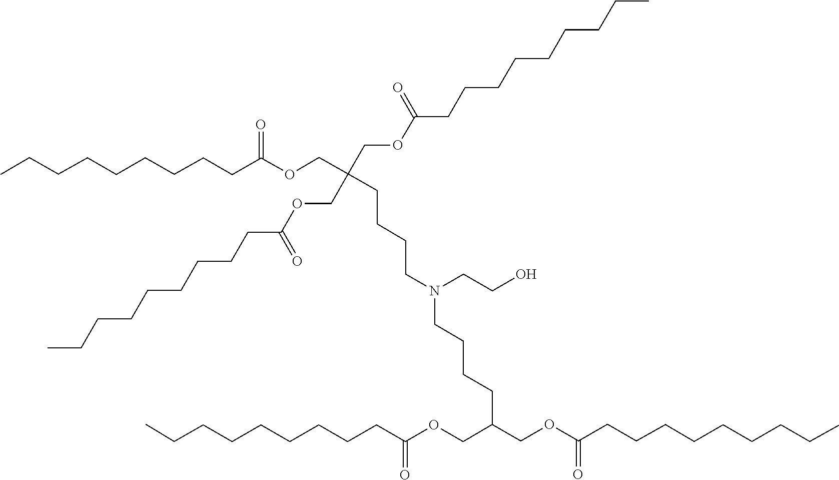 [SMILES: CCCCCCCCCC(=O)OCC(CCCCN(CCO)CCCCC(COC(=O)CCCCCCCCC)(COC(=O)CCCCCCCCC)COC(=O)CCCCCCCCC)COC(=O)CCCCCCCCC] |
| 7 | 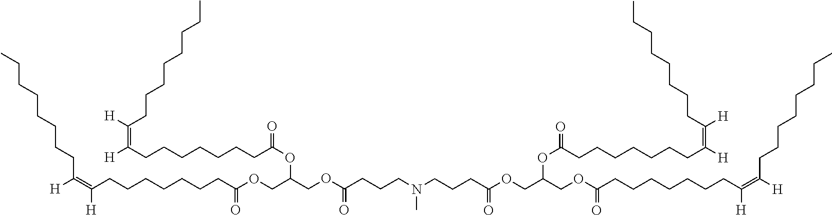 [SMILES: CCCCCCCC/C=C\CCCCCCCC(=O)OCC(COC(=O)CCCN(C)CCCC(=O)OCC(COC(=O)CCCCCCC/C=C\CCCCCCCC)OC(=O)CCCCCCC/C=C\CCCCCCCC)OC(=O)CCCCCCC/C=C\CCCCCCCC] |
| 8 | 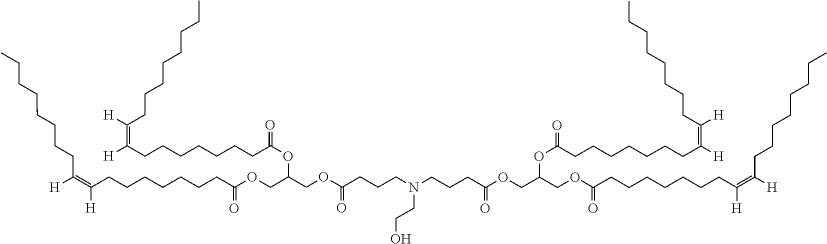 [SMILES: CCCCCCCC/C=C\CCCCCCCC(=O)OCC(COC(=O)CCCN(CCO)CCCC(=O)OCC(COC(=O)CCCCCCC/C=C\CCCCCCCC)OC(=O)CCCCCCC/C=C\CCCCCCCC)OC(=O)CCCCCCC/C=C\CCCCCCCC] |
| 28 | 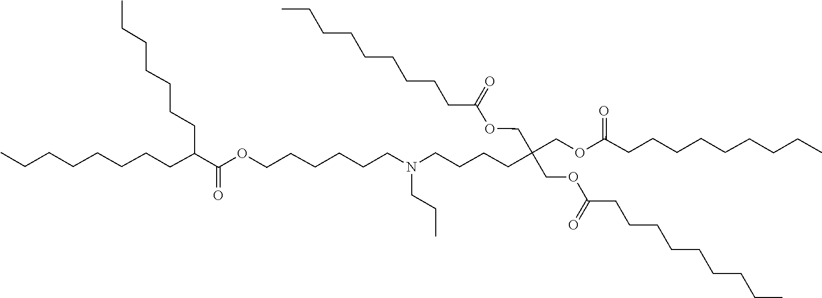 [SMILES: CCCCCCCCCC(=O)OCC(CCCCN(CCC)CCCCCCOC(=O)C(CCCCCCC)CCCCCCCC)(COC(=O)CCCCCCCCC)COC(=O)CCCCCCCCC] |
| 11 | 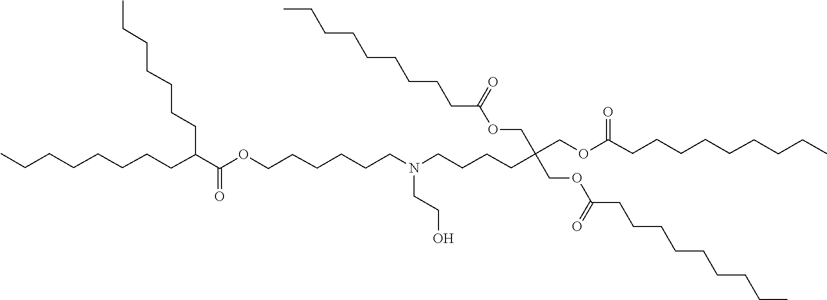 [SMILES: CCCCCCCCCC(=O)OCC(CCCCN(CCO)CCCCCCOC(=O)C(CCCCCCC)CCCCCCCC)(COC(=O)CCCCCCCCC)COC(=O)CCCCCCCCC] |
| 20 | 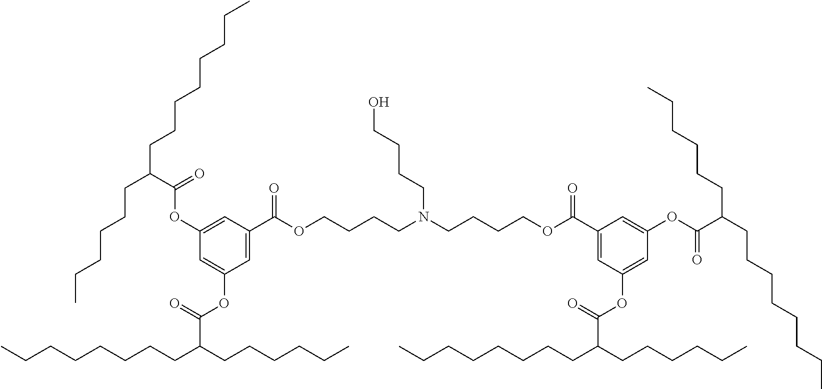 [SMILES: CCCCCCCCC(CCCCCC)C(=O)Oc1cc(OC(=O)C(CCCCCC)CCCCCCCC)cc(C(=O)OCCCCN(CCCCO)CCCCOC(=O)c2cc(OC(=O)C(CCCCCC)CCCCCCCC)cc(OC(=O)C(CCCCCC)CCCCCCCC)c2)c1] |
| 29 | 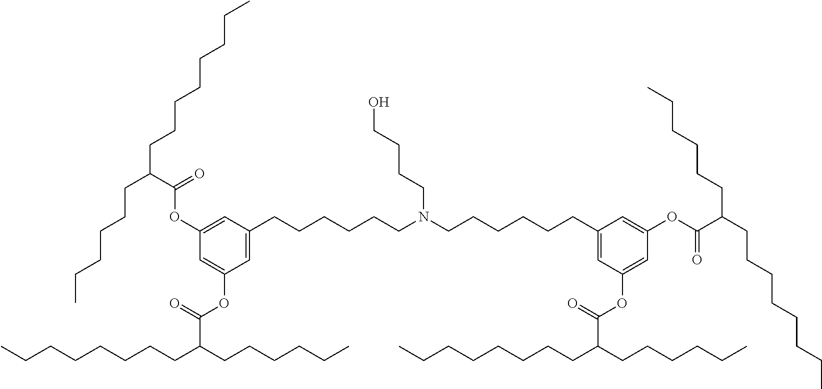 [SMILES: CCCCCCCCC(CCCCCC)C(=O)Oc1cc(CCCCCCN(CCCCO)CCCCCCc2cc(OC(=O)C(CCCCCC)CCCCCCCC)cc(OC(=O)C(CCCCCC)CCCCCCCC)c2)cc(OC(=O)C(CCCCCC)CCCCCCCC)c1] |
| 21 | 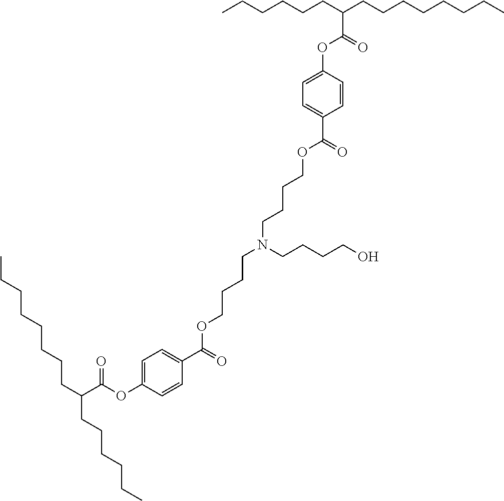 [SMILES: CCCCCCCCC(CCCCCC)C(=O)Oc1ccc(C(=O)OCCCCN(CCCCO)CCCCOC(=O)c2ccc(OC(=O)C(CCCCCC)CCCCCCCC)cc2)cc1] |
| 23 | 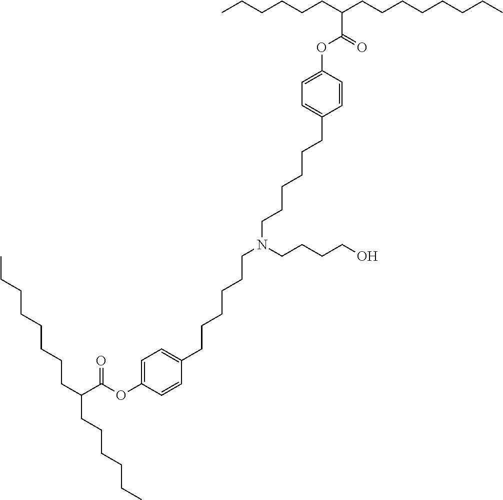 [SMILES: CCCCCCCCC(CCCCCC)C(=O)Oc1ccc(CCCCCCN(CCCCO)CCCCCCc2ccc(OC(=O)C(CCCCCC)CCCCCCCC)cc2)cc1] |
| 17 | 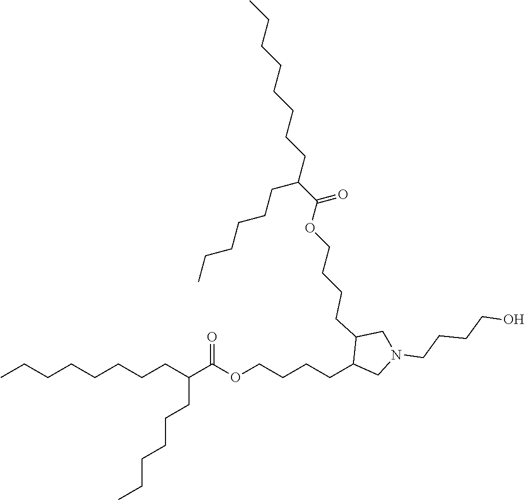 [SMILES: CCCCCCCCC(CCCCCC)C(=O)OCCCCC1CN(CCCCO)CC1CCCCOC(=O)C(CCCCCC)CCCCCCCC] |
| 15 | 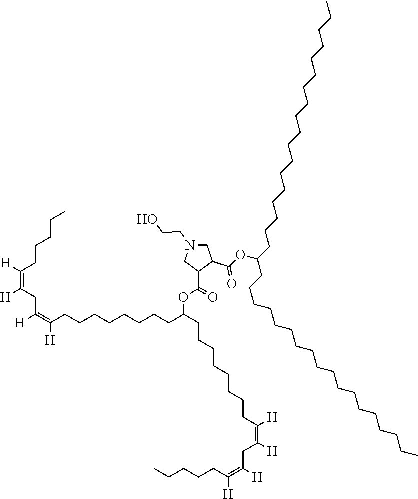 [SMILES: CCCCC/C=C\C/C=C\CCCCCCCCC(CCCCCCCC/C=C\C/C=C\CCCCC)OC(=O)C1CN(CCO)CC1C(=O)OC(CCCCCCCCCCCCCCCCCC)CCCCCCCCCCCCCCCCCC] |
| 30 | 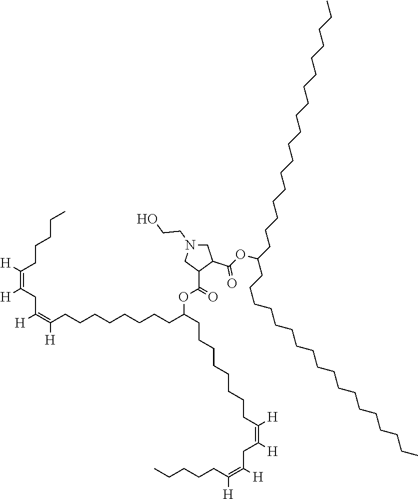 [SMILES: CCCCC/C=C\C/C=C\CCCCCCCCC(CCCCCCCC/C=C\C/C=C\CCCCC)OC(=O)C1CN(CCO)CC1C(=O)OC(CCCCCCCCCCCCCCCCCC)CCCCCCCCCCCCCCCCCC] |
| 14 | 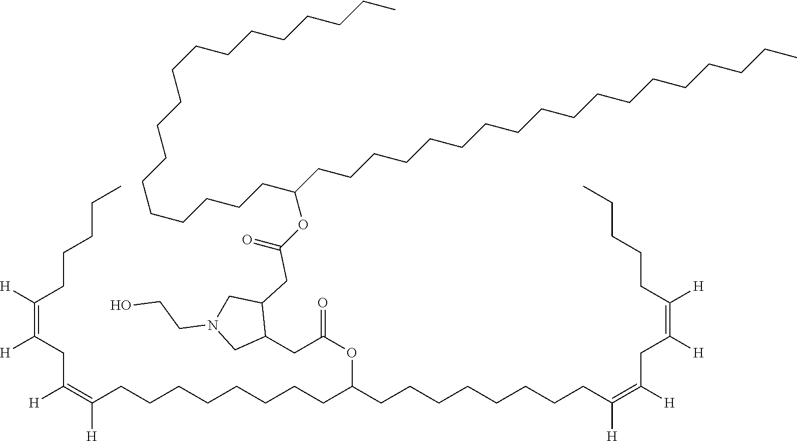 [SMILES: CCCCC/C=C\C/C=C\CCCCCCCCC(CCCCCCCC/C=C\C/C=C\CCCCC)OC(=O)CC1CN(CCO)CC1CC(=O)OC(CCCCCCCCCCCCCCCCCC)CCCCCCCCCCCCCCCCCC] |
| 18 | 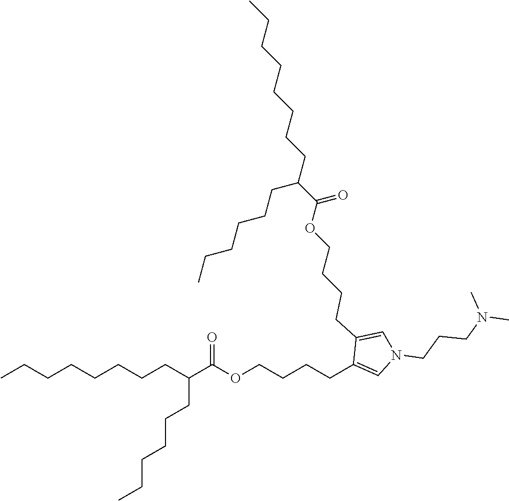 [SMILES: CCCCCCCCC(CCCCCC)C(=O)OCCCCc1cn(CCCN(C)C)cc1CCCCOC(=O)C(CCCCCC)CCCCCCCC] |
| 16 | 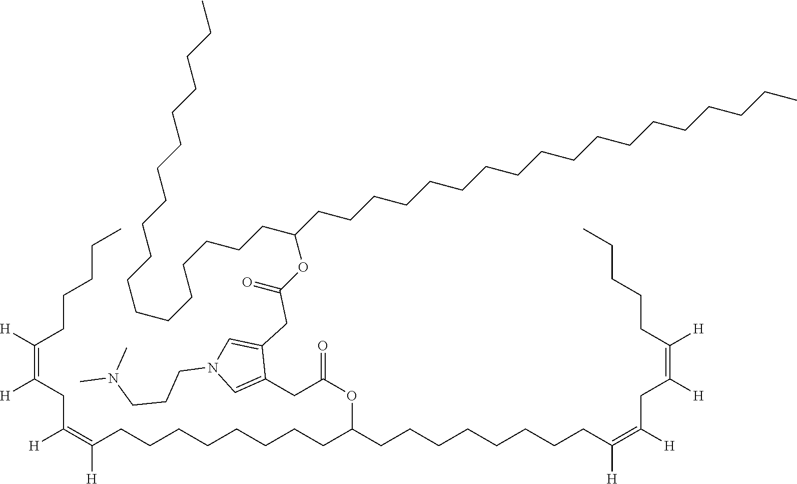 [SMILES: CCCCC/C=C\C/C=C\CCCCCCCCC(CCCCCCCC/C=C\C/C=C\CCCCC)OC(=O)Cc1cn(CCCN(C)C)cc1CC(=O)OC(CCCCCCCCCCCCCCCCCC)CCCCCCCCCCCCCCCCCC] |

In one embodiment, a compound has the following structure of Formula (XIX):

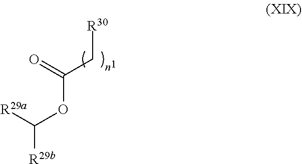

or a stereoisomer, salt or tautomer thereof, wherein: R^{29a} and R^{29b} are each independently linear C₆-C₁₀ alkyl or linear C₁₂-C₂₀ alkylene; R³⁰ is aryl or C₃-C₆ heterocycle, wherein the aryl or C₃-C₆ heterocycle is substituted with —OC(═O)R³¹, C₁-C₄ alkyl, or C₁-C₄ heteroalkyl; R³¹ is C₁-C₆ heteroalkyl; and n 1 is an integer from 1 to 6.

In cone embodiment, R^{29a} and R^{29b} are each independently linear C₆-C₁₀ alkyl or linear C₁₂-C₂₀ alkylene. In some embodiments, R^{29a} and R^{29b} are each linear C₆-C₁₀ alkyl. In some embodiments, R^{29a} and R^{29b} are each linear C₁₂-C₂₀ alkylene. In some certain embodiments, R^{29a} and R^{29b} each independently have one of the following structures:

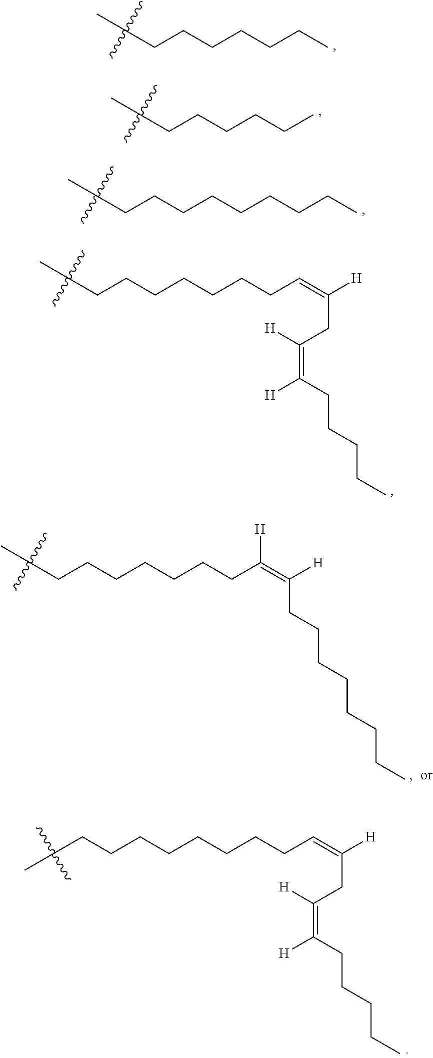

In some embodiment, R^{29a} or R^{29b} is

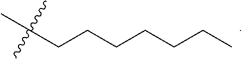 [SMILES: *CCCCCCC ; "."]

In some embodiment, R^{29a} or R^{29b} is

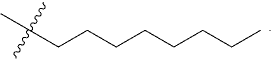 [SMILES: *CCCCCCCC ; "."]

In some embodiment, R^{29a} or R^{29b} is

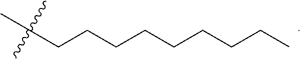 [SMILES: *CCCCCCCCC ; "."]

In some embodiment, R^{29a} or R^{29b} is

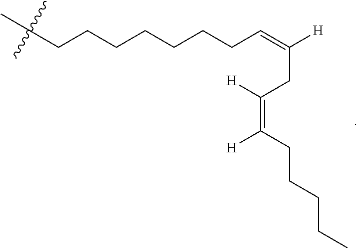 [SMILES: *CCCCCCC/C=C\C/C=C\CCCCC ; "."]

In some embodiment, R^{29a} or R^{29b} is

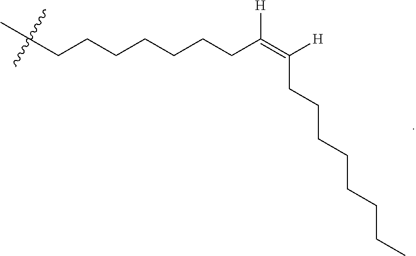 [SMILES: *CCCCCCC/C=C\CCCCCCCC ; "."]

In some embodiment, R^{29a} or R^{29b} is

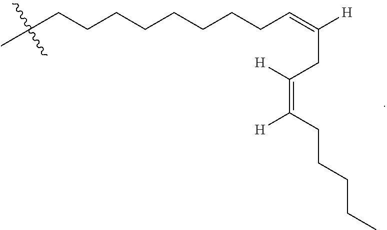 [SMILES: *CCCCCCCC/C=C\C/C=C\CCCCC ; "."]

In some certain embodiments, R^{29a} and R^{29b} are each

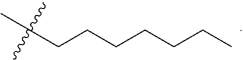 [SMILES: *CCCCCCC ; "."]

In some other certain embodiments, R^{29a} and R^{29b} are each

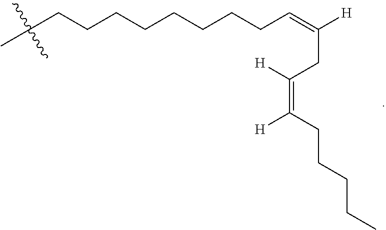 [SMILES: *CCCCCCCC/C=C\C/C=C\CCCCC ; "."]

In one embodiment, R³⁰ is aryl or C₃-C₆ heterocycle, wherein the aryl or C₃-C₆ heterocycle is substituted with —OC(═O)R³¹, C₁-C₄ alkyl, or C₁-C₄ heteroalkyl. In some embodiments, the aryl of R³⁰ is phenyl or naphthalene. In some embodiments, the C₃-C₆ heterocycle of R³⁰ is azetidine, pyrrolidine, imidazolidine, pyrazolidine, piperidine, diazinane, triazinane, or azepane. In some embodiments, R³⁰ is aryl substituted with —OC(═O)R³¹ or C₃-C₆ heterocycle substituted with C₁-C₄ alkyl, or C₁-C₄ heteroalkyl. In some embodiments, a phenyl substituted with —OC(═O)R³¹ or C₃-C₆ N-heterocycle substituted with C₁-C₄ alkyl, or C₁-C₄ heteroalkyl. In some embodiments, R³⁰ has one of the following structures:

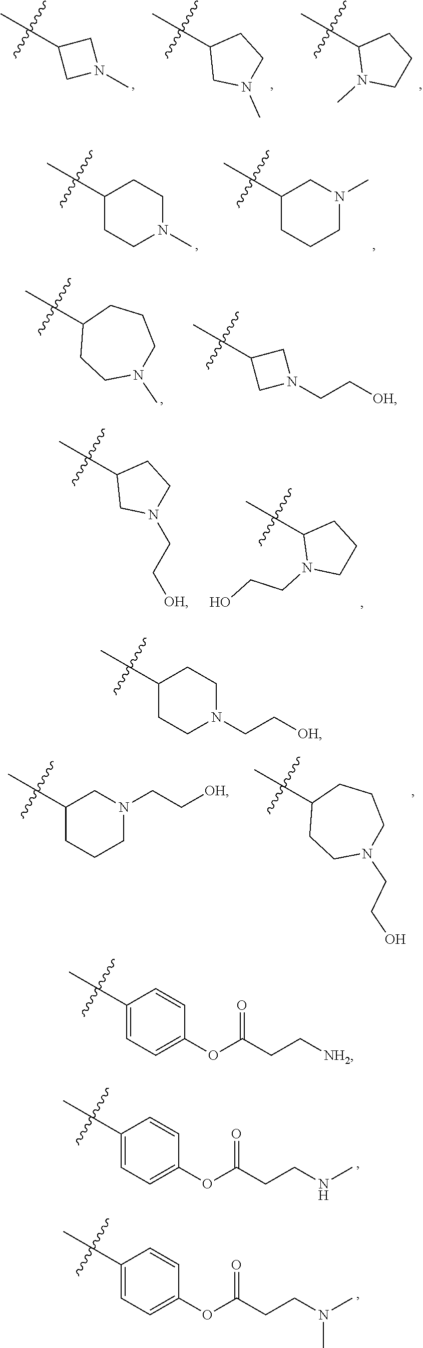

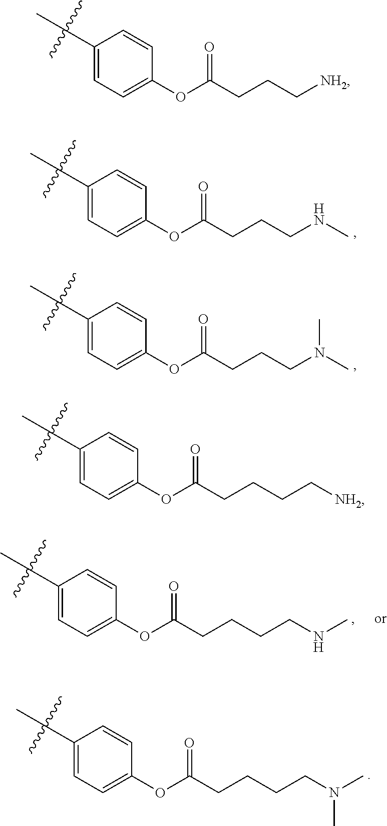 [SMILES: *c1ccc(OC(=O)CCCN)cc1 ; *c1ccc(OC(=O)CCCNC)cc1 ; *c1ccc(OC(=O)CCCN(C)C)cc1 ; *c1ccc(OC(=O)CCCCN)cc1 ; *c1ccc(OC(=O)CCCCNC)cc1 ; *c1ccc(OC(=O)CCCCN(C)C)cc1]

In some embodiments, R³⁰ is

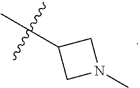 [SMILES: *C1CN(C)C1 ; "."]

In some embodiments, R³⁰ is

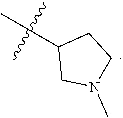 [SMILES: *C1CCN(C)C1 ; "."]

In some embodiments, R³⁰ is

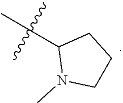 [SMILES: *C1CCCN1C ; "."]

In some embodiments, R³⁰ is

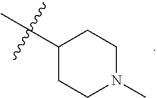 [SMILES: *C1CCN(C)CC1 ; "."]

In some embodiments, R³⁰ is

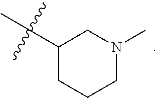 [SMILES: *C1CCCN(C)C1 ; "."]

In some embodiments, R³⁰ is

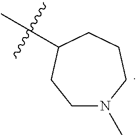 [SMILES: *C1CCCN(C)CC1]

In some embodiments, R³⁰ is

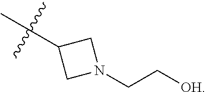 [SMILES: *C1CN(CCO)C1]

In some embodiments, R³⁰ is

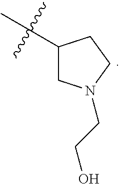 [SMILES: *C1CCN(CCO)C1]

In some embodiments, R³⁰ is

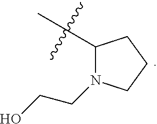 [SMILES: *C1CCCN1CCO]

In some embodiments, R³⁰ is

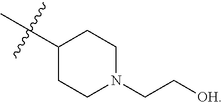 [SMILES: *C1CCN(CCO)CC1]

In some embodiments, R³⁰ is

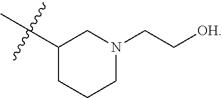 [SMILES: *C1CCCN(CCO)C1]

In some embodiments, R³⁰ is

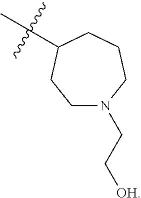 [SMILES: *C1CCCN(CCO)CC1]

In some embodiments, R³⁰ is

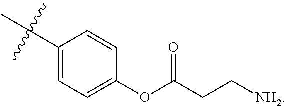 [SMILES: *c1ccc(OC(=O)CCN)cc1]

In some embodiments, R³⁰ is

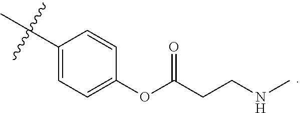 [SMILES: *c1ccc(OC(=O)CCNC)cc1]

In some embodiments, R³⁰ is

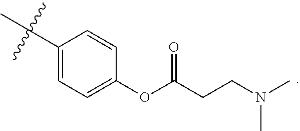 [SMILES: *c1ccc(OC(=O)CCN(C)C)cc1]

In some embodiments, R³⁰ is

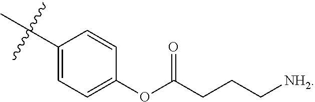 [SMILES: *c1ccc(OC(=O)CCCN)cc1]

In some embodiments, R³⁰ is

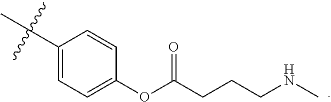 [SMILES: *c1ccc(OC(=O)CCCNC)cc1]

In some embodiments, R³⁰ is In some embodiments, R³⁰ is

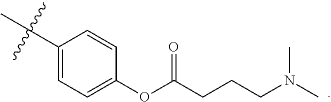 [SMILES: *c1ccc(OC(=O)CCCN(C)C)cc1]

In some embodiments, R³⁰ is

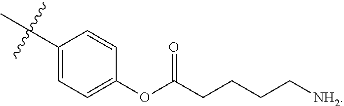 [SMILES: *c1ccc(OC(=O)CCCCN)cc1]

In some embodiments R³⁰ is

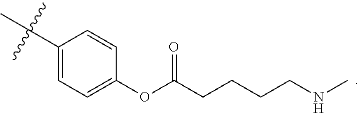 [SMILES: *c1ccc(OC(=O)CCCCNC)cc1]

In some embodiments, R³⁰ is

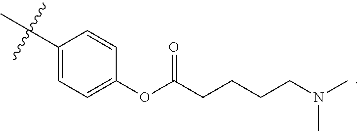 [SMILES: *c1ccc(OC(=O)CCCCN(C)C)cc1]

In some embodiments, n¹ is an integer from 1 to 4. In some embodiments, n¹ is an integer of 1. In some embodiments, n¹ is an integer of 2. In some embodiments, n¹ is an integer of 3. In some embodiments, n¹ is an integer of 4. In some embodiments, n¹ is an integer of 1 or 4.

In one embodiment, the compound has one of the following structures shown in Table C below.

TABLE C
List of Compounds of (XIX)

| Compound | |
|---|---|
| ID | Structure |
| 12 | 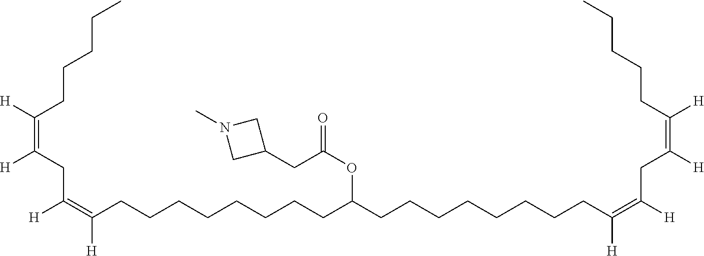 [SMILES: CCCCC/C=C\C/C=C\CCCCCCCCC(CCCCCCCC/C=C\C/C=C\CCCCC)OC(=O)CC1CN(C)C1] |
| 13 | 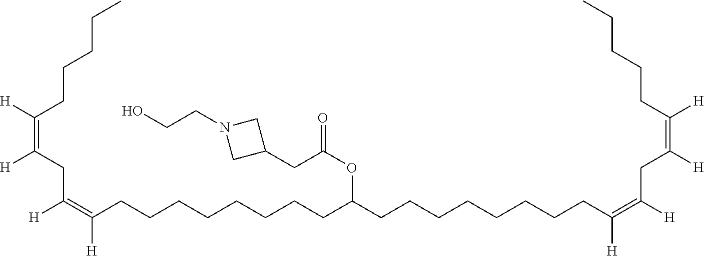 [SMILES: CCCCC/C=C\C/C=C\CCCCCCCCC(CCCCCCCC/C=C\C/C=C\CCCCC)OC(=O)CC1CN(CCO)C1] |
| 19 | 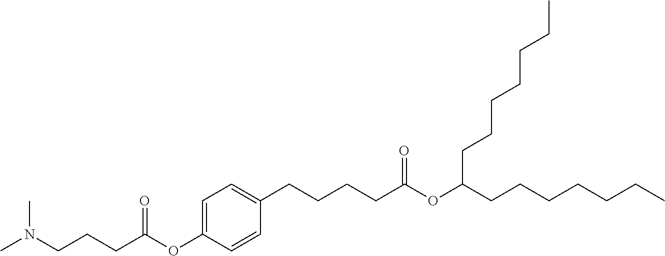 [SMILES: CCCCCCCC(CCCCCCC)OC(=O)CCCCc1ccc(OC(=O)CCCN(C)C)cc1] |

In one embodiment, an ionizable lipid of Formula (XXI):

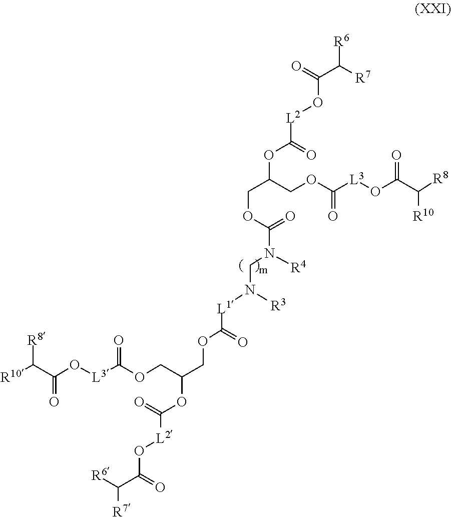

or a pharmaceutically acceptable salt or a stereoisomer thereof, wherein:

L¹ is C₁-C₆ alkylene; L^{1′} is C₁-C₆ alkylene; L² is C₁-C₈ alkylene; L^{2′} is C₁-C₈ alkylene; L³ is C₁-C₈ alkylene; L^{1′} is C₁-C₈ alkylene; R³ and R⁴ are each independently H, C₁-C₄ alkyl, —CH₂— cyclopropyl, or —(CH₂)ₙOH; R⁶ is C₄-C₂₀ alkyl; R^{6′} is C₄-C₂₀ alkyl; R⁷ is C₄-C₂₀ alkyl; R^{7′} is C₄-C₂₀ alkyl; R⁸ is C₄-C₂₀ alkyl; R¹⁰ is C₄-C₂₀ alkyl; R¹⁰ is C₄-C₂₀ alkyl; R^{10′} is C₄-C₂₀ alkyl; n is 2, 3, or 4; and m is 1, 2, 3, 4, or 5.

In one embodiment, the compound has one of the following structures shown in Table D below.

TABLE D
List of Compounds of (XXI)

| Compound | |
|---|---|
| ID | Structure |
| 56 | 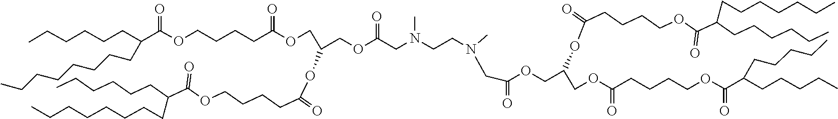 |

In one embodiment, a compound the following structure of Formula (XX):

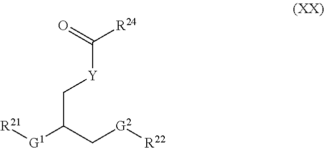

or a stereoisomer, salt or tautomer thereof, wherein: G¹ and G² are each independently —OC(═O)— or —NR²⁵C(═O)—; R²¹ and R²² are each independently C₁-C₆ alkyl, linear C₁₀-C₂₀ alkyl, linear C₁₀-C₂₀ alkenyl, or branched C₁₀-C₃₅ alkenyl, wherein the C₁-C₆ alkyl is substituted with —OC(═O)R²⁶; R²⁴ is C₁-C₆ heteroalkyl, aryl or C₁-C₄ alkyl substituted with a 4-8 membered heterocycloalkyl; R²⁵ is C₁-C₆ heteroalkyl, aryl or C₁-C₄ alkyl; R²⁶ is branched C₁₀-C₃₀ alkyl; and Y is O or NR³², wherein R³ is H or C₁-C₄ alkyl.

In one embodiment, G¹ and G² are each independently —OC(═O)— or —NR²⁵C(═O)—. In some embodiments, G¹ and G² are each —OC(═O)—. In some embodiments, G¹ and G² are each —NR²⁵C(═O)—. In some embodiments, one of G¹ or G² is —NR²⁵C(═O)— and the other one of G¹ or G² is —OC(═O)—.

In one embodiment, the compound has one of the following structures of Formula (XXA)-(XXB):

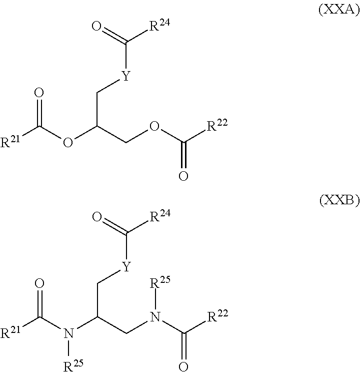

or a stereoisomer, salt or tautomer thereof. In some embodiments, the compound is

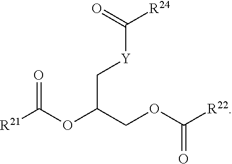

In some embodiments, the compound is

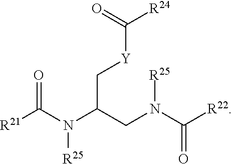

In one embodiment, Y is O. In other embodiments, Y is NR³², wherein R³² is H or C₁-C₄ alkyl. In some certain embodiments, Y is NR³² and R³² is H. In some other embodiments, Y is NR³² and R³² is C₁-C₄ alkyl. In some embodiments, C₁-C₄ alkyl of R³² is methyl, ethyl, n-propyl, iso-propyl, n-butyl, sec-butyl, or tert-butyl.

In one embodiment, the compound has one of the following structures of Formula (XXA-1)-(XXB-2):

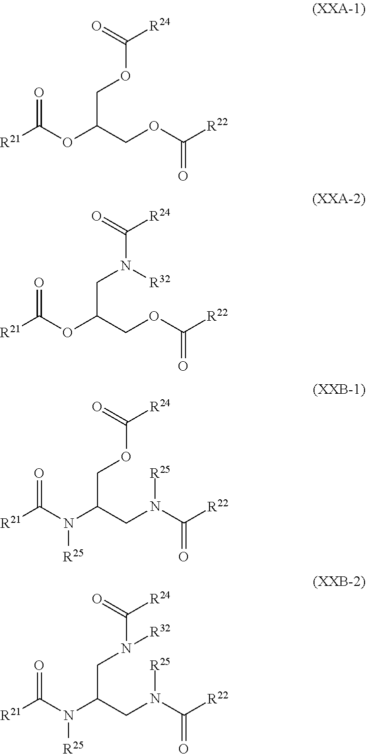

or a stereoisomer, salt or tautomer thereof. In one embodiment, the compound is

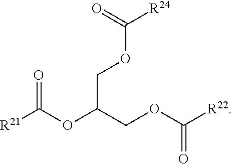

In some embodiments, the compound is

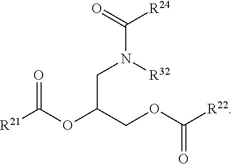

In some embodiments, the compound is

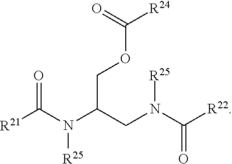

In some embodiments, the compound is

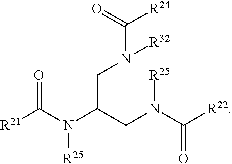

In one embodiment, R²⁴ is C₁-C₆ heteroalkyl. In some embodiments, R²⁴ is C₃-C₆ heteroalkyl. In some embodiments, R²⁴ is C₃ heteroalkyl. In some embodiments, R²⁴ is C₄ heteroalkyl. In some embodiments, R²⁴ is C₅ heteroalkyl. In some embodiments, R²⁴ is C₆ heteroalkyl. In some embodiments, R²⁴ is C₅ alkylamine. In some certain embodiments, R²⁴ is

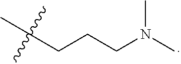 [SMILES: *CCCN(C)C]

In one embodiment, R²⁴ is an aryl. In some embodiments, the aryl of R²⁴ is substituted with C₁-C₆ heteroalkyl. In some embodiments, the aryl of R²⁴ is phenyl or naphthalene. In some certain embodiments, the aryl of R²⁴ is phenyl. In some other embodiments, the aryl of R²⁴ is substituted with C₂-C₄ heteroalkyl. In some embodiments, for example, C₂-C₄ heteroalkyl is a substituted amine. In some embodiments, R²⁴ is a phenyl substituted with C₂-C₄ heteroalkyl. In some embodiments, R²⁴ is a phenyl substituted with C₃ heteroalkyl. In some certain embodiments, the substituted aryl of R²⁴ is

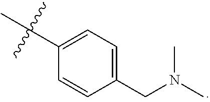 [SMILES: *c1ccc(CN(C)C)cc1]

In one embodiment, R²⁴ is C₁-C₄ alkyl substituted with a 4-8 membered heterocycloalkyl. In some embodiments, R²⁴ is C₁-C₃ alkyl substituted with a 4-6 membered heterocycloalkyl. In some embodiments, for example, C₁-C₃ alkyl of R²⁴ is methyl, ethyl, or n-propyl. In some embodiments, for example, the 4-6 membered heterocycloalkyl of R²⁴ is azetidine, oxetane, phosphetane, thietane, diazetidine, dioxetane, dithietane, pyrrolidine, tetrahydrofuran, phospholane, tetrahydrothiophene, imidazolidine, pyrazolidine, ozathiolidine, isoxthiolidine, ozazolidine, isoxazolidine, thiazolidine, dioxolane, dithiolane, piperidine, oxane, phosphinane, thiane, diazinane, morpholine, oxathiane, dioxane, or dithiane. In some certain embodiments, the 4-6 membered heterocycloalkyl of R²⁴ is azetidine or 1,4-diazinane. In some embodiments, R²⁴ has one of the following structures:

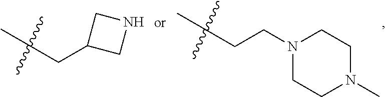

In some embodiments, R²⁴ is

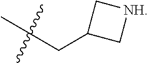 [SMILES: *CC1CNC1]

In some embodiments, R²⁴ is

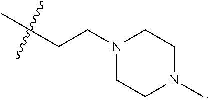 [SMILES: *CCN1CCN(C)CC1]

In one embodiment, R²⁵ is H or C₁-C₄ alkyl. In some embodiments, R²⁵ is H. In some embodiments, R²⁵ is C₁-C₄ alkyl. In some embodiments, R²⁵ is C₁ alkyl. In some embodiments, R²⁵ is C₂ alkyl. In some embodiments, R²⁵ is C₃ alkyl. In some embodiments, R²⁵ is C₄ alkyl. In some certain embodiments, R²⁵ is —CH₃. In some other certain embodiments, R²⁵ is —CH₂CH₃.

In one embodiment, R²¹ and R²² are each independently C₁-C₆ alkyl, linear C₁₀-C₂₀ alkyl, linear C₁₀-C₂₀ alkenyl, or branched C₁₀-C₃₅ alkenyl, wherein the C₁-C₆ alkyl is substituted with —OC(═O)R²⁶. In some embodiments, R²¹ and R²² are each independently C₂-C₅ alkyl substituted with —OC(═O)R²⁶, linear C₁₂-C₁₈ alkyl, linear C₁₂-C₁₈ alkenyl, or branched C₁₄-C₃₂ alkenyl. In some embodiments, one of R²¹ or R²² is linear C₁₂-C₁₈ alkyl and the other one of R²¹ or R²² is linear C₁₂-C₁₈ alkenyl. In some embodiments, one of R²¹ or R²² is C₂-C₅ alkyl substituted with —OC(═O)R²⁶ and the other one of R²¹ or R²² is linear C₁₂-C₁₈ alkyl. In some embodiments, one of R²¹ or R²² is linear C₁₂-C₁₈ alkyl and the other one of R²¹ or R²² is branched C₁₄-C₃₂ alkenyl. In some embodiments, R²¹ and R²² are each C₂-C₅ alkyl substituted with —OC(═O)R²⁶. In some embodiments, R²¹ and R²² are each C₂ alkyl substituted with —OC(═O)R²⁶. In some embodiments, R²¹ and R²² are each C₃ alkyl substituted with —OC(═O)R²⁶. In some embodiments, R²¹ and R²² are each C₄ alkyl substituted with —OC(═O)R²⁶. In some embodiments, R²¹ and R²² are each C₅ alkyl substituted with —OC(═O)R²⁶. In some embodiments, R²¹ and R²² are each linear C₁₂-C₁₈ alkenyl. In some embodiments, R²¹ and R²² are each linear C₁₂ alkenyl. In some embodiments, R²¹ and R²² are each linear C₁₃ alkenyl. In some embodiments, R²¹ and R²² are each linear C₁₄ alkenyl. In some embodiments, R²¹ and R²² are each linear C₁₅ alkenyl. In some embodiments, R²¹ and R²² are each linear C₁₆ alkenyl. In some embodiments, R²¹ and R²² are each linear C₁₇ alkenyl. In some embodiments, R²¹ and R²² are each linear C₁₈ alkenyl.

In one embodiment, R²⁶ is branched C₁₀-C₃₀ alkyl. In some embodiments, R²⁶ is branched C₁₀-C₂₀ alkyl. In some certain embodiments, R²⁶ is branched C₁₂-C₁₈ alkyl. In some certain embodiments, R²⁶ is branched C₁₂ alkyl. In some certain embodiments, R²⁶ is branched C₁₃ alkyl.

In some certain embodiments, R²⁶ is branched C₁₄ alkyl. In some certain embodiments, R²⁶ is branched C₁₅ alkyl. In some certain embodiments, R²⁶ is branched C₁₆ alkyl. In some certain embodiments, R²⁶ is branched C₁₇ alkyl. In some certain embodiments, R²⁶ is branched C₁₈ alkyl.

In some embodiments, R²¹ and R²² each independently have the following structure:

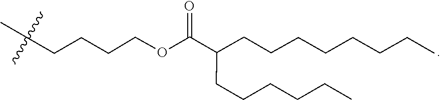 [SMILES: *CCCCCOC(=O)C(CCCCCC)CCCCCCCC]

In one embodiment, the compound has one of the following structures shown in Table E below.

TABLE E
List of Compounds of (XX)-(XXB-2)

| Compound | |
|---|---|
| ID | Structure |
| 40 | 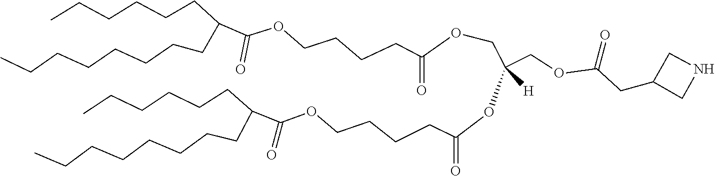 [SMILES: CCCCCCCCC(CCCCCC)C(=O)OCCCCC(=O)OC[C@H](COC(=O)CC1CNC1)OC(=O)CCCCOC(=O)C(CCCCCC)CCCCCCCC] |
| 41 | 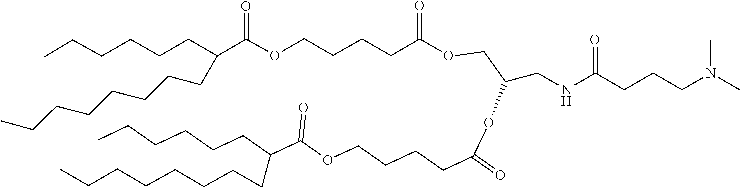 |
| 42 | 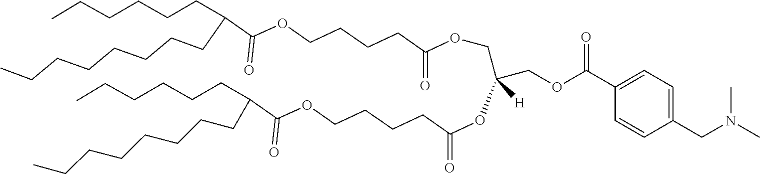 [SMILES: CCCCCCCCC(CCCCCC)C(=O)OCCCCC(=O)OC[C@H](COC(=O)c1ccc(CN(C)C)cc1)OC(=O)CCCCOC(=O)C(CCCCCC)CCCCCCCC] |
| 43 | 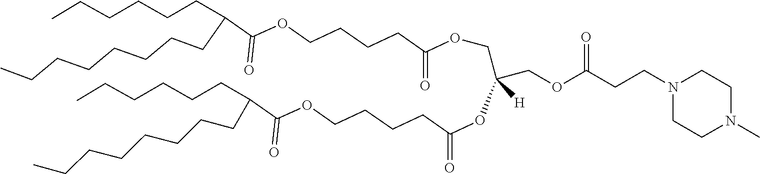 [SMILES: CCCCCCCCC(CCCCCC)C(=O)OCCCCC(=O)OC[C@H](COC(=O)CCN1CCN(C)CC1)OC(=O)CCCCOC(=O)C(CCCCCC)CCCCCCCC] |
| 44 | 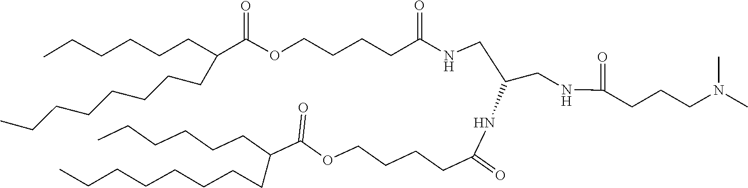 |

### III. NUCLEIC ACIDS

In one aspect, the ionizable lipids of the disclosure are useful for the delivery of nucleic acids. The nucleic acids include, but are not limited to small interfering RNA (siRNA), an asymmetrical interfering RNA (aiRNA), a microRNA (miRNA), miRNA inhibitors (antagomirs/antimirs), a dicer-substrate RNA (dsRNA), a small hairpin RNA (shRNA), a messenger RNA (mRNA), multivalent RNA, and mixtures thereof. In some aspects, the nucleic acids or mRNA include self-amplifying RNA (saRNA), polycistronic RNA, circular RNA, and mixtures thereof.

In one aspect, the nucleic acid comprises an interfering RNA molecule such as, e.g., an siRNA, aiRNA, miRNA, or mixtures thereof. In certain other aspects, the nucleic acid comprises one or more mRNA molecules (e.g., a cocktail).

The interfering RNA molecule includes “small-interfering RNA” or “siRNA,” e.g., interfering RNA of about 15-60, 15-50, or 15-40 (duplex) nucleotides in length, more typically about 15-30, 15-25, or 19-25 (duplex) nucleotides in length, and is preferably about 20-24, 21-22, or 21-23 (duplex) nucleotides in length (e.g., each complementary sequence of the double-stranded siRNA is 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 18-22, 19-20, or 19-21 base pairs in length). siRNA duplexes may comprise 3′ overhangs of about 1 to about 4 nucleotides or about 2 to about 3 nucleotides and 5′ phosphate termini.

Examples of siRNA include, without limitation, a double-stranded polynucleotide molecule assembled from two separate stranded molecules, wherein one strand is the sense strand and the other is the complementary antisense strand; a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule.

siRNA can also be chemically synthesized. siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., Proc. Natl. Acad. Sci. USA, 99:9942-9947 (2002); Calegari et al., Proc. Natl. Acad. Sci. USA, 99: 14236 (2002); Byrom et al., Ambion TechNotes, 10(1):4-6 (2003); Kawasaki et al., Nucleic Acids Res., 31:981-987 (2003); Knight et al., Science, 293:2269-2271 (2001); and Robertson et al., J. Biol. Chem., 243:82 (1968)). Preferably, dsRNA are at least 50 nucleotides to about 100, 200, 300, 400, or 500 nucleotides in length. A dsRNA may be as long as 1000, 1500, 2000, 5000 nucleotides in length, or longer. The dsRNA can encode for an entire gene transcript or a partial gene transcript. In certain instances, siRNA may be encoded by a plasmid (e.g., transcribed as sequences that automatically fold into duplexes with hairpin loops).

In one embodiment, the nucleic acid comprises an siRNA. In one embodiment, the siRNA molecule comprises a double-stranded region of about 15 to about 60 nucleotides in length (e.g., about 15-60, 15-50, 15-40, 15-30, 15-25, or 19-25 nucleotides in length, or 15, 16, 17, 18, 19, 20, 21, 22, 23, 24, or 25 nucleotides in length). The siRNA molecules can silence the expression of a target sequence in vitro and/or in vivo.

In other embodiments, the siRNA molecule comprises modified nucleotides including, but not limited to, 2′-O-methyl (2′OMe) nucleotides, 2′-deoxy-2′-fluoro (2′F) nucleotides, 2′-deoxy nucleotides, 2′-O-(2-methoxyethyl) (MOE) nucleotides, locked nucleic acid (LNA) nucleotides, and mixtures thereof. In some other embodiments, the siRNA comprises 2′-OMe nucleotides (e.g., 2-′OMe purine and/or pyrimidine nucleotides) such as, for example, 2′-OMe-guanosine nucleotides, 2′-OMe-uridine nucleotides, 2′OMe-adenosine nucleotides, 2′-OMe-cytosine nucleotides, and mixtures thereof. In certain instances, the siRNA does not comprise 2′-OMe-cytosine nucleotides. In other embodiments, the siRNA comprises a hairpin loop structure.

The nucleic acids may be prepared according to any available technique. For mRNA, the primary methodology of preparation is, but is not limited to, enzymatic synthesis (also termed in vitro transcription) to produce long sequence-specific mRNA. In vitro transcription describes a process of template-directed synthesis of RNA molecules from an engineered DNA template comprised of an upstream bacteriophage promoter sequence (e.g., including but not limited to that from the T7, T3 and SP6 coliphage) linked to a downstream sequence encoding the gene of interest.

Transcription of the RNA occurs in vitro using the linearized DNA template in the presence of the corresponding RNA polymerase and adenosine, guanosine, uridine and cytidine ribonucleoside triphosphates (rNTPs) under conditions that support polymerase activity while minimizing potential degradation of the resultant mRNA transcripts. In vitro transcription can be performed using a variety of commercially available kits including, but not limited to RiboMax Large Scale RNA Production System (Promega), MegaScript Transcription kits (Life Technologies) as well as with commercially available reagents including RNA polymerases and rNTPs. The methodology for in vitro transcription of mRNA is well known in the art. (see, e.g. Losick, R., 1972, In vitro transcription, Ann Rev Biochem v. 41 409-46; Kamakaka, R. T. and Kraus, W. L. 2001. In Vitro Transcription. Current Protocols in Cell Biology. 2:11.6:11.6.1-11.6.17; Beckert, B. And Masquida, B., (2010) Synthesis of RNA by In Vitro Transcription in RNA in Methods in Molecular Biology v. 703 (Neilson, H. Ed), New York, N.Y. Humana Press, 2010; Brunelle, J. L. and Green, R., 2013, Chapter Five—In vitro transcription from plasmid or PCR-amplified DNA, Methods in Enzymology v. 530, 101-114; all of which are incorporated herein by reference).

The desired in vitro transcribed mRNA is then purified from the undesired components of the transcription or associated reactions (including unincorporated rNTPs, protein enzyme, salts, short RNA oligos etc.). Techniques for the isolation of the mRNA transcripts are well known in the art. Well known procedures include phenol/chloroform extraction or precipitation with either alcohol (ethanol, isopropanol) in the presence of monovalent cations or lithium chloride. Additional, non-limiting examples of purification procedures which can be used include size exclusion chromatography (Lukaysky, P. J. and Puglisi, J. D., 2004, Large-scale preparation and purification of polyacrylamide-free RNA oligonucleotides, RNA v. 10, 889-893), silica-based affinity chromatography and polyacrylamide gel electrophoresis (Bowman, J. C., Azizi, B., Lenz, T. K., Ray, P., and Williams, L. D. in RNA in vitro transcription and RNA purification by denaturing PAGE in Recombinant and in vitro RNA syntheses Methods v. 941 Conn G. L. (ed), New York, N.Y. Humana Press, 2012). Purification can be performed using a variety of commercially available kits including, but not limited to SV Total Isolation System (Promega) and In Vitro Transcription Cleanup and Concentration Kit (Norgen Biotek).

A significant variety of modifications have been described in the art which are used to alter specific properties of in vitro transcribed mRNA and improve its utility. These include but are not limited to modifications to the 5′ and 3′ termini of the mRNA. Endogenous eukaryotic mRNA typically contain a cap structure on the 5′-end of a mature molecule which plays an important role in mediating binding of the mRNA Cap Binding Protein (CBP), which is in turn responsible for enhancing mRNA stability in the cell and efficiency of mRNA translation. Therefore, highest levels of protein expression are achieved with capped mRNA transcripts. The 5′-cap contains a 5′-5′-triphosphate linkage between the 5′-most nucleotide and guanine nucleotide. Additional modifications include methylation of the ultimate and penultimate most 5′-nucleotides on the 2′-hydroxyl group.

Other components of mRNA which can be modified to provide benefit in terms of translatability and stability include the 5′ and 3′ untranslated regions (UTR). Optimization of the UTRs (favorable 5′ and 3′ UTRs can be obtained from cellular or viral RNAs), either both or independently, have been shown to increase mRNA stability and translational efficiency of in vitro transcribed mRNA (see e.g., Pardi, N., Muramatsu, H., Weissman, D., Kariko, K., In vitro transcription of long RNA containing modified nucleosides in Synthetic Messenger RNA and Cell Metabolism Modulation in Methods in Molecular Biology v. 969 (Rabinovich, P. H. Ed), 2013).

In some embodiments, the nucleic acid is RNA is monocistronic or polycistronic, wherein polycistronic RNA has 2, 3, 4, 5, 6 or more coding sequences expressing 2, 3, 4, 5, 6 or more different proteins or peptides or different subunits or fragments of one or more proteins or peptides. In some aspects, the nucleic acid is a cocktail of multiple mRNAs, each encoding at least one protein or peptide or subunit or fragment of a protein or peptide. In some aspects, the nucleic acid is a cocktail of multiple mRNAs, each encoding at least one reprogramming factor such as, but not limited to OCT, SOX, KLF, Lin, Nanog, Myc, or GLis1. In some embodiments, the reprogramming factor is LIN28 or NANOG. In some aspects, the reprogramming factor is a Yamanaka factor such as OCT4, SOX2, c-Myc, and KLF4. In some embodiments, the reprogramming factors are the combination of OCT4, SOX2, and c-Myc. In some embodiments, the reprogramming factors are the combination of OCT4, SOX2, KLF4, and c-Myc. In some embodiments, the reprogramming factors are the combination of OCT4, SOX2, KLF4, Lin28, Nanog, and c-Myc. In some embodiments, the reprogramming factors are the combination of OCT4, SOX2, KLF4, Lin28, Nanog, and c-Myc. In some embodiments, c-Myc in any of the combinations above is replaced by Glis1. In some embodiments, each mRNA encodes one reprogramming factor. In some embodiments, each mRNA encodes two or more reprogramming factors. In some embodiments, each mRNA encodes two reprogramming factors. In some embodiments, each mRNA encodes three reprogramming factors. In some embodiments, the nucleic acid is a single mRNA molecule that encodes two, three, four, five, six, or more reprogramming factors. In some embodiments, the nucleic acid is a single mRNA molecule that encodes three reprogramming factors. In some embodiments, the nucleic acid is a single mRNA molecule that encodes four reprogramming factors. In some embodiments, the nucleic acid is a single mRNA molecule that encodes five reprogramming factors. In some embodiments, the nucleic acid is a single mRNA molecule that encodes six reprogramming factors.

In some embodiments, the RNA is a polycistronic RNA coding for one or more reprogramming factors such as, but not limited to OCT, SOX, KLF, Lin, Nanog, Myc, or GLis1. In some embodiments, the reprogramming factor is LIN28 or NANOG. In some aspects, the reprogramming factor is a Yamanaka factor such as OCT4, SOX2, c-Myc, and KLF4. In some embodiments, the reprogramming factors are the combination of OCT4, SOX2, and c-Myc. In some embodiments, the reprogramming factors are the combination of OCT4, SOX2, KLF4, and c-Myc.

In some embodiments, the reprogramming factors are the combination of OCT4, SOX2, KLF4, Lin28, Nanog, and c-Myc. In some embodiments, the reprogramming factors are the combination of OCT4, SOX2, KLF4, Lin28, Nanog, and c-Myc. In some embodiments, c-Myc in any of the combinations above is replaced by Glis1.

In some embodiments, the RNA is a self-replicating RNA. Self-replicating constructs are described, for example, in U.S. Patent Publication Nos. 2018/0216079 and 2021/0108179, which are incorporated by reference herein. In some embodiments, the self-replicating RNA has increased half-life in a mammal, e.g., a human. In some aspects, the self-amplifying RNA is polycistronic. In some aspects, the self-amplifying RNA is a trans-amplifying RNA, where the amplification polymerase is coded by a different RNA strand from the strand or strands targeted for amplification.

In some embodiments, the RNA is a circular polyribonucleotide, or circular RNA. The circular polyribonucleotide, or circular RNA, is a polyribonucleotide that forms a circular structure through covalent or non-covalent bonds. In some embodiments, the circular polyribonucleotide is non-immunogenic in a mammal, e.g., a human. In some embodiments, the circular polyribonucleotide has increased half-life in a mammal, e.g., a human. In some embodiments, the circular polyribonucleotide is capable of replicating or replicates in a cell. In some aspects, the circular RNA is polycistronic.

In some embodiments, the RNA comprises a regulatory element, e.g., a sequence that modifies expression of a coding sequence within the RNA. A regulatory element may include a sequence that is located adjacent to a coding sequence that encodes an expression product. A regulatory element may be linked operatively to the adjacent sequence. A regulatory element may increase an amount of product expressed as compared to an amount of the expressed product when no regulatory element exists. In addition, one regulatory element can increase the amount of products expressed for multiple expression sequences attached in tandem. Hence, one regulatory element can enhance the expression of one or more expression sequences. Multiple regulatory elements are well-known to persons of ordinary skill in the art. In some embodiments, the regulatory element is an IRES or 2A element. In some embodiments, the IRES or 2A element is present upstream of the coding sequence. In some embodiments, the sequence of the IRES or 2A element is modified or optimized to achieve a desired expression profile. In some embodiments of polycistronic RNA, each coding sequence is separately regulated by a different IRES or 2A element, i.e., each gene being expressed has its own IRES or 2A element.

In some embodiments, one or more of the RNA molecules encodes b18r, b19r, E3, K3, or other “decoy molecules” to neutralize the type I interferon gamma response to the transfected RNA, thereby blunting the cell's immune response to the transfected RNA and resulting in increased translation of the therapeutic molecule encoded by the RNA, for example the reprogramming factors described above.

In some embodiments, the RNA has a length from about 100 to about 300 nucleotides, from about 300 to about 1,000 nucleotides, from about 1,000 to about 3,000 nucleotides, from about 3,000 to about 5,000 nucleotides, from about 5,000 to about 7,000 nucleotides, from about 7,000 to about 10,000 nucleotides, from about 10,000 to about 13,000 nucleotides, or about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 10100, 10200, 10300, 10400, 10500, 10600, 10700, 10800, 10900, 11000, 11100, 11200, 11300, 11400, 11500, 11600, 11700, 11800, 11900, 12000, 12100, 12200, 12300, 12400, 12500, 12600, 12700, 12800, 12900, 13000 base pairs.

In some embodiments, the nucleic acid (RNA or DNA) is cloned into a vector. In some aspects, the vector is an RNA vector that generates a monocistronic mRNA or a polycistronic mRNA, wherein the vector is linear or circular. In one embodiment, the vector is an mRNA producing vector that produces mRNA by in vitro transcription of a DNA vector. The DNA vector can be monocistronic or polycistronic (with 2, 3, 4, 5, 6 or more DNA sequences encoding for a reprogramming factor).

In some embodiments, the RNA vector is a polycistronic RNA vector. Such polycistronic RNA vectors can encode one or more reprogramming factors such as, but not limited to OCT, SOX, KLF, Lin, Nanog, Myc, or Glis1. In some embodiments, the one or more reprogramming factors comprise LIN28 or NANOG. In some aspects, the one or more reprogramming factors comprise a Yamanaka factor such as OCT4, SOX2, c-Myc, and KLF4. In some embodiments, the polycistronic RNA vector encodes OCT4, SOX2, and c-Myc. In some embodiments, the polycistronic RNA vector encodes OCT4, SOX2, KLF4, and c-Myc. In some embodiments, the polycistronic RNA vector encodes OCT4, SOX2, KLF4, c-Myc, LIN28, and NANOG. In any of the embodiments above, c-Myc may be replaced by Glis1.

In some aspects, the RNA vector is a self-replicating vector. In some aspects, the self-replicating vector has an off switch. Self-replicating and polycistronic constructs, and those with on/off switches, are described, for example, in U.S. Patent Publication Nos. 2018/0216079 and 2021/0108179, which are incorporated by reference herein, or in U.S. patent application Ser. Nos. 17/812,709, 17/812,711, and 17/812,710, which are incorporated by reference herein.

In some embodiments, the RNA vector is a circular polyribonucleotide, or circular RNA. The circular polyribonucleotide, or circular RNA, is a polyribonucleotide that forms a circular structure through covalent or non-covalent bonds. In some embodiments, the circular polyribonucleotide is non-immunogenic in a mammal, e.g., a human. In some embodiments, the circular polyribonucleotide is capable of replicating or replicates in a cell.

In some embodiments, the circular polyribonucleotide comprises a regulatory element, e.g., a sequence that modifies expression of an expression sequence within the circular polyribonucleotide. A regulatory element may include a sequence that is located adjacent to an expression sequence that encodes an expression product. A regulatory element may be linked operatively to the adjacent sequence. A regulatory element may increase an amount of product expressed as compared to an amount of the expressed product when no regulatory element exists. In addition, one regulatory element can increase an amount of products expressed for multiple expression sequences attached in tandem. Hence, one regulatory element can enhance the expression of one or more expression sequences. Multiple regulatory element are well-known to persons of ordinary skill in the art.

### IV. LIPID-NANOPARTICLE COMPOSITIONS

This disclosure features the ionizable lipids and compositions involving the same. Such compositions can be, but not limited to nanoparticle compositions. The lipid-nanoparticle composition of the disclosure may include the ionizable lipid of any one of Formula (I)-(XI), as well as additional lipids such as helper lipids, stabilization lipids and/or structural lipids.

Without wishing to be bound by any theory, it is thought that these lipid nanoparticles shield nucleic acids from degradation in the serum and provide for effective delivery of nucleic acids to cells in vitro and in vivo.

The lipid-nanoparticle composition of the disclosure may further include a nucleic acid, such as RNA as a therapeutic and/or a prophylactic and/or diagnostic for delivery to mammalian cells or organs to regulate or polypeptide, protein or gene expression.

The lipid compositions may be prepared by mixing the ionizable lipid of Formula (I)-(XIX), or a combination thereof, with a helper lipid or a combination thereof, a stabilization lipid and/or a structural lipid or a combination thereof in solvents, such as ethanol and water to yield the desired molar ratio.

The helper lipids for use in the lipid-nanoparticle compositions of the disclosure may include lipids that may assemble into one or more lipid bilayers. The helper lipids for use in the lipid-nanoparticle compositions of the disclosure may include lipids that increase the stability or delivery efficiency of lipid nanoparticles.

Exemplary examples of helper lipids that can also be used in the lipid nanoparticle compositions of the disclosure can include, but not limited to the following:

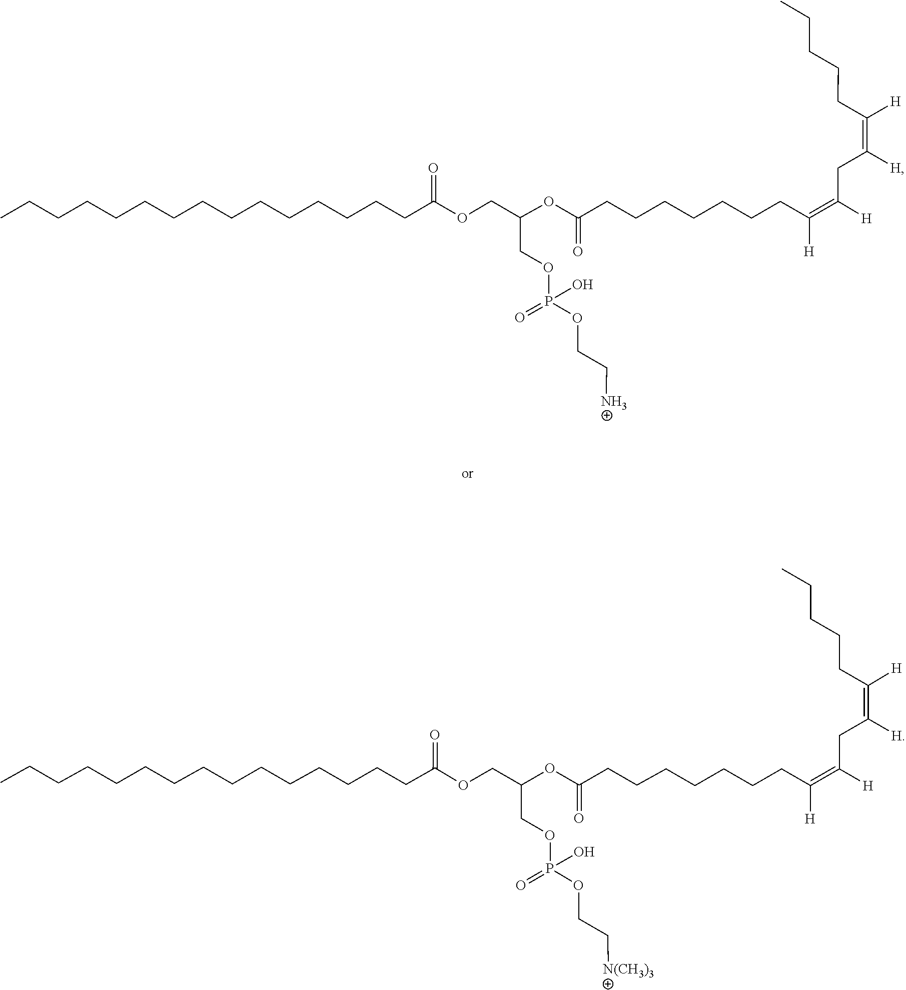

Other exemplary examples of helper lipids that can be used in the lipid nanoparticle compositions of the disclosure can include, but not limited to the following:

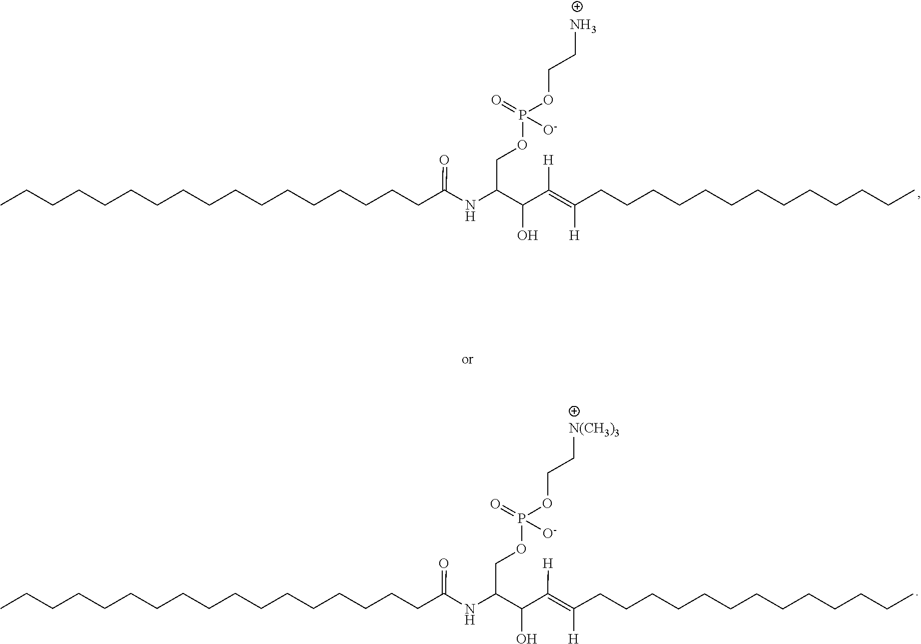

Such exemplary helper lipids can be prepared using methods described in J. Org. Chem. 1994, Vol. 59, 4805-4820, Org. Lett. 2005, Vol. 7, 2063-2065 and Tet. Lett. 1993, Vol. 34, 6881-6884.

In yet another embodiment, the helper lipids useful in the compositions may be selected from the group of phospholipids consisting of, but not limited to 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and mixtures thereof.

The stabilization lipid for use in the lipid-nanoparticle compositions may include 1-(monomethoxy-polyethyleneglycol)-2,3-dimyristoylglycerol (PEG-DMG), with an average PEG molecular weight of 2000.

The lipid component of a lipid-nanoparticle composition may include one or more structural lipids. Structural lipids can be selected from the group consisting of, but not limited to cholesterol, fecosterol, sitosterol (including beta sitosterol), ergosterol, campesterol, stigmasterol, brassicasterol, tomatidine, ursolic acid, alpha-tocopherol, and mixtures thereof. In some embodiments, the structural lipid is cholesterol. In some embodiments, the structural lipid includes cholesterol and a corticosteroid (such as prednisolone, dexamethasone, prednisone, and hydrocortisone), or a combination thereof.

The structural lipids can also include natural and synthetic cholesterol derivatives. Some examples of natural cholesterol derivatives include, but not limited to, (70-OHC, 22 (R)-hydroxycholesterol (22R-OHC), 24 (S)-hydroxycholesterol (24 (S)-OHC)). Synthetic cholesterol derivates may include, but not limited to, (22(R)-hydroxy-Δ9-cholestanol (22R-ISO-OHC), ((23-(4-Methylfuran-2,5-dione)-3α-hydroxy-24-nor-50-cholane (LITHO 1a), 23-(4-Methylfuran-2,5-dione)-3α,7α-dihydroxy-24-nor-5β-cholane (CHENO 1b), 23-(4-Methyl-1H-pyrrole-2,5-dione)-3α-hydroxy-24-nor-50-cholane (LITOMAL 7a), 23-(4-Methyl-1H-pyrrole-2,5-dione)-3α,7α, 12α-trihydroxy-24-nor-50-cholane (COLMAL 7f) and ethanol maleimide derivatives of litocholic and chenodeoxycholic acid (LITOMET, CHENOMET)) (146,147). The systematic name of LITOMET is (23-((2-hydroxyethyl)-4-methyl-1H-pyrrole-2,5-dione)-3α-hydroxy-24-nor-50-cholane) and the systematic name of CHENOMET is (23-((2-hydroxyethyl)-4-methyl-1H-pyrrole-2,5-dione)-3α,7α-dihydroxy-24-nor-5β-cholane).

The lipid-nanoparticle composition of the disclosure, include nucleic acids, such as, but not limited to, small interfering RNA (siRNA), an asymmetrical interfering RNA (aiRNA), a microRNA (miRNA), miRNA inhibitors (antagomirs/antimirs), a dicer-substrate RNA (dsRNA), a small hairpin RNA (shRNA), a messenger RNA (mRNA), multivalent RNA, and mixtures thereof.

The nucleic acid or mRNA may be a self-amplifying RNA, a polycistronic RNA, a self-amplifying polycistronic RNA, or a circular RNA. In some aspects, the nucleic acid or mRNA expresses a protein or peptide. In some aspects, the protein or peptide expressed from the nucleic acid or mRNA is an antibody, human antibody, camelid antibody, nanobody, humanized antibody, bispecific antibody, enzyme, genome editing enzyme or nuclease, growth factor, cytokine, chemokine, small molecule-mimicking peptide, transcription factor, structural molecule, signaling molecule, reprogramming factor, vaccine antigen, or combination thereof. In some aspects, the mRNA encodes a protein or peptide that acts intracellularly. In some aspects, the mRNA encodes at least one reprogramming factor.

The protein or peptide expressed from the nucleic acid or mRNA of the present technology may be at least one extracellular matrix protein such as collagen, laminin, elastin, fibronectin, integrin, tenascin, proteoglycan, fibrin, or combinations thereof. The collagen may be collagen I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, or combinations thereof. In some embodiments, the collagen is collagen VII. In some embodiments, the collagen VII is used in methods of rejuvenating, treating, remodeling, or improving skin or extracellular matrix. In some embodiments, the collagen VII is used in methods of wound healing.

A protein or peptide expressed from a nucleic acid or mRNA of the present technology may also be a growth factor, cytokine, or combination thereof, such as EGF, FGF, NGF, CNTF, PDGF, VEGF, IGF, GMCSF, GCSF, TGF, Erythropoietin, Ephrin, GDNF, GDF9, KGF, Angiopoeitin, TPO, BMP, HGF, BDNF, GDF, HGH (somatotropin), Neurotrophins, MSF, SGF, GDF (including GDF11), TGF (including TGF-b), or combinations thereof. In some embodiments, the cytokine is IL-1, IL-2, IL-3, IL-4, IL-5, IL-6, IL-7, IL-8, IL-9, IL-10, IL-11, TNF-α, IL-12, IL-13, IL-14, IL-15, IL-16, IL-17, CXCL8 (formerly IL-18), IL-19, IL-20, IL-21, IL-22, IL-23, IL-24, IL-25, IL-26, IL-27, IL-28, IL-29, IL-30, IL-31, IL-32, IL-33, or combinations thereof.

The protein or peptide expressed from the nucleic acid or mRNA of the present technology may be a human antibody, humanized antibody, camelid antibody, companion animal antibody, or nanobody. In some embodiments, the protein or peptide expressed from the nucleic acid or mRNA is an enzyme such as a nuclease, for example a nuclease used in genome editing. In some aspects, the protein or peptide expressed from the nucleic acid or mRNA acts intracellularly. In some aspects, the protein or peptide expressed from the nucleic acid or mRNA is secreted. In some embodiments, the antibody is at least one of or substantially similar to Trastuzumab, Glofitamab, Mirikizumab, Mirvetuximab, Nirsevimab, Tremelimumab, Teclistamab, Donanemab, Spesolimab, Lecanemab, Tislelizumab, Penpulimab, Sintilimab, Teplizumab, Toripalimab, Omburtamab, Retifanlimab, Ublituximab, Inolimomab, Oportuzumab, Narsoplimab, Mosunetuzumab, Tixagevimab, Cilgavimab, Relatlimab, Tebentafusp, Faricimab, Sutimlimab, Sotrovimab, Regdanvimab, Casirivimab, Imdevimab, Tezepelumab, Tisotumab, Amivantamab, Anifrolumab, Loncastuximab, Bimekizumab, Tralokinumab, Evinacumab, Sacituzumab, Teprotumumab, Isatuximab, Eptinezumab, Dostarlimab, Ansuvimab, Margetuximab, Naxitamab, Atoltivimab, Maftivimab, Odesivimab, Belantamab, Tafasitamab, Satralizumab, Inebilizumab, Enfortumab, Crizanlizumab, Brolucizumab, Polatuzumab, Risankizumab, Romosozumab, Caplacizumab, Ravulizumab, Emapalumab, Cemiplimab, Fremanezumab, Moxetumomab, Galcanezumab, Lanadelumab, Mogamulizumab, Erenumab, Tildrakizumab, Ibalizumab, Burosumab, Durvalumab, Emicizumab, Benralizumab, Ocrelizumab, Guselkumab, Inotuzumab, Sarilumab, Dupilumab, Avelumab, Brodalumab, Atezolizumab, Bezlotoxumab, Olaratumab, Reslizumab, Obiltoxaximab, Ixekizumab, Daratumumab, Elotuzumab, Necitumumab, Idarucizumab, Alirocumab, Mepolizumab, Evolocumab, Dinutuximab, Secukinumab, Nivolumab, Blinatumomab, Pembrolizumab, Ramucirumab, Vedolizumab, Siltuximab, Obinutuzumab, Raxibacumab, Pertuzumab, Brentuximab, Belimumab, Ipilimumab, Denosumab, Tocilizumab, Ofatumumab, Canakinumab, Golimumab, Ustekinumab, Certolizumab, Catumaxomab, Eculizumab, Ranibizumab, Panitumumab, Natalizumab, Bevacizumab, Cetuximab, Efalizumab, Omalizumab, Tositumomab, Ibritumomab, Adalimumab, Alemtuzumab, Gemtuzumab, Infliximab, Palivizumab, Basiliximab, Daclizumab, Rituximab, Abciximab, Edrecolomab, Nebacumab, or Muromonab. In some embodiments, the protein or peptide is used in methods of treating human or veterinary diseases.

The lipid-nanoparticle composition of the disclosure may be prepared by mixing processes such as, but not limited to, microfluidics and T-junction mixing of two fluid streams, one of which contains the nucleic acid and the other has the lipid components. Such mixing process induces nano-precipitation and particle formation.

The lipid-nanoparticle composition of the disclosure can be characterized using a Zetasizer to determine the particle size, the polydispersity index (PDI) and zeta potential. In some embodiments, the zeta potential of the lipid-nanoparticle composition may be from about −10 mV to about +20 mV, from about −10 mV about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 m V to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

Ultraviolet-visible spectroscopy can be used to determine the concentration of the nucleic acid in the nanoparticle compositions.

The lipid-nanoparticle composition can induce expression of a desired protein both in vitro and in vivo by contacting cells with a lipid nanoparticle comprising one or more of the ionizable lipids described herein, wherein the lipid nanoparticle is encapsulated or is associated with a nucleic acid that is expressed to produce the desired protein (e.g., mRNA).

The lipid-nanoparticle composition can decrease the expression of target genes and proteins both in vitro and in vivo by contacting cells with a lipid nanoparticle comprising one or more of the ionizable lipids described herein, wherein the lipid nanoparticle is encapsulated or is associated with a nucleic acid that reduces target gene expression (e.g., siRNA).

The lipid-nanoparticle composition can down-regulate or silence the expression of target genes and proteins both in vitro and in vivo by contacting cells with a lipid nanoparticle comprising one or more of the ionizable lipids described herein, wherein the lipid nanoparticle is encapsulated or is associated with a nucleic acid that down-regulate or silence target gene expression.

The methods and compositions provided herein are applied to cells, tissue, or organs of the nervous system, muscular system, respiratory system, cardiovascular system, skeletal system, reproductive system, integumentary system, lymphatic system, excretory system, immune system, endocrine system (e.g., endocrine and exocrine), or digestive system. Any type of cell can potentially be rejuvenated, as described herein, including, but not limited to, epithelial cells (e.g., squamous, cuboidal, columnar, and pseudostratified epithelial cells), endothelial cells (e.g., vein, artery, and lymphatic vessel endothelial cells), and cells of connective tissue, muscles, and the nervous system. Such cells may include, but are not limited to, epidermal cells, fibroblasts, chondrocytes, skeletal muscle cells, satellite cells, heart muscle cells, smooth muscle cells, keratinocytes, basal cells, ameloblasts, exocrine secretory cells, myoepithelial cells, osteoblasts, osteoclasts, neurons (e.g., sensory neurons, motor neurons, and interneurons), glial cells (e.g., oligodendrocytes, astrocytes, ependymal cells, microglia, Schwann cells, and satellite cells), pillar cells, adipocytes, pericytes, stellate cells, pneumocytes, blood and immune system cells (e.g., erythrocytes, monocytes, dendritic cells, macrophages, neutrophils, eosinophils, mast cells, T cells, B cells, natural killer cells), hormone-secreting cells, germ cells, interstitial cells, lens cells, photoreceptor cells, taste receptor cells, and olfactory cells; as well as cells and/or tissue from the kidney, liver, pancreas, stomach, spleen, gall bladder, intestines, bladder, lungs, prostate, breasts, urogenital tract, pituitary cells, oral cavity, esophagus, skin, hair, nail, thyroid, parathyroid, adrenal gland, eyes, nose, or brain.

Cells that may be treated in accordance with the present technology may be selected from fibroblasts, endothelial cells, chondrocytes, skeletal muscle stem cells, keratinocytes, mesenchymal stem cells and corneal epithelial cells. In embodiments, the cells are fibroblasts. In embodiments, the cells are endothelial cells. In embodiments, the cells are chondrocytes. In embodiments, the cells are skeletal muscle stem cells. In embodiments, the cells are keratinocytes. In embodiments, the cells are mesenchymal stem cells. In embodiments, the cells are corneal epithelial cells.

Methods and compositions of the present technology may also be applied to immune cells including, but not limited to, lymphocytes, granulocytes, monocytes, macrophages, microglia, or dendritic cells. In some embodiments, the lymphocyte is a T-cell, a B-cell or a natural killer (NK) cell. In some embodiments, the lymphocyte is a tumor-infiltrating lymphocyte.

Methods and compositions of the present technology may also be applied to lymphocytes wherein the lymphocyte is a T-cell. In some embodiments, the T-cell is a cytotoxic T cell (CD8+), a helper T cell (CD4+), a suppressor or regulatory T cell (Treg), a memory T cell, a natural killer T cell (NKT cell), or a gamma delta T cell. In other embodiments, the helper T cell is a Th1, Th2, Th17, Th9, or Tfh T-cell. In some embodiments, the memory T cell is a central memory T cell, an effector memory T cell, a tissue resident memory T cell, or a virtual memory T cell. In some embodiments, suppressor or regulatory T cells of the present technology are FOXP3+ T cells or FOXP3− T cells. In some embodiments, the NKT cell is a subset of CD1d-restricted T cells.

Methods and compositions of the present technology may also be applied to granulocytes wherein the granulocyte is a neutrophil, an eosinophil, a basophil, or a mast cell.

Methods and compositions of the present technology may also be applied to lymphocytes wherein the lymphocyte is a B-cell, such as a memory B-cell or a plasma cell.

Methods and compositions of the present technology may also be applied to immune cells wherein the immune cell is a monocyte, macrophage, microglial cell, or dendritic cell.

The methods and compositions described herein may be used wherein the cell is an immune cell, such as a natural immune cell or an engineered immune cell. In some embodiments, the methods and compositions described herein are used in parallel or in series with methods of engineering cells, including engineered immune cells, such that the methods are performed before, during, and/or after the engineering of the cells. In some embodiments, the methods and compositions described herein are used for engineering cells, including engineered immune cells. In some embodiments, such engineering includes engineering so that the cells express chimeric antigen receptors, such as chimeric antigen expressing immune cells. In some embodiments, such chimeric antigen receptors target at least one of CD19, CD20, CD22, CD30, CD33, CD123, FLT3, BCMA, GD2, HER2, MUC1, B7-H3, IL13Ra2, TAG72, MUC16, BCMA, or any other antigen suitable for immunotherapy. In some embodiments, such engineering includes engineering cells, including immune cells, to express other proteins or peptides, such as growth factors and cytokines. In some embodiments, said cytokines include IL-15. In some embodiments, such engineering of cells, such as immune cells, is performed ex vivo, e.g., in the manufacturing of a cellular therapy product, such as an autologous or allogenic chimeric antigen receptor (CAR)-T, CAR-NK, CAR-M, or CAR-NKT cells. In some embodiments, the CAR-T cells provided herein are targeted to at least one of CD19, CD20, CD22, HER2, MUC1, CD30, CD33, CD123, FLT3, B7-H3, IL13Ra2, GD2, TAG72, MUC16, or BCMA by the chimeric antigen receptor. In some embodiments, the CAR-T cells provided herein are targeted to at least one of CD19 or BCMA by the chimeric antigen receptor. In some embodiments, the CAR-T cells provided herein are targeted to CD19 by the chimeric antigen receptor. In some embodiments, the CAR-T cells provided herein are targeted to BCMA by the chimeric antigen receptor. In some embodiments, the CAR-NK cells provided herein are targeted to at least one of CD19, FLT3, CD20, CD38, CD138, BCMS, CS1, CD3, CD5, CD7, NKG2D, HER2, EGFR, EpCAM, TF, B7-H6, HLA-G, CD24, CD44, CD133, mesothelin, or alphaFR by the chimeric antigen receptor. In some embodiments, the CAR-M cells provided herein are targeted to at least one of CD19, HER2, CD22, or ALK19 by the chimeric antigen receptor. In some embodiments, the CAR-NK cells provided herein are engineered to express IL-2 and/or IL-15. In some embodiments, the CAR-NKT cells provided herein are targeted to GD2, by the chimeric antigen receptor, and engineered to express IL-15. In some embodiments, the immune cell rejuvenation methods described herein are performed ex vivo during or after the manufacturing of the cell therapy product. In other embodiments, such engineering of cells, and/or immune cells is performed ex vivo, e.g., in so-called “in situ” generation of CAR-engineered cells. In such embodiments, RNA and/or mRNA encoding CARs or growth factors or cytokines contained in lipid-containing compositions or lipid-nanoparticle compositions of the disclosure is injected in vivo into a subject or patient, for example for CAR engineering of the patient's immune cells, such as T cells, NK cells, macrophages, tumor infiltrating lymphocytes, dendritic cells and/or NKT cells “in situ,” i.e., inside the patient's body without having to remove cells for ex vivo transfection. In such embodiments, the immune cell rejuvenation methods described herein are also performed in vivo, where mRNA encoding the reprogramming factor or factors is injected into the patient before, concurrently with, or after the mRNA encoding CARs or other cell engineering molecules. In some embodiments, the lipids and lipid-nanoparticle compositions of the disclosure are selected for targeted delivery to any cell, including immune cells, in vivo, such as T cells, NK cells, macrophages, tumor infiltrating cells, dendritic cells, and/or NKT cells in vivo. In still other embodiments, in vivo treatment is performed in the absence of any other in vivo cell engineering, to enhance or restore the potency of the immune system and treat diseases associated with immune dysfunction or dysregulation, such as improving the effect of the immune system against cancer or infection, or reducing inflammation.

Methods and compositions of the present technology may also be applied to rejuvenation of immune cells wherein the immune cell to be rejuvenated is a non-adherent cell, such as a non-adherent immune cell. In some embodiments, non-adherent cells, including non-adherent immune cells, are treated, transiently reprogrammed, rejuvenated, or manufactured in a manner wherein the cells remain non-adherent, without adhering to a tissue culture substrate or forming or giving rise to cells or colonies of cells that adhere to a tissue culture substrate. In some embodiments, the reprogramming interval and factors are selected such that cells are rejuvenated with retention of cellular identity, wherein the cells remain non-adherent, without adhering to a tissue culture substrate or forming or giving rise to cells or colonies of cells that adhere to a tissue culture substrate. Accordingly, in some embodiments, the present technology provides lipid-containing compositions and lipid-nanoparticle compositions to be used for delivering mRNA encoding at least one reprogramming factor for cell rejuvenation wherein cells, including any non-adherent cells and/or non-adherent immune cells (e.g., non-adherent T cells, NK cells, macrophages, tumor infiltrating cells, dendritic cells, and/or NKT cells), are reprogrammed in a manner wherein the cells are rejuvenated with retention of cellular identity, and wherein the cells stay in suspension and are not adherent, nor do they become or give rise to cells that are adherent, become adherent, or form adherent colonies.

The methods described herein, including methods of rejuvenating immune cells; methods of reversing, preventing, or inhibiting exhaustion in immune cells; or inducing proliferation in immune cells comprise administering the lipid-containing compositions or lipid-nanoparticle compositions of the disclosure comprising providing mRNA encoding at least one reprogramming factor to an immune cell about 1, 2, 3, 4, 5, 6, 7, 8, 9, or 10 times over a period of about 1, 2, 3, 4, 5, 6, 7, 8, 9, or 10 days. For example, the mRNA could be administered once on the first or second day of a five- or six-day period, or it could be administered once on the first day and once on the third day of a five- or six-day period, or it could be administered once in a one-day period. In some embodiments, the mRNA is administered to an immune cell 1, 2, 3, 4, 5, or 6 times over a period of 1, 2, 3, 4, 5, or 6 days. In some embodiments, the mRNA is administered after an immune cell activation step. In some embodiments, the immune cell activation step comprises activating the immune cells for 1, 2, or 3 days. In some embodiments, the immune cell activation step comprises activating the immune cells using at least one of CD3, CD28, and IL-2. In some embodiments, the immune cells are activated with CD3 and CD28. In some embodiments, the mRNA administration period occurs immediately after the immune cell activation step. In some embodiments, the mRNA administration period occurs 1, 2, 3, 4, 5, 6, 7, 8, 9, 10, 11, 12, 13, or 14 days after the immune cell activation step. In some embodiments, the administration of the mRNA encoding reprogramming factors reverses immune cell exhaustion caused by the immune cell activation step. In some embodiments, the administration of the mRNA encoding reprogramming factors reverses immune cell exhaustion in immune cells from an aged patient or donor. In some embodiments, the administration of the mRNA is performed during a manufacturing process to make immune cells for transplantation, for example CAR-T, CAR-M, or CAR-NK cells.

Use of the lipids or lipid-nanoparticles of the present technology for delivery of mRNA provides enhanced rejuvenation, proliferation, recovery from or prevention of exhaustion, therapeutic effects, anti-pathogenic effects, anti-cancer effects, anti-immunogenic effects or anti-inflammatory effects in a cell treated or rejuvenated using the methods or compositions herein compared to using a different delivery mechanism for the mRNA. In some embodiments, such enhanced rejuvenation, proliferation, recovery from or prevention of exhaustion, therapeutic effects, anti-pathogenic effects, anti-cancer effects, anti-immunogenic effects or anti-inflammatory effects result from lower toxicity, immunogenicity, and/or lower physiological impact on the cell when compared to the different delivery mechanism. In some embodiments, the different delivery mechanism is electroporation such that the use of the lipids or lipid-nanoparticles compositions of the disclosure for delivery of the mRNA results in enhanced rejuvenation, proliferation, recovery from or prevention of exhaustion, therapeutic effects, anti-pathogenic effects, anti-cancer effects, anti-immunogenic effects or anti-inflammatory effects in cells treated or rejuvenated using the methods or compositions herein compared to when using electroporation. This improvement compared to electroporation can result from reduced toxicity or reduced physiological impact on the cell compared to electroporation.

The lipids or lipid-nanoparticle compositions of the present technology may be used in methods of delivery of therapeutic or diagnostic agents to the skin, such as administering lipid-containing compositions or lipid-nanoparticle compositions comprising at least one therapeutic or diagnostic agent. In some embodiments, the lipids or lipid-nanoparticle compositions of the present technology provide delivery of therapeutic or diagnostic agents, such as reprogramming factors, in a manner that achieves transient reprogramming of a cell, such as a skin cell or immune cell. In some embodiments, transient reprogramming of a cell provides transient expression of a therapeutic or diagnostic agent, such as a reprogramming factor, wherein the agent is expressed in a cell for a duration sufficient to reprogram and/or rejuvenate without changing the identity of the cell, i.e., rejuvenating a skin or immune cell to exhibit features or a younger skin or immune cell while retaining the identity of a skin or immune cell.

The therapeutic agent of the present technology, in some instances, is mRNA as disclosed herein. In some aspects the lipids or lipid-nanoparticle compositions of the disclosure are used in methods of treating or preventing dermatological diseases or conditions, treating or preventing diseases or conditions of the skin, or for cosmetic applications in skin, comprising administering lipid-containing compositions or lipid-nanoparticle compositions comprising at least one therapeutic or diagnostic agent. In some aspects the lipids or lipid-nanoparticle compositions of the disclosure are used in methods of rejuvenating skin, comprising administering lipid-containing compositions or lipid-nanoparticle compositions comprising a therapeutic or diagnostic agent. In some aspects the lipids or lipid-nanoparticle compositions of the disclosure are used in methods of rejuvenating skin, comprising administering lipid-containing compositions or lipid-nanoparticle compositions comprising mRNA encoding at least one reprogramming factor. In such methods, the mRNA can be bound to the lipids or contained in the lipid-nanoparticles. In some aspects, such methods further comprise transfecting skin cells with the lipid-containing compositions or lipid-nanoparticle compositions to deliver the mRNA. In some aspects, the lipids or lipid-nanoparticle compositions of the disclosure are used in methods of rejuvenating skin, comprising administering lipid-containing compositions or lipid-nanoparticle compositions comprising mRNA encoding at least one reprogramming factor to achieve rejuvenation of the skin while retaining cell identity. In such methods, the mRNA can be bound to the lipids or contained in the lipid-nanoparticles. In some aspects, the lipids or lipid-nanoparticle compositions of the disclosure are used in methods of rejuvenating skin, comprising administering lipid-containing compositions or lipid-nanoparticle compositions comprising mRNA encoding at least one reprogramming factor to skin cells, wherein expression of the at least one reprogramming factor in skin cells results in increased fibroblast proliferation with retention of skin cell identity. In such methods, the mRNA can be bound to the lipids or contained in the lipid-nanoparticles.

The lipids or lipid-nanoparticle compositions may be used in methods of wound healing, comprising administering lipid-containing compositions or lipid-nanoparticle compositions comprising a therapeutic or diagnostic agent. In some aspects, the lipids or lipid-nanoparticle compositions are used in methods of wound healing, comprising administering lipid-containing compositions or lipid-nanoparticle compositions comprising mRNA encoding at least one reprogramming factor. In such methods, the mRNA can be bound to the lipids or contained in the lipid-nanoparticles. In some aspects the lipids or lipid-nanoparticle compositions are used in methods of wound healing, wherein lipid-containing compositions or lipid-nanoparticle compositions deliver mRNA encoding at least one reprogramming factor to achieve wound healing while retaining cell identity. In such methods, the mRNA can be bound to the lipids or contained in the lipid-nanoparticles. In some aspects, the lipids or lipid-nanoparticle compositions of the disclosure are used in methods of wound healing, comprising administering lipid-containing compositions or lipid-nanoparticle compositions comprising mRNA encoding at least one reprogramming factor to skin cells, wherein expression of the at least one reprogramming factor in skin cells results in increased fibroblast proliferation. In such methods, the mRNA can be bound to the lipids or contained in the lipid-nanoparticles.

The lipids or lipid-nanoparticle compositions may be used in methods of treating or preventing dermatological diseases or conditions, treating or preventing diseases or conditions of the skin, or for cosmetic applications in skin, comprising administering lipid-containing compositions or lipid-nanoparticle compositions comprising a therapeutic agent to achieve reversal of at least one skin aging marker. In some aspects, the lipids or lipid-nanoparticle compositions are used in methods of rejuvenating skin, comprising administering lipid-containing compositions or lipid-nanoparticle compositions to deliver mRNA encoding at least one reprogramming factor to skin cells to achieve reversal of at least one skin aging marker while retaining cell identity. In some aspects, the lipids or lipid-nanoparticle compositions are used in methods of wound healing, comprising administering lipid-containing compositions or lipid-nanoparticle compositions to deliver mRNA encoding at least one reprogramming factor to skin cells to achieve reversal of at least one skin aging marker while retaining cell identity. In some embodiments, reversal of at least one skin aging marker refers to producing a rejuvenated cell that expresses at least one skin aging marker in a manner similar to the expression of said marker seen in a youthful skin cell as compared to an aged skin cell.

The markers which may be impacted in accordance with the present technology includes mRNA or protein expression of IL6, CXCL8, CSF3, CXCL1, SERPINB2, LIF, IL11, CXCL2, IL24, PTGS2, MMP3, CCL2, TFPI2, IER3, ACKR3, PTGES, SLC16A6, TNFAIP6, PTPRN, IL1RN, IL1B, CXCL5, CXCL6, HAS1, HSD11B1, CH₂₅H, ADGRD1, C₃, RASD1, NR4A3, STC1, TCIM, SRGN, AC003092.1, LRRN3, CHI3L¹, NR4A2, NAMPT, PRSS23, MMP1, SOD2, LOXL4, MMP11, ELN, CREG1, C₁₅orf48, NFKBIZ, PID1, or any combination thereof. In some aspects, reversal of at least one skin aging marker is downregulation of mRNA or protein expression of IL6, CXCL8, CSF3, CXCL1, SERPINB2, LIF, IL11, CXCL2, IL24, PTGS2, MMP3, CCL2, TFPI2, IER3, ACKR3, PTGES, SLC16A6, TNFAIP6, PTPRN, IL1RN, IL1B, CXCL5, CXCL6, HAS1, HSD11B1, CH₂₅H, ADGRD1, C₃, RASD1, NR4A3, STC1, TCIM, SRGN, AC003092.1, LRRN3, CHI3L¹, NR4A2, NAMPT, MMP1, SOD2, CREG1, C₁₅orf48, NFKBIZ, PID1, or any combination thereof. In some aspects, reversal of at least one skin aging marker is upregulation of mRNA or protein expression of PRSS23, LOXL4, MMP11, ELN, or any combination thereof. In some aspects, reversal of at least one skin aging marker is upregulation of mRNA or protein expression of PRSS23. In some aspects, reversal of at least one skin aging marker is upregulation of mRNA or protein expression of LOXL4. In some aspects, reversal of at least one skin aging marker is upregulation of mRNA or protein expression of MMP11. In some aspects, reversal of at least one skin aging marker is upregulation of mRNA or protein expression of ELN. In some aspects, reversal of at least one skin aging marker is downregulation of mRNA or protein expression of MMP3, MMP1, SOD2, or any combination thereof. In some aspects, reversal of at least one skin aging marker is downregulation of mRNA or protein expression of MMP3. In some aspects, reversal of at least one skin aging marker is downregulation of mRNA or protein expression of MMP1. In some aspects, reversal of at least one skin aging marker is downregulation of mRNA or protein expression of SOD2. In some aspects, reversal of at least one skin aging marker is upregulation of mRNA or protein expression of at least one of PRSS23, LOXL4, MMP11, or ELN; downregulation of mRNA or protein expression of at least one of MMP3, MMP1, SOD2; or any combination thereof.

The lipids or lipid-nanoparticle compositions may be used in methods of treating or preventing dermatological diseases or conditions, treating or preventing diseases or conditions of the skin, or for cosmetic applications in skin, comprising administering lipid-containing compositions or lipid-nanoparticle compositions comprising a therapeutic agent to achieve improvement of at least one skin quality marker. In some aspects, the lipids or lipid-nanoparticle compositions are used in methods of rejuvenating skin, comprising administering lipid-containing compositions or lipid-nanoparticle compositions comprising mRNA encoding at least one reprogramming factor to skin cells to achieve improvement of at least one skin quality marker. In some embodiments, the lipids or lipid-nanoparticle compositions are used in methods of rejuvenating skin, comprising administering lipid-containing compositions or lipid-nanoparticle compositions comprising mRNA encoding at least one reprogramming factor to skin cells to achieve improvement of at least one skin quality marker, while retaining cell identity.

The lipids or lipid-nanoparticle compositions may be used in methods of wound healing, comprising administering lipid-containing compositions or lipid-nanoparticle compositions comprising mRNA encoding at least one reprogramming factor to skin cells to achieve improvement of at least one skin quality marker. In some aspects, the marker is mRNA or protein expression of Collagen Type I, Collagen Type III, Collagen Type V, Collagen Type VI, Collagen Type XI, Elastin, Microfibril-Associated Protein 5, Periostin, Versican, Connective Tissue Growth Factor, Lysyl Oxidase, SPARC, Secreted Phosphoprotein 1, Cartilage Oligomeric Matrix Protein, MMP1, MMP3, MMP12, SOD2 or any combination thereof. In some aspects, improvement of at least one skin quality marker is downregulation of mRNA or protein expression of MMP1, MMP3, MMP12, SOD2 or any combination thereof. In some aspects, improvement of at least one skin quality marker is upregulation of mRNA or protein expression of Collagen Type I, Collagen Type III, Collagen Type IV, Collagen Type V, Collagen Type VI, Collagen Type XI, Elastin, Microfibril-Associated Protein 5, Periostin, Versican, Connective Tissue Growth Factor, Lysyl Oxidase, SPARC, Secreted Phosphoprotein 1, Cartilage Oligomeric Matrix Protein, or any combination thereof. In some aspects, improvement of at least one skin quality marker is upregulation of mRNA or protein expression of Collagen. In some aspects, improvement of at least one skin quality marker is upregulation of mRNA or protein expression of Collagen Type I. In some aspects, improvement of at least one skin quality marker is upregulation of mRNA or protein expression of Collagen Type III. In some aspects, improvement of at least one skin quality marker is upregulation of mRNA or protein expression of Collagen Type IV. In some aspects, improvement of at least one skin quality marker is upregulation of mRNA or protein expression of Collagen Type V. In some aspects, improvement of at least one skin quality marker is upregulation of mRNA or protein expression of Collagen Type VI. In some aspects, improvement of at least one skin quality marker is upregulation of mRNA or protein expression of Collagen Type XI. In some aspects, improvement of at least one skin quality marker is upregulation of mRNA or protein expression of Collagen Type XI. In some aspects, improvement of at least one skin quality marker is upregulation of mRNA or protein expression of Elastin. In some aspects, improvement of at least one skin quality marker is upregulation of mRNA or protein expression of Microfibril-associated Protein 5. In some aspects, improvement of at least one skin quality marker is upregulation of mRNA or protein expression of Periostin. In some aspects, improvement of at least one skin quality marker is upregulation of mRNA or protein expression of Versican. In some aspects, improvement of at least one skin quality marker is upregulation of mRNA or protein expression of Connective Tissue Growth Factor. In some aspects, improvement of at least one skin quality marker is upregulation of mRNA or protein expression of Lysyl Oxidase. In some aspects, improvement of at least one skin quality marker is upregulation of mRNA or protein expression of SPARC. In some aspects, improvement of at least one skin quality marker is upregulation of mRNA or protein expression of Secreted Phosphoprotein 1. In some aspects, improvement of at least one skin quality marker is upregulation of mRNA or protein expression of Cartilage Oligomeric Matrix Protein. In some aspects, improvement of at least one skin quality marker is downregulation of mRNA or protein expression of MMP3, MMP1, SOD2, or any combination thereof. In some aspects, improvement of at least one skin quality marker is downregulation of mRNA or protein expression of MMP3. In some aspects, improvement of at least one skin quality marker is downregulation of mRNA or protein expression of MMPL. In some aspects, improvement of at least one skin quality marker is downregulation of mRNA or protein expression of SOD2. In some aspects, improvement of at least one skin quality marker is upregulation of mRNA or protein expression of Collagen VII and Elastin. In some aspects, improvement of at least one skin quality marker is upregulation of mRNA or protein expression of at least one of Collagen VII and Elastin; downregulation of mRNA or protein expression of at least one of MMP3, MMP1, SOD2; or any combination thereof.

The lipid-nanoparticle compositions or compositions containing the lipids of the disclosure may be administered to the skin topically. In some aspects, the lipid-nanoparticle compositions of the disclosure or compositions containing the lipids of the disclosure are administered to the skin in an ointment, cream, or salve. In some aspects, the lipid-nanoparticle compositions of the disclosure or compositions containing the lipids of the disclosure are administered to the skin via dermal, intradermal, or subcutaneous injection. In some aspects, the lipid-nanoparticle compositions of the disclosure or compositions containing the lipids of the disclosure are administered to the skin via a gel. In some aspects, the lipid-nanoparticle compositions of the disclosure or compositions containing the lipids of the disclosure are administered in vivo, in vitro, or ex vivo. In some aspects, the lipid-nanoparticle compositions of the disclosure or compositions containing the lipids of the disclosure are administered in vivo. In some aspects, the lipid-nanoparticle compositions of the disclosure or compositions containing the lipids of the disclosure are to a human or animal subject.

The lipid-nanoparticle compositions or compositions containing the lipids of the disclosure may transfect skin cells to deliver at least one therapeutic or diagnostic agent into skin cells. In some aspects, the therapeutic agent is a nucleic acid. In some aspects, the therapeutic agent is mRNA. In some aspects, the therapeutic agent is a combination of mRNA and siRNA. In some aspects, the therapeutic agent is a combination of mRNA and miRNA. In some aspects, the skin cell is a keratinocyte, melanocyte, Langerhans cell, follicle cell, fibroblast, endothelial cell, smooth muscle cell, Merkel cell, basal cell, squamous cell, apocrine gland cell, eccrine gland cell, sebaceous gland cell, lymphatic endothelial cell, or combination thereof. In some aspects, the lipid or lipid-nanoparticle composition is selected to provide selective transfection to a particular cell type or cell types. In some aspects, the lipid or lipid-nanoparticle composition is selected to provide diffusion within the skin or within at least one layer of the skin.

The diseases or conditions that may be treated or prevented include dermatological diseases or conditions or diseases or conditions of the skin treated that may be treated or prevented using the lipid compositions or lipid-nanoparticle compositions of the present technology. For example, dermatoporosis or chronic wounds may be treated in accordance with the present technology. In some aspects the dermatoporosis or chronic wound is a diabetic, ischemic, and pressure ulcer. In some aspects, the dermatological disease or condition or disease or condition of the skin treated or prevented using the lipid compositions or lipid-nanoparticle compositions of the disclosure is an inflammatory skin disease. In some aspects, the inflammatory skin disease is psoriasis, atopic dermatitis, vitiligo, alopecia areata, or hidradenitis suppurativa. In some aspects, the dermatological disease or condition or disease or condition of the skin treated or prevented using the lipid compositions or lipid-nanoparticle compositions of the disclosure is a hair disorder. In some aspects, the hair disorder is non-scarring or scarring alopecia, hair greying, hirsutism. In some aspects, the hair disorder is non-scarring alopecia is androgenic alopecia. In some aspects, scarring alopecia is lichen planopilaris. In some aspects, the dermatological disease or condition or disease or condition of the skin treated or prevented using the lipid compositions or lipid-nanoparticle compositions of the disclosure is a skin cancer. In some aspects, the skin cancer is basal cell carcinoma, squamous cell carcinoma, or actinic keratosis. In some aspects, the dermatological disease or condition or disease or condition of the skin treated or prevented using the lipid compositions or lipid-nanoparticle compositions of the disclosure is prurigo nodularis, acne, rosacea, or solar lentigines. In some aspects, a method of treating any of the above dermatological diseases or conditions or diseases comprises administering compositions containing lipids of the disclosure or lipid-nanoparticle compositions of the disclosure comprising a therapeutic agent. In some aspects, the therapeutic agent is mRNA. In some aspects, the therapeutic agent is mRNA encoding an antibody, human antibody, humanized antibody, nanobody, camelid antibody, bispecific antibody, enzyme, genome editing enzyme or nuclease, growth factor, cytokine, chemokine, transcription factor, structural molecule, signaling molecule, reprogramming factor. In some aspects, the therapeutic agent is mRNA encoding a protein or peptide that acts intracellularly.

In some aspects, the therapeutic agent is mRNA encoding at least one reprogramming factor. The lipid compositions or lipid-nanoparticle compositions may be used in methods of wound healing wherein the wound is dermatoporosis or a chronic wound. In some aspects the dermatoporosis or chronic wound is a diabetic, ischemic, and pressure ulcer. In some aspects, the lipid compositions or lipid-nanoparticle compositions of the disclosure are used in methods of wound healing wherein the wound is a lesion from a skin cancer. In some aspects, the skin cancer is basal cell carcinoma, squamous cell carcinoma, or actinic keratosis. In some aspects, any of the above wound healing methods comprises administering compositions containing lipids of the disclosure or lipid-nanoparticle compositions comprising a therapeutic agent. In some aspects, the therapeutic agent is mRNA. In some aspects, the therapeutic agent is mRNA encoding an antibody, human antibody, humanized antibody, nanobody, camelid antibody, bispecific antibody, enzyme, genome editing enzyme or nuclease, growth factor, cytokine, chemokine, transcription factor, structural molecule, signaling molecule, reprogramming factor. In some aspects, the therapeutic agent is mRNA encoding a protein or peptide that acts intracellularly. In some aspects, the therapeutic agent is mRNA encoding at least one reprogramming factor.

Lipid-nanoparticle compositions may contain lipid formula (II) and have higher transfection efficiency in skin cells compared to other lipid-nanoparticle compositions containing lipid formula (II) or to other lipid-nanoparticle compositions containing other lipids described herein. Such compositions comprising lipid formula (II), when used to transfect at least one reprogramming factor, may produce greater improvement of at least one skin quality marker as compared to other formulations or compared to lipid-nanoparticle compositions containing other lipids described herein.

In embodiments, lipid-nanoparticle compositions containing lipid formula (I-B) provide higher transfection efficiency compared to lipid-nanoparticle compositions containing other lipids described herein. In embodiments, lipid-nanoparticle compositions containing lipid formula (I-B) provide higher transfection efficiency compared to lipid-nanoparticle compositions containing lipid formula (I) with the same head group. In embodiments, lipid-nanoparticle compositions containing lipid formula (I-B) provide higher transfection efficiency in skin cells compared to lipid-nanoparticle compositions containing other lipids described herein. In embodiments, lipid-nanoparticle compositions containing lipid formula (I-B) provide higher transfection efficiency in skin cells compared to lipid-nanoparticle compositions containing lipid formula (I) with the same head group. In embodiments, lipid-nanoparticle compositions containing lipid formula (I-B) provide higher transfection efficiency in fibroblasts compared to lipid-nanoparticle compositions containing other lipids described herein. In embodiments, lipid-nanoparticle compositions containing lipid formula (I-B) provide higher transfection efficiency in fibroblasts compared to lipid-nanoparticle compositions containing lipid formula (I) with the same head group. In embodiments, lipid-nanoparticle compositions containing lipid formula (I-B) provide higher transfection efficiency in immune cells compared to lipid-nanoparticle compositions containing other lipids described herein. In embodiments, lipid-nanoparticle compositions containing lipid formula (I-B) provide higher transfection efficiency in immune cells compared to lipid-nanoparticle compositions containing lipid formula (I) with the same head group. In embodiments, lipid-nanoparticle compositions containing lipid formula (I-B) provide higher transfection efficiency in T cells compared to lipid-nanoparticle compositions containing other lipids described herein. In embodiments, lipid-nanoparticle compositions containing lipid formula (I-B) provide higher transfection efficiency in T cells compared to lipid-nanoparticle compositions containing lipid formula (I) with the same head group. In embodiments, the tail structure of lipid formula (I-B) provides increased transfection efficiency for lipid-nanoparticle compositions. In embodiments, the tail structure of lipid formula (I-B) provides increased transfection efficiency compared to an unbranched alkyl or alkenyl tail structure for lipid-nanoparticle compositions. In embodiments, the tail structure of lipid formula (I-B) provides increased transfection efficiency for lipid-nanoparticle compositions compared to the tail structure of lipid formula (I) with the same head group. In embodiments, ester groups adjacent to branching of alkyl groups in the tail structure of lipids described herein provide increased transfection efficiency for lipid-nanoparticle compositions. In embodiments, ester groups adjacent to branching of alkyl groups in the tail structure of lipids described herein provide increased transfection efficiency for lipid-nanoparticle compositions compared to alkyl or alkenyl tail structures without branching.

In embodiments, lipid-nanoparticle compositions containing lipid formula (I-B) provide higher viability compared to lipid-nanoparticle compositions containing other lipids described herein. In embodiments, lipid-nanoparticle compositions containing lipid formula (I-B) provide higher viability compared to lipid-nanoparticle compositions containing lipid formula (I) with the same head group. In embodiments, lipid-nanoparticle compositions containing lipid formula (I-B) provide higher viability in skin cells compared to lipid-nanoparticle compositions containing other lipids described herein. In embodiments, lipid-nanoparticle compositions containing lipid formula (I-B) provide higher viability in skin cells compared to lipid-nanoparticle compositions containing lipid formula (I) with the same head group. In embodiments, lipid-nanoparticle compositions containing lipid formula (I-B) provide higher viability in fibroblasts compared to lipid-nanoparticle compositions containing other lipids described herein. In embodiments, lipid-nanoparticle compositions containing lipid formula (I-B) provide higher viability in fibroblasts compared to lipid-nanoparticle compositions containing lipid formula (I) with the same head group. In embodiments, lipid-nanoparticle compositions containing lipid formula (I-B) provide higher viability in immune cells compared to lipid-nanoparticle compositions containing other lipids described herein. In embodiments, lipid-nanoparticle compositions containing lipid formula (I-B) provide higher viability in immune cells compared to lipid-nanoparticle compositions containing lipid formula (I) with the same head group. In embodiments, lipid-nanoparticle compositions containing lipid formula (I-B) provide higher viability in T cells compared to lipid-nanoparticle compositions containing other lipids described herein. In embodiments, lipid-nanoparticle compositions containing lipid formula (I-B) provide higher viability in T cells compared to lipid-nanoparticle compositions containing lipid formula (I) with the same head group. In embodiments, the tail structure of lipid formula (I-B) provides increased viability for lipid-nanoparticle compositions. In embodiments, the tail structure of lipid formula (I-B) provides increased viability for lipid-nanoparticle compositions compared to an unbranched alkyl or alkenyl tail structure. In embodiments, the tail structure of lipid formula (I-B) provides increased viability for lipid-nanoparticle compositions compared to the tail structure of lipid formula (I). In embodiments, ester groups adjacent to branching of alkyl groups in the tail structure of lipids described herein provide increased viability for lipid-nanoparticle compositions. In embodiments, ester groups adjacent to branching of alkyl groups in the tail structure of lipids described herein provide increased viability for lipid-nanoparticle compositions compared to alkyl or alkenyl tail structures without branching. In embodiments, the ester groups adjacent to R6 and R7 and to R8 and R10 together with the branching of R6, R7, R8, and R10 in the tail structure of lipid formula (I-B) provide increased viability for lipid-nanoparticle compositions. In embodiments, the ester groups adjacent to R6 and R7 and to R8 and R10 together with the branching of R6, R7, R8, and R10 in the tail structure of lipid formula (I-B) provide increased viability for lipid-nanoparticle compositions compared to an alkyl or alkenyl tail structure without branching.

In embodiments, lipid-nanoparticle compositions containing lipid formula (I-B) provide both higher viability and higher transfection efficiency compared to lipid-nanoparticle compositions containing other lipids described herein. In embodiments, lipid-nanoparticle compositions containing lipid formula (I-B) provide both higher viability and higher transfection efficiency compared to lipid-nanoparticle compositions containing lipid formula (I) with the same head group. In embodiments, lipid-nanoparticle compositions containing lipid formula (I-B) provide both higher viability and higher transfection efficiency in skin cells compared to lipid-nanoparticle compositions containing other lipids described herein. In embodiments, lipid-nanoparticle compositions containing lipid formula (I-B) provide both higher viability and higher transfection efficiency in skin cells compared to lipid-nanoparticle compositions containing lipid formula (I) with the same head group. In embodiments, lipid-nanoparticle compositions containing lipid formula (I-B) provide both higher viability and higher transfection efficiency in fibroblasts compared to lipid-nanoparticle compositions containing other lipids described herein. In embodiments, lipid-nanoparticle compositions containing lipid formula (I-B) provide both higher viability and higher transfection efficiency in fibroblasts compared to lipid-nanoparticle compositions containing lipid formula (I) with the same head group. In embodiments, lipid-nanoparticle compositions containing lipid formula (I-B) provide both higher viability and higher transfection efficiency in immune cells compared to lipid-nanoparticle compositions containing other lipids described herein. In embodiments, lipid-nanoparticle compositions containing lipid formula (I-B) provide both higher viability and higher transfection efficiency in immune cells compared to lipid-nanoparticle compositions containing lipid formula (I) with the same head group.

In embodiments, lipid-nanoparticle compositions containing lipid formula (I-B) provide both higher viability and higher transfection efficiency in T cells compared to lipid-nanoparticle compositions containing other lipids described herein. In embodiments, lipid-nanoparticle compositions containing lipid formula (I-B) provide both higher viability and higher transfection efficiency in T cells compared to lipid-nanoparticle compositions containing lipid formula (I) with the same head group. In embodiments, the tail structure of lipid formula (I-B) provides both increased transfection efficiency and increased viability. In embodiments, the tail structure of lipid formula (I-B) provides both increased transfection efficiency and increased viability compared to an alkyl or alkenyl tail structure without branching. In embodiments, the tail structure of lipid formula (I-B) provides both increased transfection efficiency and increased viability for lipid-nanoparticle compositions compared to the tail structure of lipid formula (I). In embodiments, ester groups adjacent to branching of alkyl groups in the tail structure of lipids described herein provide both increased transfection efficiency and increased viability for lipid-nanoparticle compositions. In embodiments, the ester groups adjacent to R6 and R7 and to R8 and R10 together with the branching of R6, R7, R8, and R10 in the tail structure of lipid formula (I-B) provide both increased transfection efficiency and increased viability for lipid-nanoparticle compositions. In embodiments, the ester groups adjacent to R6 and R7 and to R8 and R10 together with the branching of R6, R7, R8, and R10 in the tail structure of lipid formula (I-B) provide both increased transfection efficiency and increased viability for lipid-nanoparticle compositions compared to an alkyl or alkenyl tail structure without branching.

Such compositions comprising lipid formula (I-B), when used to transfect at least one reprogramming factor into skin cells, may produce greater improvement of at least one skin rejuvenation marker or skin quality marker as compared to lipid-nanoparticle compositions containing other lipids described herein. Such compositions comprising lipid formula (I-B), when used to transfect at least one reprogramming factor into skin cells, may produce greater improvement of at least one skin rejuvenation marker or skin quality marker as compared to lipid-nanoparticle compositions containing lipid formula (I) with the same head group. Such compositions comprising lipid formula (I-B), when used to transfect at least one reprogramming factor into immune cells, may produce greater improvement of at least one immune cell rejuvenation marker or immune cell stemness marker as compared to lipid-nanoparticle compositions containing other lipids described herein. Such compositions comprising lipid formula (I-B), when used to transfect at least one reprogramming factor into immune cells, may produce greater improvement of at least one immune cell rejuvenation marker or stemness rejuvenation marker as compared to lipid-nanoparticle compositions containing lipid formula (I), with the same head group.

In embodiments, lipid-nanoparticle compositions containing lipid formula (I) where q1 is absent and q2 is 1 provide higher transfection efficiency compared to lipid-nanoparticle compositions containing other lipids described herein. In embodiments, lipid-nanoparticle compositions containing lipid formula (I) where q1 is absent and q2 is 1 provide higher transfection efficiency compared to lipid-nanoparticle compositions containing (I) where q1 is 1 and q2 is absent. In embodiments, lipid-nanoparticle compositions containing lipid formula (I) where q1 is absent and q2 is 1 provide higher transfection efficiency in skin cells compared to lipid-nanoparticle compositions containing other lipids described herein. In embodiments, lipid-nanoparticle compositions containing lipid formula (I) where q1 is absent and q2 is 1 provide higher transfection efficiency in skin cells compared to lipid-nanoparticle compositions containing lipid formula (I) where q1 is 1 and q2 is absent. In embodiments, lipid-nanoparticle compositions containing lipid formula (I) where q1 is absent and q2 is 1 provide higher transfection efficiency in fibroblasts compared to lipid-nanoparticle compositions containing other lipids described herein. In embodiments, lipid-nanoparticle compositions containing lipid formula (I) where q1 is absent and q2 is 1 provide higher transfection efficiency in fibroblasts compared to lipid-nanoparticle compositions containing lipid formula (I) where q1 is 1 and q2 is absent. In embodiments, lipid-nanoparticle compositions containing lipid formula (I) where q1 is absent and q2 is 1 provide higher transfection efficiency in immune cells compared to lipid-nanoparticle compositions containing other lipids described herein. In embodiments, lipid-nanoparticle compositions containing lipid formula (I) where q1 is absent and q2 is 1 provide higher transfection efficiency in immune cells compared to lipid-nanoparticle compositions containing lipid formula (I) where q2 is absent and q1 is 1. In embodiments, lipid-nanoparticle compositions containing lipid formula (I) where q1 is absent and q2 is 1 provide higher transfection efficiency in T cells compared to lipid-nanoparticle compositions containing other lipids described herein. In embodiments, lipid-nanoparticle compositions containing lipid formula (I) where q1 is absent and q2 is 1 provide higher transfection efficiency in T cells compared to lipid-nanoparticle compositions containing lipid formula (I) where q1 is 1 and q2 is absent.

In embodiments, lipid-nanoparticle compositions containing lipid formula (I) where q1 is absent and q2 is 1 provide higher viability compared to lipid-nanoparticle compositions containing other lipids described herein. In embodiments, lipid-nanoparticle compositions containing lipid formula (I) where q1 is absent and q2 is 1 provide higher viability compared to lipid-nanoparticle compositions containing lipid formula (I) where q1 is 1 and q2 is absent. In embodiments, lipid-nanoparticle compositions containing lipid formula (I) where q1 is absent and q2 is 1 provide higher viability in skin cells compared to lipid-nanoparticle compositions containing other lipids described herein. In embodiments, lipid-nanoparticle compositions containing lipid formula (I) where q1 is absent and q2 is 1 provide higher viability in skin cells compared to lipid-nanoparticle compositions containing lipid formula (I) where q1 is 1 and q2 is absent. In embodiments, lipid-nanoparticle compositions containing lipid formula (I) where q1 is absent and q2 is 1 provide higher viability in fibroblasts compared to lipid-nanoparticle compositions containing other lipids described herein.

In embodiments, lipid-nanoparticle compositions containing lipid formula (I) where q1 is absent and q2 is 1 provide higher viability in fibroblasts compared to lipid-nanoparticle compositions containing lipid formula (I) where q1 is 1 and q2 is absent. In embodiments, lipid-nanoparticle compositions containing lipid formula (I) where q1 is absent and q2 is 1 provide higher viability in immune cells compared to lipid-nanoparticle compositions containing other lipids described herein.

In embodiments, lipid-nanoparticle compositions containing lipid formula (I) where q1 is absent and q2 is 1 provide higher viability in immune cells compared to lipid-nanoparticle compositions containing lipid formula (I) where q1 is 1 and q2 is absent. In embodiments, lipid-nanoparticle compositions containing lipid formula (I) where q1 is absent and q2 is 1 provide higher viability in T cells compared to lipid-nanoparticle compositions containing other lipids described herein. In embodiments, lipid-nanoparticle compositions containing lipid formula (I) where q1 is absent and q2 is 1 provide higher viability in T cells compared to lipid-nanoparticle compositions containing lipid formula (I) where q1 is 1 and q2 is absent.

In embodiments, lipid-nanoparticle compositions containing lipid formula (I) where q1 is absent and q2 is 1 provide both higher viability and higher transfection efficiency compared to lipid-nanoparticle compositions containing other lipids described herein. In embodiments, lipid-nanoparticle compositions containing lipid formula (I) where q1 is absent and q2 is 1 provide both higher viability and higher transfection efficiency compared to lipid-nanoparticle compositions containing lipid formula (I) where q1 is 1 and q2 is absent. In embodiments, lipid-nanoparticle compositions containing lipid formula (I) where q1 is absent and q2 is 1 provide both higher viability and higher transfection efficiency in skin cells compared to lipid-nanoparticle compositions containing other lipids described herein. In embodiments, lipid-nanoparticle compositions containing lipid formula (I) where q1 is absent and q2 is 1 provide both higher viability and higher transfection efficiency in skin cells compared to lipid-nanoparticle compositions containing lipid formula (I) where q1 is 1 and q2 is absent. In embodiments, lipid-nanoparticle compositions containing lipid formula (I) where q1 is absent and q2 is 1 provide both higher viability and higher transfection efficiency in fibroblasts compared to lipid-nanoparticle compositions containing other lipids described herein. In embodiments, lipid-nanoparticle compositions containing lipid formula (I) where q1 is absent and q2 is 1 provide both higher viability and higher transfection efficiency in fibroblasts compared to lipid-nanoparticle compositions containing lipid formula (I) where q1 is 1 and q2 is absent. In embodiments, lipid-nanoparticle compositions containing lipid formula (I) where q1 is absent and q2 is 1 provide both higher viability and higher transfection efficiency in immune cells compared to lipid-nanoparticle compositions containing other lipids described herein. In embodiments, lipid-nanoparticle compositions containing lipid formula (I) where q1 is absent and q2 is 1 provide both higher viability and higher transfection efficiency in immune cells compared to lipid-nanoparticle compositions containing lipid formula (I) where q1 is 1 and q2 is absent. In embodiments, lipid-nanoparticle compositions containing lipid formula (I-B) provide both higher viability and higher transfection efficiency in T cells compared to lipid-nanoparticle compositions containing other lipids described herein. In embodiments, lipid-nanoparticle compositions containing lipid formula (I) where q1 is absent and q2 is 1 provide both higher viability and higher transfection efficiency in T cells compared to lipid-nanoparticle compositions containing lipid formula (I) where q1 is 1 and q2 is absent.

Such compositions comprising lipid formula (I) where q1 is absent and q2 is 1, when used to transfect at least one reprogramming factor into skin cells, may produce greater improvement of at least one skin rejuvenation marker or skin quality marker as compared to lipid-nanoparticle compositions containing other lipids described herein. Such compositions comprising lipid formula (I) where q1 is absent and q2 is 1, when used to transfect at least one reprogramming factor into skin cells, may produce greater improvement of at least one skin rejuvenation marker or skin quality marker as compared to lipid-nanoparticle compositions containing lipid formula (I) where q1 is 1 and q2 is absent. Such compositions comprising lipid formula (I) where q1 is absent and q2 is 1, when used to transfect at least one reprogramming factor into immune cells, may produce greater improvement of at least one immune cell rejuvenation marker or immune cell stemness marker as compared to lipid-nanoparticle compositions containing other lipids described herein. Such compositions comprising lipid formula (I) where q1 is absent and q2 is 1, when used to transfect at least one reprogramming factor into immune cells, may produce greater improvement of at least one immune cell rejuvenation marker or stemness rejuvenation marker as compared to lipid-nanoparticle compositions containing lipid formula (I) where q1 is 1 and q2 is absent with the same head group

The methods provided herein include using the lipids or lipid-nanoparticle compositions to transfect cells with one or more non-integrative messenger RNAs encoding one or more cellular reprogramming factors, thereby producing rejuvenated cells. The cells to be rejuvenated may be of any cell type. In embodiments, the cells are contacted with, exposed to, or transfected with the mRNA for not more than about 20, 19, 18, 17, 16, 15, 14, 13, 12, 11, 10, 9, 8, 7, 6, 5, 4, 3, 2, or 1 days, or for less than 1 day. In embodiments, the cells are contacted with, exposed to, or transfected with the mRNA for not more than about 14, 13, 12, 11, 10, 9, 8, 7, 6, 5, 4, 3, 2, or 1 days, or for less than one day. In embodiments, the cells are contacted with, exposed to, or transfected with the mRNA for not more than about 10, 9, 8, 7, 6, 5, 4, 3, 2, or 1 days, or for less than one day. In embodiments, the cells are contacted with, exposed to, or transfected with the mRNA for not more than about 7, 6, 5, 4, 3, 2, or 1 days, or for less than one day. In embodiments, the cells are contacted with, exposed to, or transfected with the mRNA for not more than about 5, 4, 3, 2, or 1 days, or for less than one day. In embodiments, the at least one reprogramming factor is expressed from the transfected mRNA within cells, or the cells are exposed to the at least one reprogramming factor expressed from the transfected mRNA, for not more than about 20, 19, 18, 17, 16, 15, 14, 13, 12, 11, 10, 9, 8, 7, 6, 5, 4, 3, 2, or 1 days, or for less than 1 day. In embodiments, the at least one reprogramming factor is expressed from the transfected mRNA within cells, or the cells are exposed to the at least one reprogramming factor expressed from the transfected mRNA, for not more than about 14, 13, 12, 11, 10, 9, 8, 7, 6, 5, 4, 3, 2, or 1 days, or for less than one day. In embodiments, the at least one reprogramming factor is expressed from the transfected mRNA within cells, or the cells are exposed to the at least one reprogramming factor expressed from the transfected mRNA, for at least about 2 days and not more than about 14, 13, 12, 11, 10, 9, 8, 7, 6, 5, 4, 3, or 2 days. In embodiments, the at least one reprogramming factor is expressed from the transfected mRNA within cells, or the cells are exposed to the at least one reprogramming factor expressed from the transfected mRNA, for not more than about 10, 9, 8, 7, 6, 5, 4, 3, 2, or 1 days, or for less than one day. In embodiments, the at least one reprogramming factor is expressed from the transfected mRNA within cells, or the cells are exposed to the at least one reprogramming factor expressed from the transfected mRNA, for at least about 2 days and not more than about 10, 9, 8, 7, 6, 5, 4, 3, or 2 days. In embodiments, the at least one reprogramming factor is expressed from the transfected mRNA within cells, or the cells are exposed to the at least one reprogramming factor expressed from the transfected mRNA, for not more than about 7, 6, 5, 4, 3, 2, or 1 days, or for less than one day. In embodiments, the at least one reprogramming factor is expressed from the transfected mRNA within cells, or the cells are exposed to the at least one reprogramming factor expressed from the transfected mRNA, for at least about 2 days and not more than about 7, 6, 5, 4, 3, or 2 days. In embodiments, the at least one reprogramming factor is expressed from the transfected mRNA within cells, or the cells are exposed to the at least one reprogramming factor expressed from the transfected mRNA, for not more than about 5, 4, 3, 2, or 1 days, or for less than one day. In embodiments, the at least one reprogramming factor is expressed from the transfected mRNA within cells, or the cells are exposed to the at least one reprogramming factor expressed from the transfected mRNA, for at least about 2 days and not more than about 5, 4, 3, or 2 days. In embodiments, the rejuvenated cells have a phenotype or activity profile similar to a young cell. The phenotype or activity profile includes one or more of the transcriptomic profile, gene expression of one or more nuclear and/or epigenetic markers, proteolytic activity, mitochondrial health and function, SASP cytokine expression, and methylation landscape.

The rejuvenated cells described herein may have a transcriptomic profile that is more similar to the transcriptomic profile of young cells. In embodiments, the transcriptomic profile of the rejuvenated cells includes an increase in gene expression of one or more genes selected from RPL37, RHOA, SRSF3, EPHB4, ARHGAP18, RPL31, FKBP2, MAP1LC3B2, Elfl, Phf8, Pol2s2, Tafl and Sin3a.

The rejuvenated cells described herein may also exhibit increased gene expression of one or more nuclear and/or epigenetic markers compared to a reference value. In embodiments, the one or more nuclear and/or epigenetic markers is selected from Hplgamma, H3K9me3, lamina support protein LAP2alpha, and SIRTl protein. In embodiments, the rejuvenated cells have a proteolytic activity that is more similar to the proteolytic activity of young cells. In embodiments, the proteolytic activity is measured as increased cell autophagosome formation, increased chymotrypsin-like proteasome activity, or a combination thereof. In embodiments, the rejuvenated cells exhibit improved mitochondria health and function compared to a reference value. In embodiments, improved mitochondria health and function is measured as increased mitochondria membrane potential, decreased reactive oxygen species (ROS), or a combination thereof.

The rejuvenated cells described herein may also exhibit decreased expression of one or more SASP cytokines compared to a reference value. In embodiments, the one or more SASP cytokines include IL18, ILIA, GROA, IL22, and IL9. In embodiments, the rejuvenated cells exhibit reversal of the methylation landscape. In embodiments, the reversal of the methylation landscape is measured by Horvath clock estimation. In some embodiments, a reference value is obtained from an aged cell.

As described herein, cells may be rejuvenated by transient reprogramming with mRNAs encoding one or more cellular reprogramming factors transfected into the cells using the lipids or lipid-nanoparticle compositions of the disclosure. Transient reprogramming is accomplished, in some embodiments, by transfecting cells with non-integrative mRNAs for not more than about 20, 19, 18, 17, 16, 15, 14, 13, 12, 11, 10, 9, 8, 7, 6, 5, 4, 3, 2, or 1 days, or less than 1 day. Transient reprogramming is accomplished, in some embodiments, by transfecting cells with non-integrative mRNAs for not more than about 14, 13, 12, 11, 10, 9, 8, 7, 6, 5, 4, 3, 2, or 1 days, or less than 1 day. Transient reprogramming is accomplished, in some embodiments, by transfecting cells with non-integrative mRNAs for not more than about 9, 8, 7, 6, 5, 4, 3, 2, or 1 days, or less than 1 day. Transient reprogramming is accomplished, in some embodiments, by transfecting cells with non-integrative mRNAs for not more than about 6, 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for not more than about 20, 19, 18, 17, 16, 15, 14, 13, 12, 11, 10, 9, 8, 7, 6, 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for at least 2 days and not more than about 20, 19, 18, 17, 16, 15, 14, 13, 12, 11, 10, 9, 8, 7, 6, 5, 4, 3, or 2 days. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for not more than about 14, 13, 12, 11, 10, 9, 8, 7, 6, 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for at least 2 days and not more than about 14, 13, 12, 11, 10, 9, 8, 7, 6, 5, 4, 3, or 2 days. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for not more than about 10, 9, 8, 7, 6, 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for at least 2 days and not more than about 10, 9, 8, 7, 6, 5, 4, 3, or 2 days. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for not more than about 7, 6, 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for at least 2 days and not more than about 7, 6, 5, 4, 3, or 2 days. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for not more than about 5, 4, 3, 2, or 1 days, or less than 1 day. Transient reprogramming is accomplished, in some embodiments, by expressing at least one reprogramming factor from non-integrative mRNAs transfected in cells, or exposing cells to at least one reprogramming factor expressed from non-integrative mRNAs transfected in cells, for at least 2 days and not more than about 5, 4, 3, or 2 days. In embodiments, transient reprogramming of cells eliminates various hallmarks of aging while avoiding complete dedifferentiation of the cells into stem cells.

The methods and compositions provided herein achieve cellular age-reversal, or rejuvenation, by transient overexpression of one or more mRNAs encoding cellular reprogramming factors, delivered by the lipids or lipid-nanoparticle compositions of the disclosure. Such cellular reprogramming factors may include transcription factors, epigenetic remodelers, or small molecules affecting mitochondrial function, proteolytic activity, heterochromatin levels, histone methylation, nuclear lamina polypeptides, cytokine secretion, or senescence. In embodiments, the cellular reprogramming factors include one or more of OCT4, SOX2, KLF4, c-MYC, LIN28 and NANOG. In embodiments, the cellular reprogramming factors are applied in different molar ratios, for example OCT4, SOX2, KLF4, c-MYC, LIN28, and NANOG at molar ratios of a:b:c:d:e:f, wherein a, b, c, d, e, and f can all be the same number (for example, 1:1:1:1:1:1), some the same number and some different numbers (for example, 3:1:1:1:1:1, 2:1:1:1:1:1, 2:2:1:1:1:1, 2:2:2:1:1:1, 2:2:2:2:1:1, 2:2:2:2:2:1, 3:3:3:3:2:2), or all different numbers (for example 6:4:5:3:2:1). In embodiments, a, b, c, d, e, and/or f are each between 1-7, i.e., 1-7:1-7:1-7:1-7:1-7:1-7 (or 1-7:1-7:1-7:1-7:1-7, 1-7:1-7:1-7:1-7, 1-7:1-7:1-7, 1-7:1-7, or 1-7:1 in the case of combinations with fewer than 6 factors). In embodiments, the cellular reprogramming factors are applied in different weight ratios, for example, OCT4, SOX2, KLF4, c-MYC, LIN28, and NANOG at weight ratios of a:b:c:d:e:f, wherein a, b, c, d, e, and f can each be the same (for example, 1:1:1:1:1:1), each of a, b, c, d, e, and f can be the same or different numbers (for example, 3:1:1:1:1:1, 2:1:1:1:1:1, 2:2:1:1:1:1, 2:2:2:1:1:1, 2:2:2:2:1:1, 2:2:2:2:2:1, 3:3:3:3:2:2), or each can be a different number (for example 6:4:5:3:2:1). In embodiments, a, b, c, d, e, and/or f are each between 1-7, i.e., 1-7:1-7:1-7:1-7:1-7:1-7 (or 1-7:1-7:1-7:1-7:1-7, 1-7:1-7:1-7:1-7, 1-7:1-7:1-7, 1-7:1-7, or 1-7:1 in the case of combinations with fewer than 6 factors).

The methods and compositions provided herein may be applied to any type of cell, tissue or organs in need of rejuvenation. The methods and compositions of the disclosure can be used to rejuvenate cells in culture (e.g., ex vivo or in vitro) to improve function and potency for use in cell therapy. The cells used in treatment of a patient may be autologous or allogeneic. The cells can be derived from the patient or a matched donor, or they can be obtained from a cell bank or derived from iPS cells. For example, in autologous ex vivo therapy, cells can be obtained directly from the patient to be treated, transfected with mRNAs encoding cellular reprogramming factors, as described herein, and reimplanted in the patient. Such cells can be obtained, for example, from a biopsy or surgical procedure performed on the patient. Alternatively, in allogeneic ex vivo therapy, cells can be obtained from a cell bank or a cell line derived from iPS cells, transfected with mRNAs encoding cellular reprogramming factors, as described herein, and reimplanted in the patient. Alternatively, cells in need of rejuvenation can be transfected directly in vivo with mRNAs encoding cellular reprogramming factors.

Lipid-containing compositions or lipid-nanoparticle compositions of the disclosure may be used for delivery of mRNA expressing reprogramming factors that provide more robust cellular rejuvenation because the reprogramming factors have been optimized to decrease any triggered immune response to the protein/polypeptide, increase stability of the protein/polypeptide, and altered protein/polypeptide activity, such as increased activity when compared to wild-type reprogramming factors.

The methods provided herein comprise administering lipid-containing compositions or lipid-nanoparticle compositions of the disclosure comprising RNA to a cell or subject, or treating or transfecting a cell with lipid-containing compositions or lipid-nanoparticle compositions comprising RNA for a dosing interval of not more than 25, 24, 23, 22, 21, 20, 19, 18, 17, 16, 15, 14, 13, 12, 11, 10, 9, 8, 7, 6, 5, 4, 3, or 2 consecutive days. In some embodiments, the dosing interval is one day. In some embodiments, the compositions are administered once. In some embodiments, the dosing of the lipid-containing compositions or lipid-nanoparticle compositions comprising RNA is performed at least once daily during the dosing interval. In some embodiments, the dosing is performed less frequently than once per day during the dosing interval, for example once every two days, once every three days, once every four days, once every x days, where x is a number from 4 to 25. Thus, in such embodiments, for example, dosing lipid-containing compositions or lipid-nanoparticle compositions comprising RNA once every 5 days in a 5 day dosing interval means that the RNA is dosed once in the interval, i.e., once in the total treatment period of 5 days, whereas dosing RNA twice daily in a 5 day dosing interval means that the RNA is dosed 10 times in the interval, i.e., 10 times in the 5 days. In some embodiments, methods comprise administering lipid-containing compositions or lipid-nanoparticle compositions comprising RNA to a cell or subject, or treating or transfecting a cell with lipid-containing compositions or lipid-nanoparticle compositions comprising RNA for not more than 21, 18, 14, 10, 7, or 5 consecutive days. In some embodiments, methods comprise administering lipid-containing compositions or lipid-nanoparticle compositions comprising RNA to a cell or subject, or treating or transfecting a cell with lipid-containing compositions or lipid-nanoparticle compositions comprising RNA, for not more than 18 consecutive days. In some embodiments, methods comprise administering lipid-containing compositions or lipid-nanoparticle compositions comprising RNA to a cell or subject, or treating or transfecting a cell with lipid-containing compositions or lipid-nanoparticle compositions of the disclosure comprising RNA for not more than 14 consecutive days. In some embodiments, methods comprise administering lipid-containing compositions or lipid-nanoparticle compositions comprising RNA to a cell or subject, or treating or transfecting a cell with lipid-containing compositions or lipid-nanoparticle compositions comprising RNA for not more than 10 consecutive days. In some embodiments, methods comprise administering lipid-containing compositions or lipid-nanoparticle compositions comprising RNA to a cell or subject, or treating or transfecting a cell with lipid-containing compositions or lipid-nanoparticle compositions comprising RNA for not more than 7 consecutive days. In some embodiments, methods comprise administering lipid-containing compositions or lipid-nanoparticle compositions comprising RNA to a cell or subject, or treating or transfecting a cell with lipid-containing compositions or lipid-nanoparticle compositions comprising RNA for not more than 5 consecutive days. In other embodiments, said exposing comprises interrupting said exposing and repeating said exposing after said interrupting. In some embodiments, said exposing, treating, transfecting, expressing, or administering comprises exposing, treating, transfecting, expressing, or administering for between about 2-5 consecutive days, between about 5-7 consecutive days, between about 7-10 consecutive days, between about 10-12 consecutive days, between about 12-14 consecutive days, between about 14-17 consecutive days, between about 17-19 consecutive, or between about 19-21 consecutive days and in some embodiments, further comprises interrupting said exposing and repeating said exposing after said interrupting.

Duration of exposure is controlled by mechanisms such as self-amplifying RNA, circular RNA, B18R and other decoys, and/or on/off switches such as L7Ae or its family members. In some embodiments, said repeating is performed any number of times, for example 1, 2, 3, 4, 5, 6, 7, 8, 9, or 10 times, or up to 20 times, or up to 30 times, or more. For in vivo applications, said repeating may continue for any duration of time, for example until a disease is successfully treated or cured, or throughout the life of a subject or patient. In some embodiments, said repeating is performed any time after said interrupting, for example 0, 1, 2, 3, 4, 5, 6, 7, 8, 9, or 10 days, up to 20 days, up to 30 days, up to 3 months, up to 6 months, or up to 1 year after said interrupting. One exposure period is considered to be a dosing interval, such that, for example, a sequence of exposure-interruption-repeat exposure contains two dosing intervals.

### V. PHARMACEUTICAL COMPOSITIONS

The present disclosure also provides a pharmaceutical composition comprising the nanoparticle composition as described herein and a pharmaceutically acceptable carrier thereof. The pharmaceutical compositions are particularly useful for delivering a nucleic acid to a patient (e.g., a human) or to a cell for treating a particular disease or condition of interest. Appropriate concentrations and dosages can be readily determined by one skilled in the art.

Pharmaceutical compositions including rejuvenated cells may be obtained by transfecting cells with lipid-containing compositions or lipid-nanoparticle compositions of the disclosure comprising one or more non-integrative messenger RNAs encoding one or more cellular reprogramming factors for not more than 4, 5, 6, 7, 8, 9, 10, 11, 12, 13, or 14 continuous days, to transiently reprogram the cells for rejuvenation. It is also contemplated to treat cells by transfecting with lipid-containing compositions or lipid-nanoparticle compositions of the disclosure comprising a therapeutic mRNA, where, in embodiments, the transfection is conducted one time to obtain a treated cell, such as a rejuvenated cell. A singe transfection step is contemplated for example for self-replicating RNA constructs. In another aspect, provided herein are pharmaceutical compositions including rejuvenated cells obtained by transfecting cells with lipid-containing compositions or lipid-nanoparticle compositions of the disclosure comprising one or more non-integrative messenger RNAs encoding one or more cellular reprogramming factors for not more than 4, 5, 6, or 7 continuous days, to transiently reprogram the cells for rejuvenation.

The pharmaceutical compositions of the disclosure may be formulated into preparations in solid, semi-solid, liquid or gaseous forms, such as tablets, capsules, powders, granules, ointments, solutions, suspensions, suppositories, injections, inhalants, gels, microspheres, and aerosols. Typical routes of administering such pharmaceutical compositions include, without limitation, local, oral, topical, transdermal, inhalation, parenteral, sublingual, buccal, rectal, vaginal, and intranasal. The term parenteral as used herein includes subcutaneous injections, intravenous, intramuscular, intradermal, intrasternal injection or infusion techniques. Pharmaceutical compositions of the disclosure are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the disclosure in aerosol form may hold a plurality of dosage units. Actual methods of preparing such dosage forms are known, or will be apparent, to those skilled in this art; for example, see Remington: The Science and Practice of Pharmacy, 20th edition (Philadelphia College of Pharmacy and Science, 2000). The composition to be administered will include a therapeutically effective amount of the nanoparticle composition for treatment of a disease or condition of interest.

A pharmaceutical composition of the disclosure may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers.

In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the disclosure, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose; agents to act as cryoprotectants such as sucrose or trehalose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the disclosure intended for either parenteral or oral administration should contain an amount of a compound of the disclosure such that a suitable dosage will be obtained.

The pharmaceutical composition of the disclosure may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition of the disclosure may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the disclosure may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the disclosure in solid or liquid form may include an agent that binds to the compound of the disclosure and thereby assists in the delivery of the compound. Suitable agents that may act in this capacity include a monoclonal or polyclonal antibody, or a protein.

The pharmaceutical composition of the disclosure may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients.

Aerosols of compounds of the disclosure may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the disclosure may be prepared by methodology well known in the pharmaceutical art. For example, a pharmaceutical composition intended to be administered by injection can be prepared by combining the lipid nanoparticles of the disclosure with sterile, distilled water, buffer or other carrier to form a solution. A surfactant may be added to facilitate the formation of a homogeneous solution or suspension. Surfactants are compounds that non-covalently interact with the compound of the disclosure so as to facilitate dissolution or homogeneous suspension of the compound in the aqueous delivery system.

The compositions of the disclosure comprising the compounds described herein or their pharmaceutically acceptable salts, are administered in a therapeutically effective amount, which will vary depending upon a variety of factors including the activity of the specific therapeutic agent employed; the metabolic stability and length of action of the therapeutic agent; the age, body weight, general health, sex, and diet of the patient; the mode and time of administration; the rate of excretion; the drug combination; the severity of the particular disorder or condition; and the subject undergoing therapy.

Compositions of the disclosure may also be administered simultaneously with, prior to, or after administration of one or more other therapeutic agents. Such combination therapy includes administration of a single pharmaceutical dosage formulation of a composition of the disclosure and one or more additional active agents, as well as administration of the composition of the disclosure and each active agent in its own separate pharmaceutical dosage formulation. For example, a composition of the disclosure and the other active agent can be administered to the patient together in a single oral dosage composition such as a tablet or capsule or a single injection such as a subcutaneous, intradermal, or intravenous injection, or each agent can be administered in separate oral dosage formulations or separate injections. Where separate dosage formulations are used, the compounds of the disclosure and one or more additional active agents can be administered at essentially the same time, i.e., concurrently, or at separately staggered times, i.e., sequentially; combination therapy is understood to include all these regimens.

### VI. EXAMPLES

The present disclosure provides lipid-containing nanoparticle compositions to traffic biologically active substances such as a nucleic acid (e.g., RNA) into the cells and or intracellular compartments to facilitate the delivery of a therapeutic/prophylactic. Such compositions generally include at least one ionizable lipids.

The ionizable lipids of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein.

Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5th edition, John Wiley & Sons: New York, 2001; Greene, T. W., Wuts, P. G. M., Protective Groups in Organic Synthesis, 3rd edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art. The following descriptions of synthetic methods are designed to illustrate, but not to limit, general procedures for the preparation of compounds of the present disclosure.

It should be noted that the reaction schemes provided herein provides an exemplary method for the preparation of ionizable lipids of the disclosure. The use of protecting groups as needed and other modification to the general reaction schemes mentioned in the below examples will be readily apparent to one of ordinary skill in the art. A list of protecting groups and how to introduce and remove these groups can be found in Greene, T. W., Wuts, P. G. M., Protective Groups in Organic Synthesis, 3rd edition, John Wiley & Sons: New York, 1999.

In the reaction schemes described herein, multiple stereoisomers may be produced. When no particular stereoisomer is indicated, it is understood to mean all possible stereoisomers that could be produced from the reaction. A person of ordinary skill in the art will recognize that the reactions can be optimized to give one isomer preferentially, or new schemes may be devised to produce a single isomer. If mixtures are produced, techniques such as preparative thin layer chromatography, preparative HPLC, preparative chiral HPLC, or preparative SFC may be used to separate the isomers.

The following examples of ionizable lipids of the disclosure are illustrative in nature and are in no way intended to be limiting.

### Example 1: Synthesis of Ionizable Lipid of Formula (I)

The ionizable lipids of Formula (I) is based on cephalin scaffold. The synthesis of ionizable lipid of Formula (I) is depicted in Scheme 1 respectively. The procedures and methods are well known in the literature and are described in Chem. Sci., 2021, 12, 2549-2557. Analogous cephalin based ionizable lipids of Formula (I) can be synthesized using the methods that are well known in the art.

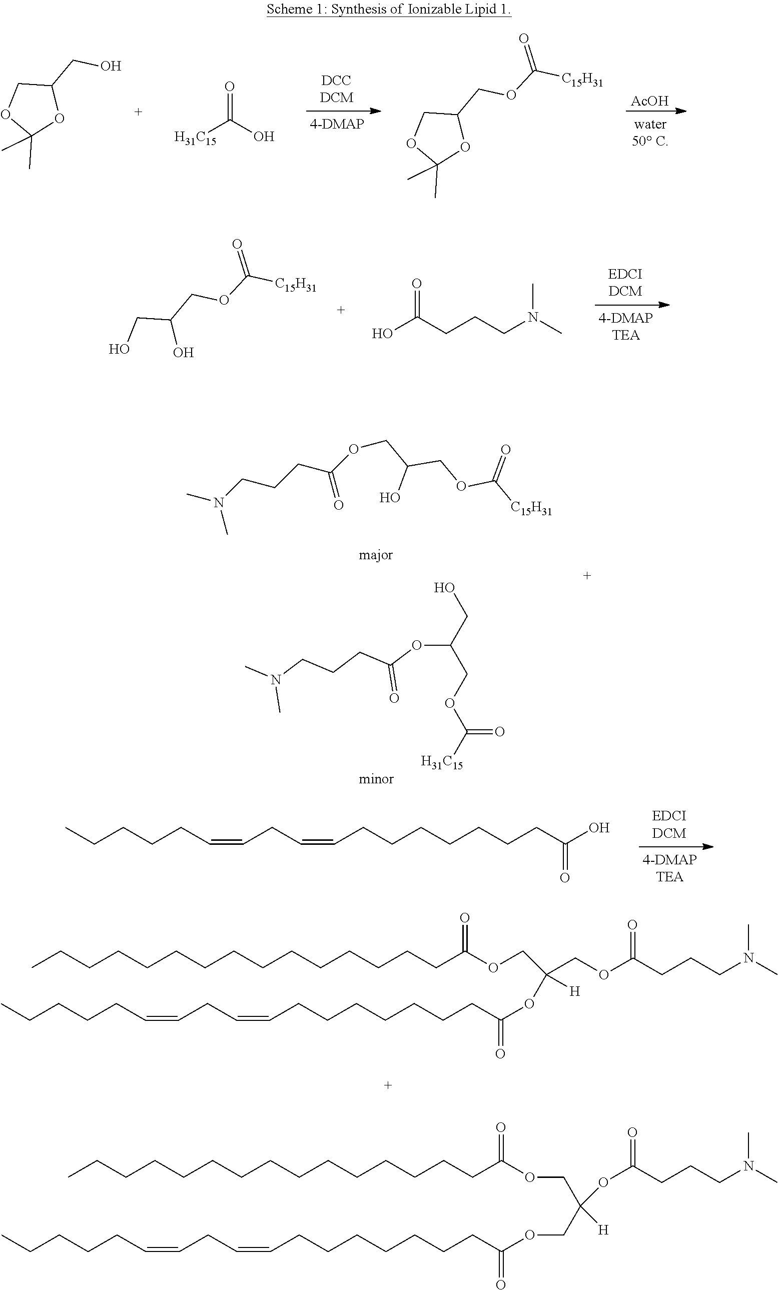

### Example 2: Synthesis of Ionizable Lipid of Formula (II)

The ionizable lipids of Formula (II) is based on sphingomyelin scaffold. The synthesis of ionizable lipid of Formula (II) is depicted in Scheme 2 and Scheme 3 respectively. The procedures and methods are well known in the literature and are described in Org. Lett. 2006, 8, 5569 (for Scheme 2) and in Chem. Eur. J. 2011, 17, 8568-8575 (for Schemes 2 and 3). Analogous sphingomyelin based ionizable lipids of Formula (II) can be synthesized using the methods that are well known in the art.

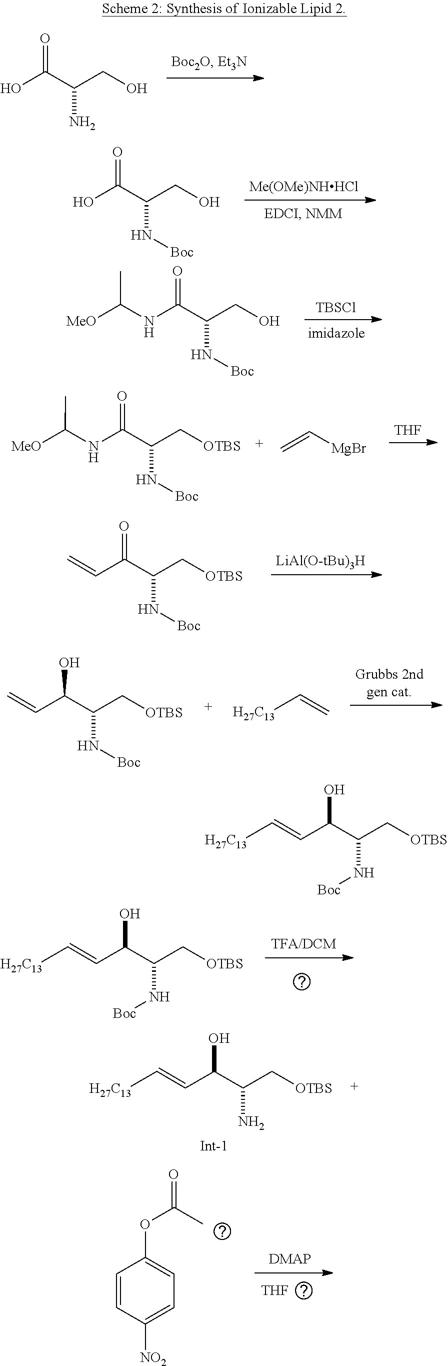

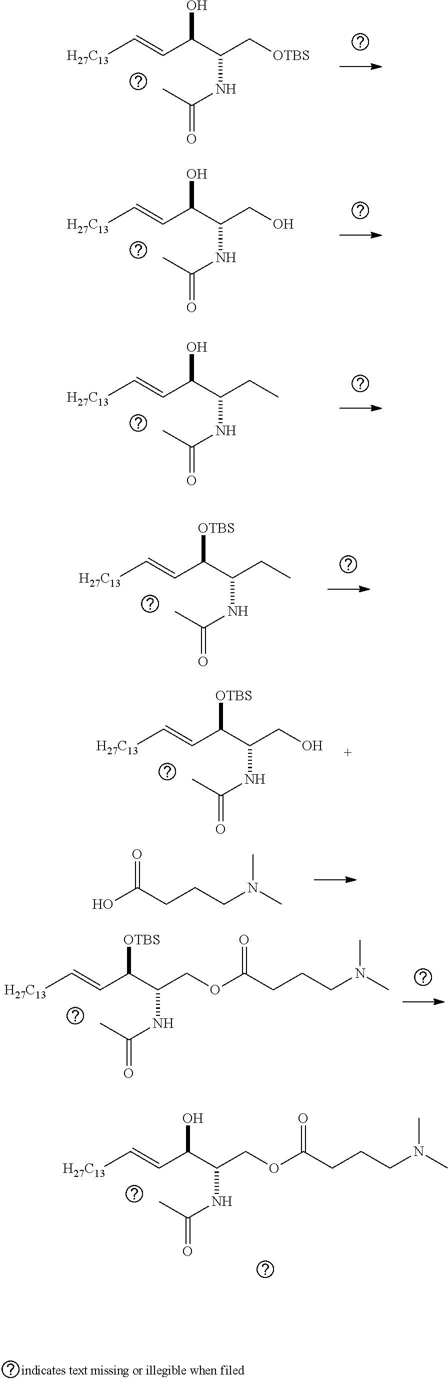

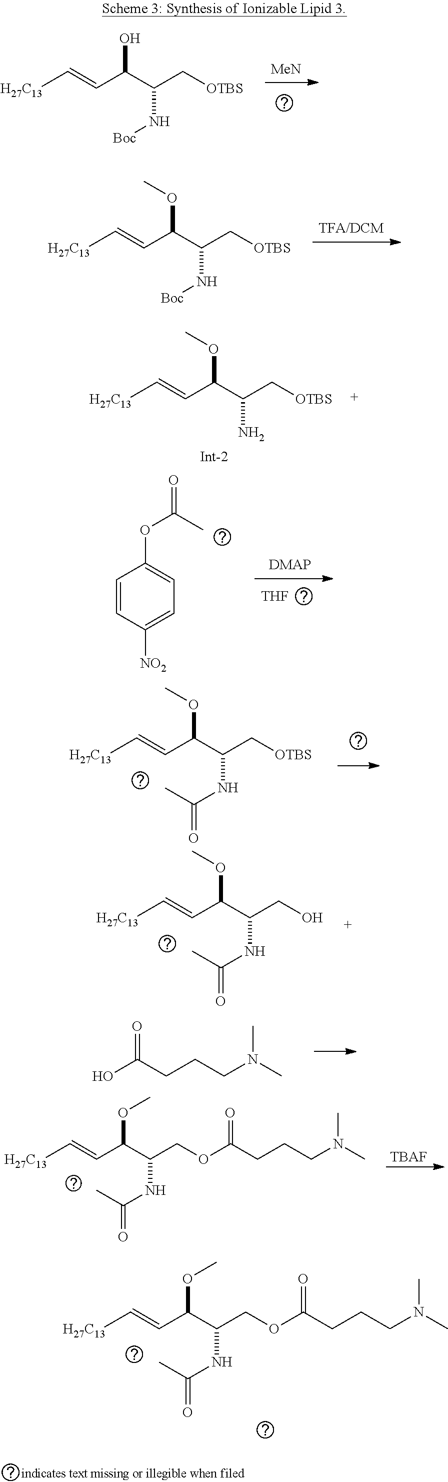

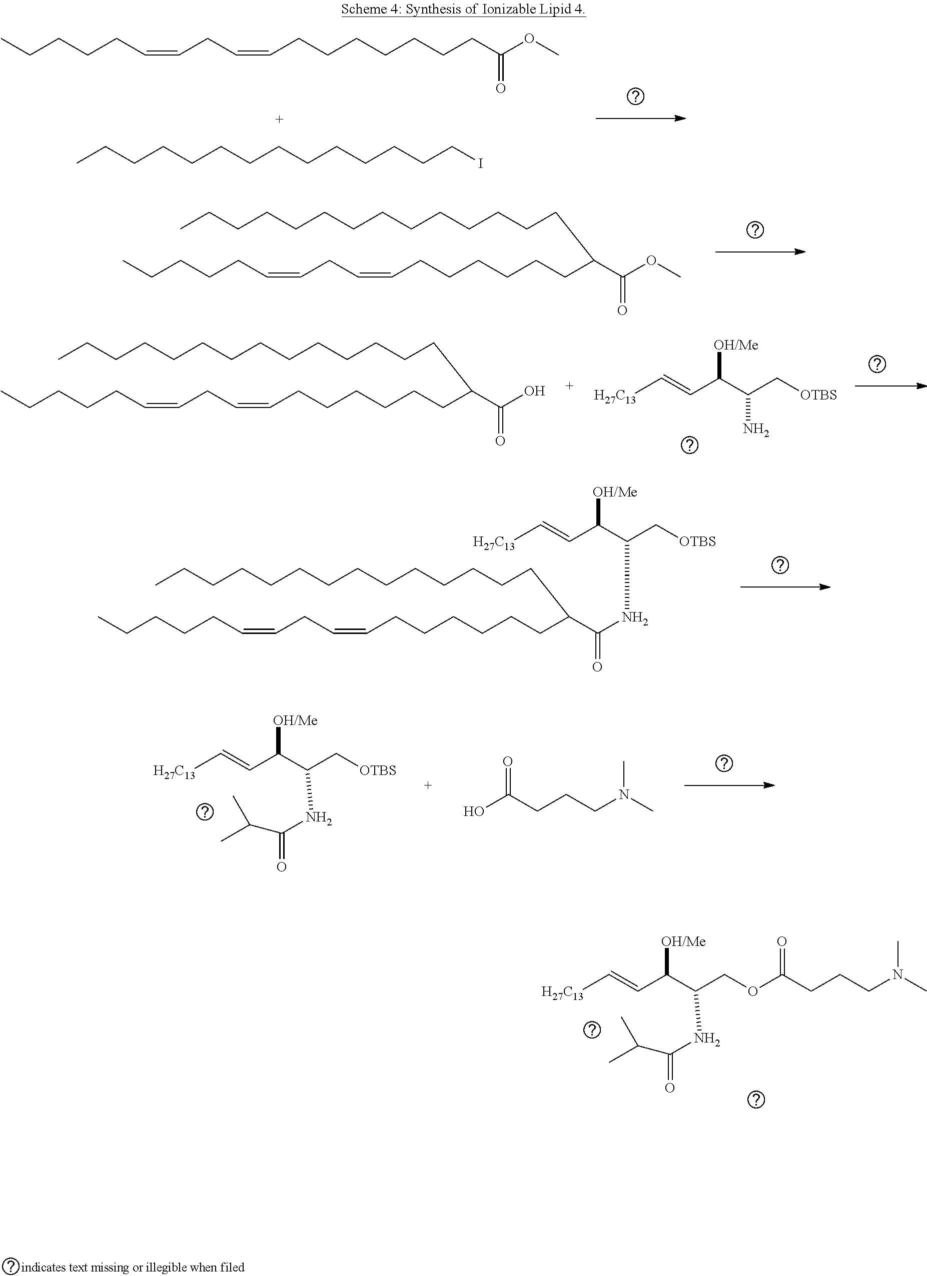

### Example 4: Synthesis of Ionizable Lipid of Formula (IV)

The ionizable lipids of Formula (IV) is based on sphingomyelin scaffold. The synthesis of ionizable lipid of Formula (IV) is depicted in Scheme 5 and Scheme 6 respectively. The procedures and methods are well known in the literature and are described in Chem. Eur. J. 2011, 17, 8568-8575 and in WO 2017/075531. Analogous sphingomyelin based ionizable lipids of Formula (IV) can be synthesized using the methods that are well known in the art.

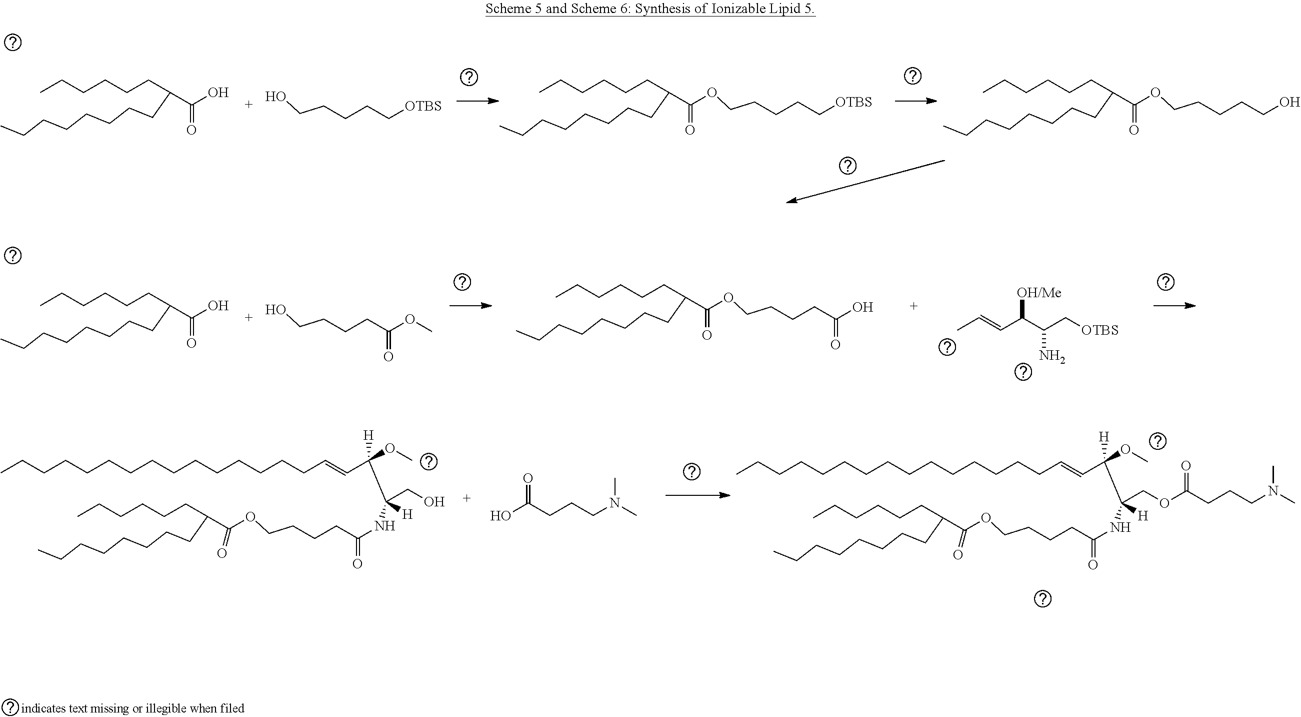

### Example 5: Synthesis of Ionizable Lipid of Formula (V)

The ionizable lipids of Formula (V) have a DOPE core. The synthesis of ionizable lipid of Formula (V) is depicted in Scheme 7 respectively. The procedures and methods are well known in the literature and are described in J. Med. Chem. 2005, 48, 7305-7314. Analogous DOPE core based ionizable lipids of Formula (V) can be synthesized using the methods that are well known in the art.

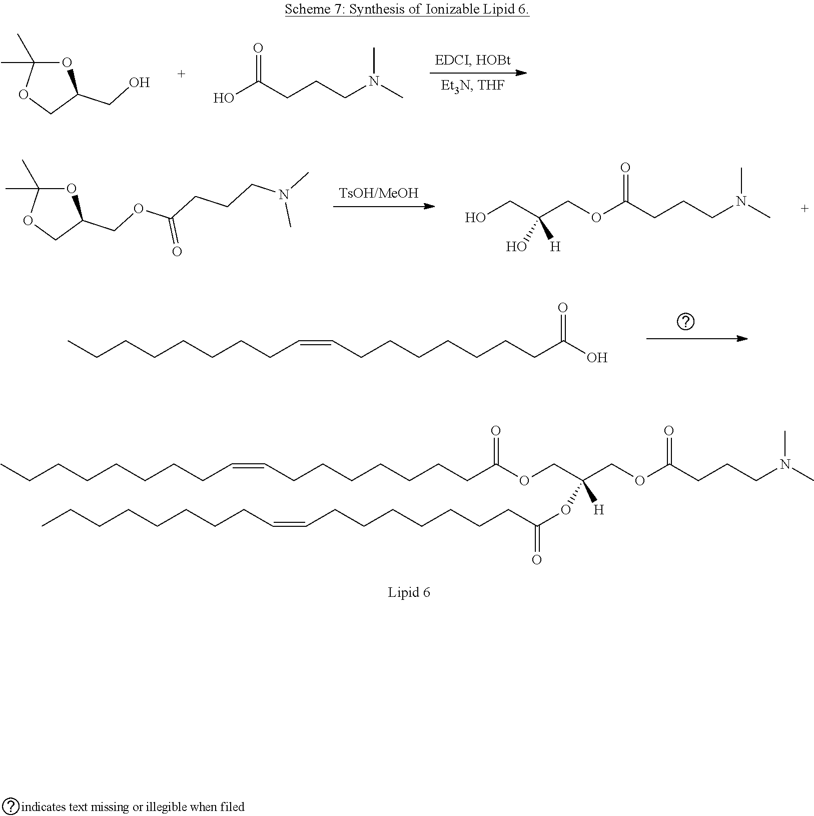

### Example 6: Synthesis of Ionizable Lipid of Formula (VI)

The ionizable lipids of Formula (VI) have a DOPE core. The synthesis of ionizable lipid of Formula (VI) is depicted in Scheme 8 and Scheme 9 respectively. Analogous DOPE core based ionizable lipids of Formula (VI) can be synthesized using synthetic protocols that are well known in the art.

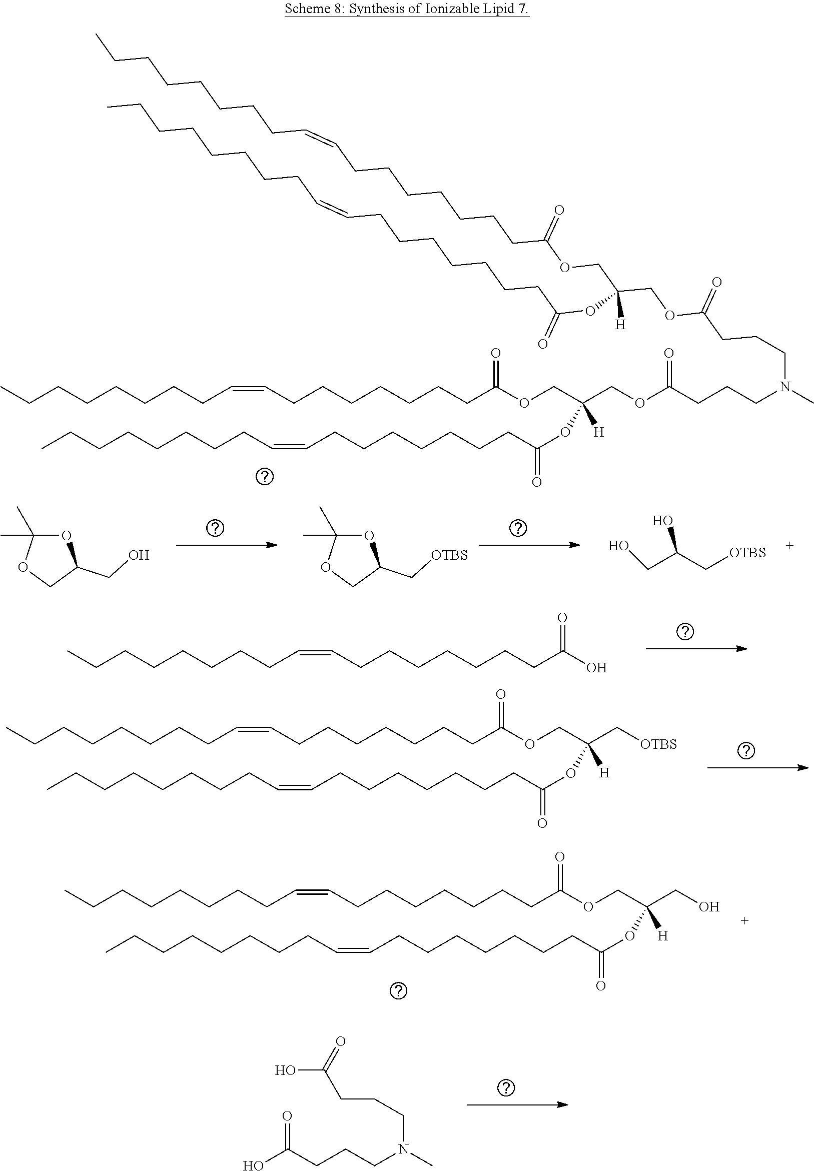

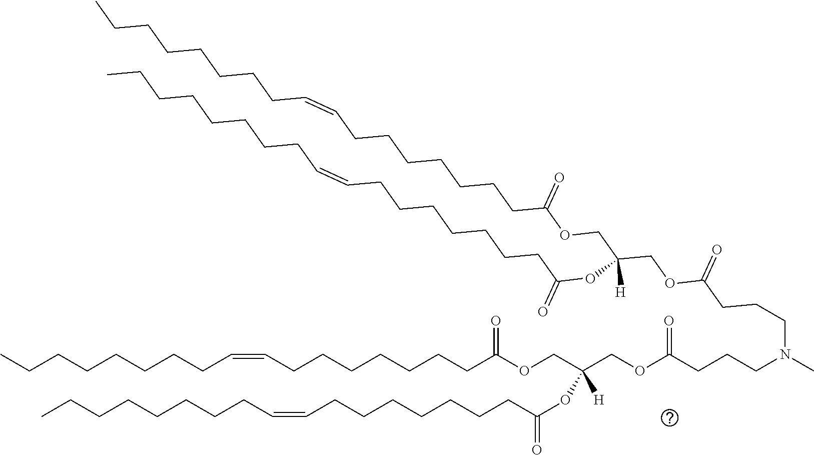 [SMILES: CCCCCCCC/C=C\CCCCCCCC(=O)OC[C@H](COC(=O)CCCN(C)CCCC(=O)OC[C@@H](COC(=O)CCCCCCC/C=C\CCCCCCCC)OC(=O)CCCCCCC/C=C\CCCCCCCC)OC(=O)CCCCCCC/C=C\CCCCCCCC]

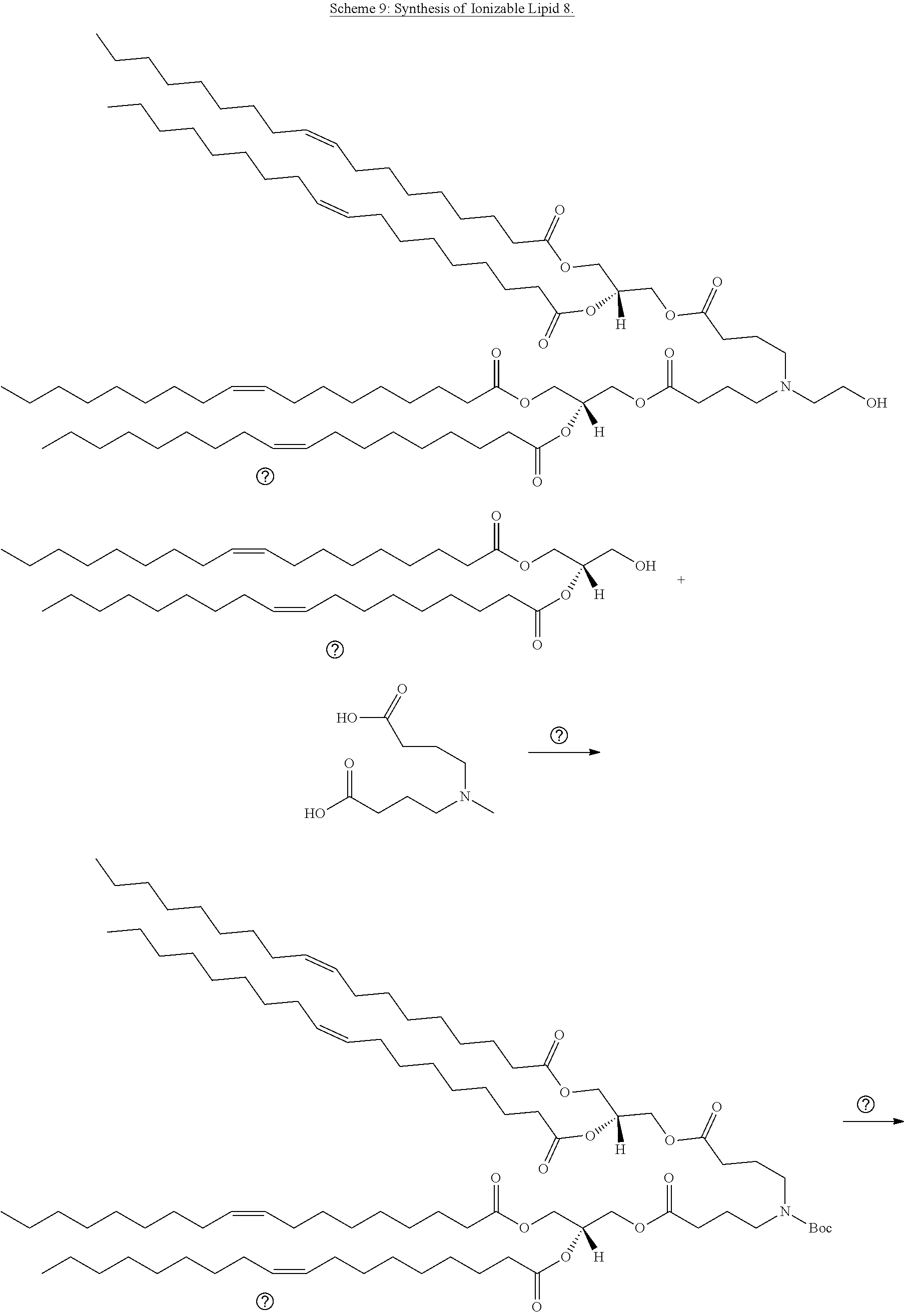 [SMILES: CCCCCCCC/C=C\CCCCCCCC(=O)OC[C@H](COC(=O)CCCN(CCO)CCCC(=O)OC[C@@H](COC(=O)CCCCCCC/C=C\CCCCCCCC)OC(=O)CCCCCCC/C=C\CCCCCCCC)OC(=O)CCCCCCC/C=C\CCCCCCCC ; CCCCCCCC/C=C\CCCCCCCC(=O)OC[C@H](CO)OC(=O)CCCCCCC/C=C\CCCCCCCC ; CN(CCCC(=O)O)CCCC(=O)O ; CCCCCCCC/C=C\CCCCCCCC(=O)OC[C@H](COC(=O)CCCN(CCCC(=O)OC[C@@H](COC(=O)CCCCCCC/C=C\CCCCCCCC)OC(=O)CCCCCCC/C=C\CCCCCCCC)C(=O)OC(C)(C)C)OC(=O)CCCCCCC/C=C\CCCCCCCC]

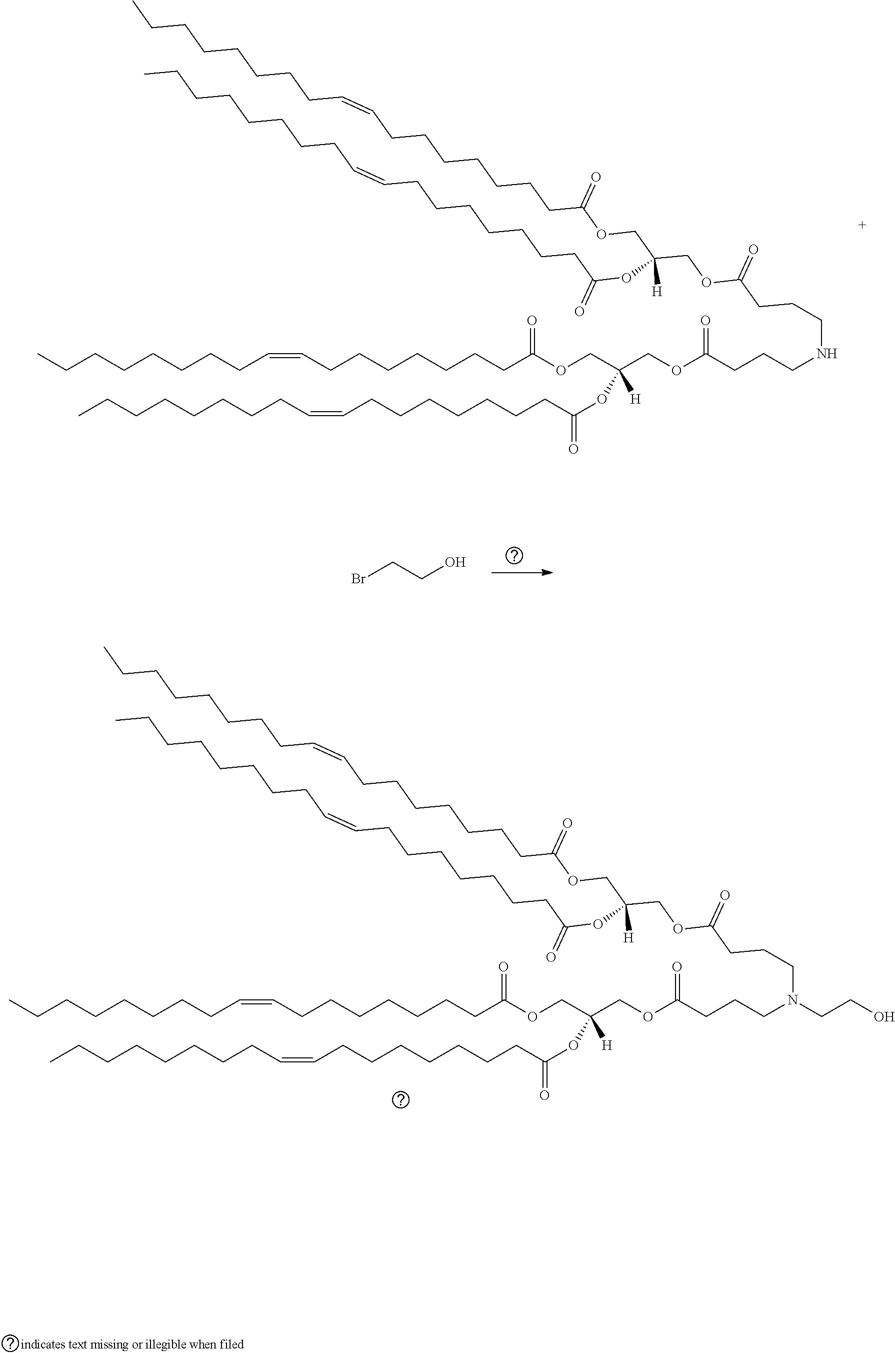

### Example 7: Synthesis of Ionizable Lipid of Formula (VII)

The ionizable lipids of Formula (VII) is based on triglyceride scaffold. The synthesis of ionizable lipid of Formula (VII) is depicted in Scheme 10 and Scheme 11 respectively. Analogous triglyceride-based modifications of ionizable lipids of Formula (VII) can be synthesized using the methods that are well known in the art.

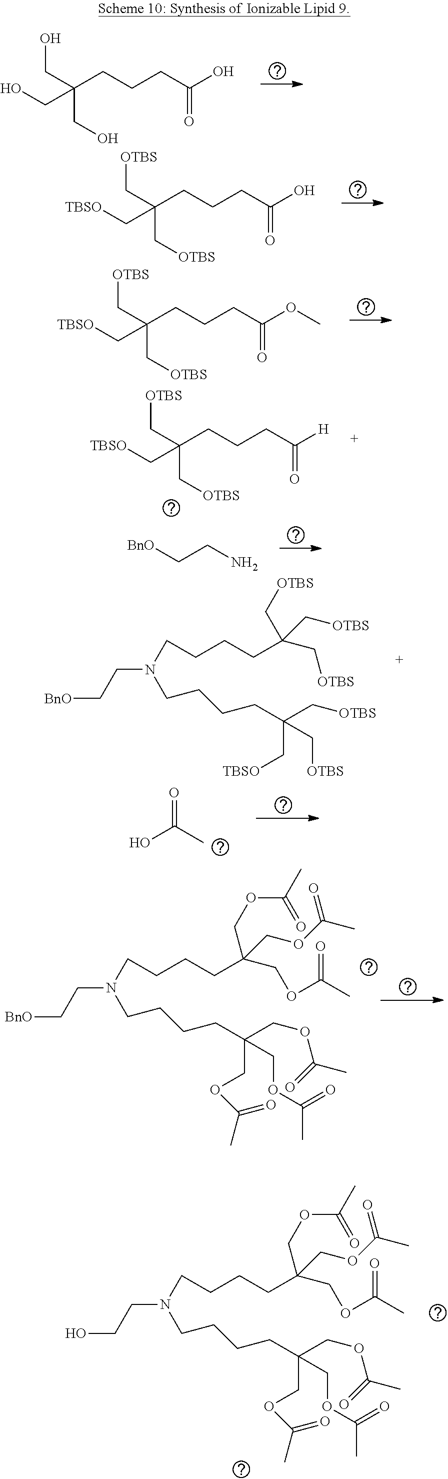

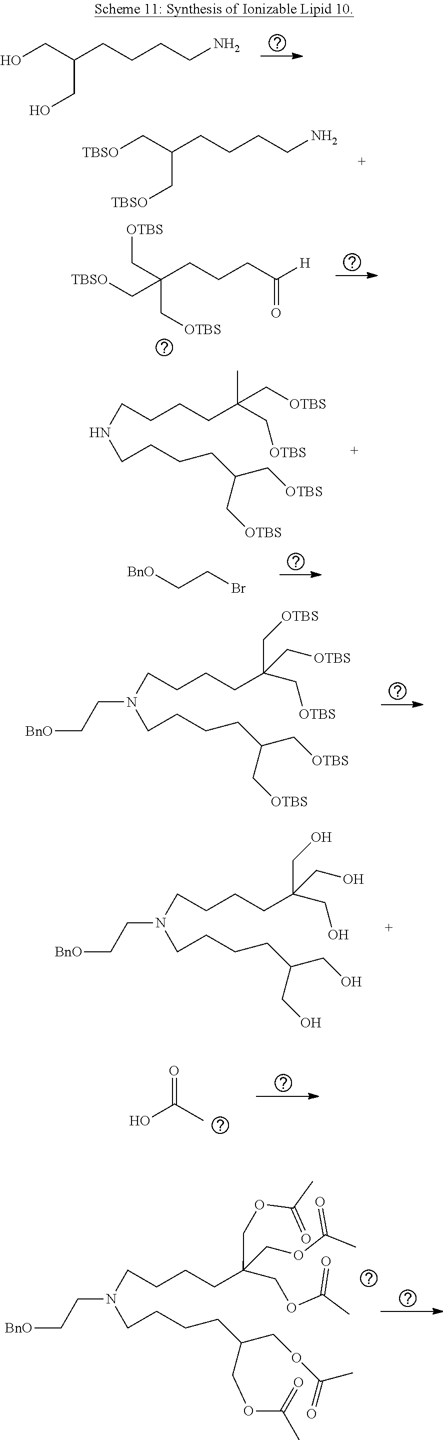

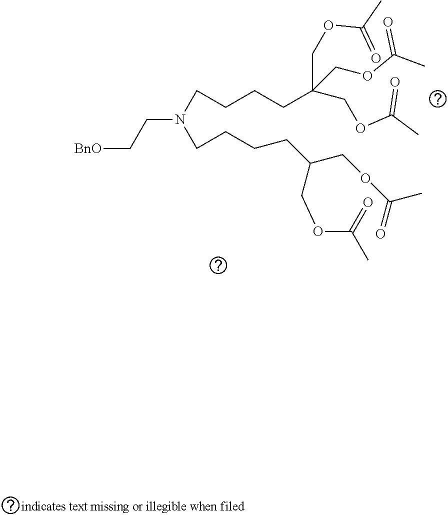 [SMILES: CC(=O)OCC(CCCCN(CCCCC(COC(C)=O)(COC(C)=O)COC(C)=O)CCOCc1ccccc1)COC(C)=O]

### Example 8: Synthesis of Ionizable Lipid of Formula (VIII)

The ionizable lipids of Formula (VIII) is based on triglyceride scaffold. The synthesis of ionizable lipid of Formula (VIII) is depicted in Scheme 12 respectively. Analogous triglyceride-based modifications of ionizable lipids of Formula (VIII) can be synthesized using the methods that are well known in the art.

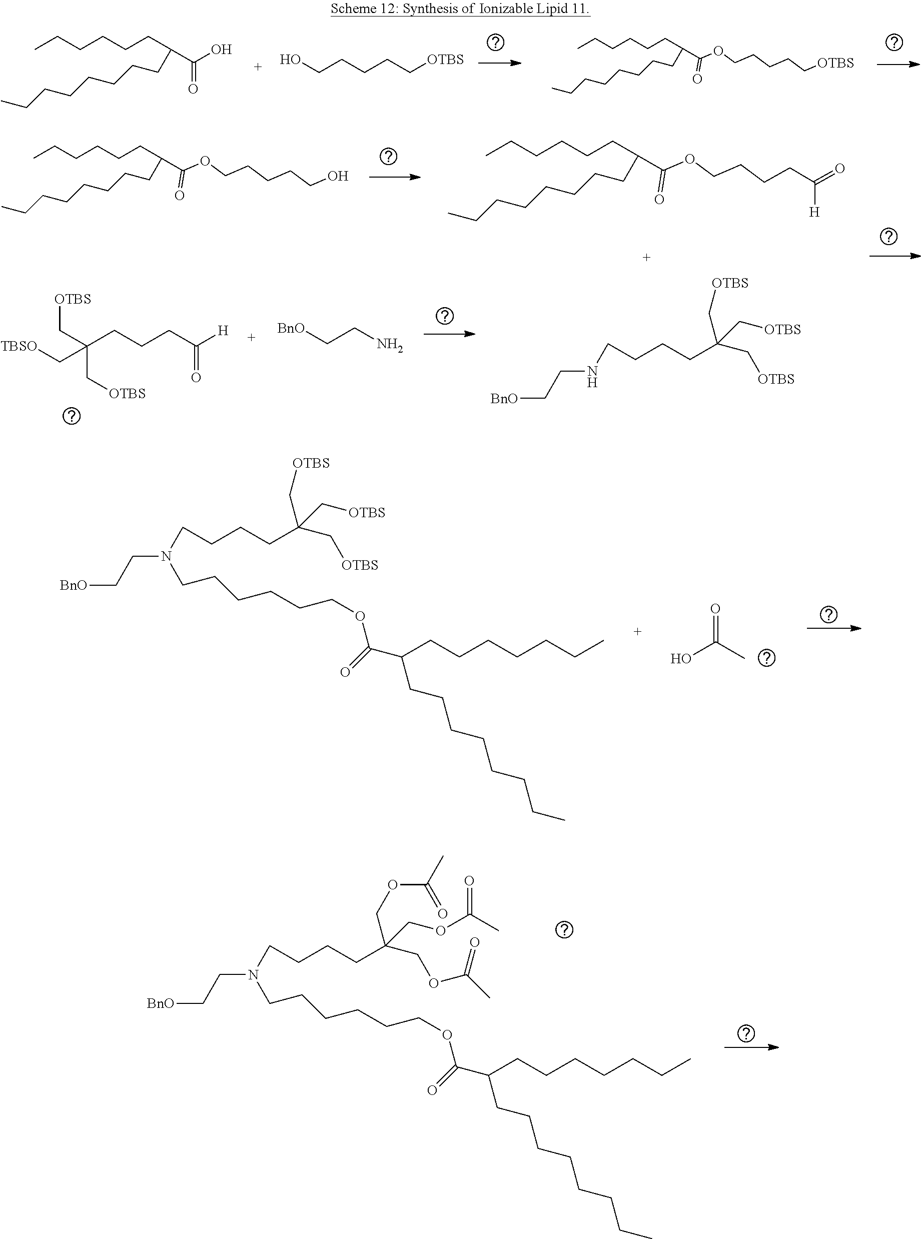

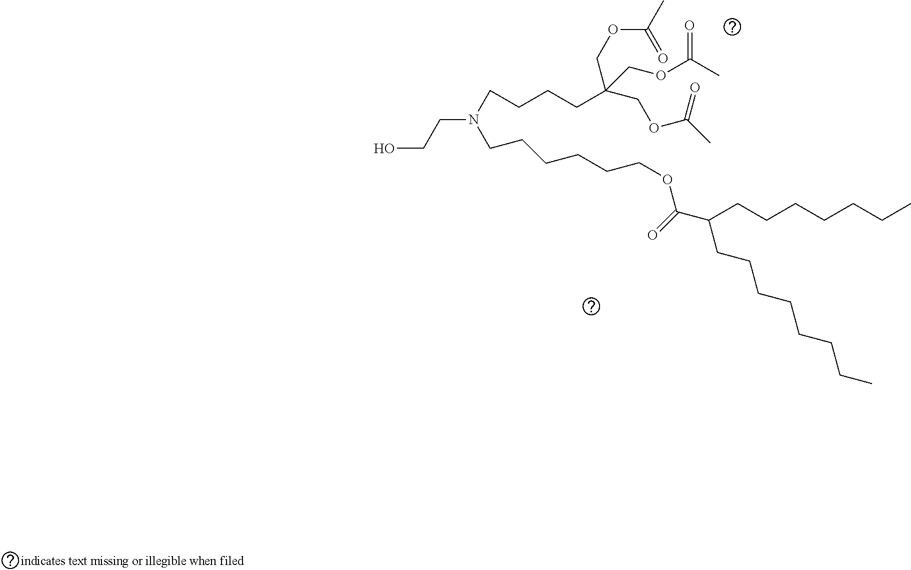 [SMILES: CCCCCCCCC(CCCCCCC)C(=O)OCCCCCCN(CCO)CCCCC(COC(C)=O)(COC(C)=O)COC(C)=O]

### Example 9: Synthesis of Ionizable Lipid of Formula (IX)

The ionizable lipids of Formula (IX) have cyclic head groups in the scaffold. The synthesis of ionizable lipid of Formula (IX) is depicted in Scheme 13 and Scheme 14 respectively. Analogous cyclic head group-based modifications of ionizable lipids of Formula (IX) can be synthesized using the methods that are well known in the art.

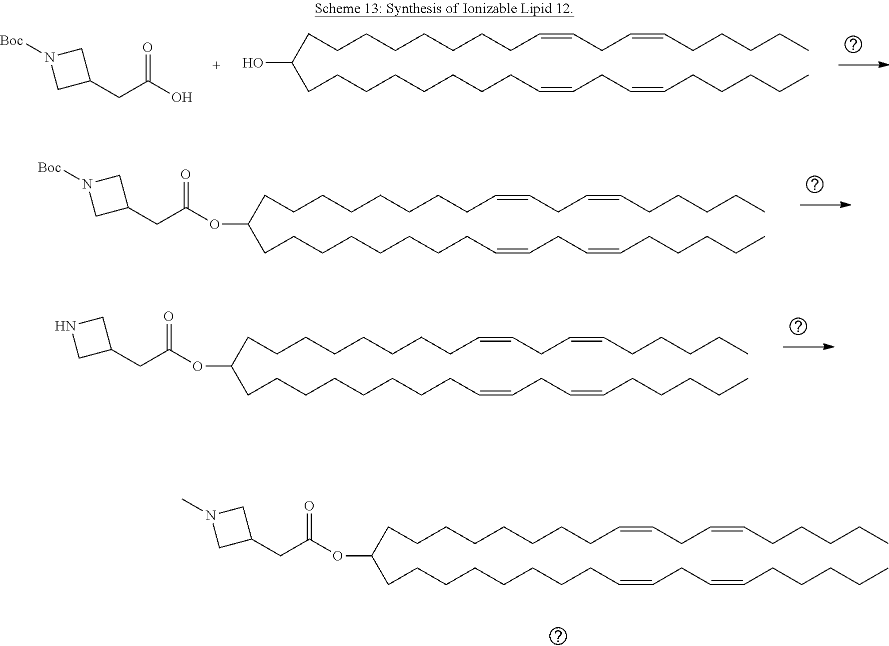

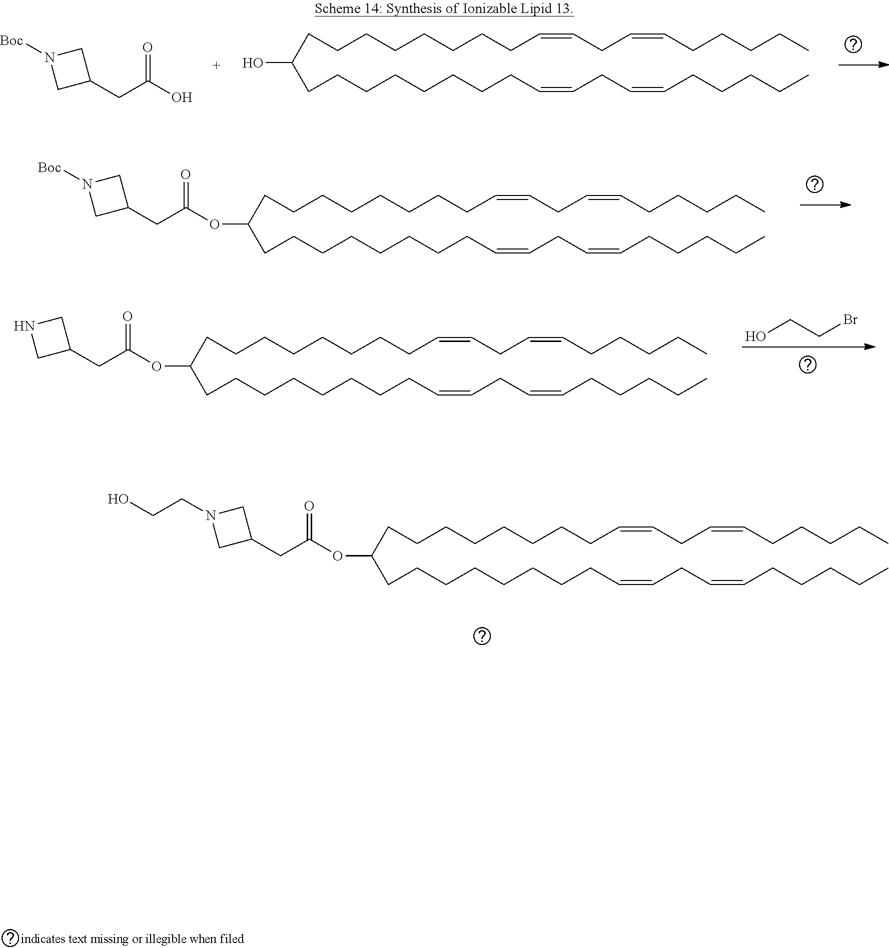

### Example 10: Synthesis of Ionizable Lipid of Formula (X)

The ionizable lipids of Formula (X) have cyclic head groups in the scaffold. The synthesis of ionizable lipid of Formula (X) is depicted in Scheme 15 and Scheme 16 respectively. Analogous cyclic head group-based modifications of ionizable lipids of Formula (X) can be synthesized using the methods that are well known in the art.

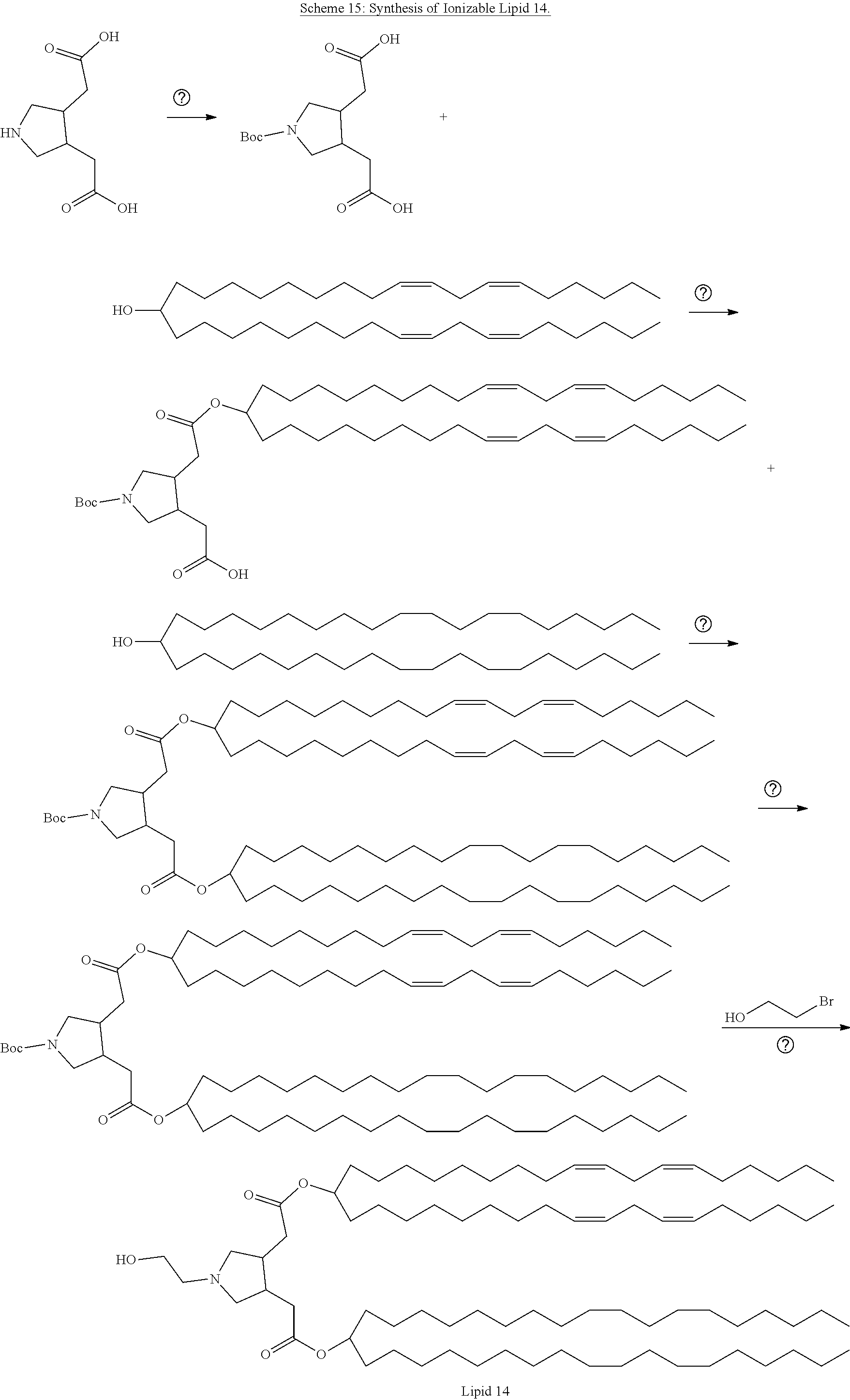

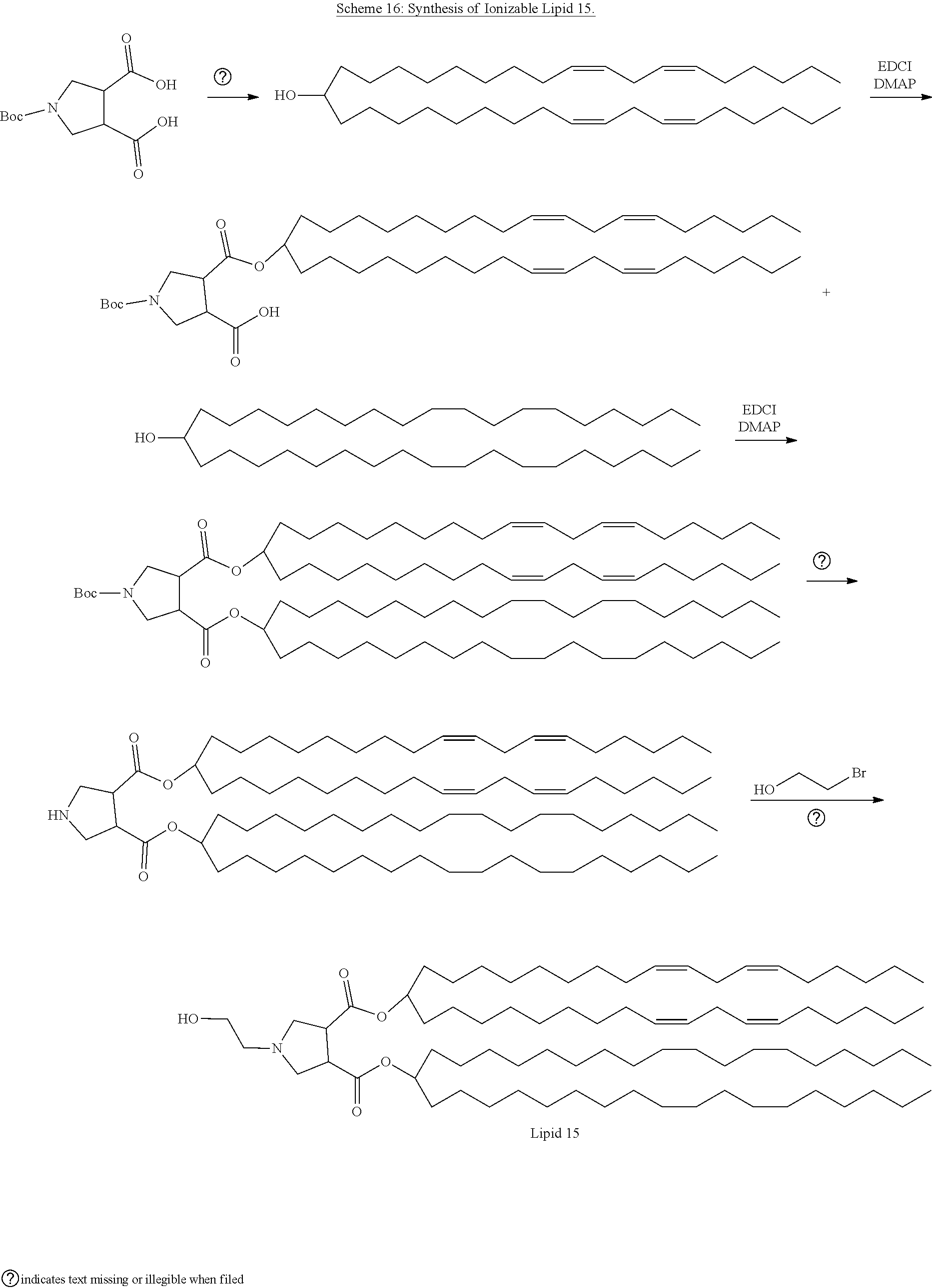

### Example 11: Synthesis of Ionizable Lipid of Formula (XI)

The ionizable lipids of Formula (XI) have cyclic head groups in the scaffold. The synthesis of ionizable lipid of Formula (XI) is depicted in Scheme 17 respectively. Analogous cyclic head group-based modifications of ionizable lipids of Formula (XI) can be synthesized using the methods that are well known in the art.

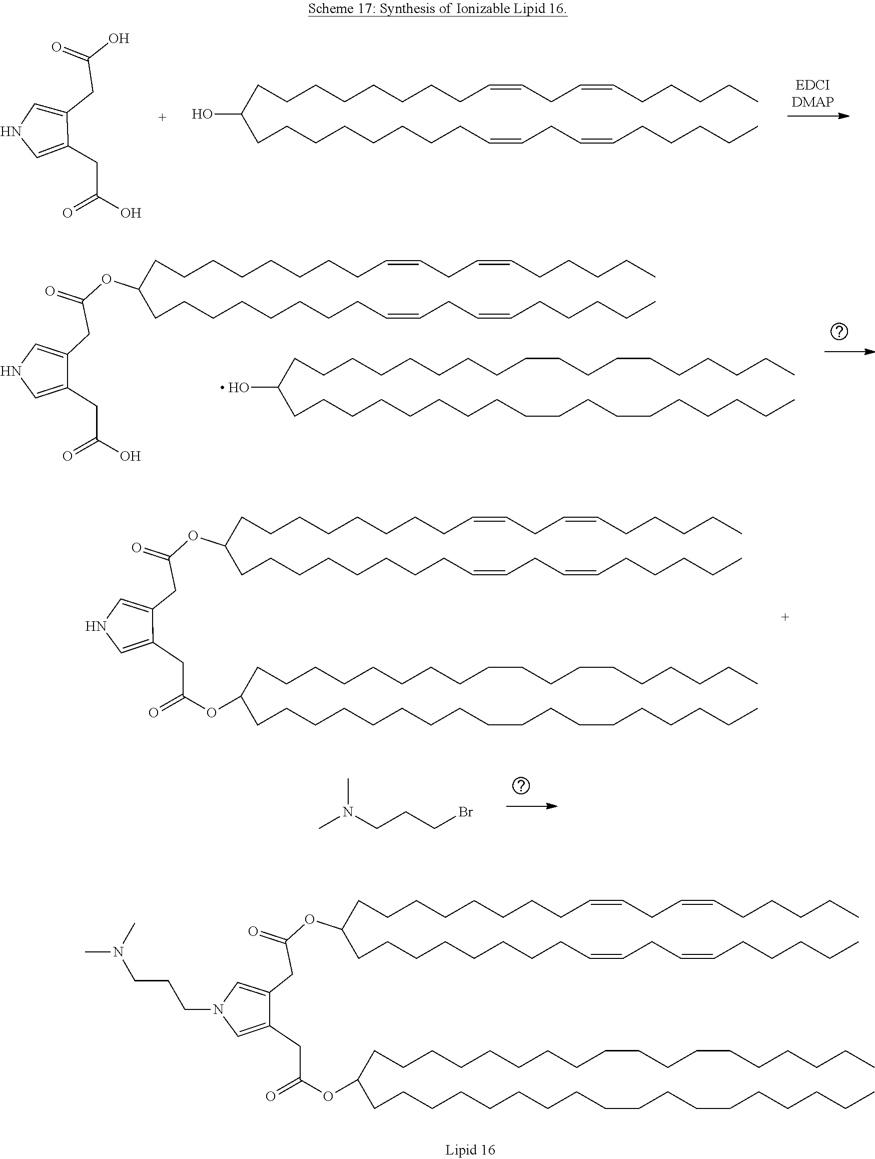

### Example 12: Synthesis of Ionizable Lipid of Formula (XII)

The ionizable lipids of Formula (XII) have cyclic head groups in the scaffold. The synthesis of ionizable lipid of Formula (XII) is depicted in Scheme 18 respectively. Analogous cyclic head group-based modifications of ionizable lipids of Formula (XII) can be synthesized using the methods that are well known in the art.

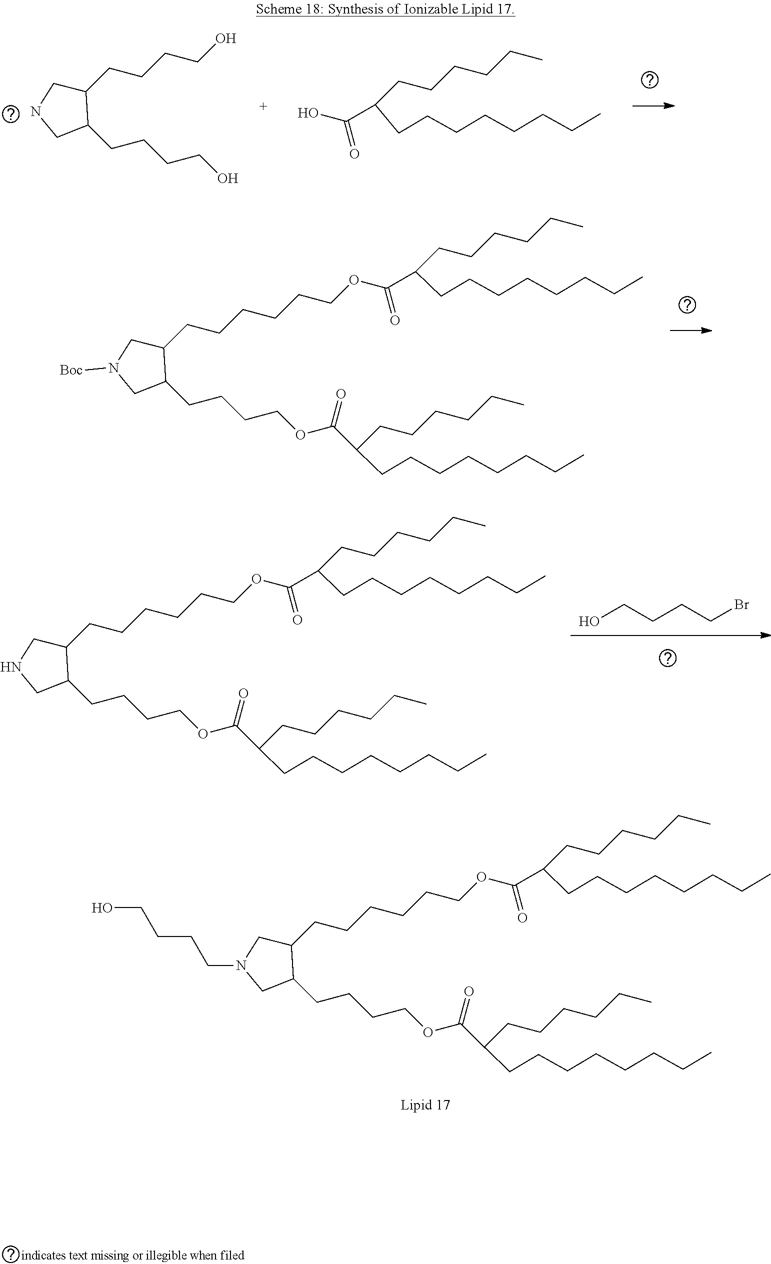

### Example 13: Synthesis of Ionizable Lipid of Formula (XIII)

The ionizable lipids of Formula (XIII) have cyclic head groups in the scaffold. The synthesis of ionizable lipid of Formula (XIII) is depicted in Scheme 19 respectively. Analogous cyclic head group-based modifications of ionizable lipids of Formula (XIII) can be synthesized using the methods that are well known in the art.

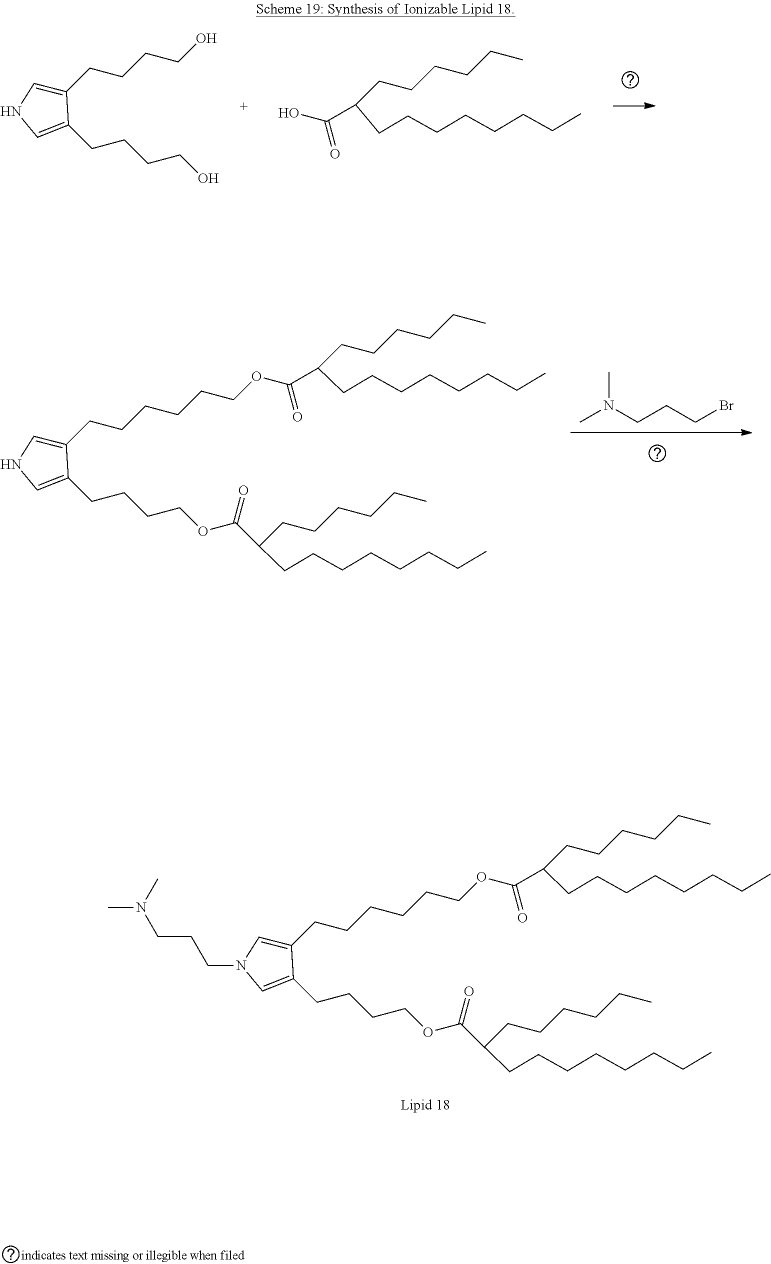

### Example 14: Synthesis of Ionizable Lipid of Formula (XIV)

The ionizable lipids of Formula (XIV) have aromatic rings in the scaffold. The synthesis of ionizable lipid of Formula (XIV) is depicted in Scheme 20 respectively. Analogous ionizable lipids containing aromatic rings of Formula (XIV) can be synthesized using the methods that are well known in the art.

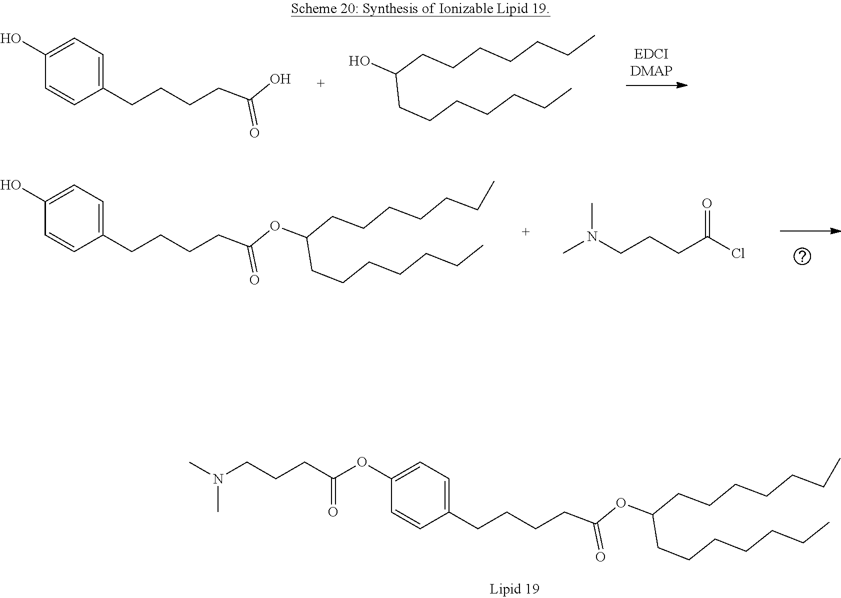

### Example 15: Synthesis of Ionizable Lipid of Formula (XV)

The ionizable lipids of Formula (XV) have aromatic rings in the scaffold. The synthesis of ionizable lipid of Formula (XV) is depicted in Scheme 21 and Scheme 22 respectively. Analogous ionizable lipids containing aromatic rings of Formula (XV) can be synthesized using the methods that are well known in the art.

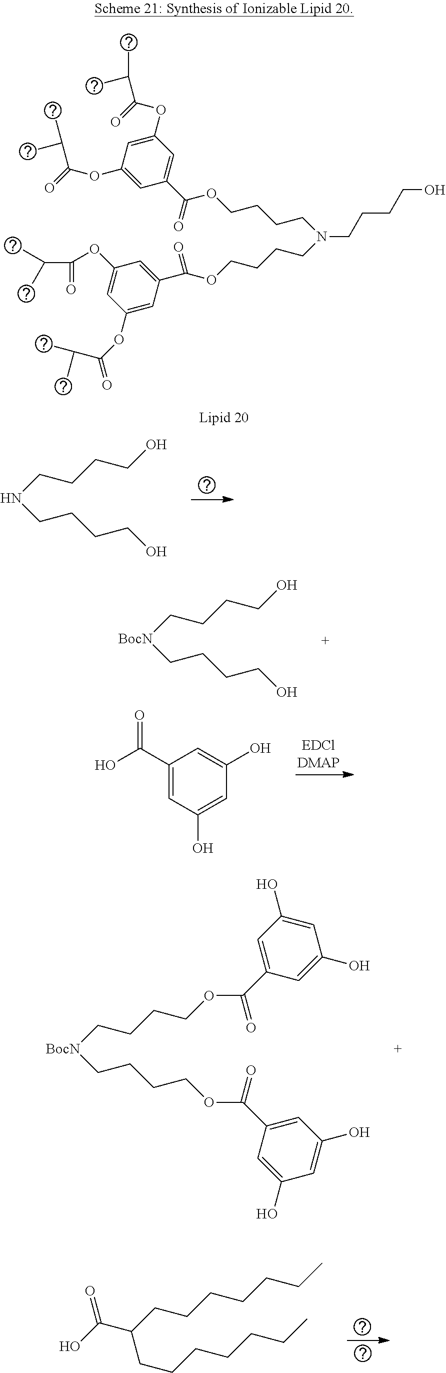 [SMILES: CC(C)C(=O)Oc1cc(OC(=O)C(C)C)cc(C(=O)OCCCCN(CCCCO)CCCCOC(=O)c2cc(OC(=O)C(C)C)cc(OC(=O)C(C)C)c2)c1 ; OCCCCNCCCCO ; CC(C)(C)OC(=O)N(CCCCO)CCCCO ; O=C(O)c1cc(O)cc(O)c1 ; CC(C)(C)OC(=O)N(CCCCOC(=O)c1cc(O)cc(O)c1)CCCCOC(=O)c1cc(O)cc(O)c1 ; CCCCCCCC(CCCCCCC)C(=O)O]

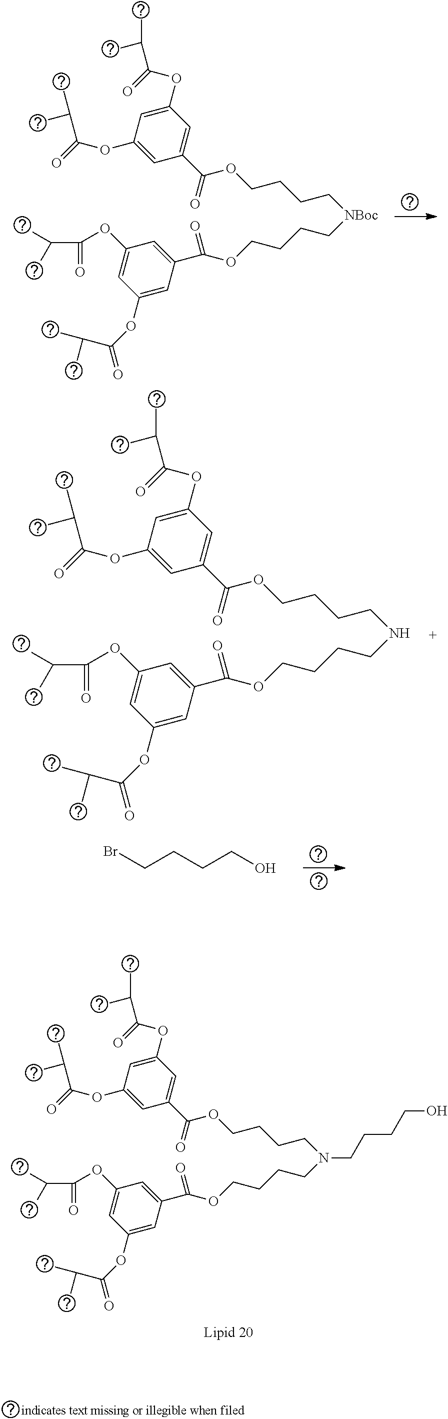

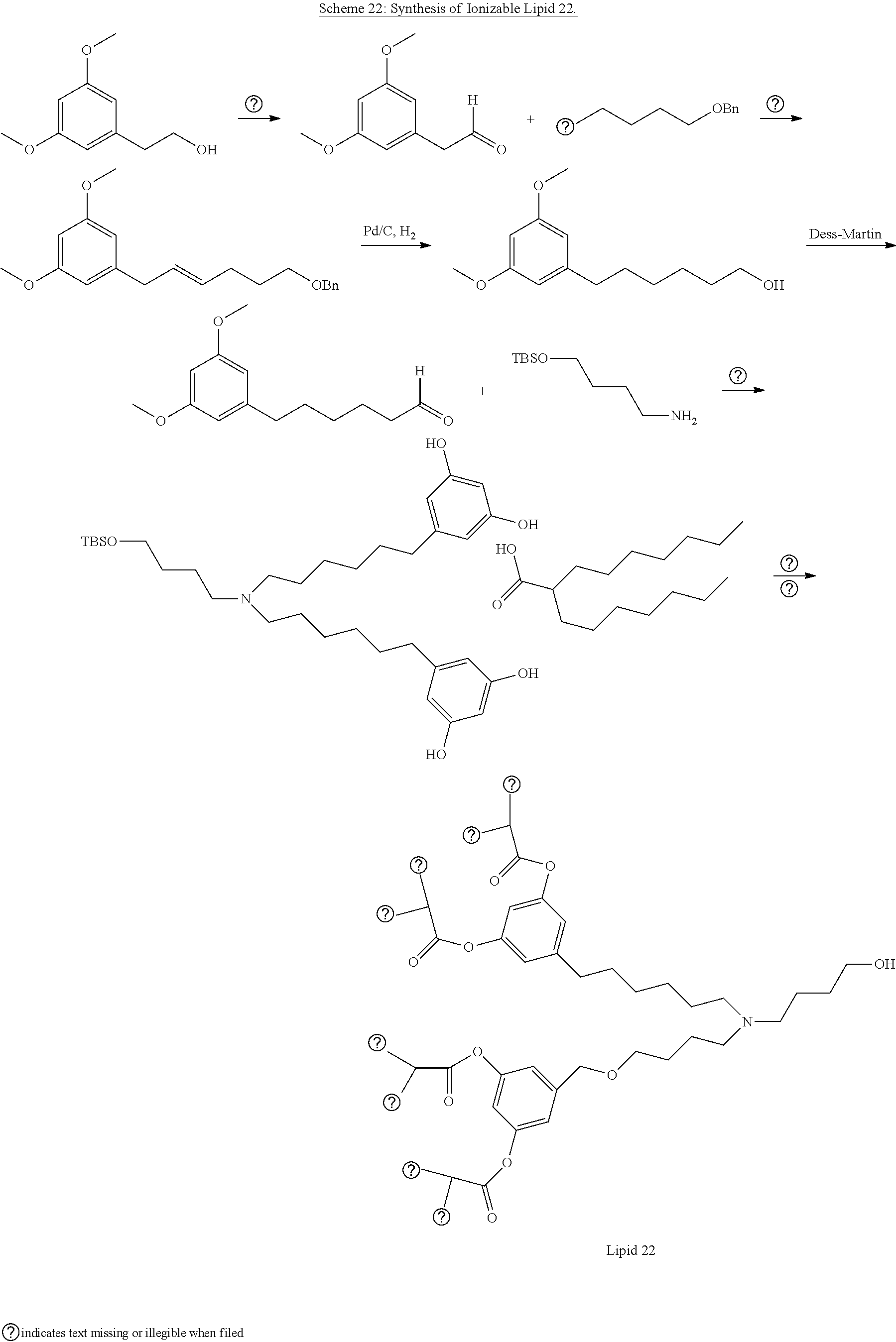

### Example 16: Synthesis of Ionizable Lipid of Formula (XVI) and Formula (XVI-a)

The ionizable lipids of Formula (XVI) and Formula (XVI-A) have aromatic rings in the scaffold. The synthesis of ionizable lipid of Formula (XVI) and Formula (XVI-A) is depicted in Scheme 23 and Scheme 24 respectively. Analogous ionizable lipids containing aromatic rings of Formula (XVI) and Formula (XVI-A) can be synthesized using the methods that are well known in the art.

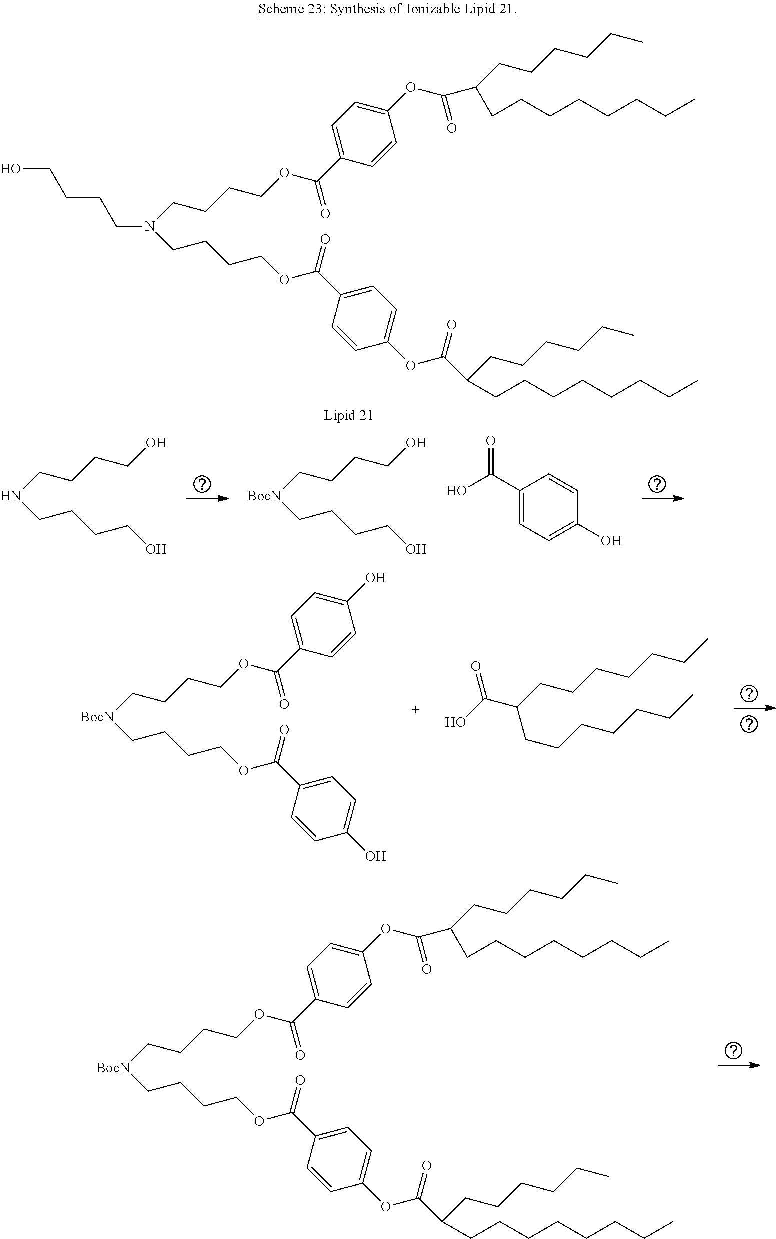

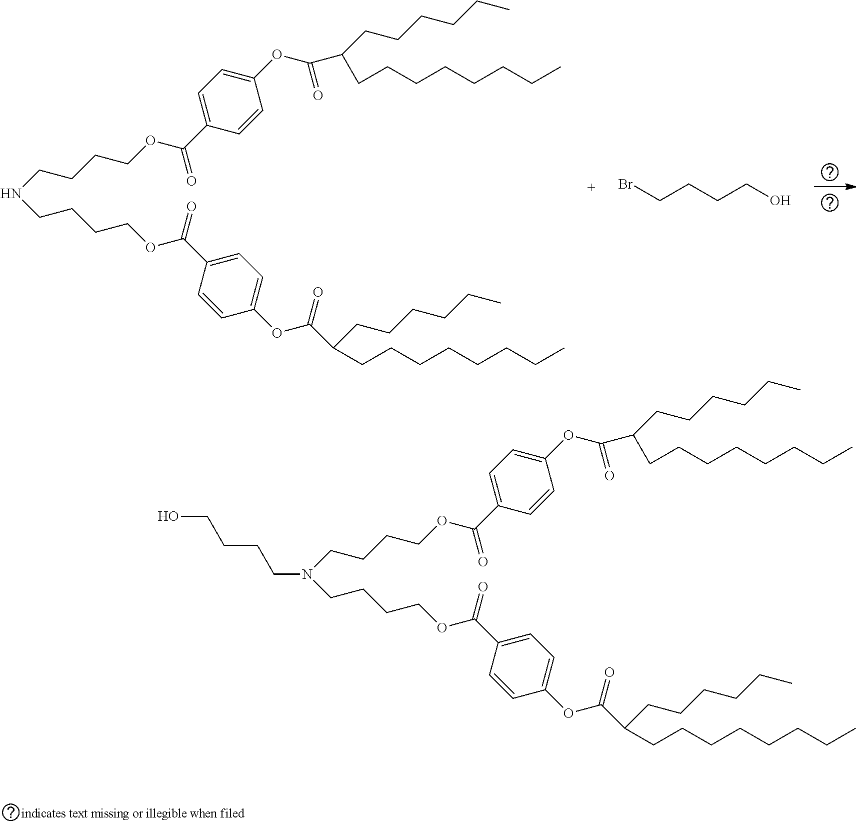 [SMILES: CCCCCCCCC(CCCCCC)C(=O)Oc1ccc(C(=O)OCCCCNCCCCOC(=O)c2ccc(OC(=O)C(CCCCCC)CCCCCCCC)cc2)cc1 ; OCCCCBr ; CCCCCCCCC(CCCCCC)C(=O)Oc1ccc(C(=O)OCCCCN(CCCCO)CCCCOC(=O)c2ccc(OC(=O)C(CCCCCC)CCCCCCCC)cc2)cc1]

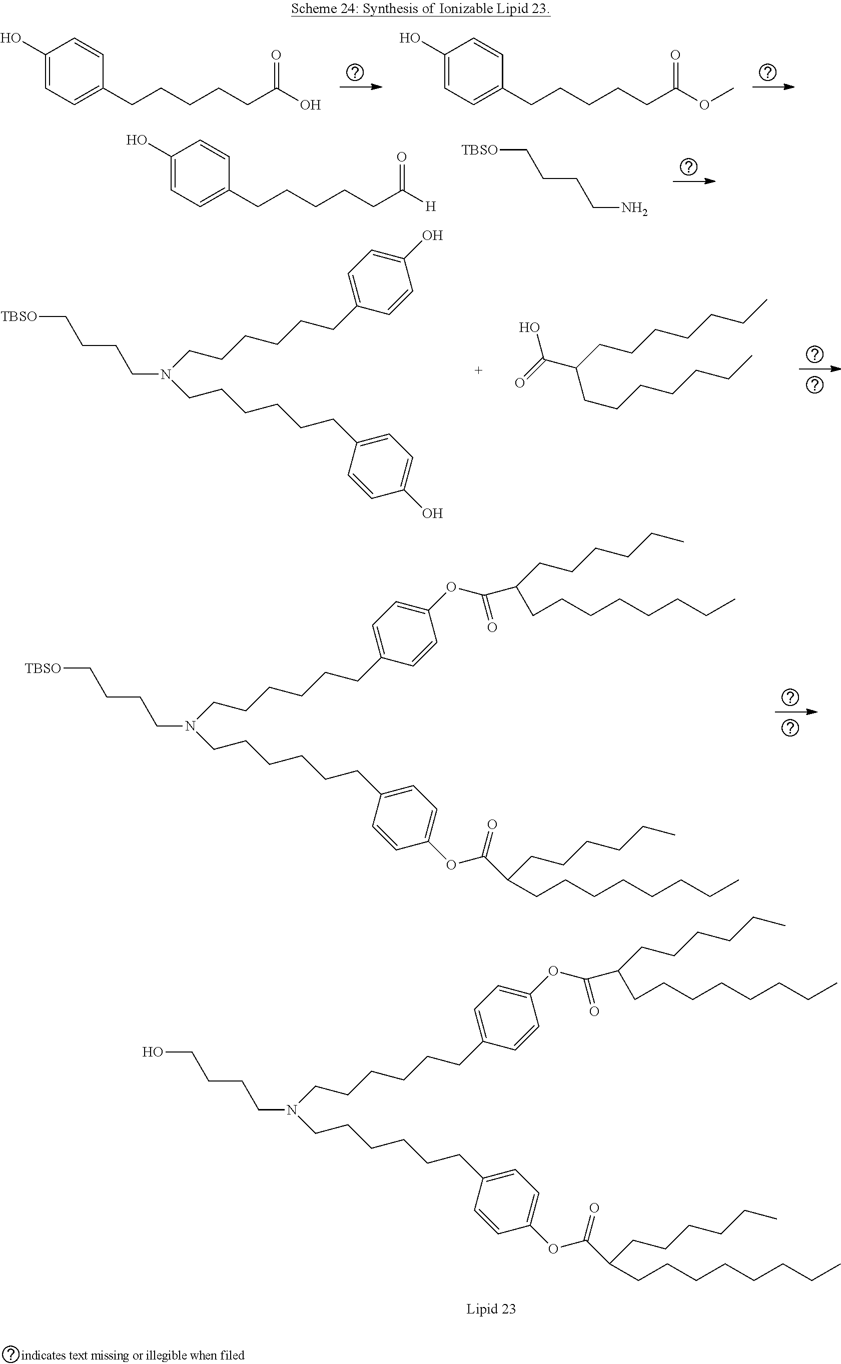

### Example 16: Transfection and Rejuvenation of Aged Human Dermal Fibroblasts

Primary human dermal fibroblasts from a 40-year-old female donor were seeded in 6-well plates at density of 200,000 to 250,000 cells/well. Some wells were left treated, some wells were transfected with lipid nanoparticles carrying mCherry-expressing mRNA (a reporter gene) as a control, and some wells were transfected with lipid nanoparticles comprising a monocistronic mRNA cocktail, where each mRNA molecule expressed one of the reprogramming factors O, S, K, L, M, or N. Each well was transfected with 1 microgram of RNA. The lipid nanoparticles contained lipid formula (II) of the disclosure, together with additional components, such as DOPC, DMG-PEG, and cholesterol, for example at molar ratios of 50:10:1.5:38.5.

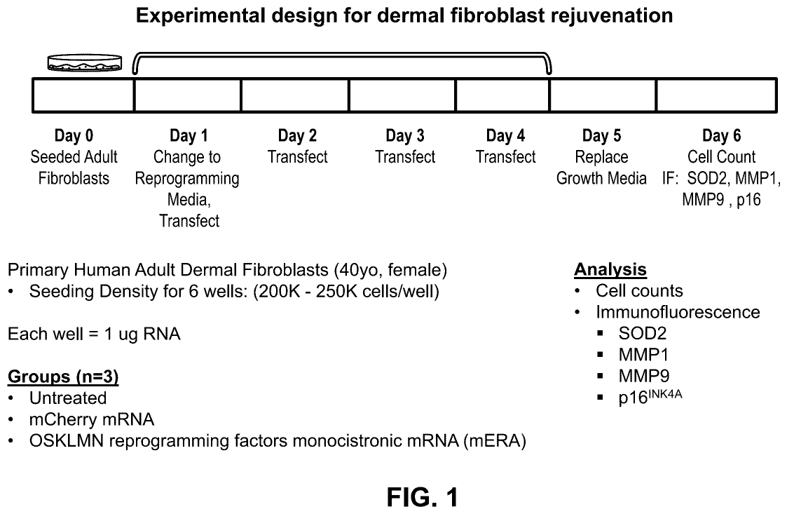

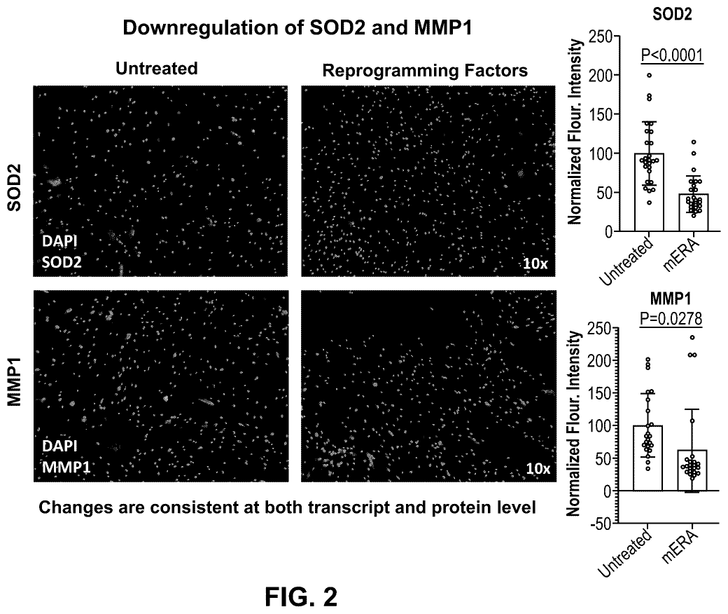

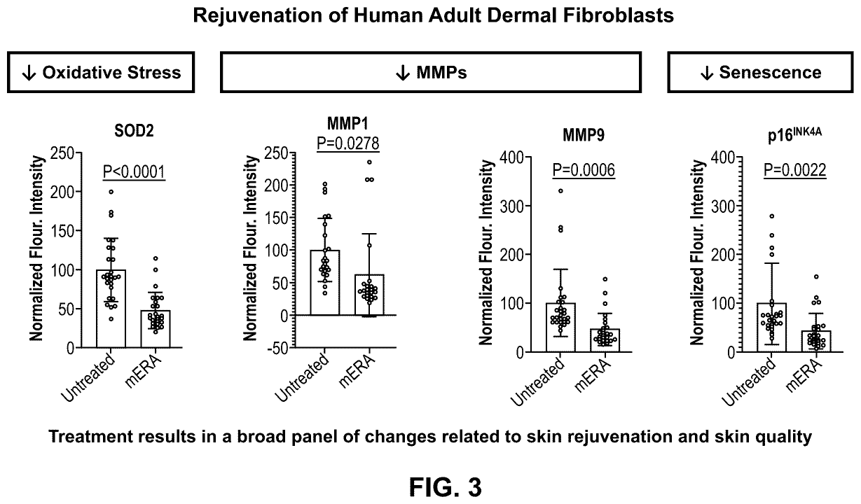

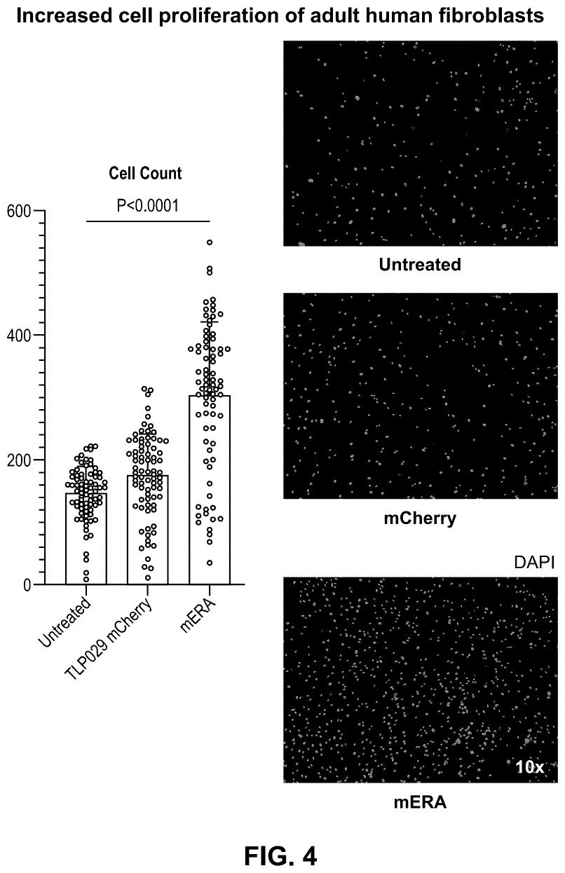

Transfections were performed on Days 1, 2, 3, and 4. On Day 6, cells were counted and immunofluorescence analysis was performed for SOD2, MMP1, MMP9, and p16^{INK4A} as markers of skin rejuvenation, skin quality, and/or senescence. The experimental design is shown in FIG. 1. Immunofluorescence revealed downregulation of SOD2, MMP1, MMP9, and p^{16INK4A} when cells were transiently reprogrammed using lipid nanoparticles containing lipid formula (II) and the mRNA cocktail encoding O, S, K, L, M, and N, compared to untreated cells. The data are shown in FIGS. 2-3. Cell proliferation was also increased compared to untreated cells as shown in FIG. 4. This is representative of skin rejuvenation, increased skin quality, decreased oxidative stress, and decreased senescence.

### Example 17: Transfection and Rejuvenation of Human Skin Explants

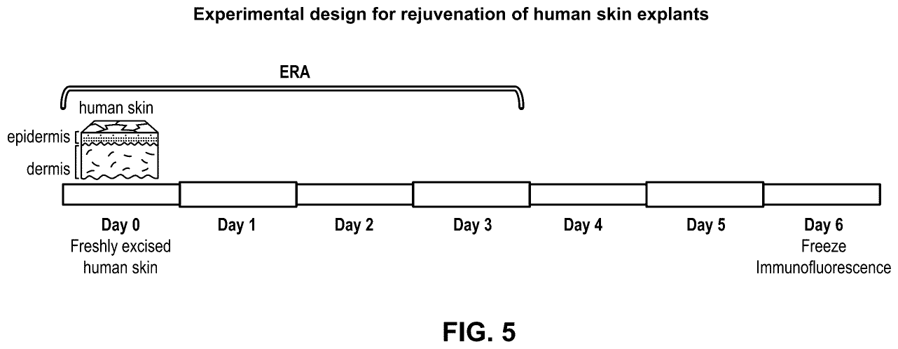

Human skin samples were obtained from a 52-year-old female patient with 12 mm punch biopsies and prepared for culture. They were left untreated; treated with RENOVA® (a retinoid cream containing tretinoin used clinically to improve the appearance of the skin by reducing fine lines and wrinkles, reducing roughness, and improving skin color); or injected subcutaneously with lipid nanoparticles comprising a monocistronic mRNA cocktail, where each mRNA molecule expressed one of the reprogramming factors O, S, K, L, M, or N. Each injection included 2 micrograms of RNA. The experimental design is shown in FIG. 5.

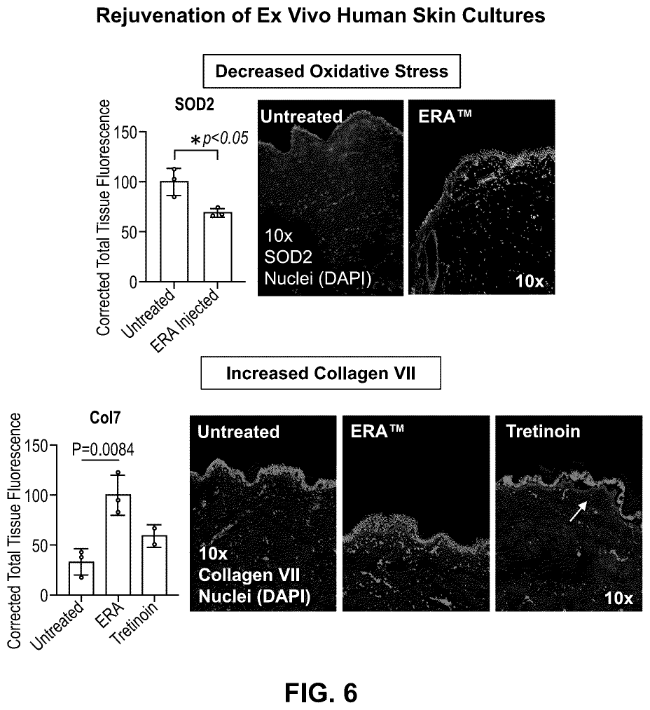

Transfections were performed on Days 0, 1, 2, and 3. On Day 6, the skin explants were frozen for analysis by immunohistochemistry for SOD2 and Collagen VII as markers of markers of skin rejuvenation and skin quality. Immunohistochemistry revealed downregulation of SOD2 and upregulation of Collagen VII when skin explants were transiently reprogrammed using lipid nanoparticles containing lipid formula (II) and the mRNA cocktail encoding O, S, K, L, M, and N, compared to untreated cells. This indicates skin rejuvenation, increased skin quality, and decreased oxidative stress in human skin. The lipid nanoparticles containing lipid formula (II) and the mRNA cocktail encoding O, S, K, L, M, and N induced higher Collagen VII expression than RENOVA.® The data are shown in FIG. 6.

### Example 18: Transfection with Lipid Nanoparticle Compositions

Lipid nanoparticle compositions were prepared for in vitro transfection studies using primary human dermal fibroblast cells. mRNA expressing mCherry as a reporter gene was used as a payload in the formulations and its fluorescence was used as a direct measure of transfection efficiency. The lipid used in the lipid nanoparticle formulations is set forth in Table 18-1, and each formulation included a phospholipid and a hydrophilic polymer.

TABLE 18-1

| Formulation ID No. | Lipid in Formulation |
|---|---|
| #29 | Formula I-B |
| #30 | Formula I-B |
| #32 | Formula I |
| #33 | Formula I |
| #29S | Formula II |
| #30S | Formula II |

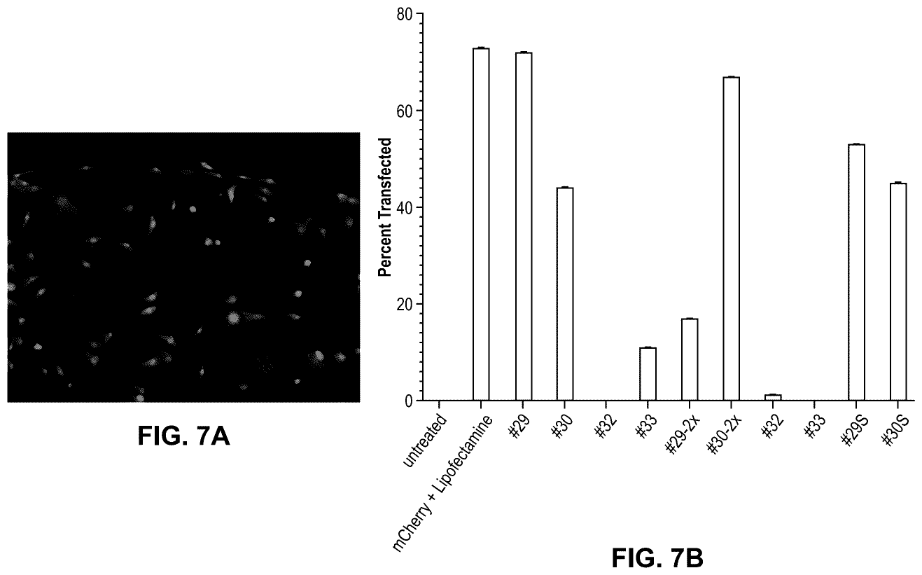

For transfection, the cells were plated in 96-well plates at about 17,000-25,000 cells per well. The cells were transfected the following day with a lipid nanoparticle formulation. Based on the mCherry mRNA concentration for each formulation, a serial dilution was made for each formulation to provide an mRNA dose per well of 100 ng, 50 ng or 25 ng. At 24 hours post transfection, transfection efficiency was determined by imaging the cells and counting the percent of cells (counterstained with DAPI) expressing the mCherry reporter (detected in red). Relative fluorescence was also quantified using a SpectraMax Plate Reader (Molecular Devices) at 587 nm/610 nm. Background fluorescence from a blank was subtracted from raw data. Data from the cells dosed with 100 ng mRNA is presented in FIGS. 7A-7B, where FIG. 7A is an image of cells treated with Formulation ID No. #29 and FIG. 7B is a bar graph showing the percentage of cells transfected for the identified formulations, each dosed at 100 ng mRNA. The formulations with a “2×’ indication were prepared with twice the amount of lipid components than the formulations without the 2× indicator. Untreated cells and cells treated with mRNA expressing mCherry and the transfection reagent Lipofectamine™ served as controls.

Lipid nanoparticles containing ionizable lipid of Formula I-B (Formulation ID Nos. #29 and #30) provided higher transfection efficiency than lipid nanoparticles containing ionizable lipid of Formula I (Formulation ID Nos. #32 and #33, respectively) in primary human dermal fibroblasts, where the difference in ionizable lipids was the only difference in components between Formulation ID Nos. #29 and #32, and between Formulation ID Nos. #30 and #33. This improvement was observed for two different lipid nanoparticle compositions, each containing a different helper lipid, i.e., #29 and #32 contained one helper lipid, and Formulation ID No. #30 and Formulation ID No. #33 contained a different helper lipid.

### Example 19: Transfection with Lipid Nanoparticle Compositions

Lipid nanoparticle compositions were prepared for transfection of enhanced green fluorescent protein (eGFP) mRNA. The compositions are set forth in Table 19-1. Compositions identified as Composition ID No. 19-5A and Composition ID No. 19-5B comprised the ionizable lipid shown below, in its S-isomer form, a lipid of Formula I and corresponding to ionizable lipid 1 in Example 1:

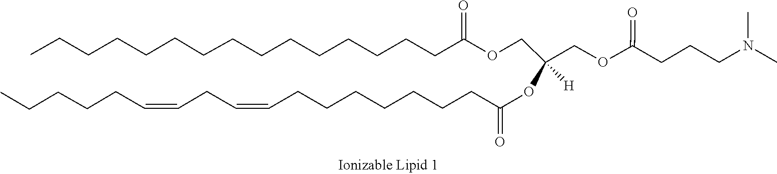 [SMILES: CCCCC/C=C\C/C=C\CCCCCCCC(=O)O[C@@H](COC(=O)CCCCCCCCCCCCCCC)COC(=O)CCCN(C)C]

The compositions identified as Composition ID No. 19-6A and Composition ID No. 19-6B comprised the ionizable lipid shown below, a lipid of Formula I-B having the structure of Compound 27 of Table A, in its R-isomer form:

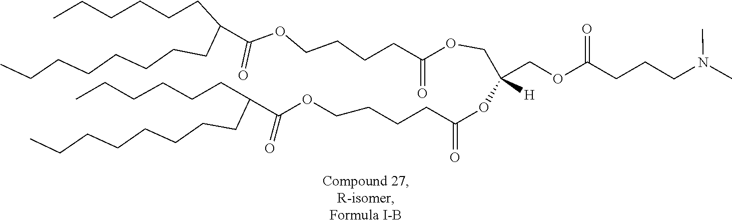 [SMILES: CCCCCCCCC(CCCCCC)C(=O)OCCCCC(=O)OC[C@H](COC(=O)CCCN(C)C)OC(=O)CCCCOC(=O)C(CCCCCC)CCCCCCCC]

All lipid nanoparticle compositions comprised eGFP mRNA, the indicated ionizable lipid (45-50 wt %), a helper lipid (such as DSCP, DOPC, DOPE), a stabilizing lipid (e.g., DMG-PEG), and a structural lipid (sphingomyelin, cholesterol).

TABLE 19-1

| Composition | |
|---|---|
| ID No. | Ionizable Lipid |
| 19-1A | Ionizable Lipid 1 |
| | (Formula I) |
| 19-1B | Ionizable Lipid 1 |
| | (Formula I) |
| 19-2A | Compound 27 |
| | R-isomer |
| | Formula I-B |
| 19-2B | Compound 27 |
| | R-isomer |
| | Formula I-B |

Jurkat cells and activated Pan T-cells were plated in 96 we plates at about 1×10⁶ cells per well. Each composition was applied to a well (n=3) in an amount needed to provide a dose of 3 μg eGFP mRNA. The cells were incubated at 37° C. for 10 minutes and then transferred into culture media. 18-24 hours later, expression of eGFP protein was measured by flow cytometry and the cells where stained with propidium iodide to evaluate toxicity.

For comparison with the lipid nanoparticle compositions, cells were also transfected by electroporation (1350V or 1600V, for 10 ms, and 3 pulses) and Lipofectamine™ MessengerMAX™ where 1.25 μL Lipofectamine was used per 1×10⁶ cells.

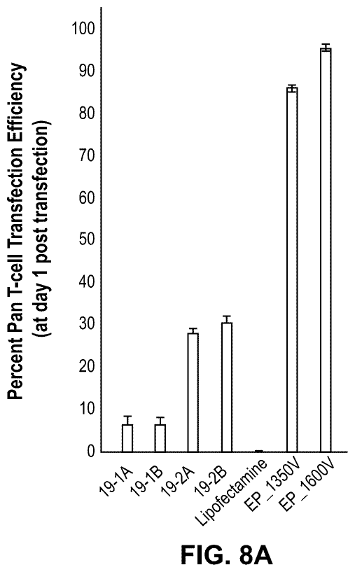

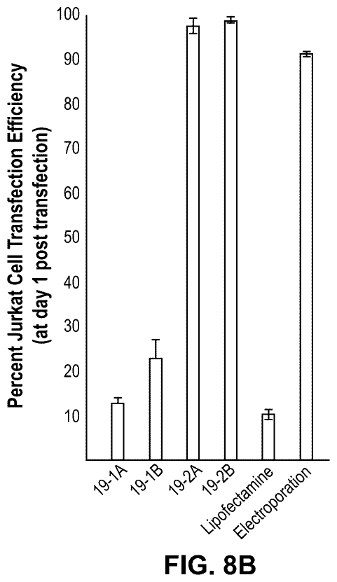

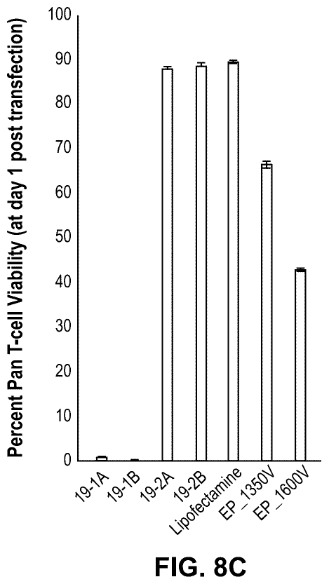

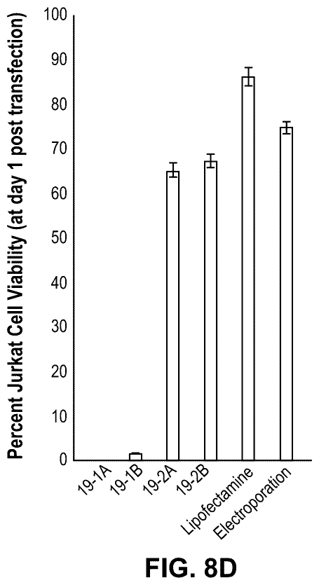

Results are shown in FIGS. 8A-8D. Lipid nanoparticles compositions containing ionizable lipid of Formula I-B, Composition ID Nos. 19-2A and 19-2B, provided higher transfection efficiency than lipid nanoparticles containing ionizable lipid of Formula I, Composition ID Nos. 19-1A and 19-1B, in both Jurkat and primary T cells, where the difference in ionizable lipids was the only difference in components between Composition ID Nos. 19-1A and 19-2A and between Composition ID Nos. 19-1B and 19-2B. This improvement was observed for two different lipid nanoparticle compositions, each containing a different helper lipid, i.e., Composition ID Nos. 19-1A and 19-2A contained one helper lipid, and Composition ID Nos. 19-1B and 19-2B contained a different helper lipid. Similarly, the lipid nanoparticles containing ionizable lipid of Formula I-B, Composition ID Nos. 19-2A and 19-2B) provided higher viability in Jurkat and primary T cells than lipid nanoparticles containing ionizable lipid of Formula I, Composition ID Nos. 19-1A and 19-1B; notably, lipid nanoparticles containing ionizable lipid of Formula I-B were practically non-toxic in primary T cells, whereas lipid nanoparticles containing ionizable lipid of Formula I were toxic.

The transfection efficiency for lipid nanoparticles containing the ionizable lipid of Formula I-B was higher than that for Lipofectamine™. Viability was also improved compared to electroporation.

### Example 20: Transfection with Lipid Nanoparticle Compositions

Lipid nanoparticle compositions were prepared for transfection of enhanced green fluorescent protein (eGFP) mRNA. The compositions are set forth in Table 20-1.

Compositions identified as Composition ID No. 20-2A and 20-2B comprised the ionizable lipid shown below, a lipid of Formula I-B having the structure of Compound 27 of Table A, in its R-isomer form:

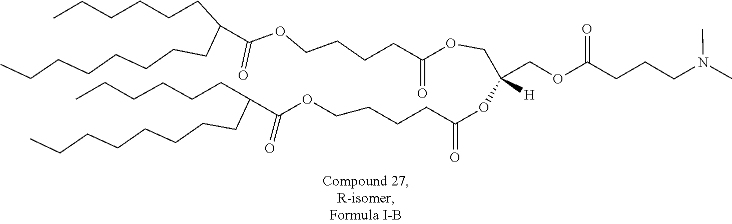 [SMILES: CCCCCCCCC(CCCCCC)C(=O)OCCCCC(=O)OC[C@H](COC(=O)CCCN(C)C)OC(=O)CCCCOC(=O)C(CCCCCC)CCCCCCCC]

The compositions identified as Composition ID No. 20-3A and Composition ID No. 20-3B comprised the ionizable lipid shown below, a lipid of Formula I having the structure of Compound 24 (Table A), in its R-isomer form:

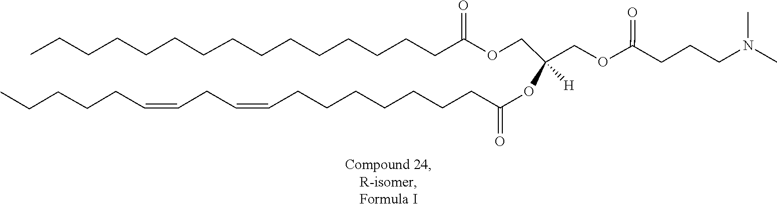 [SMILES: CCCCC/C=C\C/C=C\CCCCCCCC(=O)O[C@@H](COC(=O)CCCCCCCCCCCCCCC)COC(=O)CCCN(C)C]

All lipid nanoparticle compositions comprised eGFP mRNA, the indicated ionizable lipid (50 wt %), a helper lipid (such as DSCP, DOPC, DOPE), a stabilizing lipid (e.g., DMG-PEG), and a structural lipid (sphingomyelin or cholesterol).

TABLE 20-1

| Composition | |
|---|---|
| ID No. | Ionizable Lipid |
| 20-1A | Compound 27 |
| | R-isomer |
| | (Formula I-B |
| 20-1B | Compound 27 |
| | R-isomer |
| | (Formula I-B) |
| 20-2A | Compound 24 |
| | R-isomer |
| | (Formula I) |
| 20-2B | Compound 24 |
| | R-isomer |
| | (Formula I) |
| 20-2C | Compound 24 |
| | R-isomer |
| | (Formula I) |
| 20-2D | Compound 24 |
| | R-isomer |
| | (Formula I) |

Activated Pan T-cells were plated in 96 well plates at about 1×10⁶ cells per well. Each composition was applied to a well (n=3) in an amount needed to provide a dose of 3 μg eGFP mRNA. The cells were incubated at 37° C. for 10 minutes and then transferred into culture media. 24 hours later, expression of eGFP protein was measured by flow cytometry and the cells were stained with propidium iodide to evaluate toxicity.

For comparison, cells were also transfected with eGFP mRNA by electroporation (1350V, for 10 ms, and 3 pulses).

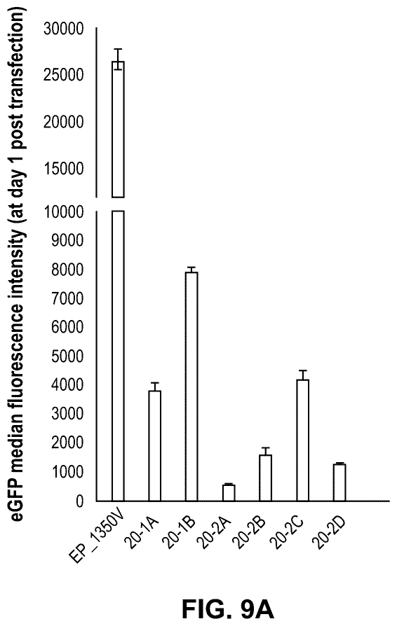

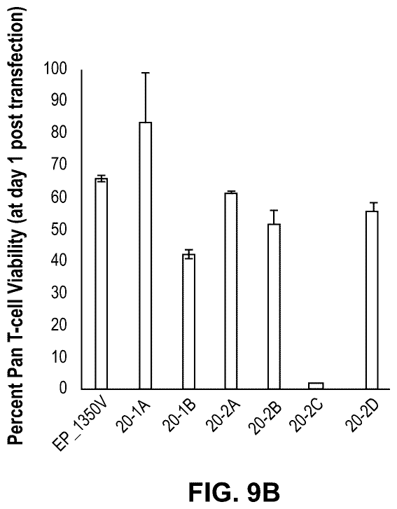

Results are shown in FIGS. 9A-9B. The lipid nanoparticle compositions identified as Composition ID Nos. 20-1A and 20-1B that comprised ionizable lipid of Formula I-B provided higher transfection efficiency in primary T cells than lipid nanoparticles containing ionizable lipid of Formula I, Composition ID Nos. 20-2B and 20-2C, respectively, where the difference in ionizable lipids was the only difference in components between Composition ID Nos. 20-1(A, B), and Composition ID Nos. 20-2 (B, C). This improvement was observed for two different lipid nanoparticle compositions, each containing a different helper lipid, i.e., Composition ID No. 20-1A and Composition ID No. 20-2B contained one helper lipid, and Composition ID No. 20-1B and Composition ID No. 20-2C contained a different helper lipid. Similarly, the lipid nanoparticles containing ionizable lipid of Formula I-B (Composition ID No. 20-1A and Composition ID No. 20-1B) provided higher viability in primary T cells than lipid nanoparticles containing ionizable lipid of Formula I (Composition ID No. 20-1B and Composition ID No. 20-2C, respectively).

Thus, Examples 19 and 20 show that for four different lipid nanoparticle compositions containing four different helper lipids, the ionizable lipid of Formula I-B provided higher transfection efficiency and higher viability, i.e., lower toxicity, than the ionizable lipid of Formula I. These findings indicate that the “tail” structure of the ionizable lipid of Formula I-B provides higher transfection efficiency and lower toxicity.

Composition ID No. 20-1A, Composition ID No. 20-1B, Composition ID Nos. 20-2B, and Composition ID No. 20-2C in Example 20 comprise the same ionizable lipids as Composition ID Nos. #29, #30, #32, and #33 in Example 18, respectively. Therefore, the improvement in transfection efficiency provided by the ionizable lipid of Formula I-B was observed across different lipid nanoparticle compositions in two different cell types, both floating and adherent.

### Example 21: Transfection with Lipid Nanoparticle Compositions

Lipid nanoparticle compositions were prepared for transfection of enhanced green fluorescent protein (eGFP) mRNA. The ionizable lipid structure in each composition is set forth in Table 21-1. All lipid nanoparticle compositions comprised eGFP mRNA, the indicated ionizable lipid (50 wt %), a helper lipid (DOPC or DOPE), a stabilizing lipid (e.g., DMG-PEG), and a structural lipid (cholesterol).

TABLE 21-1

| Composition | |
|---|---|
| ID No. | Ionizable Lipid and Structure |
| 21-1A 21-1B | 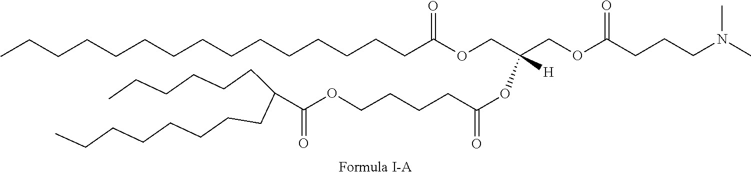 [SMILES: CCCCCCCCCCCCCCCC(=O)OC[C@H](COC(=O)CCCN(C)C)OC(=O)CCCCOC(=O)C(CCCCCC)CCCCCCCC] |
| 21-2A 21-2B | 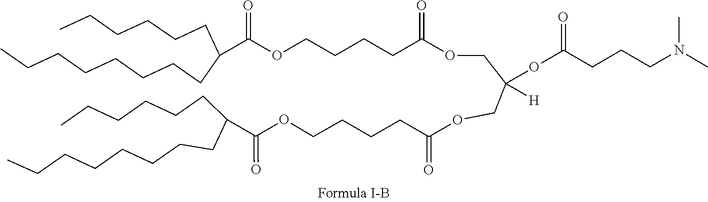 [SMILES: CCCCCCCCC(CCCCCC)C(=O)OCCCCC(=O)OCC(COC(=O)CCCCOC(=O)C(CCCCCC)CCCCCCCC)OC(=O)CCCN(C)C] |
| 21-3A 21-3B | 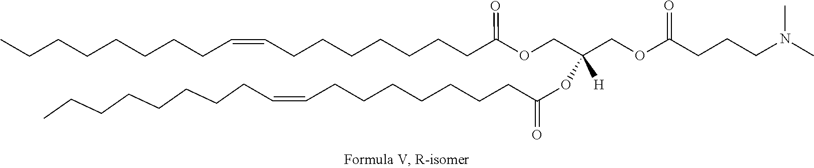 [SMILES: CCCCCCCC/C=C\CCCCCCCC(=O)OC[C@H](COC(=O)CCCN(C)C)OC(=O)CCCCCCC/C=C\CCCCCCCC] |
| 21-4A 21-4B | 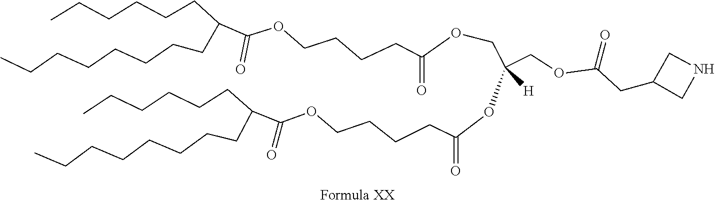 [SMILES: CCCCCCCCC(CCCCCC)C(=O)OCCCCC(=O)OC[C@H](COC(=O)CC1CNC1)OC(=O)CCCCOC(=O)C(CCCCCC)CCCCCCCC] |
| 21-5A 21-5B | 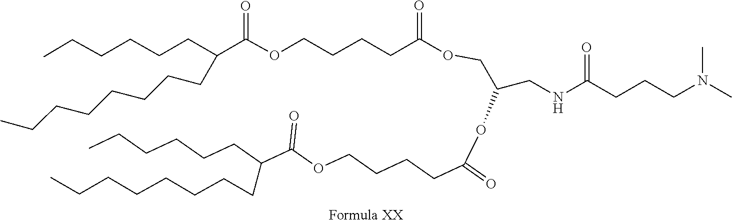 |
| 21-6A 21-6B | 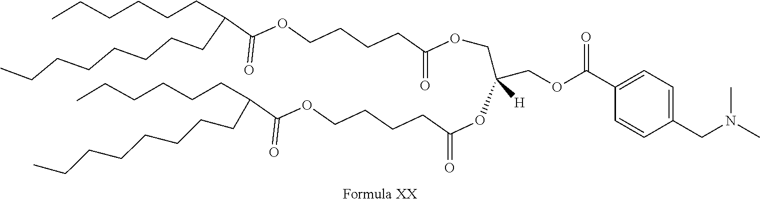 [SMILES: CCCCCCCCC(CCCCCC)C(=O)OCCCCC(=O)OC[C@H](COC(=O)c1ccc(CN(C)C)cc1)OC(=O)CCCCOC(=O)C(CCCCCC)CCCCCCCC] |
| 21-7A 21-7B | 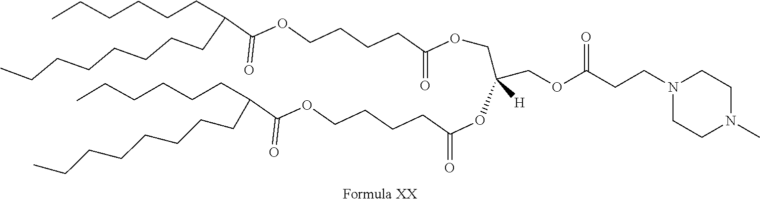 [SMILES: CCCCCCCCC(CCCCCC)C(=O)OCCCCC(=O)OC[C@H](COC(=O)CCN1CCN(C)CC1)OC(=O)CCCCOC(=O)C(CCCCCC)CCCCCCCC] |
| 21-8A 21-8B | 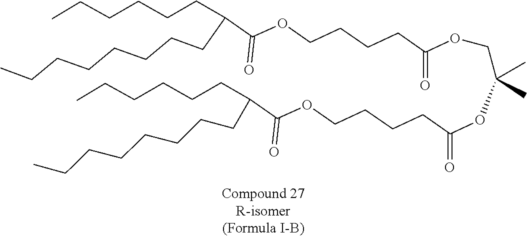 |

Activated Pan T-cells were plated in 96 well plates at about 1×10⁶ cells per well. Each composition was applied to a well (n=2) in an amount needed to provide a dose of 3 μg eGFP mRNA. The cells were incubated at 25° C. for 1 minute and then transferred into culture media. 18-24 hours later, expression of eGFP protein was measured by flow cytometry and the cells were stained with propidium iodide to evaluate toxicity.

For comparison, cells were also transfected with eGFP mRNA by electroporation (1350V, for 10 ms, and 3 pulses).

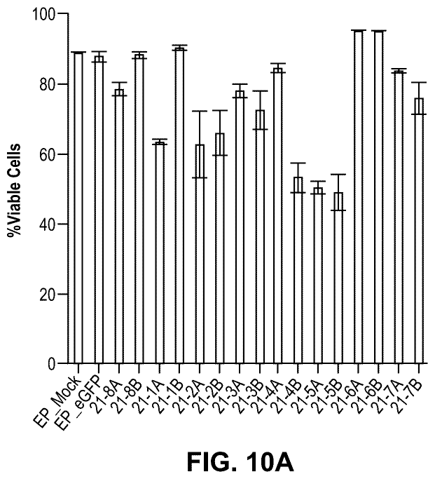

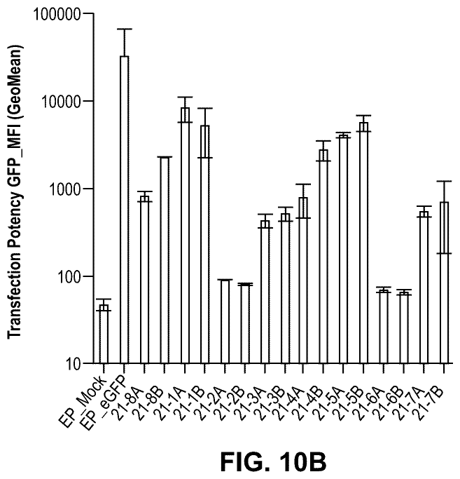

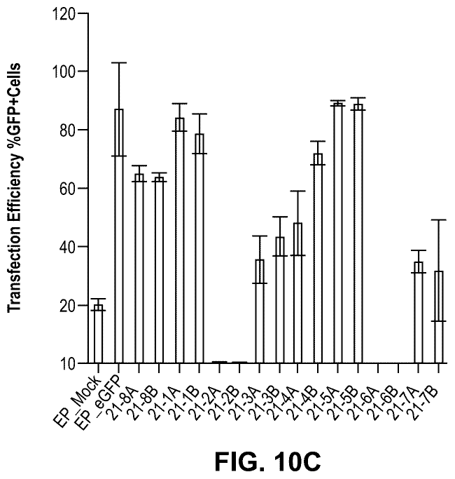

Results are shown in FIGS. 10A-10C, where cell viability is shown in FIG. 10A, transfection potency is shown in FIG. 10B, and transfection efficiency is shown in FIG. 10C.

Examples 22-38: LCMS Methods and Abbreviations: The following methods and abbreviations are referred to in Examples 22-38.
Method A: Chromolith, RP-18e, 50-4.6 mm, mobile phase A: 0.05% TFA in water, mobile phase B: 0.05% TFA in acetonitrile, 95A/5B-0A/100B over 9.5 min, hold 9.5-10.5 min, 95A/5B 10.5-12 min.
Method B: Chromolith, RP-18e, 50-4.6 mm, mobile phase A: 0.05% TFA in water, mobile phase B: 0.05% TFA in acetonitrile, 95A/5B-0A/100B over 8 min, hold 8-9 min, 95A/5B 9-9.1 min, hold 9.1-12 min.
Method C: Chromolith, RP-18e, 50-4.6 mm, mobile phase A: 0.05% TFA in water, mobile phase B: 0.05% TFA in acetonitrile, 50A/50B-0A/100B over 8 min, hold 8-9 min, 95A/5B 9-9.1 min, hold 9.1-12 min.
Method D: Chromolith, RP-18e, 50-4.6 mm, mobile phase A: 0.1% TFA in water, mobile phase B: 0.1% TFA in acetonitrile, 50A/50B-0A/100B over 3 min, hold 3-9 min, 50A/50B 9-9.1 min, hold 9.1-12 min.

ABBREVIATIONS: N,N′-dicyclohexylcarbodiimide (DCC); Dichloromethane (DCM); 4-(Dimethylamino)pyridine (4-DAMP); 1-(3-Dimethylaminopropyl)-3-ethylcarbodiimide (EDCI); Triethylamine (TEA); and 2,3-Dichloro-5,6-dicyano-1,4-benzoquinone (DDQ).

### Example 22: Synthesis of Compounds

This example demonstrates synthesis of lipid compounds:

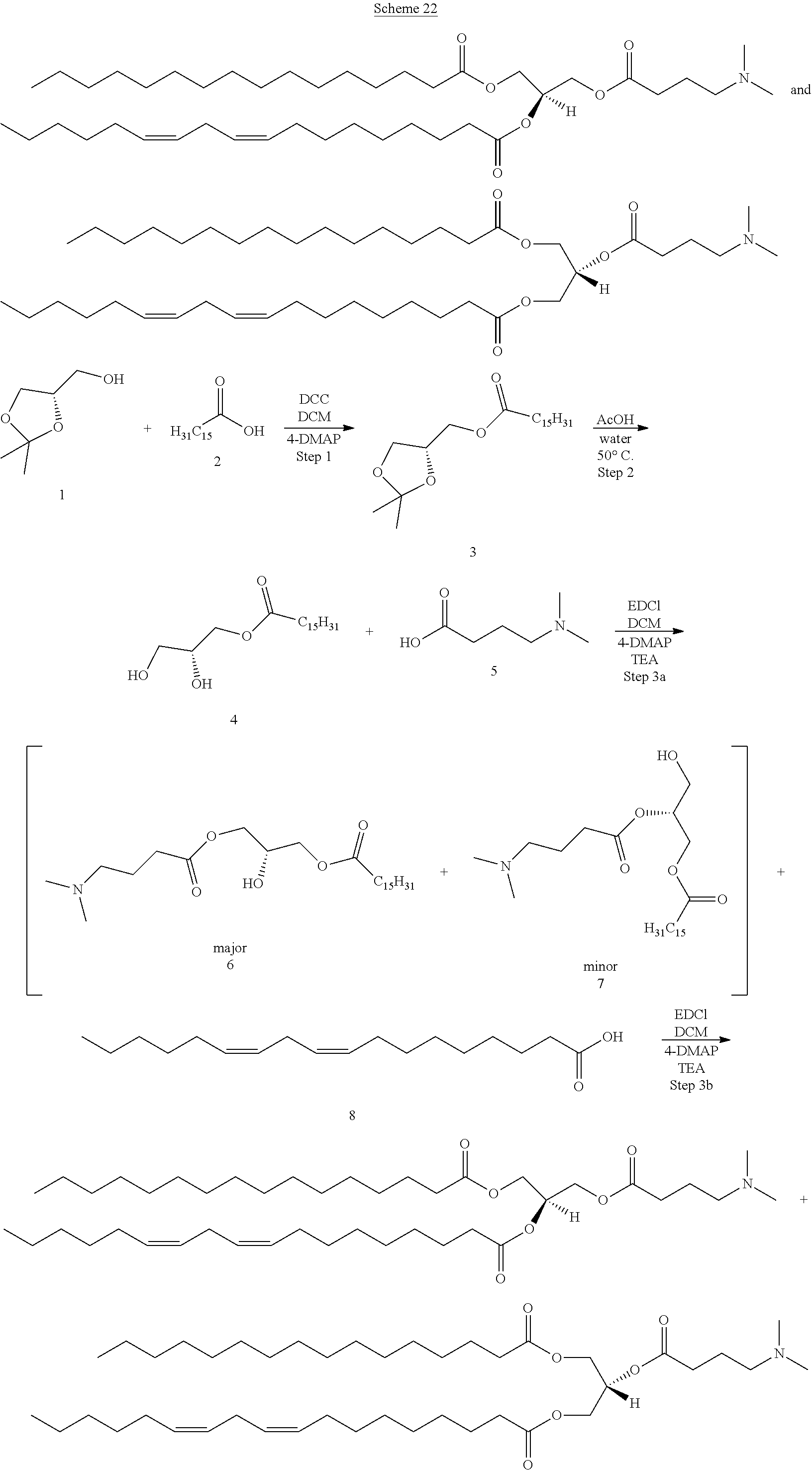

Step 1: To a solution of (S)-(+)-2,2-Dimethyl-1,3-dioxolane-4-methanol 1 (0.88 mL, 7.1 mmol, 1 eq) in anhydrous dichloromethane (25 mL) was added palmitic acid 2 (1.82 g, 7.1 mmol, 1 eq) and 4-DMAP (52 mg, 0.42 mmol, 0.06 eq). The resulting mixture was cooled to approximately 0° C. under ice/water bath. To this solution was added a mixture of N,N′-dicyclohexylcarbodiimide (2.92 g, 14.2 mmol, 2 eq) in anhydrous dichloromethane (20 mL) dropwise over 25 min. The reaction mixture was warmed up to temperature and stirred for an additional 72 h. Reaction mixture was diluted with hexanes (50 mL) and the resulting white precipitates were filtered off and washed with hexanes (2×25 mL). Filtrate was concentrated in vacuo and purified by flash column chromatography on silica gel using 0-10% ethyl acetate in hexanes over 30 min to obtain pure intermediate 3 (2.48 g, 94% yield). LCMS (M+1)=371.88, Rt=9.86 min. (method A)

Step 2: A solution of Intermediate 3 (1 g, 2.7 mmol) in acetic acid (4 mL) and water (1 mL) was stirred at 50° C. for 2 h and then at 30° C. for 48 h. Reaction mixture was then partitioned between water (100 mL) and diethyl ether (100 mL). Organic layer was separated and washed with saturated aqueous sodium bicarbonate solution (2×100 mL) and brine (50 mL). Organic layer was dried under anhydrous magnesium sulfate, filtered and concentrated in vacuo to provide crude product which was crystallized in hexanes and ethyl acetate to provide intermediate 4 (0.89 g, 79% yield). LCMS (M+Na)=353.51, Rt=5.26 min. (method B).

Step 3a: To a solution of intermediate 4 (202 mg, 0.61 mmol, 1 eq) in dichloromethane (15 mL) was added 4-dimethylaminobutyric acid hydrochloride salt (92 mg, 0.55 mmol, 0.9 eq), EDCI·HCl (140 mg, 0.73 mmol, 1.2 eq), triethylamine (0.255 mL, 1.83 mmol, 3 eq) and 4-DMAP (7 mg, 0.06 mmol, 0.1 eq). The reaction mixture was stirred at room temperature for 1.5 h.

Step 3b: Linoleic acid 8 (0.379 mL, 1.22 mmol, 2 eq), EDCI·HCl (0.175 g, 0.91 mmol, 1.5 eq), triethylamine (0.27 mL, 1.9 mmol, 3 eq) and 4-DMAP (7 mg, 0.06 mmol, 0.1 eq) was added and the resulting mixture was stirred at room temperature for 16 h. Solvent was reduced in vacuo and the crude mixture was directly purified on silica gel flash chromatography using 0-10% methanol in dichloromethane to obtain the first lipid compound shown above (37 mg, 8.6% yield over two steps). LCMS (M+1)=706.27, Rt=8.79 min (method C) and the second lipid compound shown above (17 mg, 4% yield over two steps). LCMS (M+1)=706.26, Rt=8.71 min (method C).

### Example 23: Synthesis of Compounds

This example demonstrates synthesis of lipid compounds:

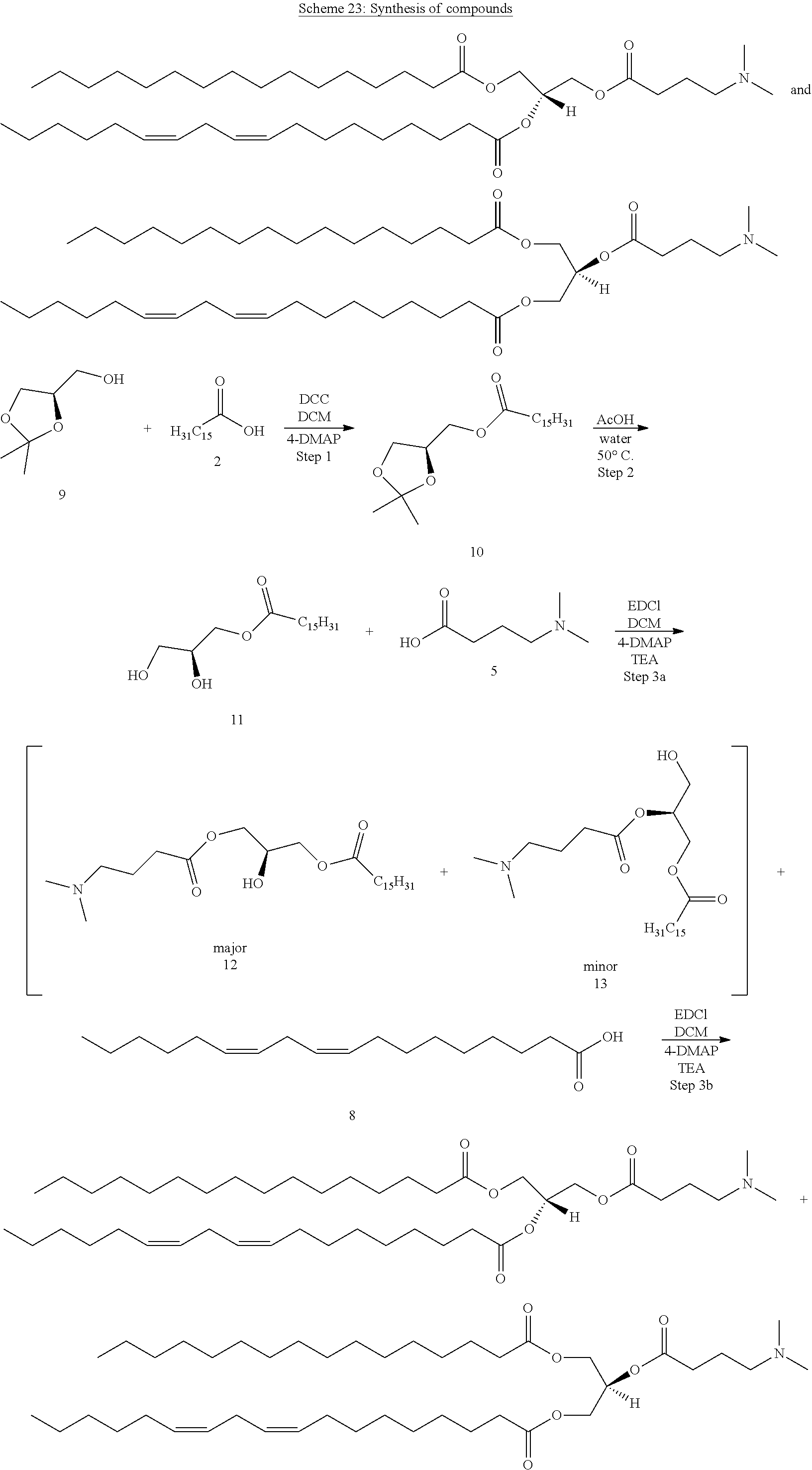

Step 1: To a solution of (R)-(+)-2,2-Dimethyl-1,3-dioxolane-4-methanol 9 (0.94 mL, 7.6 mmol, 1 eq) in anhydrous dichloromethane (20 mL) was added palmitic acid 2 (1.94 g, 7.6 mmol, 1 eq) and 4-DMAP (55 mg, 0.45 mmol, 0.06 eq). The resulting mixture was cooled to approximately 0° C. under ice/water bath. To this solution was added a mixture of N,N′-dicyclohexylcarbodiimide (3.12 g, 15.1 mmol, 2 eq) in anhydrous dichloromethane (35 mL) dropwise over 30 min. The reaction mixture was warmed up to temperature and stirred for an additional 12 h. Reaction mixture was diluted with hexanes (60 mL) and the resulting white precipitates were filtered off and washed with hexanes (2×30 mL). Filtrate was concentrated in vacuo and purified by flash column chromatography on silica gel using 0-10% ethyl acetate in hexanes over 30 min to obtain pure intermediate 10 (2.1 g, 80% yield).

Step 2: A solution of Intermediate 10 (1.5 g, 4 mmol) in acetic acid (12 mL) and water (3 mL) was stirred at 50° C. for 2 h and then at room temperature for 16 h. Reaction mixture was then partitioned between water (50 mL) and diethyl ether (150 mL). Organic layer was separated and the aqueous layer was extracted back with diethyl ether (50 mL). Combined organic layer was washed with saturated aqueous sodium bicarbonate solution (2×60 mL) and brine (50 mL). Organic layer was dried under anhydrous sodium sulfate, filtered and concentrated in vacuo to provide crude product which was crystallized in hexanes and ethyl acetate to provide intermediate 11 (1.28 g, 97% yield).

Step 3a: To a solution of intermediate 11 (500 mg, 1.5 mmol, 1 eq) in dichloromethane (70 mL) was added 4-dimethylaminobutyric acid hydrochloride salt (228 mg, 1.36 mmol, 0.9 eq), EDCI·HCl (320 mg, 1.66 mmol, 1.1 eq), triethylamine (0.675 mL, 4.84 mmol, 3.2 eq) and 4-DMAP (18 mg, 0.15 mmol, 0.1 eq). The reaction mixture was stirred at room temperature for 1.5 h.

Step 3b: Linoleic acid 8 (0.940 mL, 3.02 mmol, 2 eq), EDCI·HCl (0.638 g, 3.32 mmol, 2.2 eq), triethylamine (0.740 mL, 5.3 mmol, 3.5 eq) and 4-DMAP (18 mg, 0.15 mmol, 0.1 eq) were added and the resulting mixture was stirred at room temperature for 16 h. Solvent was reduced in vacuo and the crude mixture was directly purified on silica gel flash chromatography using 0-10% methanol in a 75:25 mixture of dichloromethane:ethylacetate to obtain the first compound shown above (81 mg, 7.6% yield over two steps). LCMS (M+1)=706.18, Rt=9.16 min and the second compound shown above (12 mg, 1.1% yield over two steps). LCMS (M+1)=706.19, Rt=8.98 min (Method C).

### Example 24: Synthesis of Compounds

This example demonstrates synthesis of lipid compounds:

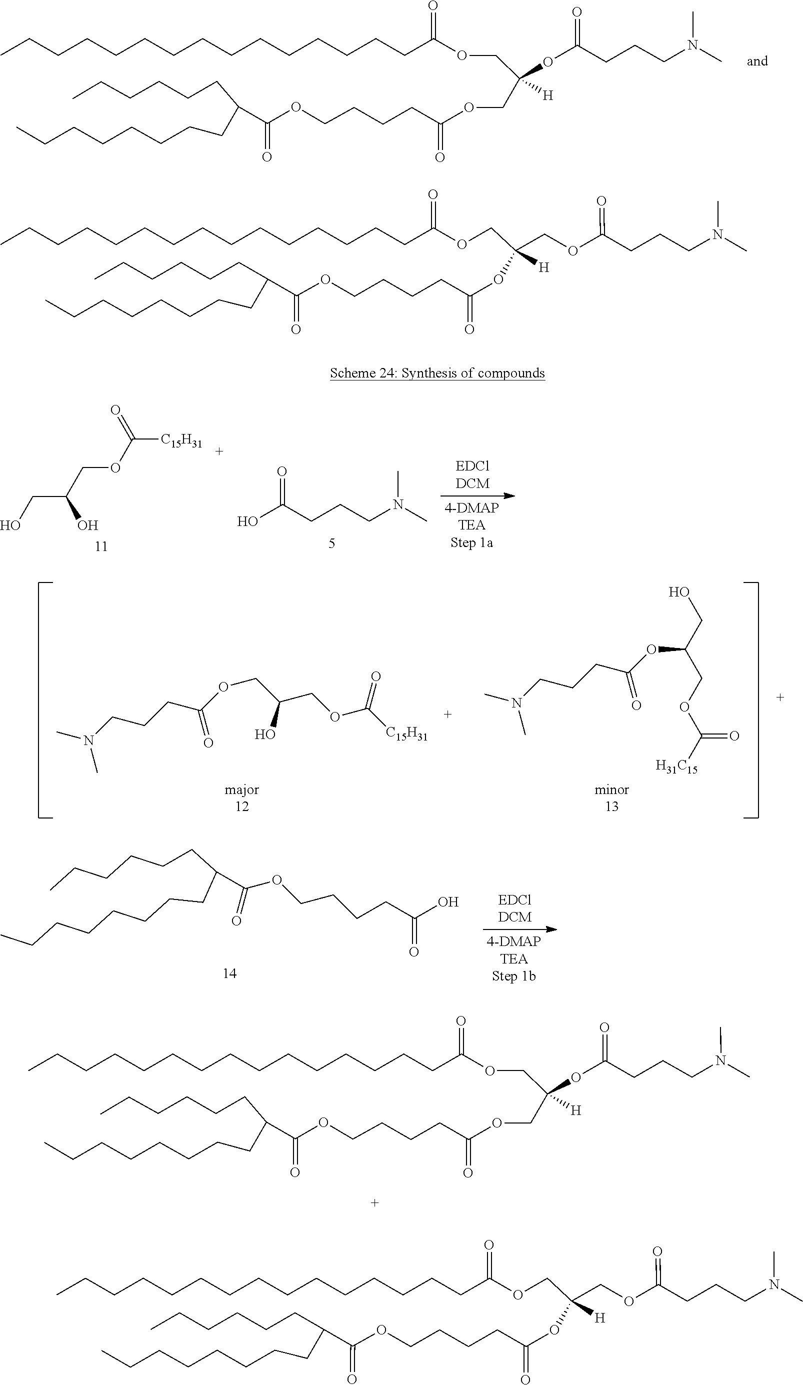

To a solution of intermediate 11 (300 mg, 0.9 mmol, 1 eq) in dichloromethane (15 mL) was added 4-dimethylaminobutyric acid hydrochloride salt (152 mg, 0.9 mmol, 1 eq), EDCI·HCl (208 mg, 1.1 mmol, 1.2 eq), triethylamine (0.443 mL, 3.17 mmol, 3.5 eq) and 4-DMAP (11 mg, 0.09 mmol, 0.1 eq). The reaction mixture was stirred at room temperature for 1.5 h.

A dichloromethane (5 mL) solution of carboxylic acid 14 (398 mg, 1.1 mmol, 1.23 eq), EDCI·HCl (0.226 g, 1.2 mmol, 1.3 eq) and triethylamine (0.380 mL, 2.7 mmol, 3 eq) was added and the resulting mixture was stirred at room temperature for 16 h. Solvent was reduced in vacuo and the crude mixture was directly purified on silica gel flash chromatography using 0-10% methanol in dichloromethane to obtain the two product compounds shown above as an approximate 45:55 mixture (29 mg, 4% yield over two steps). LCMS (M+1)=782.61, Rt=9.07 and 9.19 min respectively (method C).

### Example 25: Synthesis of Compound

This example demonstrates synthesis of lipid compound:

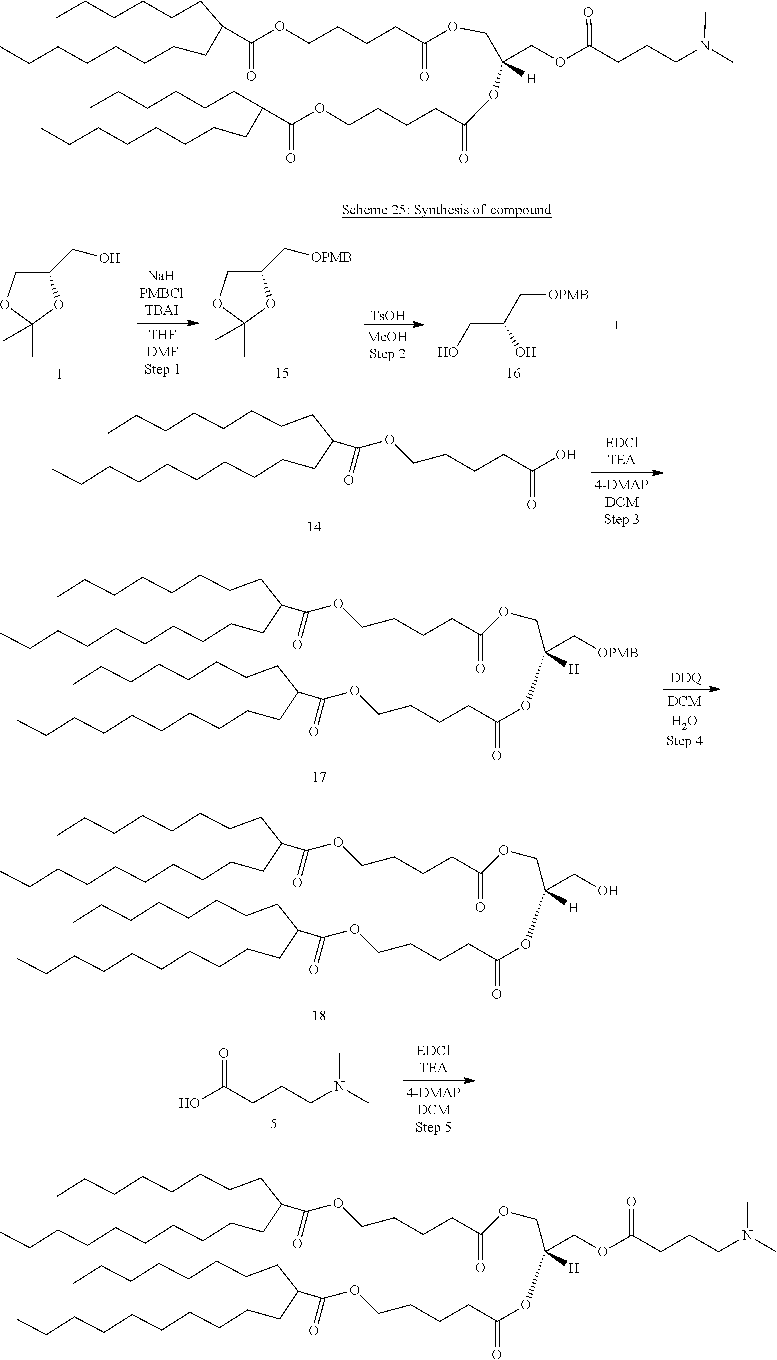

Step 1: In a dry 100 mL round bottom flask was added NaH, 60% dispersed in oil (225 mg, 5.6 mmol, 1.25 eq) under nitrogen atmosphere. Anhydrous THF (18 mL) and anhydrous DMF (5 mL) were sequentially added slowly via syringe. After 10 min, (S)-(+)-2,2-Dimethyl-1,3-dioxolane-4-methanol 1 (0.5 mL, 4.5 mmol, 1 eq) was added slowly via syringe and the resulting reaction mixture was stirred at room temperature for 1 h. 4-Methoxybenzyl chloride (0.67 mL, 5 mmol, 1.1 eq) and tetrabutylammonium iodide (59 mg, 0.16 mmol, 0.036 eq) were added and the reaction mixture was further stirred for 12 h. MeOH (2 mL) was added. The reaction mixture was then washed with saturated aqueous ammonium chloride (25 mL), water (25 mL) and brine (25 mL). Aqueous layers were combined and back extracted with DCM (2×50 mL). Organic layers were combined and dried over anhydrous sodium sulfate, filtered and concentrated in vacuo to provide crude product which was purified on silica gel flash chromatography using 0-10% ethyl acetate in hexanes to provide intermediate 15 (980 mg, 96% yield).

Step 2: To a solution of intermediate 15 (1 g, 4 mmol, 1 eq) in methanol (5 mL) was added p-toluenesulfonic acid (38 mg, 0.2 mmol, 0.05 eq) and resulting reaction mixture was stirred at room temperature for 72 h. Triethylamine (0.046 mL) was added. A slurry of the reaction mixture was prepared on silica gel and directly purified on silica gel flash chromatography using 0-100% ethyl acetate in hexanes to obtain intermediate 16 (390 mg, 46% yield).

Step 3: To a solution of diol 16 (150 mg, 0.71 mmol, 1 eq) in dichloromethane (6. mL) was added acid 14 (579 mg, 1.6 mmol, 2.3 eq), EDCI hydrochloride (338 mg, 1.8 mmol, 2.5 eq), triethylamine (0.690 mL, 4.9 mmol, 7 eq) and 4-DMAP (17 mg, 0.14 mmol, 0.2 eq) at room temperature and the resulting reaction mixture was stirred for 48 h. Reaction mixture was concentrated in vacuo and purified by silica gel flash chromatography using 0-25% ethyl acetate in hexanes to obtain pure intermediate 17 as clear light yellow syrup (232 mg, 37% yield).

Step 4: Intermediate 17 (190 mg, 0.21 mmol, 1 eq) was added to a mixture of dichloromethane (9 mL) and water (0.565 mL). DDQ (122 mg, 0.53 mmol, 2.5 eq) was slowly added in portions and the resulting reaction mixture was stirred over night at room temperature. Reaction mixture was quenched by the addition of saturated aqueous sodium bicarbonate solution (15 mL). Aqueous layer was extracted with dichloromethane (4×25 mL). Combined organic layer was washed with saturated aqueous sodium bicarbonate solution (25 mL), brine (2×25 mL), dried over anhydrous sodium sulfate, filtered and concentrated at reduced pressure to obtain crude product which was purified by flash silica gel chromatography using 0-30% ethyl acetate in hexanes. Pure fractions were concentrated to dryness to obtain desired product alcohol 18 as light-yellow clear oil (160 mg, quant.).

Step 5: To a solution of intermediate alcohol 18 (145 mg, 0.19 mmol, 1 eq) in dichloromethane (3.5 mL) was added 4-dimethylaminobutyric acid hydrochloride salt (64 mg, 0.38 mmol, 2 eq), EDCI hydrochloride (92 mg, 0.47 mmol, 2.5 eq), triethylamine (0.250 mL, 1.8 mmol, 9.4 eq), 4-DMAP (10 mg, 0.08 mmol, 0.4 eq) and resulting reaction mixture was stirred for 16 h at room temperature. LCMS indicated the formation of desired product. Reaction mixture was directly loaded on silica gel column and purified using 0-10% methanol in dichloromethane. Pure fractions were combined, concentrated in rotovap and dried overnight under high vacuum to obtain desired product (140 mg, 84% yield). LCMS (M+1)=882.68, Rt=8.89 min (method C). ¹H NMR: (500 MHz, Chloroform-d) δ 5.26 (ddd, J=10.1, 5.9, 4.2 Hz, 1H), 4.32 (dd, J=4.4, 2.2 Hz, 1H), 4.29 (dd, J=4.4, 2.2 Hz, 1H), 4.14 (ddd, J=12.0, 5.9, 1.8 Hz, 2H), 4.08 (dt, J=6.3, 3.1, 3.1 Hz, 4H), 2.36 (td, J=7.2, 7.1, 2.8 Hz, 6H), 2.30 (td, J=8.7, 8.5, 4.4 Hz, 4H), 2.22 (s, 6H), 1.79 (p, J=7.3, 7.3, 7.3, 7.3 Hz, 2H), 1.69 (dh, J=12.3, 3.6, 3.6, 3.2, 3.2, 3.2 Hz, 8H), 1.58 (h, J=7.4, 7.4, 7.4, 5.1, 5.1 Hz, 4H), 1.43 (p, J=7.1, 7.1, 5.6, 5.6 Hz, 4H), 1.25 (s, 40H), 0.92-0.83 (m, 12H).

### Example 26: Synthesis of Compound

This example demonstrates synthesis of lipid compound:

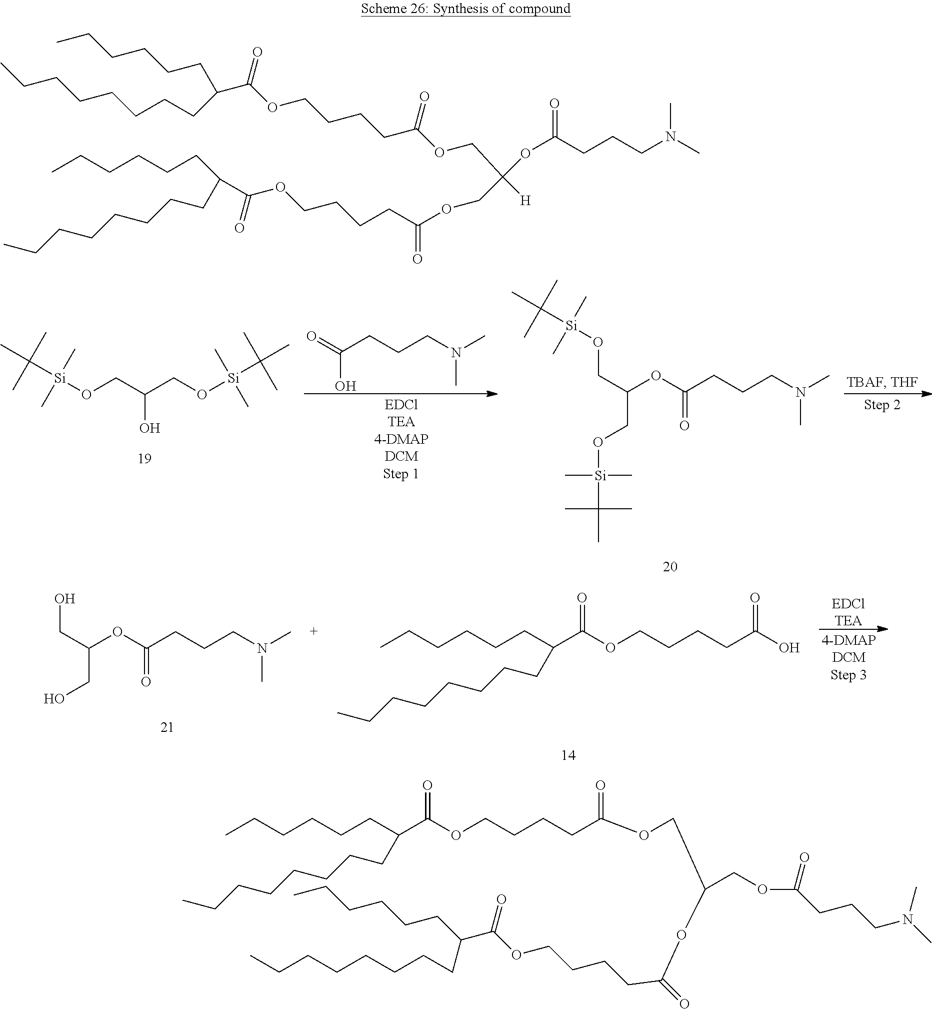

Step 1: To a solution of compound 19 (500 mg, 1.56 mmol, 1 eq) in dichloromethane (15. mL) was added 4-(dimethylamino)butyric acid (392 mg, 2.34 mmol, 1.5 eq), EDCI hydrochloride (750 mg, 3.9 mmol, 2.5 eq), triethylamine (1.2 mL, 7.8 mmol, 5 eq) and 4-DMAP (96 mg, 0.78 mmol, 0.5 eq) at room temperature and the resulting reaction mixture was stirred for 48 h. Reaction mixture was concentrated in vacuo and purified by silica gel flash chromatography using 0-25% ethyl acetate in hexanes to obtain pure intermediate 20 as solid (168 mg, 25% yield).

Step 2: To a solution of intermediate 20 (150 mg, 0.4 mmol, 1 eq) in THF (2 mL) was added TBAF (2 mL, 4.67 mmol, 10 eq) and the resulting reaction mixture was stirred at room temperature for 3 h. A slurry of the reaction mixture was prepared on silica gel and directly purified on silica gel flash chromatography using 0-10% Methanol in DCM to obtain intermediate 21 (51 mg, 54% yield).

Step 3: To a solution of diol intermediate 21 (51 mg, 0.25 mmol, 1 eq) in dichloromethane (3 mL) was added acid 14 (200 mg, 0.55 mmol, 2.1 eq), EDCI hydrochloride (120 mg, 0.625 mmol, 2.5 eq), triethylamine (0.2 mL, 1.25 mmol, 5 eq) and 4-DMAP (6 mg, 0.05 mmol, 0.2 eq) at room temperature and the resulting reaction mixture was stirred for 48 h. Reaction mixture was concentrated in vacuo and purified by silica gel flash chromatography using 0-40% acetone in hexanes to obtain pure desired product compound (92 mg, 45% yield, [M+1] 883.36.

### Example 27: Synthesis of Compound

This example demonstrates synthesis of lipid compound:

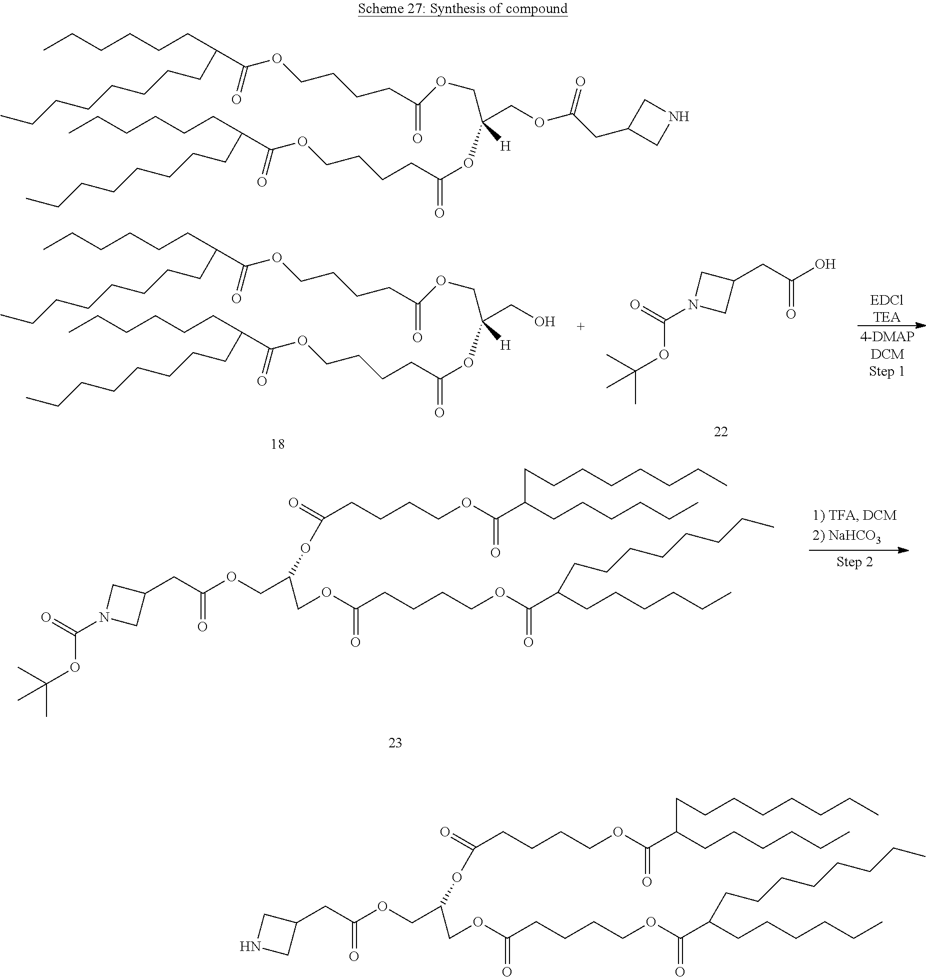

Step 1: To a solution of alcohol 18 (137 mg, 0.18 mmol, 1 eq) in dichloromethane (5 mL) was added 2 (1-tert-butoxycarbonyl-3-azetidinyl)acetic acid 22 (57 mg, 0.26 mmol, 1.5 eq), EDCI hydrochloride (57 mg, 0.29 mmol, 1.7 eq), triethylamine (0.2 mL, 0.89 mmol, 5 eq), 4-DMAP (11 mg, 0.08 mmol, 0.5 eq) and the resulting reaction mixture was stirred for 16 h at room temperature.

LCMS indicated the formation of desired product. Reaction mixture was directly loaded on silica gel column and purified using 0-40% acetone in hexane. Pure fractions were combined, concentrated in vacuo and dried overnight under high vacuum to obtain desired product 23 (132 mg, 98% yield).

Step 2: To a solution of compound 23 (157 mg, 0.16 mmol, 1 eq) in dichloromethane (5 mL) was added TFA (1 ml). The reaction was stirred for 1 hour, saturated aqueous sodium bicarbonate (5 mL) was added and the resulting mixture was extracted with dichloromethane (2×5 mL), combined organic layer was washed with brine, dried over anhydrous sodium sulfate, filtered and concentrated in vacuo and dried overnight under high vacuum to obtain desired product (32 mg, 20% yield). LCMS (M+1)=867.8, Rt=4.96 min (method D).

### Example 28: Synthesis of Compound

This example demonstrates synthesis of lipid compound:

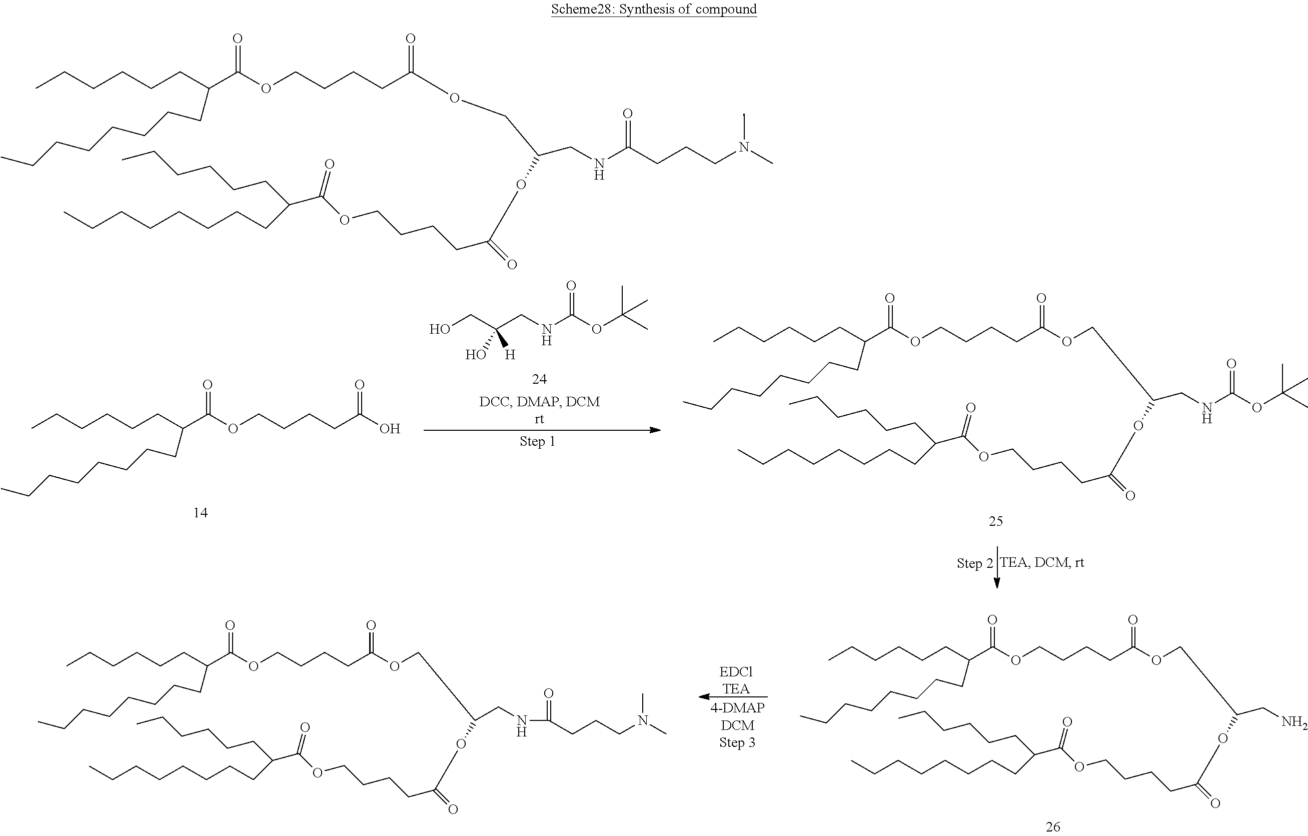

Step 1: To a solution of (S)-2,3-Dihydroxypropylamino-tert-butylformylate 24 (500 mg, 1.57 mmol, 1 eq) in dichloromethane (25 mL) was added acid 14 (1.17 g, 3.29 mmol, 2.1 eq), EDCI hydrochloride (662 mg, 3.45 mmol, 2.2 eq), triethylamine (2.3 ml, 15.7 mmol, 10 eq) and 4-DMAP (20 mg, 0.16 mmol, 0.1 eq). The resulting reaction mixture was stirred for 16 h at room temperature. LCMS indicated the formation of desired product. Reaction mixture was directly loaded on silica gel column and purified using 0-20% acetone in hexanes. Pure fractions were combined, concentrated in vacuo and dried overnight under high vacuum to obtain colorless liquid compound 25 (681 mg, 49% yield).

Step 2: To a solution of compound 25 (200 mg, 0.23 mmol, 1 eq) in dichloromethane (5 mL) was added 1.5 ml of TFA. The resulting reaction mixture was stirred for 2 h at room temperature and the reaction was monitored by the LCMS. The reaction mixture was concentrated in vacuo and dried under high vacuum to obtain colorless liquid. The reaction mixture (compound 26) was taken to the next step without purification.

Step 3: To a solution of compound 26 (180 mg, 0.23 mmol, 1 eq) in dichloromethane (4 mL) was added 4-(dimethylamino)butyric acid (79 mg, 0.47 mmol, 2 eq), EDCI hydrochloride (113 mg, 0.58 mmol, 2.5 eq), triethylamine (170 ul, 1.17 mmol, 5 eq) and 4-DMAP (15 mg, 0.12 mmol, 0.5 eq). Resulting reaction mixture was stirred for 16 h at room temperature. LCMS indicated the formation of desired product. Dichloromethane 5 mL was added and the resulting mixture was washed with water. Organic layer was separated, dried over anhydrous sodium sulfate and concentrated to about ⅓ of the total volume and directly loaded on silica gel column and purified using 0-20% acetone in hexane. Pure fractions were combined, concentrated in vacuo and dried overnight under high vacuum to obtain desired product as colorless liquid (115 mg, 56% yield). LCMS (M+1)=882.39, Rt=4.81 min (Method D).

### Example 29: Synthesis of Compound

This example demonstrates synthesis of lipid compound:

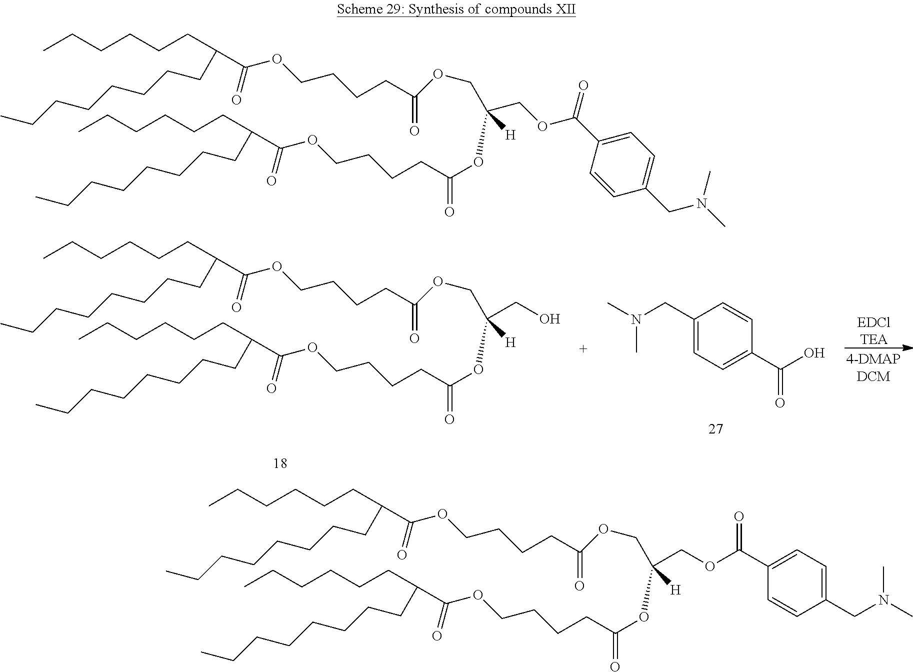

To a solution of intermediate alcohol 18 (50 mg, 0.065 mmol, 1 eq) in dichloromethane (3 mL) was added p-[(dimethylamino)methyl]benzoic acid 27 (18 mg, 0.097 mmol, 1.5 eq), EDCI hydrochloride (20 mg, 0.097 mmol, 1.5 eq), triethylamine (20 uL, 0.13 mmol, 2 eq), 4-DMAP (2 mg, 0.03 mmol, 0.5 eq) and resulting reaction mixture was stirred for 16 h at room temperature. LCMS indicated the formation of desired product. Reaction mixture was directly loaded on silica gel column and purified using 0-40% acetone in hexane. Pure fractions were combined, concentrated in vacuo and dried overnight under high vacuum to obtain desired product (46 mg, 76% yield) LCMS (M+1)=931.43, Rt=4.22 min (Method D).

### Example 30: Synthesis of Compound

This example demonstrates synthesis of lipid compound:

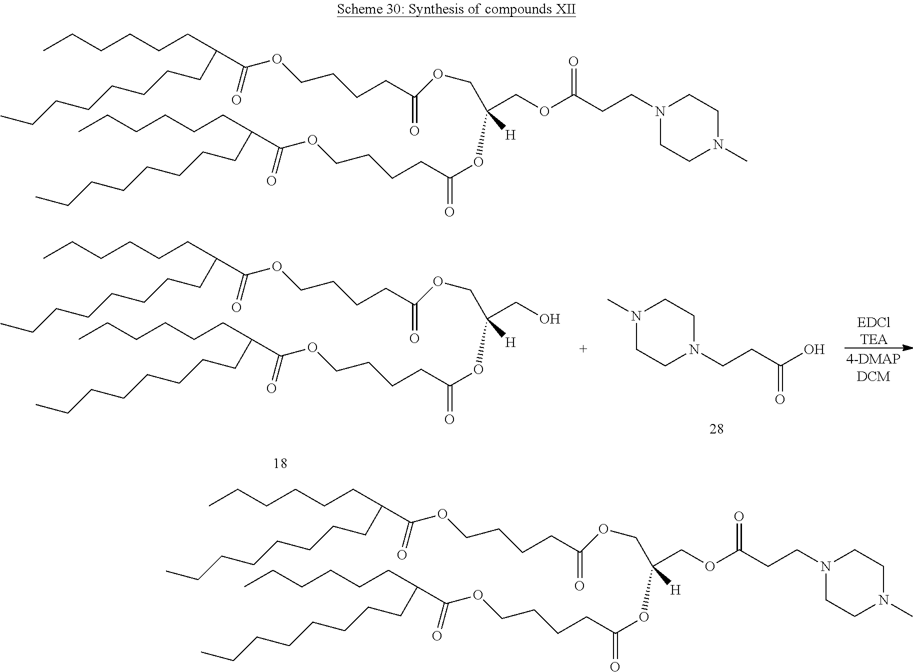

To a solution of intermediate alcohol 18 (50 mg, 0.065 mmol, 1 eq) in dichloromethane (3 mL) was added 3-(4-Methyl-1-piperazinyl)propionic acid 28 (17 mg, 0.097 mmol, 1.5 eq), EDCI hydrochloride (20 mg, 0.097 mmol, 1.5 eq), triethylamine (20 uL, 0.13 mmol, 2 eq), 4-DMAP (2 mg, 0.03 mmol, 0.5 eq) and resulting reaction mixture was stirred for 16 h at room temperature. LCMS indicated the formation of desired product. Reaction mixture was directly loaded on silica gel column and purified using 0-10% methanol in DCM. Pure fractions were combined, concentrated in vacuo and dried overnight under high vacuum to obtain desired product (27 mg, 45% yield) LCMS (M+1)=924.4, Rt=5.61 min (Method D).

### Example 31: Synthesis of Compound

This example demonstrates synthesis of lipid compound:

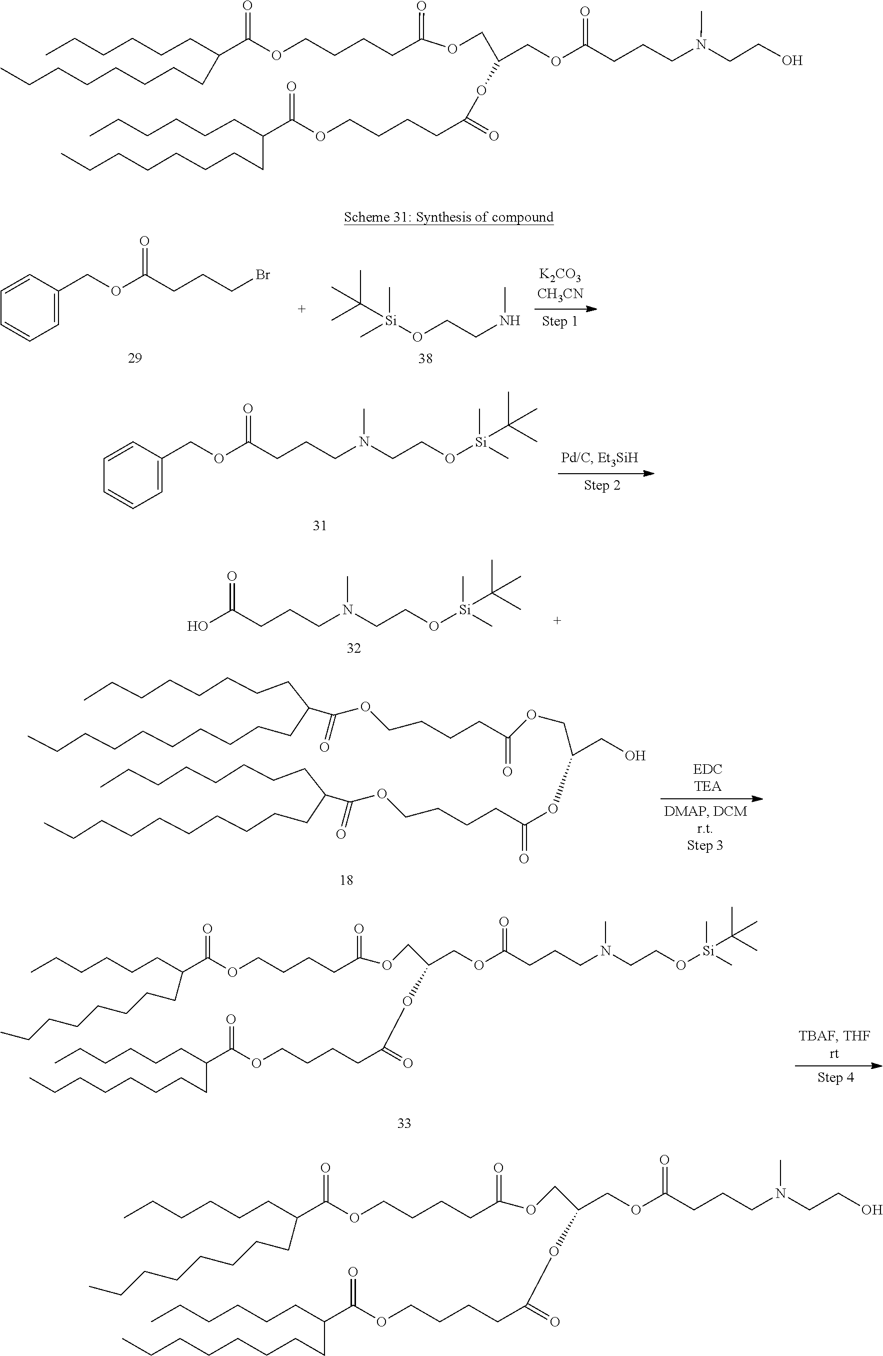

Step 1: To a solution of Benzyl 4-bromobutyrate 29 (2 g, 7.78 mmol, 1 eq) in acetonitrile (10 mL) was added N-methyl{2-[(tert-butyl)bis(methyl)siloxy]ethyl}amine 30 (1.8 g, 9.33 mmol, 1.2 eq). Potassium carbonate (2.2 g, 15.36, 2 eq) was added and the resulting reaction mixture was stirred for 16 h at room temperature. LCMS indicated the formation of desired product. A slurry of the reaction mixture was prepared on silica gel and directly purified by silica gel column chromatography using 0-30% ethyl acetate in hexanes. Pure fractions were combined, concentrated and dried overnight under high vacuum to obtain compound 31 (2.6 g, 91% yield) as white solid.

Step 2: A solution of compound 31 (780 mg, 2.14 mmol, 1 eq) in anhydrous methanol (20 mL) was flushed with argon. 10% Pd/C (30 mg, 0.214 mmol, 0.1 eq) was added and stirred for 20 min. Triethylsilane (1 ml, 6.42 mmol, 3 eq) was added in the resulting mixture. The reaction mixture was stirred for 2 h at room temperature. Reaction mixture was then filtered through a bed of celite and the filtrate was concentrated in vacuo, dried under high vacuum to obtain compound 32 as white solid (560 mg, 95% yield) which was taken to the next step without any further purification.

Step 3: To a solution of compound 18 (89 mg, 0.12 mmol, 1 eq) in dichloromethane (4 mL) was added compound 32 (40 mg, 0.14 mmol, 1.2 eq), EDC hydrochloride (56 mg, 0.29 mmol, 2.5 eq), triethylamine (167 ul, 1.17 mmol, 10 eq) and 4-DMAP (2 mg, 0.01 mmol, 0.1 eq). The resulting reaction mixture was stirred for 16 h at room temperature. LCMS indicated the formation of desired product. Water (10 mL) was added and extracted with dichloromethane (3×15 mL). Combined organic layer was washed with brine, dried over anhydrous sodium sulfate, filtered and concentrated to obtained crude product which was purified on silica gel column using 0-20% acetone in hexanes. Pure fractions were combined, concentrated in vacuo and dried overnight under high vacuum to obtain colorless liquid compound 33 (93 mg, 47% yield). LCMS (M+1)=1027.3, Rt=4.81 min (method D).

Step 4: To a solution of compound 33 (93 mg, 0.09 mmol, 1 eq) in THF (3 mL) was added TBAF (0.2 ml, 10 eq.) and the resulting reaction mixture was stirred for 2 h at room temperature. LCMS indicated the completion of reaction. Reaction mixture was directly loaded on silica gel column and purified using 0-40% acetone in hexanes to obtain desired product (57 mg, 69% yield). LCMS (M+1)=913.56, Rt=5.06 min (method D).

### Example 32: Synthesis of Compound

This example demonstrates synthesis of lipid compound:

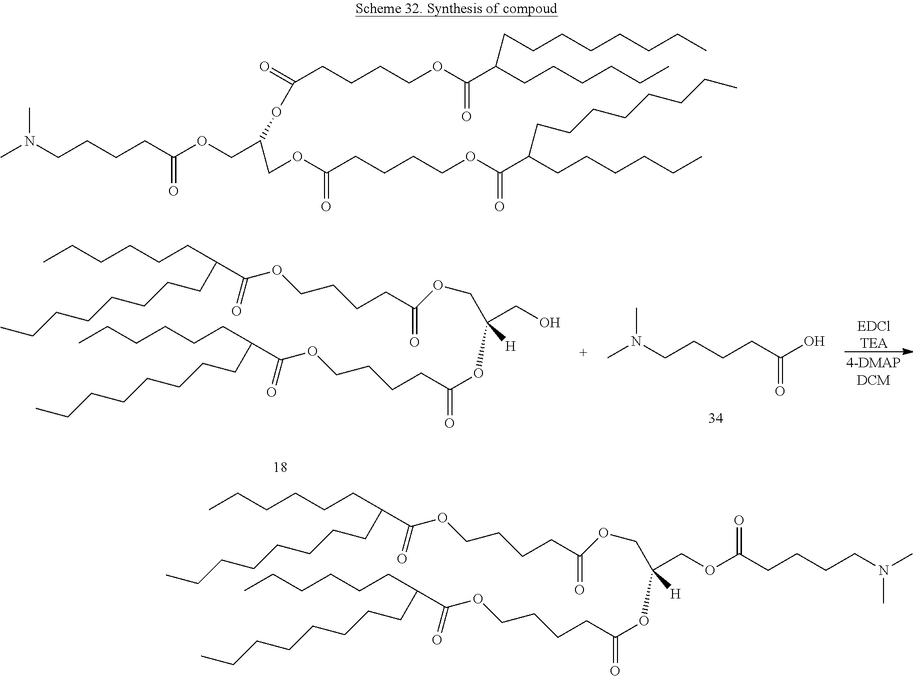

To a solution of intermediate alcohol 18 (102 mg, 0.133 mmol, 1 eq) in dichloromethane (4 mL) was added 5-(dimethylamino)valeric acid 34 (40 mg, 0.28 mmol, 2 eq), EDCI hydrochloride (64 mg, 0.33 mmol, 2.5 eq), triethylamine (96 uL, 0.66 mmol, 5 eq), 4-DMAP (10 mg, 0.06 mmol, 0.5 eq) and resulting reaction mixture was stirred for 16 h at room temperature. LCMS indicated the formation of desired product. Reaction mixture was directly loaded on silica gel column and purified using 0-60% acetone in hexane. Pure fractions were combined, concentrated in rotovap and dried overnight under high vacuum to obtain desired product (56 mg, 47% yield). LCMS (M+1)=897.34.8, Rt=4.99 min (method D).

### Example 33: Synthesis of Compound

This example demonstrates synthesis of lipid compound:

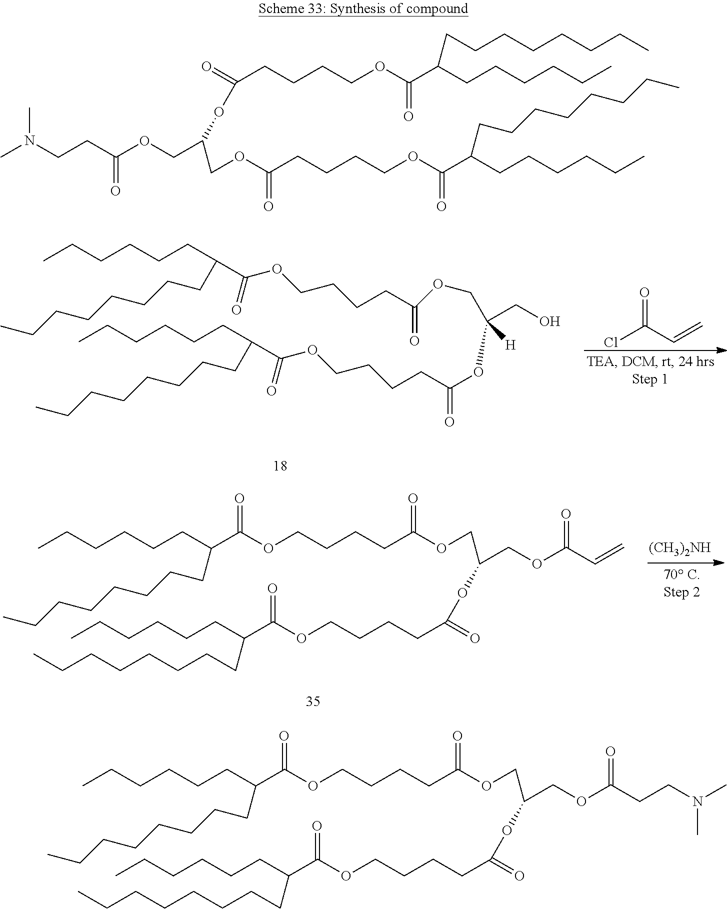

Step 1: In a dry 100 mL round bottom flask was added compound 18 (1.1 g, 1.41 mmol, 1 eq) under argon atmosphere. Anhydrous dichloromethane (18 mL) was added slowly via syringe. After 10 min, triethylamine (0.2 mL, 4.23 mmol, 3 eq) was added slowly via syringe and the resulting reaction mixture was stirred at room temperature for 10 min. Acryloyl chloride (0.34 mL, 4.23 mmol, 3 eq) was added and the reaction mixture was further stirred for 12 hours. Water (15 mL) was added and the organic layer was separated. Aqueous layer was back extracted with dichloromethane (2×15 mL) and the combined organic layer was washed with brine, dried over anhydrous sodium sulfate, filtered and concentrated in vacuo to provide crude product which was purified on silica gel flash chromatography using 0-30% ethyl acetate in hexanes to provide intermediate 35 (754 mg, 65% yield).

Step 2: General method: Appropriate amine (5 equiv.) was added to the neat intermediate 35 (1 equiv.) and the resulting reaction mixture was stirred at 70° C. for 3 hours. A slurry of the reaction mixture was prepared on silica gel and directly purified on silica gel flash chromatography using 0-40% gradient of acetone in hexanes to obtain desired product (320 mg, 96% yield) LCMS (M+1)=869.04, Rt=4.99 min (Method D) by using dimethylamine (2M solution in THF).

### Example 34: Synthesis of Compound

This example demonstrates synthesis of lipid compound:

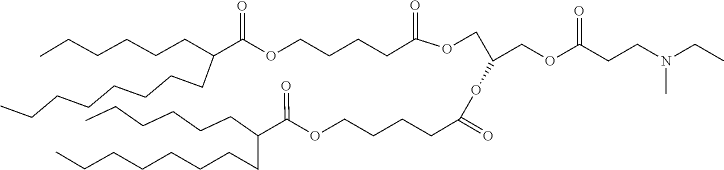

The lipid compound shown above (63 mg, 77% yield) was prepared following the procedure described for the synthesis of the lipid compound shown in Example 33, Scheme 33, by using N-ethylmethylamine and purification using 0-30% Acetone in hexanes. LCMS (M+1)=883.77, Rt=5.01 min (method D).

### Example 35: Synthesis of Compound

This example demonstrates synthesis of lipid compound:

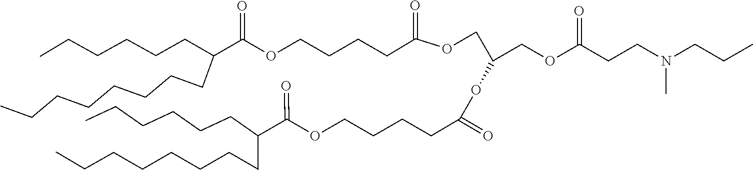

The lipid compound shown above (78 mg, 82% yield) was prepared following the procedure described for the synthesis of the lipid compound shown in Example 33, Scheme 33, by using N-propylmethylamine and purification using 0-30% Acetone in hexanes. LCMS (M+1)=897.63, Rt=5.00 min (method D).

### Example 36: Synthesis of Compound

This example demonstrates synthesis of lipid compound:

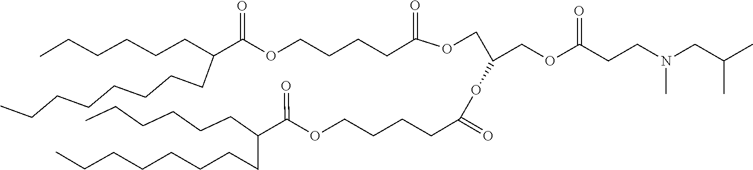

The lipid compound shown above (89 mg, 67% yield) was prepared following the procedure described for the synthesis of the lipid compound shown in Example 33, Scheme 33, by using N-methylisobutylamine and purification using 0-30% Acetone in hexanes. LCMS (M+1)=911.59 Rt=5.04 min (method D).

### Example 37: Synthesis of Compound

This example demonstrates synthesis of lipid compound:

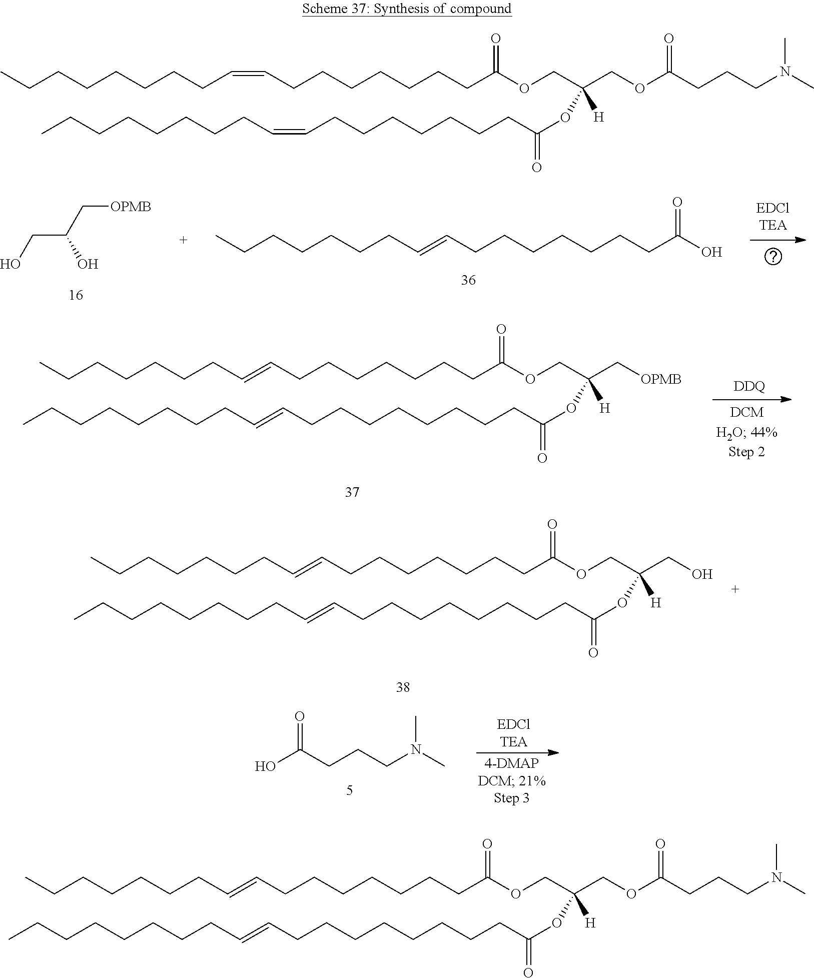

Step 1: To a solution of diol 16 (500 mg, 2.35 mmol, 1 eq) in dichloromethane (20. mL) was added acid 36 (1.5 g, 5.18 mmol, 2.2 eq), EDCI hydrochloride (1.13 g, 5.80 mmol, 2.5 eq), triethylamine (3.4 mL, 23.54 mmol, 10 eq) and 4-DMAP (144 mg, 1.17 mmol, 0.5 eq) at room temperature and the resulting reaction mixture was stirred for 48 h. Reaction mixture was concentrated in vacuo and purified by silica gel flash chromatography using 0-25% ethyl acetate in hexanes to obtain pure intermediate 37 as colorless liquid (1.355 g, 81% yield).

Step 2: Intermediate 37 (1.355 g, 1.9 mmol, 1 eq) was added to a mixture of dichloromethane (15 mL) and water (2 mL). DDQ (1.1 g, 4.75 mmol, 2.5 eq) was slowly added in portions and the resulting reaction mixture was stirred over night at room temperature. Reaction mixture was quenched by the addition of saturated aqueous sodium bicarbonate solution (15 mL). Aqueous layer was extracted with dichloromethane (4×25 mL). Combined organic layer was washed with saturated aqueous sodium bicarbonate solution (25 mL), brine (2×25 mL), dried over anhydrous sodium sulfate, filtered and concentrated at reduced pressure to obtain crude product which was purified by flash silica gel chromatography using 0-10% Acetone in hexanes. Pure fractions were concentrated to dryness to obtain desired product alcohol 38 as clear oil (750 mg, 44% yield.).

Step 3: To a solution of intermediate alcohol 38 (125 mg, 0.2 mmol, 1 eq) in dichloromethane (5 mL) was added 4-dimethylaminobutyric acid hydrochloride salt (71 mg, 0.42 mmol, 2.1 eq), EDCI hydrochloride (97 mg, 0.5 mmol, 2.5 eq), triethylamine (0.3 mL, 2 mmol, 10 eq), 4-DMAP (13 mg, 0.1 mmol, 0.5 eq) and resulting reaction mixture was stirred for 16 h at room temperature. LCMS indicated the formation of desired product. Reaction mixture was directly loaded on silica gel column and purified using 0-40% Acetone in Hexane. Pure fractions were combined, concentrated in rotovap and dried overnight under high vacuum to obtain desired product (33 mg, 24% yield). LCMS (M+1)=734.91, Rt=5.14 min (method D).

### Example 38: Synthesis of Intermediate 14

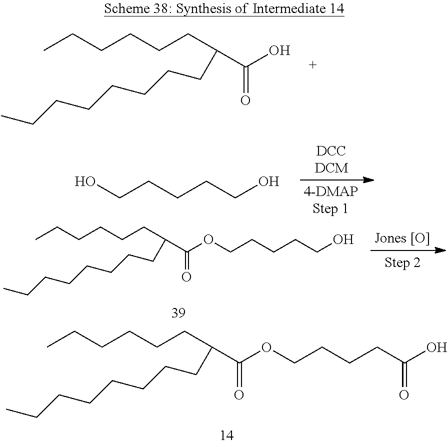

Step 1: To a solution of 2-hexyldecanoic acid (5 g, 19.5 mmol, 1 eq) in dichloromethane (120 mL) was added pentane-1,5-diol (6.1 g, 58.5 mmol, 3 eq), DCC (4.6 g, 22.2 mmol, 1.14 eq) and 4-DMAP (2.85 g, 23.4 mmol, 1.2 eq). The reaction mixture was stirred at room temperature for 12 h. Hexanes (100 mL) was added, the resulting precipitates were filtered off and the filtrate was concentrated in vacuo to obtain crude material which was purified by silica gel chromatography using 0-15% ethyl acetate in hexanes to obtain pure intermediate 39 (3.5 g, 52% yield).

Step 2: A solution of intermediate alcohol 39 (918 mg, 2.7 mmol, 1 eq) in acetone (10 mL) was cooled to approximately 0° C. under an ice/water bath. Jones reagent (2M, 2.16 mL, 4.32 mmol, 1.6 eq) was added slowly via syringe and the resulting reaction mixture was gradually warmed up to room temperature and stirred for 16 h. Methanol (0.5 mL) was added to quench the reaction. Reaction mixture was concentrated under reduced pressure, diluted with water (200 mL) and extracted by ethyl acetate (100 mL). Aqueous layer was extracted back with additional ethyl acetate (100 mL). Combined organic layer was dried over anhydrous sodium sulfate, filtered and concentrated in vacuo. Crude product was purified by silica gel column chromatography using 0-15% ethyl acetate in hexanes to obtain intermediate 14 as clear colorless oil (710 mg, 74% yield).

### EQUIVALENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

1.-47. (canceled)

48. A compound having the following structure of Formula (XVII): 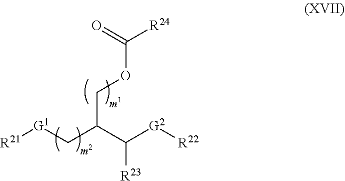
or a stereoisomer, salt or tautomer thereof, wherein:
G¹ and G² are each independently —OC(═O)—, —NR²⁵C(═O)—, or —CH═CH—;
R²¹ and R²² are each independently C₁-C₆ alkyl, linear C₁₀-C₂₀ alkyl, linear C₁₀-C₂₀ alkenyl, or branched C₁₀-C₃₅ alkenyl, wherein the C₁-C₆ alkyl is substituted with —OC(═O)R²⁶;
R²³ is H, OH, or OCH₃;
R²⁴ is C₁-C₈ heteroalkyl;
R²⁵ is H or C₁-C₄ alkyl;
R²⁶ is branched C₁₀-C₃₀ alkyl; and
m¹ and m² are each independently an integer of 0 or 1.

49. The compound of claim 48, wherein G¹ and G² are each —OC(═O)—.

50. (canceled)

51. The compound of claim 48, wherein m² is 0.

52. The compound of claim 51, wherein the compound has one of the following structures of Formula (XVIIA)-(XVIIB): 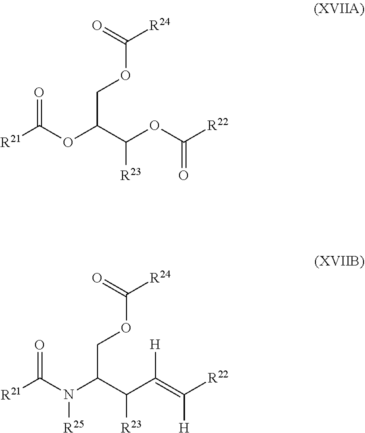
or a stereoisomer, salt or tautomer thereof.

53. (canceled)

54. (canceled)

55. The compound of claim 48, wherein R²⁴ is C₄ alkylamine, C₅ alkylamine, C₆ alkylamine, or C₇ alkylamine.

56. The compound of claim 55, wherein R²⁴ is

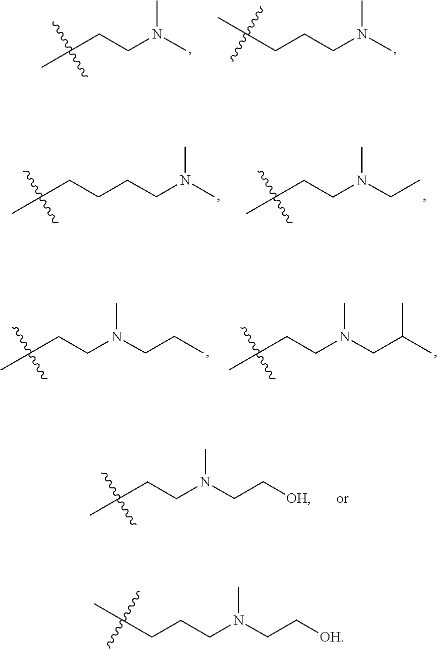

57. The compound of claim 48, wherein C₁-C₈ heteroalkyl of R²⁴ is further substituted with a cycloalkyl.

58. The compound of claim 57, wherein R²⁴ is

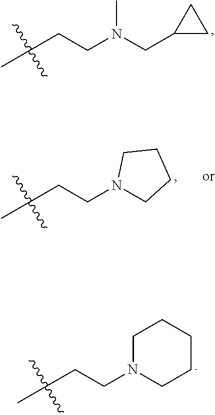

59.-62. (canceled)

63. The compound of claim 48, wherein R²¹ and R²² are each independently C₂-C₅ alkyl substituted with —OC(═O)R²⁶, linear C₁₂-C₁₈ alkyl, linear C₁₂-C₁₈ alkenyl, or branched C₁₄-C₃₂ alkenyl.

64.-69. (canceled)

70. The compound of claim 48, wherein R²⁶ is branched C₁₂-C₁₈ alkyl.

71. The compound of claim 48, wherein R²¹ and R²² each independently have one of the following structures:

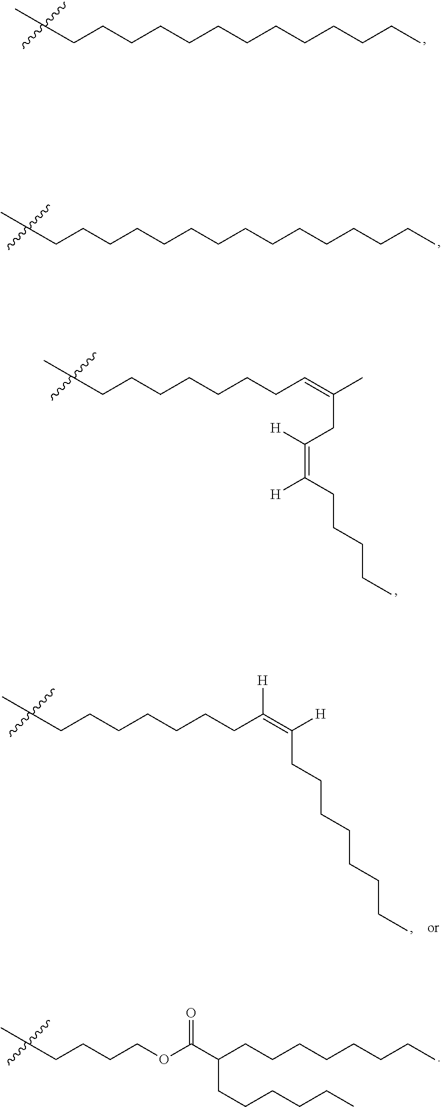 [SMILES: *CCCCCCCCCCCCCC ; *CCCCCCCCCCCCCCCC ; *CCCCCCCC/C=C(/C)C/C=C\CCCCC ; *CCCCCCCC/C=C\CCCCCCCC ; *CCCCCOC(=O)C(CCCCCC)CCCCCCCC]

72. The compound of claim 48, wherein the compound has one of the following structures:

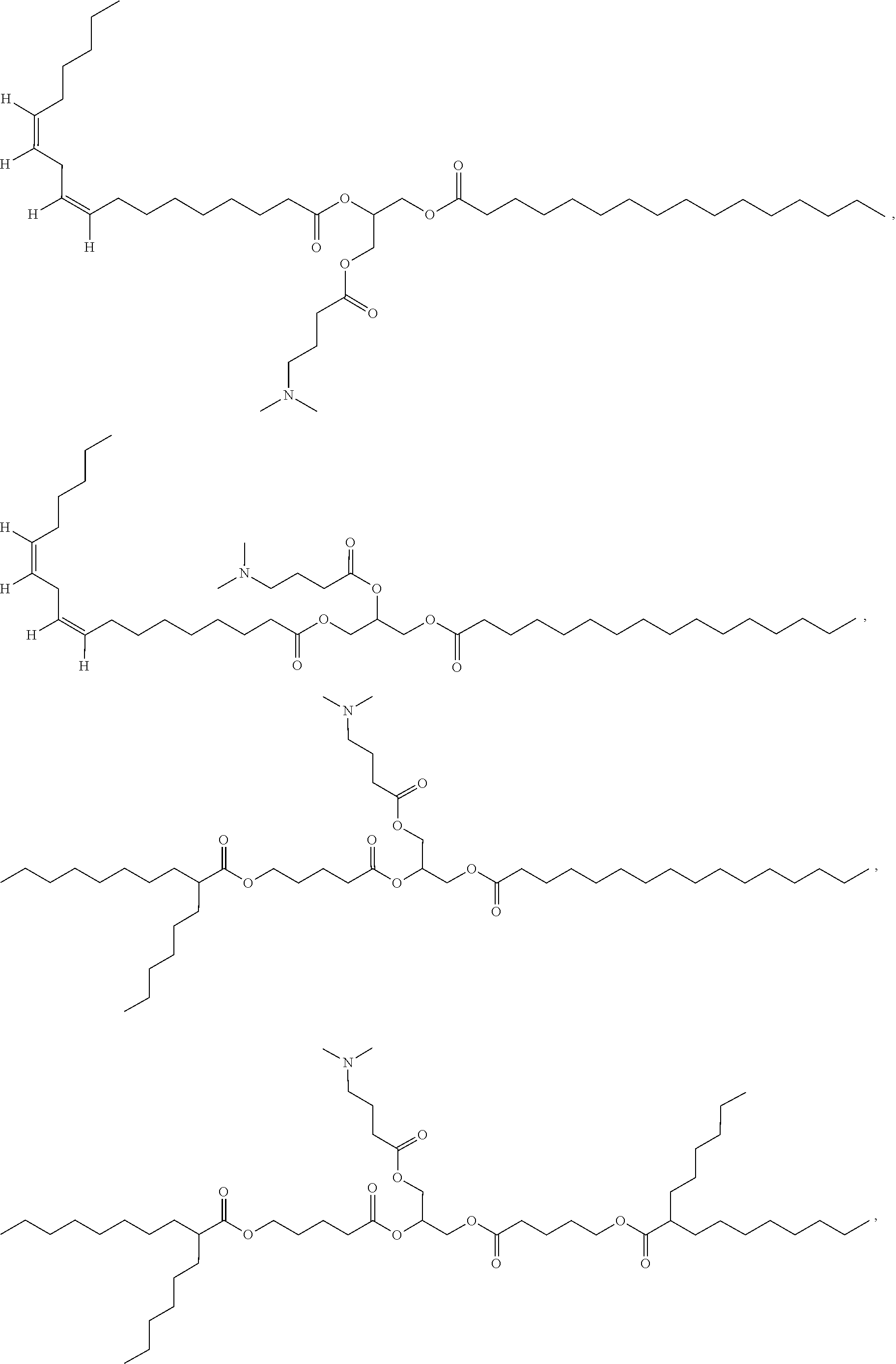

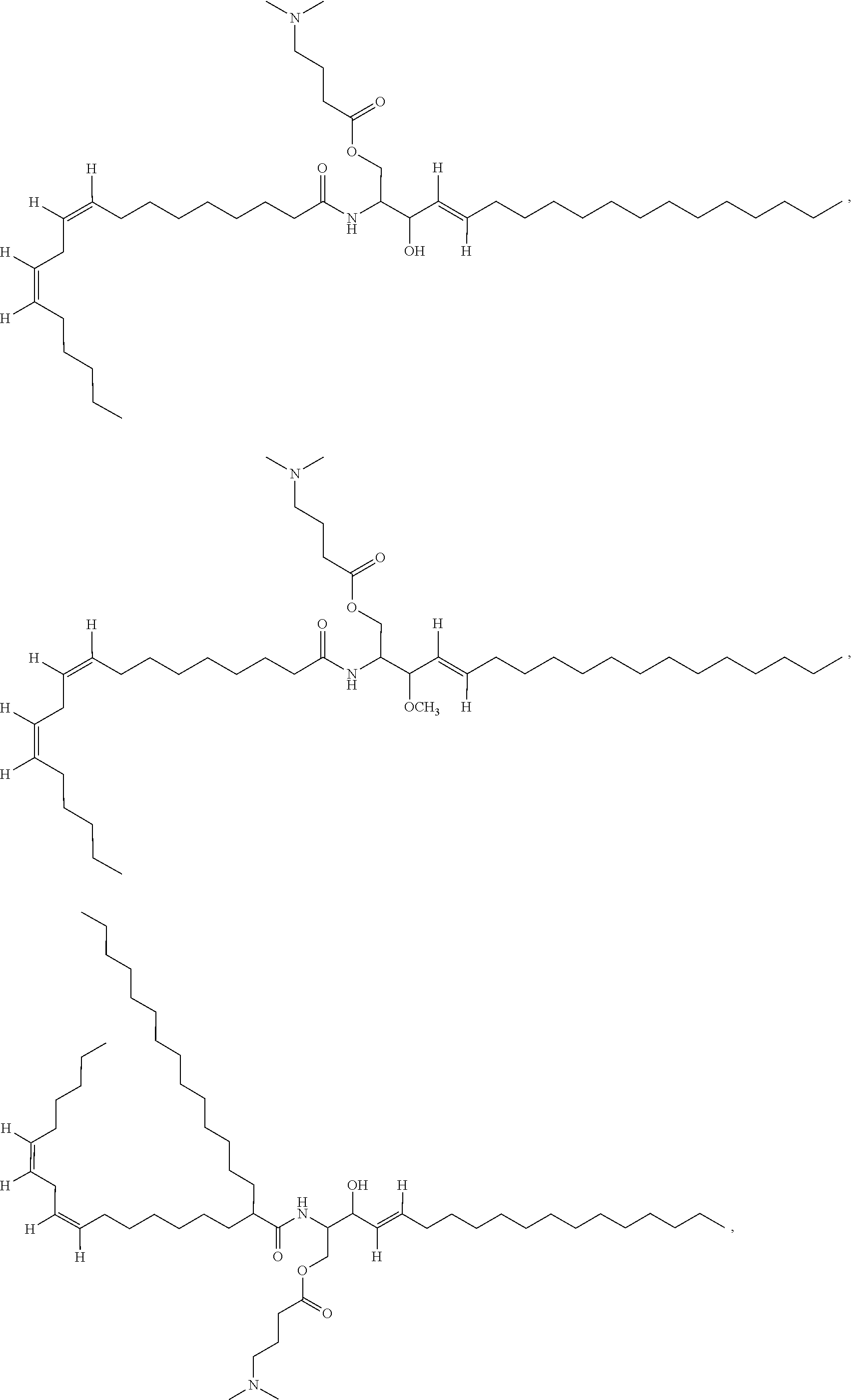 [SMILES: CCCCC/C=C\C/C=C\CCCCCCCC(=O)NC(COC(=O)CCCN(C)C)C(O)/C=C/CCCCCCCCCCCCC ; CCCCC/C=C\C/C=C\CCCCCCCC(=O)NC(COC(=O)CCCN(C)C)C(/C=C/CCCCCCCCCCCCC)OC ; CCCCC/C=C\C/C=C\CCCCCCC(CCCCCCCCCCCCCC)C(=O)NC(COC(=O)CCCN(C)C)C(O)/C=C/CCCCCCCCCCCCC]

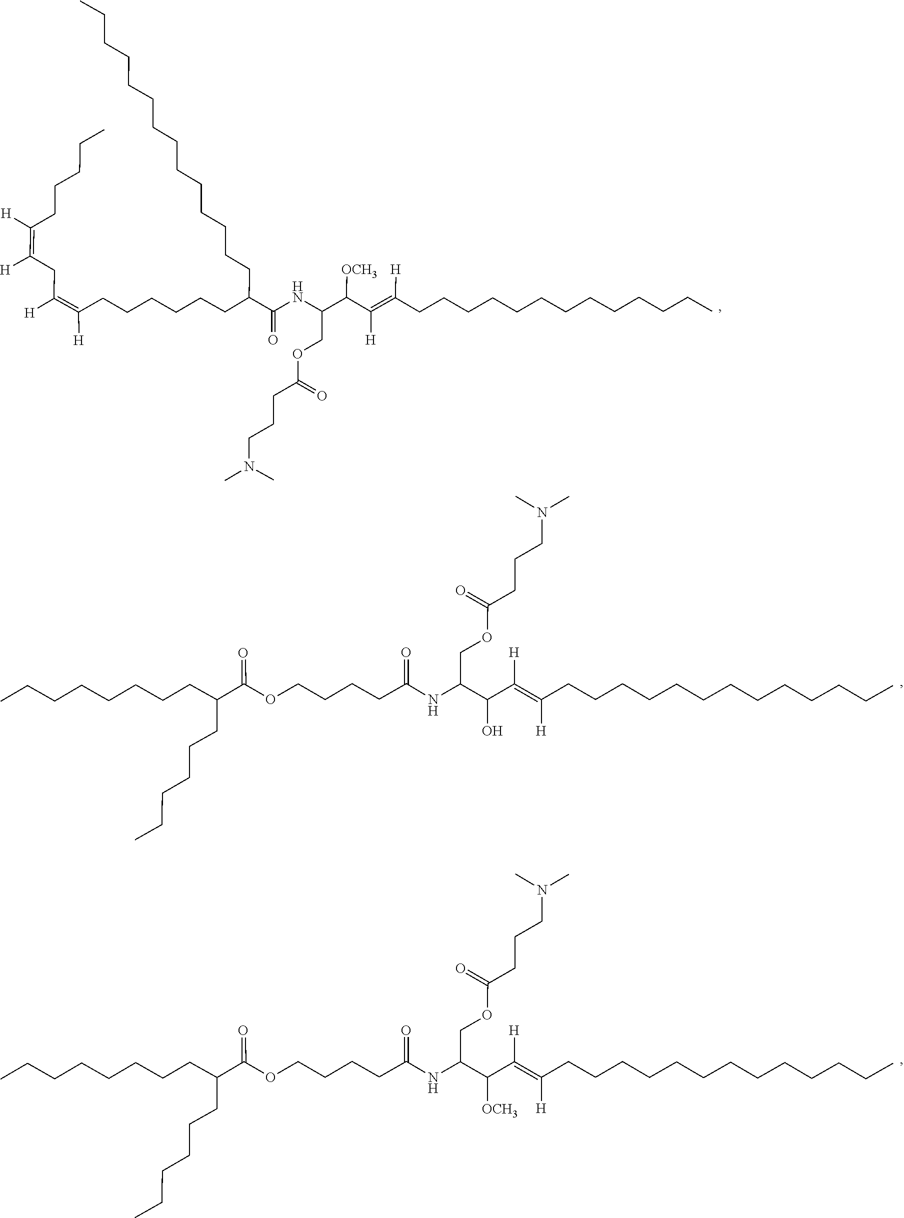

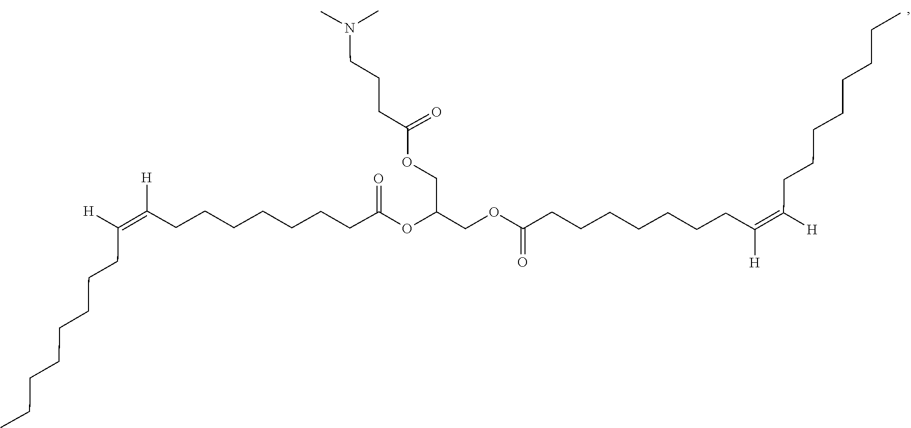 [SMILES: CCCCCCCC/C=C\CCCCCCCC(=O)OCC(COC(=O)CCCN(C)C)OC(=O)CCCCCCC/C=C\CCCCCCCC]

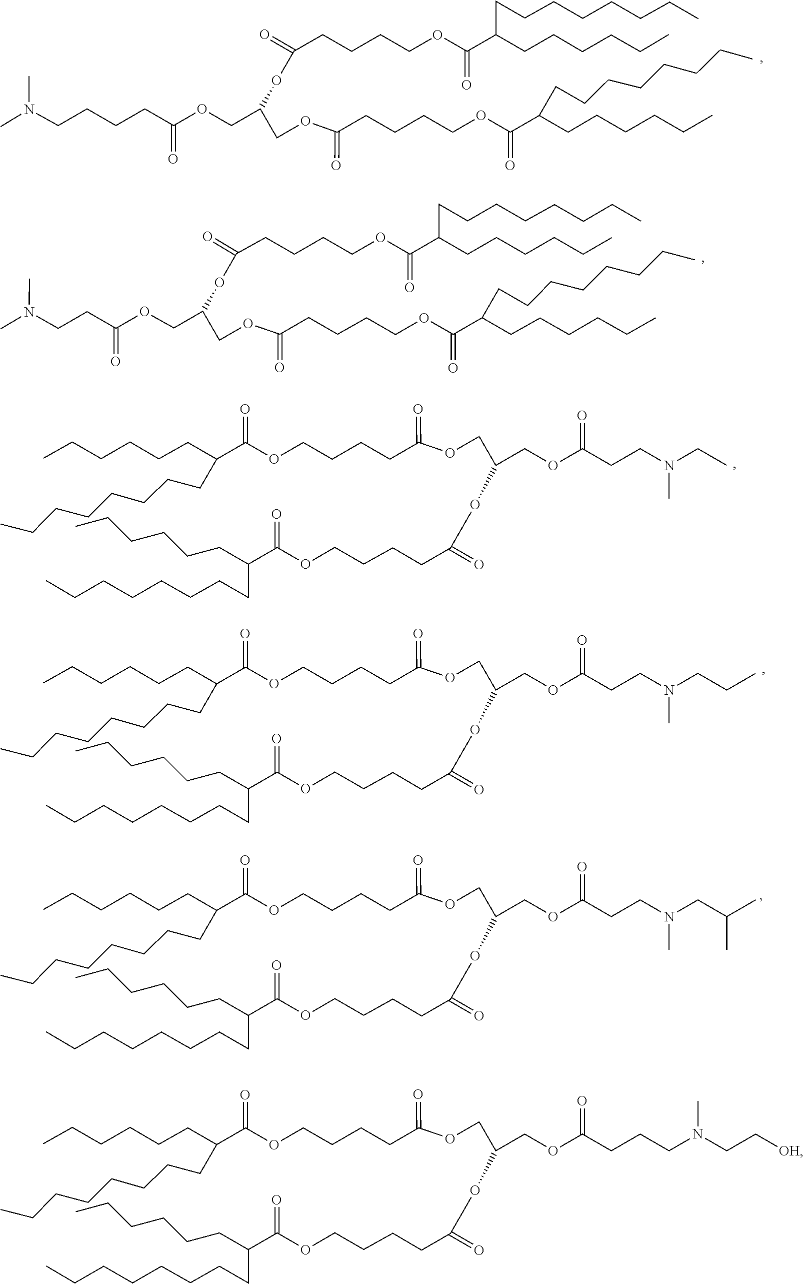

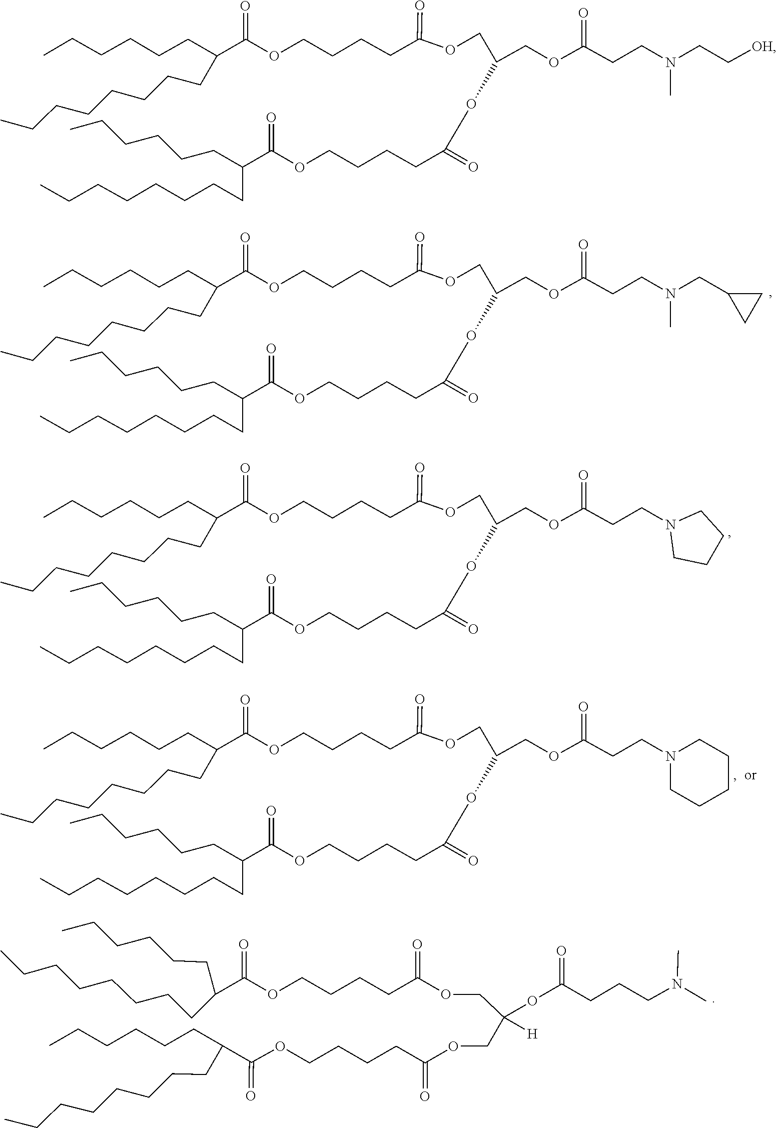

73.-98. (canceled)

99. A pharmaceutical composition comprising the compound of claim 48 and a therapeutic agent comprising a nucleic acid.

100. The pharmaceutical composition of claim 99, further comprises a helper lipid, a stabilization lipid, or a structural lipid.

101. The pharmaceutical composition of claim 100, wherein the helper lipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and mixtures thereof.

102. The pharmaceutical composition of claim 100, wherein the stabilization lipid is 1-(monomethoxy-polyethyleneglycol)-2,3-dimyristoylglycerol (PEG-DMG), with an average PEG molecular weight of 2000.

103. The pharmaceutical composition of claim 100, wherein the structural lipid is selected from the group consisting of cholesterol, cholesterol derivatives, fecosterol, sitosterol, ergosterol, campesterol, stigmasterol, brassicasterol, tomatidine, ursolic acid, alpha tocopherol, and mixtures thereof.

104. The pharmaceutical composition of claim 99, wherein the nucleic acid is selected from small interfering RNA (siRNA), an asymmetrical interfering RNA (aiRNA), a microRNA (miRNA), a Dicer-substrate RNA (dsRNA), a small hairpin RNA (shRNA), or a messenger RNA (mRNA).

105. (canceled)

106. A method for administering a therapeutic agent comprising a nucleic acid to a patient in need thereof, the method comprising administering the composition of claim 99 to the patient.

107. A lipid nanoparticle comprising the compound of claim 48 and a therapeutic agent comprising a nucleic acid.

108. The lipid nanoparticle of claim 107, wherein the therapeutic agent is mRNA.

109.-139. (canceled)
